(19) United States
(12) Patent Application Publication
(10) Pub. No.: US 2002/0177568 A1
(43) Pub. Date: Nov. 28, 2002

(54) Enzymatic nucleic acid treatment of diseases or conditions related to levels of NF-kappa B

(76) Inventors: Dan T. Stinchcomb, Ft. Collins, CO (US); James McSwiggen, Boulder, CO (US); Kenneth G. Draper, Reno, NV (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/864,785

(22) Filed: May 23, 2001

Related U.S. Application Data
(63) Continuation-in-part of application No. 08/777,916, filed on Dec. 23, 1996.
Application No. 08/777,916 is a continuation of application No. 08/291,932, filed on Aug. 15, 1994.
Application No. 08/291,932 is a continuation-in-part of application No. 08/245,466, filed on May 18, 1994, now abandoned.
Application No. 08/245,466 is a continuation-in-part of application No. 07/987,132, filed on Dec. 7, 1992, now abandoned.

(51) Int. Cl. C07H 21/04; A61K 48/00

(52) U.S. Cl. 514/44; 536/23.2

(57) ABSTRACT

The present invention relates to nucleic acid molecules, including antisense and enzymatic nucleic acid molecules, such as hammerhead ribozymes, DNAzymes, allozymes and antisense, which modulate the expression or function of NFKB genes, such as REL-A, REL-B, REL (c-rel), NFKB1 (p105/p50) and NFKB2 (p100)/p52/p49).

RELATED APPLICATIONS

This application is a continuation-in-part of Stinchcomb et al., U.S. Ser. No. 08/777,916, filed Dec. 23, 1996 entitled “Ribozyme Treatment of Diseases or Conditions Related to Levels of NFKB”, which is a continuation of Stinchcomb et al., U.S. Ser. No. 08/291,932, filed Aug. 15, 1994, now U.S. Pat. No. 5,658,780 entitled “REL A Targeted Ribozymes”, which is a continuation-in-part of U.S. Ser. No. 08/245,466 filed May 18, 1994, entitled “Method and Composition for Treatment of Restenosis and Cancer Using Ribozymes,” which is a continuation-in-part of Draper, U.S. Ser. No. 07/987,132 filed Dec. 7, 1992, entitled “Method and Reagent for Treatment of a Stenotic Condition”. These applications are hereby incorporated by reference herein in their entirety including the drawings.

### SEQUENCE LISTING

The Sequence Listing file named “MBHB00-812D.SeqListing” submitted in duplicate on Compact Disc-Recordable (CD-R) medium (CD entitled “010518_{—}1002”) in compliance with 37 C.F.R. §1.52(e) is incorporated herein by reference. The sequence listing file is 1,021,000 bytes in size.

### FIELD OF THE INVENTION

The present invention relates to therapeutic compositions and methods for the treatment or diagnosis of diseases or conditions related to NF-kappa B (NFKB) levels, such as cancer, inflammatory, and autoimmune diseases and/or disorders.

### BACKGROUND OF THE INVENTION

The following is a brief description of the physiological role of NFKB. The discussion is provided only for understanding the invention that follows. This summary is not an admission that any of the work described below is prior art to the claimed invention.

Nuclear factor kappa B (NFKB) is a multiunit transcription factor which regulates the expression of genes involved in a number of physiologic and pathologic processes. NFKB is a key component of the TNF signaling pathway. These processes include, but are not limited to: apoptosis, immune, inflammatory and acute phase responses. The REL-A gene product (a.k.a. RelA or p65), and p50 subunits of NFKB, have been implicated in the induction of inflammatory responses and cellular transformation. NFKB exists in the cytoplasm as an inactive heterodimer of the p50 and p65 subunits. NFKB is complexed with its inhibitory protein, IkappaB, until activated by the appropriate stimuli. NFKB activation can occur following stimulation of a variety of cell types by inflammatory mediators, for example TNF and IL-1, and reactive oxygen intermediates. In response to induction, NFKB can stimulate production of pro-inflammatory cytokines such as TNF-alpha, IL-1-beta, IL-6 and iNOS, thereby perpetuating a positive feedback loop. NFKB appears to play a role in a number of disease processes including: ischemia/reperfusion injury (CNS and myocardial), glomerulonephritis, sepsis, allergic airway inflammation, inflammatory bowel disease, infection, arthritis, and cancer.

The nuclear DNA-binding protein, NFKB, was first identified as a factor that binds and activates the immunoglobulin kappa light chain enhancer in B cells. NFKB now is known to activate transcription of a variety of other cellular genes (e.g., cytokines, adhesion proteins, oncogenes and viral proteins) in response to a variety of stimuli (e.g., phorbol esters, mitogens, cytokines and oxidative stress). In addition, molecular and biochemical characterization of NFKB has shown that the activity is due to a homodimer or heterodimer of a family of DNA binding subunits. Each subunit bears a stretch of 300 amino acids that is homologous to the oncogene, v-rel. The activity first described as NFKB is a heterodimer of p49 or p50 with p65. The p49 and p50 subunits of NFKB (encoded by the NF-kappa B2 or NF kappa B1 genes, respectively) are generated from the precursors NFKB1 (p105) or NFKB2 (p100). The p65 subunit of NFKB (now termed REL-A) is encoded by the rel-A locus.

The roles of each specific transcription-activating complex now are being elucidated in cells (Perkins, et al., 1992, Proc. Natl. Acad. Sci USA, 89, 1529-1533). For instance, the heterodimer of NFKB1 and Rel A (p50/p65) activates transcription of the promoter for the adhesion molecule, VCAM-1, while NFKB2/RelA heterodimers (p49/p65) actually inhibit transcription (Shu, et al., 1993, Mol. Cell. Biol., 13, 6283-6289). Conversely, heterodimers of NFKB2/RelA (p49/p65) act with Tat-I to activate transcription of the HIV genome, while NFKB1/RelA (p50/p65) heterodimers have little effect (Liu et al., 1992, J. Virol., 66, 3883-3887). Similarly, blocking rel A gene expression with antisense oligonucleotides specifically blocks embryonic stem cell adhesion; blocking NFKB 1 gene expression with antisense oligonucleotides had no effect on cellular adhesion (Narayanan et al., 1993, Mol. Cell. Biol., 13, 3802-3810). Thus, the promiscuous role initially assigned to NFKB in transcriptional activation (Lenardo, and Baltimore, 1989, Cell, 58, 227-229) represents the sum of the activities of the rel family of DNA-binding proteins. This conclusion is supported by recent transgenic “knock-out” mice of individual members of the rel family. Such “knock-outs” show few developmental defects, suggesting that essential transcriptional activation functions can be performed by more than one member of the rel family.

A number of specific inhibitors of NFKB function in cells exist, including treatment with phosphorothioate antisense oliogonucleotide, treatment with double-stranded NFKB binding sites, and over expression of the natural inhibitor MAD-3 (an Ikappa-B family member). These agents have been used to show that NFKB is required for induction of a number of molecules involved in cancer and/or inflammation, as described below.

NFKB is required for phorbol ester-mediated induction of IL-6 (Kitajima, et al., 1992, Science, 258, 1792-5) and IL-8 (Kunsch and Rosen, 1993, Mol. Cell. Biol., 13, 6137-46).

NFKB is required for induction of the adhesion molecules ICAM-1 (Eck, et al., 1993, Mol. Cell. Biol., 13, 6530-6536), VCAM-1 (Shu et al., supra), and E-selectin (Read, et al., 1994, J. Exp. Med., 179, 503-512) on endothelial cells.

NFKB is involved in the induction of the integrin subunit, CD18, and other adhesive properties of leukocytes (Eck et al., 1993 supra).

HER2/Neu overexpression induces NFKB via a PI3-kinase/Akt pathway involving calpain-mediated degradation of IKB-alpha. Breast cancer has been shown to typify the aberrant expression of NFKB/REL factors (Pianetti et al., 2001, Oncogene, 20, 1287-1299; Sovak et al., 1999, J. Clin. Invest., 100, 2952-2960).

Inhibition of NFKB activity has been shown to induce apoptosis in murine hepatocytes (Bellas et al., 1997, Am. J. Pathol., 151, 891-896).

NFKB has been shown to regulate cyclooxygenase-2 expression and cell proliferation in human gastric cancer cells (Joo Weon et al., 2001, Laboratory Investigation, 81, 349-360).

The above studies suggest that NFKB is integrally involved in the induction of cytokines and adhesion molecules by inflammatory mediators and is involved in the transformation of cancerous cells. Two reported studies point to another connection between NFKB and inflammation: glucocorticoids may exert their anti-inflammatory effects by inhibiting NFKB. The glucocorticoid receptor and p65 both act at NFKB binding sites in the ICAM-1 promoter (van de Stolpe, et al., 1994, J. Biol. Chem., 269, 6185-6192). Glucocorticoid receptor inhibits NFKB-mediated induction of IL-6 (Ray and Prefontaine, 1994 Proc. Natl. Acad. Sci USA, 91, 752-756). Conversely, overexpression of p65 inhibits glucocorticoid induction of the mouse mammary tumor virus promoter. Finally, protein cross-linking and co-immunoprecipitation experiments demonstrated direct physical interaction between p65 and the glucocorticoid receptor.

Stinchcomb et al., U.S. Pat. No. 5,658,780, describes ribozymes targeting NFKB. Stinchcomb et al., International PCT Publication No. WO 95/23225 describe ribozymes targeting NFKB. Sullivan et al., International PCT Publication No. WO 94/02595 describe ribozymes targeting NFKB. Handel et al., International PCT Publication No. WO 01/11023, describe a specific DNAzyme motif targeting certain sites within the Rel-A subunit of NFKB.

### SUMMARY OF THE INVENTION

The present invention features an enzymatic nucleic acid molecule which modulates expression of a sequence encoding a subunit of NFKB, for example REL-A, REL-B, REL, NFkappaB 1, or NFkappaB2, wherein the enzymatic nucleic acid molecule is for example in a an hammerhead, Inozyme, Zinzyme, G-cleaver, or Amberzyme configuration.

The present invention also features an enzymatic nucleic acid molecule which modulates expression of a sequence encoding a subunit of NFKB, for example REL-A, REL-B, REL, NFkappaB 1, or NFkappaB2, wherein the enzymatic nucleic acid molecule is a DNAzyme.

In one embodiment, the present invention features an enzymatic nucleic acid molecule comprising a sequence selected from the group consisting of SEQ ID NOs. 711-1420, 1717-2012, 2151-2656, 2994-3626, and 3770-3917.

In another embodiment, the present invention features an enzymatic nucleic acid molecule comprising at least one binding arm wherein one or more of said binding arms comprises a sequence complementary to a sequence selected from the group consisting of SEQ ID NOs. 1-710, 1421-1716, 2013-2150, 2657-2993, and 3627-3769.

The present invention also features an antisense nucleic acid molecule comprising a sequence complementary to a sequence selected from the group consisting of SEQ ID NOs. 1-710, 1421-1716, 2013-2150, 2657-2993, and 3627-3769.

In one embodiment, the nucleic acid molecule of the invention, for example an enzymatic nucleic acid molecule or antisense nucleic acid molecule is adapted to treat cancer.

In another embodiment, the enzymatic nucleic acid molecule of the invention has an endonuclease activity to cleave RNA having REL-A sequence.

In a further embodiment, the enzymatic nucleic acid molecule of the invention is in a Hammerhead, Hairpin, Inozyme, Zinzyme, G-cleaver, Amberzyme, DNAzyme, or Allozyme configuration.

In one embodiment, an Inozyme of the invention comprises a sequence complementary to a sequence selected from the group consisting of SEQ ID NOs. 1-710, 3752-3756, and 3660-3720. In another embodiment, an Inozyme of the invention comprises a sequence selected from the group consisting of SEQ ID NOs. 711-1420, 3898-3902, and 3806-3866.

In one embodiment, a Zinzyme of the invention comprises a sequence complementary to a sequence selected from the group consisting of SEQ ID NOs. 1421-1716, 3721-3746, and 3757-3761. In another embodiment, a Zinzyme of the invention comprises a sequence selected from the group consisting of SEQ ID NOs 1717-2012, 3867-3892, and 3903-3907.

In one embodiment, an Amberzyme of the invention comprises a sequence complementary to a sequence selected from the group consisting of SEQ ID NOs. 1421-1716, 2657-2993, and 3765-3769. In another embodiment, an Amberzyme of the invention comprises a sequence selected from the group consisting of SEQ ID NOs 2994-3626, and 3913-3917.

In a further embodiment, an enzymatic nucleic acid molecule of the invention comprises between 12 and 100 bases complementary to RNA sequence encoding a subunit of NFKB. In another embodiment, the enzymatic nucleic acid molecule of the invention comprises between 14 and 24 bases complementary to RNA sequence encoding a subunit of NFKB, for example REL-A, REL-B, REL, NFkappaB1, or NFkappaB2.

In one embodiment, the enzymatic nucleic acid molecule of the invention is chemically synthesized. In another embodiment, the antisense nucleic acid molecule of the invention is chemically synthesized.

In one embodiment, the enzymatic nucleic acid molecule of the invention comprises at least one 2′-sugar modification, at least one base modification, and/or at least one phosphate backbone modification. In another embodiment, the antisense nucleic acid molecule of the invention comprises at least one 2′-sugar modification, at least one base modification, and/or at least one phosphate backbone modification.

The present invention features a mammalian cell including an enzymatic nucleic acid molecule of the invention. In one embodiment, the mammalian cell of the invention is a human cell.

The present invention features a method of reducing NFKB activity in a cell, comprising contacting a cell with an enzymatic nucleic acid molecule or antisense nucleic acid molecule of the invention targeted against a subunit of NFKB, for example REL-A, REL-B, REL, NFkappaB 1, or NFkappaB2, under conditions suitable for the reduction of NFKB activity. In one embodiment, the method of the invention comprises the use of one or more drug therapies under conditions suitable for the treatment.

The present invention also features a method of treatment of a patient having a condition associated with the level of NFKB, comprising contacting cells of the patient with an enzymatic nucleic acid molecule or antisense nucleic acid molecule of the invention targeted against a subunit of NFKB, for example REL-A, REL-B, REL, NFkappaB1, or NFkappaB2, under conditions suitable for the treatment. In another embodiment, the method of the invention comprises the use of one or more drug therapies under conditions suitable for the treatment. Suitable other drug therapies contemplated by the instant invention include, for example, monoclonal antibodies, REL-A-specific inhibitors, or chemotherapy, for example paclitaxel, docetaxel, cisplatin, methotrexate, cyclophosphamide, doxorubin, fluorouracil carboplatin, edatrexate, gemcitabine, or vinorelbine.

The invention also features a method of cleaving RNA comprising a sequence of a subunit of NFKB, for example REL-A, REL-B, REL, NFkappaB1, or NFkappaB2, comprising contacting an enzymatic nucleic acid molecule of the invention with the RNA under conditions suitable for the cleavage. In one embodiment, the cleavage is carried out in the presence of a divalent cation, for example Mg2+.

In one embodiment, an enzymatic nucleic acid or antisense nucleic acid molecule of the invention comprises a cap structure, wherein the cap structure is at the 5′-end, or 3′-end, or both the 5′-end and the 3′-end. In another embodiment, the cap structure at the 5′-end, 3′-end, or both the 5′-end and the 3′-end comprises a 3′, 3′-linked or 5′,5′-linked deoxyabasic ribose derivative.

The present invention features an expression vector comprising a nucleic acid sequence encoding at least one enzymatic nucleic acid molecule of the invention, for example a hammerhead ribozyme, in a manner which allows expression of the nucleic acid molecule. In one embodiment, the invention features a mammalian cell including an expression vector of the invention, for example a human cell.

In one embodiment, an expression vector of the invention comprises a sequence for an antisense nucleic acid molecule complementary to the RNA having a sequence of a subunit of NFKB. In another embodiment, an expression vector of the invention comprises a nucleic acid sequence encoding two or more of said enzymatic nucleic acid molecules, which may be the same or different. In yet another embodiment, an expression vector of the invention comprises a sequence encoding an antisense nucleic acid molecule complementary to the RNA of a subunit of NFKB, for example REL-A, REL-B, REL, NFkappaB1, or NFkappaB2.

The present invention features a method for treatment of cancer, for example breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer, comprising administering to a patient an enzymatic nucleic acid molecule or antisense nucleic acid molecule of the invention under conditions suitable for the treatment. In one embodiment, a method of treatment contemplated by the instant invention comprises administering to a patient one or more other therapies in combination with the enzymatic nucleic acid or antisense nucleic acid molecule of the invention.

In one embodiment, a nucleic acid molecule of the invention comprises at least five ribose residues, at least ten 2′-O-methyl modifications, and/or a 3′- end modification, for example a 3′-3′ inverted abasic moiety. In anther embodiment, a nucleic acid molecule of the invention further comprises phosphorothioate linkages on at least three 5′ terminal nucleotides.

The present invention features a method for treatment of an inflammatory disease, for example rheumatoid arthritis, restenosis, asthma, Crohn's disease, diabetes, obesity, autoimmune disease, lupus, multiple sclerosis, transplant/graft rejection, gene therapy applications, ischemia/reperfusion injury, glomerulonephritis, sepsis, allergic airway inflammation, inflammatory bowel disease, or infection, comprising the step of administering to a patient an enzymatic nucleic acid molecule or antisense nucleic acid molecule of the invention under conditions suitable for the treatment, with or without the use of other therapies.

The present invention also features a pharmaceutical composition comprising an enzymatic nucleic acid molecule or antisense nucleic acid molecule of the invention in a pharmaceutically acceptable carrier.

The invention also features a method of administering to a cell, such as mammalian cell (e.g. human cell), where the cell may be in culture or in a mammal, such as a human, an enzymatic nucleic acid molecule or antisense molecule of the instant invention, comprising contacting the cell with the enzymatic nucleic acid molecule or antisense molecule under conditions suitable for such administration.

### DETAILED DESCRIPTION OF THE INVENTION

First the drawings will be described briefly.

### DRAWINGS

FIG. 1 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research 26, 4116-4120, Eckstein et al., International PCT publication No. WO 99/16871). N or n, represent independently a nucleotide which can be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

FIG. 2 shows an example of the Amberzyme ribozyme motif that is chemically stabilized (see for example Beigelman et al., International PCT publication No. WO 99/55857).

FIG. 3 shows an example of the Zinzyme A ribozyme motif that is chemically stabilized (see for example Beigelman et al., Beigelman et al., International PCT publication No. WO 99/55857).

FIG. 4 shows an example of a DNAzyme motif described by Santoro et al., 1997, PNAS, 94, 4262.

The invention features novel enzymatic nucleic acid molecules and methods to modulate gene expression, for example, genes encoding NF kappa-B (NFKB) and protein subunits of NFKB, such as REL-A, REL-B, REL, NFkappaB1, or NFkappaB2. In particular, the instant invention features nucleic-acid based molecules and methods to modulate the expression of the Rel-A, REL-B, REL, NFkappaB1, or NFkappaB2 subunit of NFKB.

The invention features one or more enzymatic nucleic acid-based molecules and methods that independently or in combination modulate the expression of gene(s) encoding NFKB. In particular embodiments, the invention features nucleic acid-based molecules and methods that modulate the expression of a subunit of NFKB, for example REL-A, REL-B, REL, NFkappaB 1, or NFkappaB2gene, for example (Genbank Accession No. NM_{—}021975); REL-B gene, for example (Genbank Accession No. NM_{—}006509), REL (c-rel), for example (Genbank Accession No. NM_{—}003998), NFKB1 (p105/p50), for example (Genbank Accession No. NM_{—}003998), and NFKB2 (p100/p52/p49), for example (Genbank Accession No. NM_{—}002502).

The description below of the various aspects and embodiments is provided with reference to the exemplary NFKB subunit REL-A gene, also known as p65 or P65. However, the various aspects and embodiments are also directed to other genes which encode REL-A proteins and other subunits of NFKB, such as p49, p50, p52, p100, or p105 protein subunits (Perkins et al., 1992, Proc, Natl. Acad. Sci. USA, 89, 1529-1533; Naumann et al., 1994, EMBO J., 13, 4597-4607; Heusch et al., 1999, Oncogene, 18, 6201-6208). Those additional genes can be analyzed for target sites using the methods described for REL-A. Thus, the inhibition and the effects of such inhibition of the other genes can be performed as described herein.

In one embodiment, the invention features the use of an enzymatic nucleic acid molecule, preferably in the hammerhead, NCH, G-cleaver, amberzyme, zinzyme and/or DNAzyme motif, to down-regulate the expression of REL-A genes.

By “inhibit” or “down-regulate” it is meant that the expression of the gene, or level of RNAs or equivalent RNAs encoding one or more protein subunits, or activity of one or more protein subunits, such as REL-A subunit(s), is reduced below that observed in the absence of the nucleic acid molecules of the invention. In one embodiment, inhibition or down-regulation with enzymatic nucleic acid molecule preferably is below that level observed in the presence of an enzymatically inactive or attenuated molecule that is able to bind to the same site on the target RNA, but is unable to cleave that RNA. In another embodiment, inhibition or down-regulation with antisense oligonucleotides is preferably below that level observed in the presence of, for example, an oligonucleotide with scrambled sequence or with mismatches. In another embodiment, inhibition or down-regulation of REL-A with the nucleic acid molecule of the instant invention is greater in the presence of the nucleic acid molecule than in its absence.

By “up-regulate” is meant that the expression of the gene, or level of RNAs or equivalent RNAs encoding one or more protein subunits, or activity of one or more protein subunits, such as REL-A subunit(s), is greater than that observed in the absence of the nucleic acid molecules of the invention. For example, the expression of a gene, such as REL-A gene, can be increased in order to treat, prevent, ameliorate, or modulate a pathological condition caused or exacerbated by an absence or low level of gene expression.

By “modulate” is meant that the expression of the gene, or level of RNAs or equivalent RNAs encoding one or more protein subunits, or activity of one or more protein subunit(s) is up-regulated or down-regulated, such that the expression, level, or activity is greater than or less than that observed in the absence of the nucleic acid molecules of the invention.

By “enzymatic nucleic acid molecule” it is meant a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

By “nucleic acid molecule” as used herein is meant a molecule having nucleotides. The nucleic acid can be single, double, or multiple stranded and can comprise modified or unmodified nucleotides or non-nucleotides or various mixtures and combinations thereof.

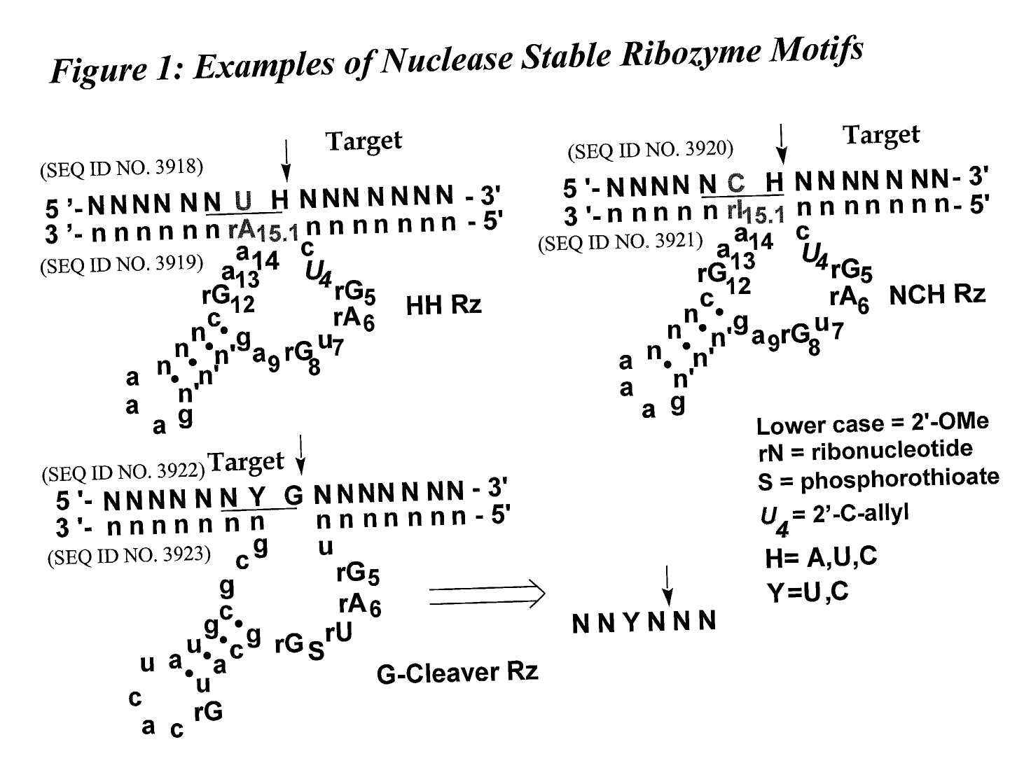

By “enzymatic portion” or “catalytic domain” is meant that portion/region of the enzymatic nucleic acid molecule essential for cleavage of a nucleic acid substrate (for example see FIGS. 1-4).

By “substrate binding arm” or “substrate binding domain” is meant that portion/region of a enzymatic nucleic acid which is able to interact, for example via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-4. That is, these arms contain sequences within a enzymatic nucleic acid which are intended to bring enzymatic nucleic acid and target RNA together through complementary base-pairing interactions. The enzymatic nucleic acid of the invention can have binding arms that are contiguous or non-contiguous and may be of varying lengths. The length of the binding arm(s) are preferably greater than or equal to three nucleotides and of sufficient length to stably interact with the target RNA; preferably 12-100 nucleotides; more preferably 14-24 nucleotides long (see for example Werner and Uhlenbeck, supra; Hammann et al., supra; Hampel et al., EP0360257; Berzal-Herranz et al., 1993, EMBO J., 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “Inozyme” or “NCH” motif or configuration is meant, an enzymatic nucleic acid molecule comprising a motif as is generally described as NCH Rz in FIG. 1. Inozymes possess endonuclease activity to cleave RNA substrates having a cleavage triplet NCH/, where N is a nucleotide, C is cytidine and H is adenosine, uridine or cytidine, and/represents the cleavage site. H is used interchangeably with X. Inozymes can also possess endonuclease activity to cleave RNA substrates having a cleavage triplet NCN/, where N is a nucleotide, C is cytidine, and/represents the cleavage site. “I” in FIG. 1 represents an Inosine nucleotide, preferably a ribo-Inosine or xylo-Inosine nucleoside.

By “G-cleaver” motif or configuration is meant, an enzymatic nucleic acid molecule comprising a motif as is generally described as G-cleaver Rz in FIG. 1. G-cleavers possess endonuclease activity to cleave RNA substrates having a cleavage triplet NYN/, where N is a nucleotide, Y is uridine or cytidine and/represents the cleavage site. G-cleavers can be chemically modified as is generally shown in FIG. 1.

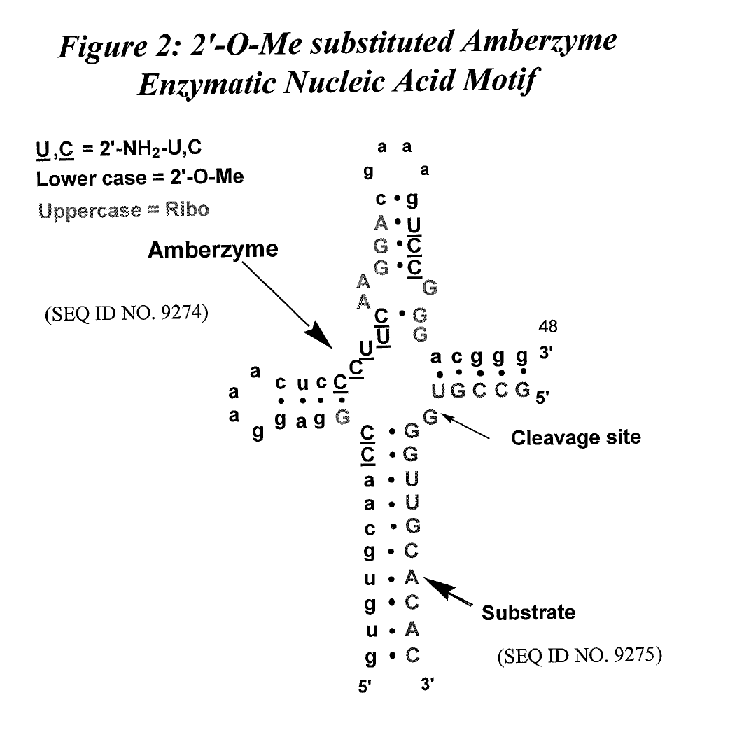

By “amberzyme” motif or configuration is meant, an enzymatic nucleic acid molecule comprising a motif as is generally described in FIG. 2. Amberzymes possess endonuclease activity to cleave RNA substrates having a cleavage triplet NG/N, where N is a nucleotide, G is guanosine, and/represents the cleavage site. Amberzymes can be chemically modified to increase nuclease stability through substitutions as are generally shown in FIG. 2. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

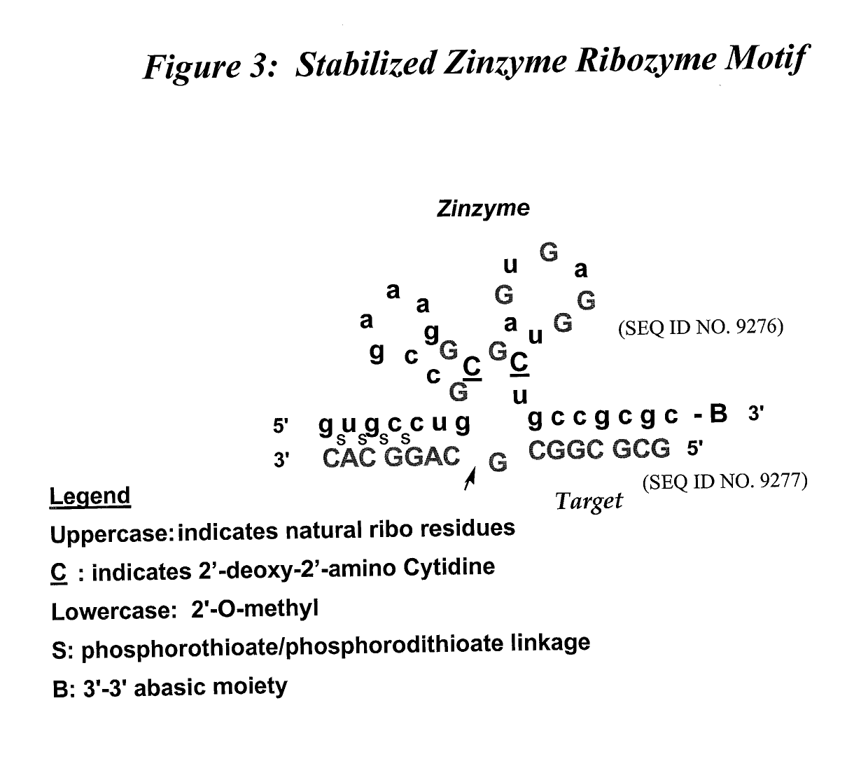

By “zinzyme” motif or configuration is meant, an enzymatic nucleic acid molecule comprising a motif as is generally described in FIG. 3. Zinzymes possess endonuclease activity to cleave RNA substrates having a cleavage triplet including but not limited to YG/Y, where Y is uridine or cytidine, and G is guanosine and/represents the cleavage site. Zinzymes can be chemically modified to increase nuclease stability through substitutions as are generally shown in FIG. 3, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

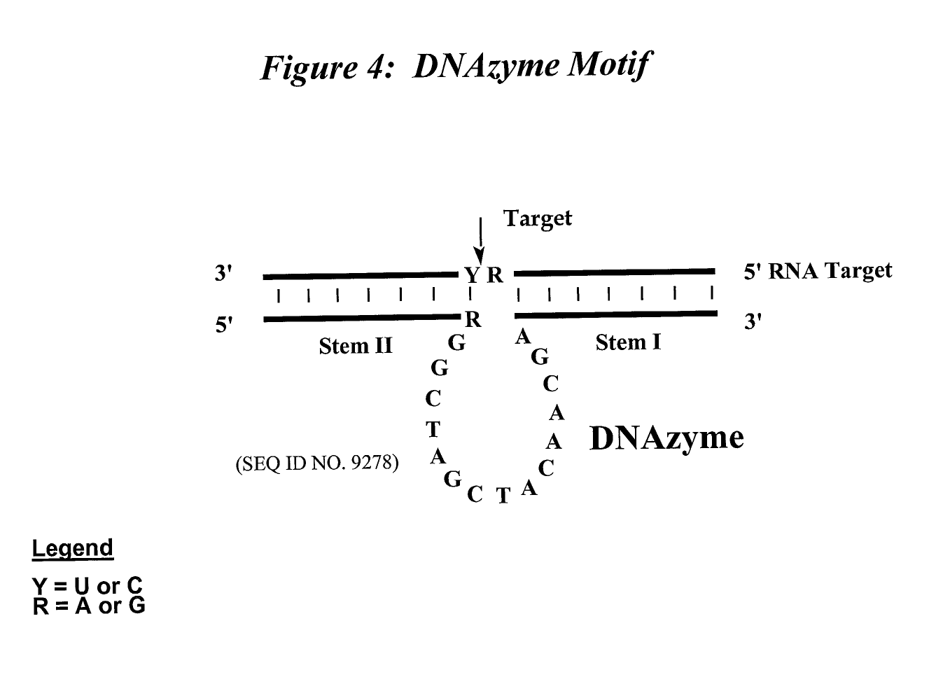

By ‘DNAzyme’ is meant, an enzymatic nucleic acid molecule that does not require the presence of a 2′-OH group within its own nucleic acid sequence for activity. In particular embodiments the enzymatic nucleic acid molecule can have an attached linker(s) or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 4 and is generally reviewed in Usman et al., U.S. Pat. No., 6,159,714; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. Additional DNAzyme motifs can be selected for using techniques similar to those described in these references, and hence, are within the scope of the present invention.

By “sufficient length” is meant an oligonucleotide of greater than or equal to 3 nucleotides that is of a length great enough to provide the intended function under the expected condition. For example, for binding arms of enzymatic nucleic acid “sufficient length” means that the binding arm sequence is long enough to provide stable binding to a target site under the expected binding conditions. Preferably, the binding arms are not so long as to prevent useful turnover of the nucleic acid molecule.

By “stably interact” is meant interaction of the oligonucleotides with target nucleic acid (e.g., by forming hydrogen bonds with complementary nucleotides in the target under physiological conditions) that is sufficient to the intended purpose (e.g., cleavage of target RNA by an enzyme).

By “equivalent” or “related” RNA to NFKB is meant to include those naturally occurring RNA molecules having homology (partial or complete) to NFKB proteins or encoding for proteins with similar function as NFKB proteins in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “gene” it is meant a nucleic acid that encodes an RNA, for example, nucleic acid sequences including but not limited to structural genes encoding a polypeptide.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90% and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′position of a β-D-ribo-furanose moiety.

By “decoy RNA” is meant an RNA molecule or aptamer that is designed to preferentially bind to a predetermined ligand. Such binding can result in the inhibition or activation of a target molecule. The decoy RNA or aptamer can compete with a naturally occurring binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628. Similarly, a decoy RNA can be designed to bind to REL-A and block the binding of REL-A or a decoy RNA can be designed to bind to REL-A and prevent interaction with the REL-A protein.

Several varieties of enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

The enzymatic nucleic acid molecule that cleave the specified sites in NFKB-specific RNAs represent a therapeutic approach to treat a variety of inflammatory diseases and conditions, including but not limited to rheumatoid arthritis, restenosis, asthma, Crohn's disease, diabetes, obesity, autoimmune disease, lupus, multiple sclerosis, transplant/graft rejection, gene therapy applications, ischemia/reperfusion injury (CNS and myocardial), glomerulonephritis, sepsis, allergic airway inflammation, inflammatory bowel disease, infection, and any other inflammatory disease or condition which respond to the modulation of REL-A NFKB function.

The enzymatic nucleic acid molecule that cleave the specified sites in NFKB-specific RNAs also represent a therapeutic approach to treat a variety of cancers, including but not limited to breast, lung, prostate, colorectal, brain, esophageal, bladder, pancreatic, cervical, head and neck, and ovarian cancer, melanoma, lymphoma, glioma, multidrug resistant cancers, and/or other cancers which respond to the modulation of NFKB function.

In one embodiment of the inventions described herein, the enzymatic nucleic acid molecule is formed in a hammerhead or hairpin motif, but can also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; Chowrira & McSwiggen, U.S. Pat. No. 5,631,359; of the hepatitis delta virus motif is described by Perrotta and Been, 1992 Biochemistry 31, 16; of the RNase P motif by Guerrier-Takada et al., 1983 Cell 35, 849; Forster and Altman, 1990, Science 249, 783; Li and Altman, 1996, Nucleic Acids Res. 24, 835; Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363); Group II introns are described by Griffin et al., 1995, Chem. Biol. 2, 761; Michels and Pyle, 1995, Biochemistry 34, 2965; Pyle et al., International PCT Publication No. WO 96/22689; of the Group I intron by Cech et al., U.S. Pat. No. 4,987,071 and of DNAzymes by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262, and Beigelman et al., International PCT publication No. WO 99/55857. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as the Aptazyme (Breaker et al., WO 98/43993), Amberzyme (Class I motif; FIG. 2; Beigelman et al., U.S. Ser. No. 09/301,511) and Zinzyme (FIG. 3) (Beigelman et al., U.S. Ser. No. 09/301,511), all included by reference herein including drawings, can also be used in the present invention. These specific motifs or configurations are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In one embodiment of the present invention, a nucleic acid molecule of the instant invention can be between about 10 and 100 nucleotides in length. Exemplary enzymatic nucleic acid molecules of the invention are shown in Tables III to VII. For example, enzymatic nucleic acid molecules of the invention are preferably between about 15 and 50 nucleotides in length, more preferably between about 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between about 15 and 40 nucleotides in length, more preferably between about 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are preferably between about 15 and 75 nucleotides in length, more preferably between about 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between about 10 and 40 nucleotides in length, more preferably between about 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is that the nucleic acid molecule be of sufficient length and suitable conformation for the nucleic acid molecule to interact with its target and/or catalyze a reaction contemplated herein. The length of the nucleic acid molecules of the instant invention are not limiting within the general limits stated.

Preferably, a nucleic acid molecule that modulates, for example, down-regulates REL-A expression comprises between 12 and 100 bases complementary to a RNA molecule of REL-A. Even more preferably, a nucleic acid molecule that modulates, for example REL-A expression comprises between 14 and 24 bases complementary to a RNA molecule of REL-A.

The invention provides a method for producing a class of nucleic acid-based gene modulating agents which exhibit a high degree of specificity for the RNA of a desired target. For example, the enzymatic nucleic acid molecule is preferably targeted to a highly conserved sequence region of target RNAs encoding REL-A (specifically REL-A genes) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

As used in herein “cell” is used in its usual biological sense, and does not refer to an entire multicellular organism. The cell can, for example, be in vitro, e.g., in cell culture, or present in a multicellular organism, including, e.g., birds, plants and mammals such as humans, cows, sheep, apes, monkeys, swine, dogs, and cats. The cell may be prokaryotic (e.g., bacterial cell) or eukaryotic (e.g., mammalian or plant cell).

By “REL-A proteins” is meant, a peptide or protein comprising a REL-A or p65 subunit of NFKB, for example a subunit involved in transcriptional activation activity.

By “highly conserved sequence region” is meant, a nucleotide sequence of one or more regions in a target gene does not vary significantly from one generation to the other or from one biological system to the other.

Nucleic acid-based inhibitors of NFKB function are useful for the prevention and/or treatment of cancers and cancerous conditions such as breast, lung, prostate, colorectal, brain, esophageal, bladder, pancreatic, cervical, head and neck, and ovarian cancer, melanoma, lymphoma, glioma, multidrug resistant cancers, and any other diseases or conditions that are related to or will respond to the levels of NFKB in a cell or tissue, alone or in combination with other therapies.

Nucleic acid-based inhibitors of NFKB function are also useful for the prevention and/or treatment of inflammatory diseases and conditions, including but not limited to rheumatoid arthritis, restenosis, asthma, Crohn's disease, diabetes, obesity, autoimmune disease, lupus, multiple sclerosis, transplant/graft rejection, gene therapy applications, ischemia/reperfusion injury (CNS and myocardial), glomerulonephritis, sepsis, allergic airway inflammation, inflammatory bowel disease, infection, and any other inflammatory disease or condition which respond to the modulation of NFKB function.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection or infusion pump, with or without their incorporation in biopolymers. In preferred embodiments, the enzymatic nucleic acid inhibitors comprise sequences, which are complementary to the substrate sequences in Tables III to VII. Examples of such enzymatic nucleic acid molecules also are shown in Tables III to VII. Examples of such enzymatic nucleic acid molecules consist essentially of sequences defined in these tables.

In another embodiment, the invention features antisense nucleic acid molecules and 2-5A chimera including sequences complementary to the substrate sequences shown in Tables III to VII. Such nucleic acid molecules can include sequences as shown for the binding arms of the enzymatic nucleic acid molecules in Tables III to VII. Similarly, triplex molecules can be provided targeted to the corresponding DNA target regions, and containing the DNA equivalent of a target sequence or a sequence complementary to the specified target (substrate) sequence. Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both.

By “consists essentially of” is meant that the active nucleic acid molecule of the invention, for example, an enzymatic nucleic acid molecule, contains an enzymatic center or core equivalent to those in the examples, and binding arms able to bind RNA such that cleavage at the target site occurs. Other sequences can be present which do not interfere with such cleavage. Thus, a core region can, for example, include one or more loop, stem-loop structure, or linker which does not prevent enzymatic activity. Thus, the underlined regions in the sequences in Table III can be such a loop, stem-loop, nucleotide linker, and/or non-nucleotide linker and can be represented generally as sequence “X”. For example, a core sequence for a hammerhead enzymatic nucleic acid can comprise a conserved sequence, such as 5′-CUGAUGAG-3′ and 5′-CGAA-3′ connected by “X”, where X is 5′-GCCGUUAGGC-3′ (SEQ ID NO 3929), or any other Stem II region known in the art, or a nucleotide and/or non-nucleotide linker. Similarly, for other nucleic acid molecules of the instant invention, such as Inozyme, G-cleaver, amberzyme, zinzyme, DNAzyme, antisense, 2-5A antisense, triplex forming nucleic acid, and decoy nucleic acids, other sequences or non-nucleotide linkers can be present that do not interfere with the function of the nucleic acid molecule.

Sequence X can be a linker of >2 nucleotides in length, preferably 3, 4, 5, 6, 7, 8, 9, 10, 15, 20, 26, 30, where the nucleotides can preferably be internally base-paired to form a stem of preferably≧2 base pairs. In yet another embodiment, the nucleotide linker X can be a nucleic acid aptamer, such as an ATP aptamer, HIV Rev aptamer (RRE), HIV Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press). A “nucleic acid aptamer” as used herein is meant to indicate a nucleic acid sequence capable of interacting with a ligand. The ligand can be any natural or a synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In yet another embodiment, alternatively or in addition, sequence X can be a non-nucleotide linker. Non-nucleotides as can include abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound which can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in a preferred embodiment, the invention features an enzymatic nucleic acid molecule having one or more non-nucleotide moieties, and having enzymatic activity to cleave an RNA or DNA molecule.

In another aspect of the invention, enzymatic nucleic acid molecules or antisense molecules that interact with target RNA molecules and down-regulate REL-A (specifically REL-A gene) activity are expressed from transcription units inserted into DNA or RNA vectors. The recombinant vectors are preferably DNA plasmids or viral vectors. Enzymatic nucleic acid molecule or antisense expressing viral vectors can be constructed based on, but not limited to, adeno-associated virus, retrovirus, adenovirus, or alphavirus. Preferably, the recombinant vectors capable of expressing the enzymatic nucleic acid molecules or antisense are delivered as described above, and persist in target cells. Alternatively, viral vectors can be used that provide for transient expression of enzymatic nucleic acid molecules or antisense. Such vectors can be repeatedly administered as necessary. Once expressed, the enzymatic nucleic acid molecules or antisense bind to the target RNA and down-regulate its function or expression. Delivery of enzymatic nucleic acid molecule or antisense expressing vectors can be systemic, such as by intravenous or intramuscular administration, by administration to target cells ex-planted from the patient followed by reintroduction into the patient, or by any other means that would allow for introduction into the desired target cell. Antisense DNA can be expressed via the use of a single stranded DNA intracellular expression vector.

By “vectors” is meant any nucleic acid- and/or viral-based technique used to deliver a desired nucleic acid.

By “patient” is meant an organism, which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

By “enhanced enzymatic activity” is meant to include activity measured in cells and/or in vivo where the activity is a reflection of both the catalytic activity and the stability of the nucleic 30 acid molecules of the invention. In this invention, the product of these properties can be increased in vivo compared to an all RNA enzymatic nucleic acid or all DNA enzyme. In some cases, the activity or stability of the nucleic acid molecule can be decreased (i.e., less than ten- fold), but the overall activity of the nucleic acid molecule is enhanced, in vivo.

The nucleic acid molecules of the instant invention, individually, or in combination or in conjunction with other drugs, can be used to treat diseases or conditions discussed above. For example, to treat a disease or condition associated with the levels of REL-A, the patient can be treated, or other appropriate cells can be treated, as is evident to those skilled in the art, individually or in combination with one or more drugs under conditions suitable for the treatment.

In a further embodiment, the described molecules, such as antisense or ribozymes, can be used in combination with other known treatments to treat conditions or diseases discussed above. For example, the described molecules can be used in combination with one or more known therapeutic agents to treat breast, lung, prostate, colorectal, brain, esophageal, bladder, pancreatic, cervical, head and neck, and ovarian cancer, melanoma, lymphoma, glioma, multidrug resistant cancers, rheumatoid arthritis, restenosis, asthma, Crohn's disease, diabetes, obesity, autoimmune disease, lupus, multiple sclerosis, transplant/graft rejection, gene therapy applications, ischemia/reperfusion injury (CNS and myocardial), glomerulonephritis, sepsis, allergic airway inflammation, inflammatory bowel disease, infection, and any other cancerous disease or inflammatory disease or condition which respond to the modulation of REL-A expression.

In another embodiment, the invention features nucleic acid-based inhibitors (e.g., enzymatic nucleic acid molecules (eg; ribozymes), antisense nucleic acids, 2-5A antisense chimeras, triplex DNA, antisense nucleic acids containing RNA cleaving chemical groups) and methods for their use to down regulate or inhibit the expression of genes (e.g., REL-A) capable of progression and/or maintenance of cancer, inflammatory diseases, and/or other disease states which respond to the modulation of REL-A expression.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of⇄ indicates that the listed elements are required or mandatory, and that no other elements may be present.

Other features and advantages of the invention will be apparent from the following description of the preferred embodiments thereof, and from the claims.

Mechanism of Action of Nucleic Acid Molecules of the Invention as Proposed in the Art

Antisense: Antisense molecules can be modified or unmodified RNA, DNA, or mixed polymer oligonucleotides and primarily function by specifically binding to matching sequences resulting in inhibition of peptide synthesis (Wu-Pong, Nov. 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). To date, the only backbone modified DNA chemistry which act as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. Ser. No. 60/101,174 which was filed on Sep. 21, 1998) all of these are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

Enzymatic Nucleic Acid: Several varieties of enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention will block to some extent REL-A and/or NFKB protein expression and can be used to treat disease or diagnose disease associated with the levels of REL-A and/or NFKB. Enzymatic nucleic acid sequences targeting REL-A RNA and sequences that can be targeted with nucleic acid molecules of the invention to down-regulate REL-A expression are shown in Tables III to VII.

The enzymatic nature of an enzymatic nucleic acid molecule can allow the concentration of enzymatic nucleic acid molecule necessary to affect a therapeutic treatment to be lower. This reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to greatly attenuate the catalytic activity of a enzymatic nucleic acid molecule.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieve efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving enzymatic nucleic acid molecules can be used as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology., 6, 237-250).

Enzymatic nucleic acid molecules of the invention that are allosterically regulated (“allozymes”) can be used to modulate NFKB expression. These allosteric enzymatic nucleic acids or allozymes (see for example George et al., U.S. Pat. Nos. 5,834,186 and 5,741,679, Shih et al., U.S. Pat. No. 5,589,332, Nathan et al., U.S. Pat. No 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842) are designed to respond to a signaling agent, for example, mutant REL-A protein, wild-type REL-A protein, mutant REL-A RNA, wild-type REL-A RNA, other proteins and/or RNAs involved in NFKB activity, compounds, metals, polymers, molecules and/or drugs that are targeted to NFKB or NFKB subunit, such as REL-A, expressing cells etc., which in turn modulates the activity of the enzymatic nucleic acid molecule. In response to interaction with a predetermined signaling agent, the allosteric enzymatic nucleic acid molecule's activity is activated or inhibited such that the expression of a particular target is selectively down-regulated. The target can comprise wild-type REL-A, mutant REL-A, a component of NFKB, and/or a predetermined cellular component that modulates REL-A/NFKB activity. In a specific example, allosteric enzymatic nucleic acid molecules that are activated by interaction with a RNA encoding a mutant REL-A protein are used as therapeutic agents in vivo. The presence of RNA encoding the mutant REL-A protein activates the allosteric enzymatic nucleic acid molecule that subsequently cleaves the RNA encoding a mutant REL-A protein resulting in the inhibition of mutant REL-A protein expression. In this manner, cells that express the mutant form of the REL-A protein are selectively targeted.

In another non-limiting example, an allozyme can be activated by a REL-A protein, peptide, or mutant polypeptide that caused the allozyme to inhibit the expression of REL-A gene, by, for example, cleaving RNA encoded by REL-A gene. In this non-limiting example, the allozyme acts as a decoy to inhibit the function of REL-A and also inhibit the expression of REL-A once activated by the REL-A protein.

The nucleic acid molecules of the instant invention are also referred to as GeneBloc reagents, which are essentially nucleic acid molecules (eg; ribozymes, antisense) capable of down-regulating gene expression.

Target Sites

Targets for useful enzymatic nucleic acid molecules and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468, and hereby incorporated by reference herein in totality. Other examples include the following PCT applications, which concern inactivation of expression of disease-related genes: WO 95/23225, WO 95/13380, WO 94/02595, incorporated by reference herein. Rather than repeat the guidance provided in those documents here, below are provided specific examples of such methods, not limiting to those in the art. Enzymatic nucleic acid molecules and antisense to such targets are designed as described in those applications and synthesized to be tested in vitro and in vivo, as also described. The sequences of human REL-A RNAs were screened for optimal enzymatic nucleic acid and antisense target sites using a computer-folding algorithm. Antisense, hammerhead, DNAzyme, NCH, amberzyme, zinzyme, or G-Cleaver enzymatic nucleic acid molecule binding/cleavage sites were identified. These sites are shown in Tables III to VII (all sequences are 5′ to 3′ in the tables; underlined regions can be any sequence “X” or linker X, the actual sequence is not relevant here). The nucleotide base position is noted in the Tables as that site to be cleaved by the designated type of enzymatic nucleic acid molecule. While human sequences can be screened and enzymatic nucleic acid molecule and/or antisense thereafter designed, as discussed in Stinchcomb et al., WO 95/23225, mouse targeted enzymatic nucleic acid molecules can be useful to test efficacy of action of the enzymatic nucleic acid molecule and/or antisense prior to testing in humans.

Antisense, hammerhead, DNAzyme, NCH, amberzyme, zinzyme or G-Cleaver enzymatic nucleic acid molecule binding/cleavage sites were identified. The nucleic acid molecules are individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core are eliminated from consideration. Varying binding arm lengths can be chosen to optimize activity.

Antisense, hammerhead, DNAzyme, NCH, amberzyme, zinzyme or G-Cleaver enzymatic nucleic acid molecule binding/cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211,3-19.

Synthesis of Nucleic Acid Molecules

Synthesis of nucleic acids greater than 100 nucleotides in length can be difficult using automated methods, and the therapeutic cost of such molecules can be prohibitive. In this invention, small nucleic acid motifs (” small refers to nucleic acid motifs less than about 100 nucleotides in length, preferably less than about 80 nucleotides in length, and more preferably less than about 50 nucleotides in length; e.g., antisense oligonucleotides, hammerhead or the NCH ribozymes) are preferably used for exogenous delivery. The simple structure of these molecules increases the ability of the nucleic acid to invade targeted regions of RNA structure. Exemplary molecules of the instant invention are chemically synthesized, and others can similarly be synthesized.

Oligonucleotides (eg; antisense, GeneBlocs) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the antisense oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20 ° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder.

The method of synthesis used for RNA and chemically modified RNA including certain enzymatic nucleic acid molecules follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20 ° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA.3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead ribozymes or binding attenuated control (BAC) oligonucleotides can be synthesized by substituting a U for G₅ and a U for A₁₄ (numbering from Hertel, K. J., et al., 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96 well format, with the ratio of chemicals being used in the reaction adjusted accordingly.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; See Wincott et al., Supra, the totality of which is hereby incorporated herein by reference) and are re-suspended in water.

The sequences of the nucleic acid molecules, including enzymatic nucleic acid molecules and antisense, that are chemically synthesized, are shown in Table VII. The sequences of the enzymatic nucleic acid and antisense constructs that are chemically synthesized, are complementary to the Substrate sequences shown in Table VII. Those in the art will recognize that these sequences are representative only of many more such sequences where the enzymatic portion of the ribozyme (all but the binding arms) is altered to affect activity. The enzymatic nucleic acid and antisense construct sequences listed in Tables III to VII can be formed of ribonucleotides or other nucleotides or non-nucleotides. Such enzymatic nucleic acid molecules with enzymatic activity are equivalent to the enzymatic nucleic acid molecules described specifically in the Tables.

Optimizing Activity of the Nucleic Acid Molecule of the Invention

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All these publications are hereby incorporated by reference herein).

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry , 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci. , 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such nucleic acid is also generally more resistant to nucleases than unmodified nucleic acid. Thus, in a cell and/or in vivo the activity may not be significantly lowered. Therapeutic nucleic acid molecules delivered exogenously are optimally stable within cells until translation of the target RNA has been inhibited long enough to reduce the levels of the undesirable protein. This period of time varies between hours to days depending upon the disease state. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein) have expanded the ability to modify nucleic acid molecules by introducing nucleotide modifications to enhance their nuclease stability as described above.

Use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules can also include combinations of different types of nucleic acid molecules.

Therapeutic nucleic acid molecules (e.g., enzymatic nucleic acid molecules and antisense nucleic acid molecules) delivered exogenously are optimally stable within cells until translation of the target RNA has been inhibited long enough to reduce the levels of the undesirable protein. This period of time varies between hours to days depending upon the disease state. These nucleic acid molecules should be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In one embodiment, nucleic acid catalysts having chemical modifications that maintain or enhance enzymatic activity are provided. Such nucleic acids are also generally more resistant to nucleases than unmodified nucleic acid. Thus, in a cell and/or in vivo the activity of the nucleic acid may not be significantly lowered. As exemplified herein such enzymatic nucleic acids are useful in a cell and/or in vivo even if activity over all is reduced about 10 fold (Burgin et al., 1996, Biochemistry, 35, 14090). Such enzymatic nucleic acids herein are said to “maintain” the enzymatic activity of an all RNA ribozyme or all DNA DNAzyme.

In another aspect the nucleic acid molecules comprise a 5′ and/or a 3′-cap structure.

By “cap structure” is meant chemical modifications, which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both terminus. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In another embodiment the 3′-cap includes, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

The term “alkyl” as used herein refers to a saturated aliphatic hydrocarbon, including straight-chain, branched-chain “isoalkyl”, and cyclic alkyl groups. The term “alkyl” also comprises alkoxy, alkyl-thio, alkyl-thio-alkyl, alkoxyalkyl, alkylamino, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Preferably, the alkyl group has 1 to 12 carbons. More preferably it is a lower alkyl of from about 1 to 7 carbons, more preferably about 1 to 4 carbons. The alkyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkenyl groups containing at least one carbon-carbon double bond, including straight-chain, branched-chain, and cyclic groups. Preferably, the alkenyl group has about 2 to 12 carbons. More preferably it is a lower alkenyl of from about 2 to 7 carbons, more preferably about 2 to 4 carbons. The alkenyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkynyl groups containing at least one carbon-carbon triple bond, including straight-chain, branched-chain, and cyclic groups. Preferably, the alkynyl group has about 2 to 12 carbons. More preferably it is a lower alkynyl of from about 2 to 7 carbons, more preferably about 2 to 4 carbons. The alkynyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Alkyl groups or moieties of the invention can also include aryl, alkylaryl, carbocyclic aryl, heterocyclic aryl, amide and ester groups. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from about 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

The term “alkoxyalkyl” as used herein refers to an alkyl-O-alkyl ether, for example methoxyethyl or ethoxymethyl.

The term “alkyl-thio-alkyl” as used herein refers to an alkyl-S-alkyl thioether, for example methylthiomethyl or methylthioethyl.

The term “amino” as used herein refers to a nitrogen containing group as is known in the art derived from ammonia by the replacement of one or more hydrogen radicals by organic radicals. For example, the terms “aminoacyl” and “aminoalkyl” refer to specific N-substituted organic radicals with acyl and alkyl substituent groups respectively.

The term “amination” as used herein refers to a process in which an amino group or substituted amine is introduced into an organic molecule.

The term “exocyclic amine protecting moiety” as used herein refers to a nucleobase amino protecting group compatible with oligonucleotide synthesis, for example an acyl or amide group.

The term “alkenyl” as used herein refers to a straight or branched hydrocarbon of a designed number of carbon atoms containing at least one carbon-carbon double bond. Examples of “alkenyl” include vinyl, allyl, and 2-methyl-3-heptene.

The term “alkoxy” as used herein refers to an alkyl group of indicated number of carbon atoms attached to the parent molecular moiety through an oxygen bridge. Examples of alkoxy groups include, for example, methoxy, ethoxy, propoxy and isopropoxy.

The term “alkynyl” as used herein refers to a straight or branched hydrocarbon of a designed number of carbon atoms containing at least one carbon-carbon triple bond. Examples of “alkynyl” include propargyl, propyne, and 3-hexyne.

The term “aryl” as used herein refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “cycloalkenyl” as used herein refers to a C3-C8 cyclic hydrocarbon containing at least one carbon-carbon double bond. Examples of cycloalkenyl include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “cycloalkyl” as used herein refers to a C3-C8 cyclic hydrocarbon. Examples of cycloalkyl include cyclopropyl, cyclobutyl, cyclopentyl, cyclohexyl, cycloheptyl and cyclooctyl.

The term “cycloalkylalkyl,” as used herein, refers to a C3-C7 cycloalkyl group attached to the parent molecular moiety through an alkyl group, as defined above. Examples of cycloalkylalkyl groups include cyclopropylmethyl and cyclopentylethyl.

The terms “halogen” or “halo” as used herein refers to indicate fluorine, chlorine, bromine, and iodine.

The term “heterocycloalkyl,” as used herein refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrolidinyl.

The term “heteroaryl” as used herein refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

The term “C1-C6 hydrocarbyl” as used herein refers to straight, branched, or cyclic alkyl groups having 1-6 carbon atoms, optionally containing one or more carbon-carbon double or triple bonds. Examples of hydrocarbyl groups include, for example, methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, vinyl, 2-pentene, cyclopropylmethyl, cyclopropyl, cyclohexylmethyl, cyclohexyl and propargyl. When reference is made herein to C1-C6 hydrocarbyl containing one or two double or triple bonds it is understood that at least two carbons are present in the alkyl for one double or triple bond, and at least four carbons for two double or triple bonds.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In one embodiment, the invention features modified enzymatic nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, for example a 3′,3′-linked or 5′,5′-linked deoxyabasic ribose derivative (for more details see Wincott et al., International PCT publication No. WO 97/26270).

By “unmodified nucleoside” is meant one of the bases adenine, cytosine, guanine, thymine, uracil joined to the 1′carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O-NH₂, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., antisense and ribozyme) structure can be made to enhance the utility of these molecules. For example, such modifications can enhance shelf-life, half-life in vitro, stability, and ease of introduction of such oligonucleotides to the target site, including e.g., enhancing penetration of cellular membranes and conferring the ability to recognize and bind to targeted cells.

Use of these molecules can lead to better treatment of the disease progression by affording the possibility of combination therapies (e.g., multiple enzymatic nucleic acid molecules targeted to different genes, enzymatic nucleic acid molecules coupled with known small molecule inhibitors, or intermittent treatment with combinations of enzymatic nucleic acid molecules (including different enzymatic nucleic acid molecule motifs) and/or other chemical or biological molecules). The treatment of patients with nucleic acid molecules can also include combinations of different types of nucleic acid molecules. Therapies can be devised which include a mixture of enzymatic nucleic acid molecules (including different enzymatic nucleic acid molecule motifs), antisense and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example, through the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. NeuroVirol., 3, 387-400. More detailed descriptions of nucleic acid delivery and administration are provided in Sullivan et al., supra, Draper et al., PCT WO93/23569, Beigelman et al., PCT WO99/05094, and Klimuk et al., PCT WO99/04819 all of which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al., 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al.,1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a patient in combination with other therapeutic compounds to increase the overall therapeutic effect. The use of multiple compounds to treat an indication can increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2,3-15; Dropulic et al., 1992, J. Virol., 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of these references are hereby incorporated in their totalities by reference herein). Those skilled in the art realize that any nucleic acid can be expressed in eukaryotic cells from the appropriate DNA/RNA vector. The activity of such nucleic acids can be augmented by their release from the primary transcript by a enzymatic nucleic acid (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein). Gene therapy approaches specific to the CNS are described by Blesch et al., 2000, Drug News Perspect., 13, 269-280; Peterson et al. 2000, Cent. Nerv. Syst. Dis., 485-508; Peel and Klein, 2000, J. Neurosci. Methods, 98, 95-104; Hagihara et al., 2000, Gene Ther., 7, 759-763; and Herrlinger et al., 2000, Methods Mol. Med., 35, 287-312. AAV-mediated delivery of nucleic acid to cells of the nervous system is further described by Kaplitt et al., U.S. Pat. No. 6,180,613.

In another aspect of the invention, RNA molecules of the present invention are preferably expressed from transcription units (see for example Couture et al., 1996, TIG., 12, 510) inserted into DNA or RNA vectors. The recombinant vectors are preferably DNA plasmids or viral vectors. Ribozyme expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

In one aspect the invention features an expression vector comprising a nucleic acid sequence encoding at least one of the nucleic acid molecules of the instant invention is disclosed. The nucleic acid sequence encoding the nucleic acid molecule of the instant invention is operable linked in a manner which allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. U S A, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. U S A, 89, 10802-6; Chen et al., 1992, Nucleic Acids Res., 20, 4581-9; Yu et al., 1993, Proc. Natl. Acad. Sci. U S A, 90, 6340-4; L'Huillier et al., 1992, EMBO J., 11, 4411-8; Lisziewicz et al., 1993, Proc. Natl. Acad. Sci. U. S. A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4,45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another embodiment the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of Antisense, hammerhead, DNAzyme, NCH, Amberzyme, Zinzyme, or G-Cleaver ribozyme molecules and binding/cleavage sites within REL-A RNA.

### Example 1

### Identification of Potential Target Sites in Human REL-A RNA

The sequence of human REL-A genes are screened for accessible sites using a computer-folding algorithm. Regions of the RNA that do not form secondary folding structures and contained potential enzymatic nucleic acid molecule and/or antisense binding/cleavage sites are identified. The sequences of these binding/cleavage sites are shown in Tables III-VII.

### Example 2

### Selection of Enzymatic Nucleic Acid Cleavage Sites in Human REL-A RNA

Enzymatic nucleic acid molecule target sites are chosen by analyzing sequences of Human REL-A (Genbank accession No: NM_{—}005228) and prioritizing the sites on the basis of folding. Enzymatic nucleic acid molecules are designed that can bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the binding arms and the catalytic core are eliminated from consideration. As noted below, varying binding arm lengths can be chosen to optimize activity. Generally, at least 5 bases on each arm are able to bind to, or otherwise interact with, the target RNA.

### Example 3

### Chemical Synthesis and Purification of Ribozymes and Antisense for Efficient Cleavage and/or blocking of REL-A RNA

Enzymatic nucleic acid molecules and antisense constructs are designed to anneal to various sites in the RNA message. The binding arms of the enzymatic nucleic acid molecules are complementary to the target site sequences described above, while the antisense constructs are fully complementary to the target site sequences described above. The enzymatic nucleic acid molecules and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%.

Enzymatic nucleic acid molecules and antisense constructs are also synthesized from DNA templates using bacteriophage T7 RNA polymerase (Milligan and Uhlenbeck, 1989, Methods Enzymol. 180, 51). Enzymatic nucleic acid molecules and antisense constructs are purified by gel electrophoresis using general methods or are purified by high pressure liquid chromatography (HPLC; See Wincott et al., supra; the totality of which is hereby incorporated herein by reference) and are resuspended in water. The sequences of the chemically synthesized enzymatic nucleic acid molecules used in this study are shown below in Table VII. The sequences of the chemically synthesized antisense constructs used in this study are complementary sequences to the Substrate sequences shown below as in Tables III to VII.

### Example 4

### Enzymatic Nucleic Acid Molecule Cleavage of REL-A RNA Target in Vitro

Enzymatic nucleic acid molecules targeted to the human REL-A RNA are designed and synthesized as described above. These enzymatic nucleic acid molecules can be tested for cleavage activity in vitro, for example, using the following procedure. The target sequences and the nucleotide location within the REL-A RNA are given in Tables III-VII.

Cleavage Reactions: Full-length or partially full-length, internally-labeled target RNA for enzymatic nucleic acid molecule cleavage assay is prepared by in vitro transcription in the presence of [a-³²P] CTP, passed over a G 50 Sephadex column by spin chromatography and used as substrate RNA without further purification. Alternately, substrates are 5′-³²P-end labeled using T4 polynucleotide kinase enzyme. Assays are performed by pre-warming a 2× concentration of purified enzymatic nucleic acid molecule in enzymatic nucleic acid molecule cleavage buffer (50 mM Tris-HCl, pH 7.5 at 37° C., 10 mM MgCl₂) and the cleavage reaction was initiated by adding the 2× enzymatic nucleic acid molecule mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM enzymatic nucleic acid molecule, i.e., enzymatic nucleic acid molecule excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by enzymatic nucleic acid molecule cleavage are visualized on an autoradiograph of the gel. The percentage of cleavage is determined by Phosphor Imager® quantitation of bands representing the intact substrate and the cleavage products.

### Example 5

### Nucleic Acid Down-regulation of REL-A target RNA in vivo

Nucleic acid molecules targeted to the human REL-A RNA are designed and synthesized as described above. These nucleic acid molecules can be tested for cleavage activity in vivo, for example using the procedures described below. The target sequences and the nucleotide location within the REL-A RNA are given in Tables III-VII.

### Example 6

### In vivo Models used to Evaluate the Down-regulation of REL-A Gene Expression

A variety of endpoints have been used in cell culture models to evaluate REL-A-mediated effects after treatment with anti-REL-A agents. Phenotypic endpoints include inhibition of cell proliferation, apoptosis assays and reduction of REL-A protein expression. Because overexpression of REL-A is directly associated with increased proliferation of tumor cells, a proliferation endpoint for cell culture assays is preferably used as a primary screen. There are several methods by which this endpoint can be measured. Following treatment of cells with nucleic acid molecules, cells are allowed to grow (typically 5 days) after which either the cell viability, the incorporation of [³H] thymidine into cellular DNA and/or the cell density can be measured. The assay of cell density is very straightforward and can be performed in a 96-well format using commercially available fluorescent nucleic acid stains (such as Syto® 13 or CyQuant®). The assay using CyQuant® is described herein

As a secondary, confirmatory endpoint a nucleic acid-mediated decrease in the level of REL-A RNA and/or REL-A protein expression can be evaluated.

Cell Culture

Cell types that express/over-express NFKB include HeLa, macrophages, peripheral blood lymphocytes, hepatocytes, fibroblasts, endothelial cells and epithelial cells. In culture, these cells can be stimulated to express/over-express NFKB by addition of TNF-alpha PMA or IL-1-beta to the culture medium. Some of these cell types also may respond with a similar activation of NFKB following LPS treatment. Activation of NFKB in cultured cells can be evaluated by electrophoretic mobility shift assay (EMSA). Delineation of alterations in the subunits can be determined by Western blot.

Primary Screen

A usefult cell culture system is human colonic epithelial cells. One suitable cell line is SW620 colon carcinoma cells (CCL227). These cells respond to stimulation with TNF-alpha, LPS and/or IL-1-beta with an increase in NFKB activation. SW620 cells are grown in MEM supplemented with 10% heat-inactivated FBS and glutamine (2 mmol/L).

TNF-alpha dose-response curves in these cells are determined by incubating cells with various concentrations of recombinant human TNF-alpha (Sigma Chemical Co.). Maximal DNA binding activity induction can occur with 150 U/ml TNF-alpha in the culture medium. Induction is typically evident within 10 minutes of treatment with TNF-alpha reaches a peak at one hour post-treatment and persists for up to 4 hours post-treatment. The primary readout can be NFKB DNA activity in nuclear extracts of SW620 cells as determined by electrophoretic mobility shift assays (EMSA). Once the appropriate TNF-alpha dose/response profile has been determined, inhibition of NFKB activation is evaluated using specific and non-specific inhibitors of activation, sultasalazine and steroids, respectively. Cells are incubated with inhibitors or control media for 30 minutes prior to stimulation with TNF-alpha Nuclear extracts are prepared and evaluated for DNA binding activity by EMSA. Once the activity of positive controls has been established, enzymatic nucleic acids targeting the REL-A subunit of NFKB are evaluated in this system. Supershift assays using polyclonal antibodies against the NFKB protein subunits can be performed to confirm down-regulation of the REL-A component of the heterodimer.

Secondary Screens

SW620 cells can be transfected with the 3xIg-kappa-B-Luc reporter construct 18 hours before challenge with TNF-alpha, LPS or PMA. The readout for this assay is luciferase activity. Test compounds are applied 17.5 hours after transfection (30 minutes before challenge). Cells are harvested 24 hours after challenge and relative changes in luciferase activity is used as the endpoint. Lastly, the activation of NFKB can be visualized fluorescently. Inactive NFKB heterodimers are held in the cytoplasm by inhibitory proteins. Once activated, the free heterodimers translocate to the nucleus. Thus, the relative change in cytoplasmic versus nuclear fluorescence can indicate the degree of NFKB activation. Cells can be grown on chamber slides, treated with TNF-alpha with and without test compounds), and the location of the REL-A subunit can be determined by immunofluorescence using a FITC-labeled antibody to REL-A.

Animal Models

Evaluating the efficacy of anti-REL-A/NFKB agents in animal models is an important prerequisite to human clinical trials. Studies have shown that human breast carcinoma cell lines express high levels of REL-A/NFKB (Sovak et al., 1997, J. Clin. Invest., 100, 2952-2960). High levels of REL-A/NFKB have also been observed in carcinogen-induced primary rat mammary tumors and in human breast cancer specimins. Additionally, HER2/neu overexpression has been shown to activate NFKB (Pianetti et al., 2001, Oncogene, 20, 1287-1299). As such, xenografts of cell lines that over-express NFKB can be used in animal models of tumorigenesis and/or inflammation to study the inhibition of REL-A/NFKB.

Oncology Animal Model Development

Tumor cell lines are characterized to establish their growth curves in mice. These cell lines are implanted into both nude and SCID mice and primary tumor volumes are measured 3 times per week. Growth characteristics of these tumor lines using a Matrigel implantation format can also be established. The use of other cell lines that have been engineered to express high levels of REL-A can also be used in the described studies. The tumor cell line(s) and implantation method that supports the most consistent and reliable tumor growth is used in animal studies testing the lead REL-A nucleic acid(s). Nucleic acids are administered by daily subcutaneous injection or by continuous subcutaneous infusion from Alzet mini osmotic pumps beginning 3 days after tumor implantation and continuing for the duration of the study. Group sizes of at least 10 animals are employed. Efficacy is determined by statistical comparison of tumor volume of nucleic acid-treated animals to a control group of animals treated with saline alone. Because the growth of these tumors is generally slow (45-60 days), an initial endpoint is the time in days it takes to establish an easily measurable primary tumor (i.e. 50-100 mm³) in the presence or absence of nucleic acid treatment.

Inflammation Animal Model Development

Chronic, sublethal administration of indomethacin to outbred rats produces an enteropathy characterized by thickening of the small intestine and mesentery, ulcerations, granulomatous inflammation, crypt abcesses and adhesions. These lesions are similar to those that are characteristic findings in human patients with Crohn's disease (CD). Thus, any beneficial therapeutic effects revealed using this model can be extrapolated to potential benefit for patients with CD.

Male Sprague-Dawley rats (200-275 g) are utilized for these studies. Chronic intestinal inflammation is induced by two subcutaneous injections of indomethacin (7.5 mg/kg in 5% NaHCO3) administered on subsequent days (Day-0 and Day-1). Animals are followed for four days following the first indomethacin injection. The mortality rate associated with this model is typically less than 10%. On the last day of the study, animals are euthanized by CO2 asphyxiation, small intestines excised and gross pathologic findings ranked according to the following criteria: 0, normal ; 1, minimal abnormalities, slight thickening of the small intestine, no adhesions; 2, obvious thickening of small intestine with 1 adhesion; 3, obvious thickening of small intestine with 2 or 3 adhesions; 4, massive adhesions to the extent that the intestine cannot be separated, contents primarily fluid; 5, severe peritonitis resulting in death. A 10-cm portion of the most affected region of the small intestine is weighed, placed in 10% neutral buffered formalin and submitted for histopathologic evaluation.

The 10 cm portion of gut from each animal is cut into five equal sections. Transverse and longitudinal sections of each portion are cut and stained with hematoxylin and eosin. All slides are read in a blinded fashion and each section is scored for necrosis (% area of involvement) and inflammatory response according to the following scale:

Necrosis 1, 10%; 2, 10-25%; 3, 25-50%; 4, 50-75%; 5, 75-100%;

Inflammation

1=minimal in mesentery and muscle or lesion

2=mild in mesentery and muscle or lesion

3=moderate in mesentery and muscle or lesion

4=marked in lesion

5=severe in lesion

The scores for each of the five sections are averaged for necrosis and for inflammation.

REL-A Protein Levels for Patient Screening and as a Potential Endpoint

Because elevated REL-A levels can be detected in cancers, cancer patients can be pre-screened for elevated REL-A prior to admission to initial clinical trials testing an anti-REL-A nucleic acid. Initial REL-A levels can be determined (by ELISA) from tumor biopsies or resected tumor samples. During clinical trials, it may be possible to monitor circulating REL-A protein by ELISA. Evaluation of serial blood/serum samples over the course of the anti-REL-A nucleic acid treatment period could be useful in determining early indications of efficacy.

### Example 7

### Activity of Nucleic Acid Molecules used to Down-regulate REL-A Gene Expression

Applicant has designed and synthesized several nucleic acid molecules targeted against REL-A RNA. These nucleic acid molecules can be tested in cell proliferation and RNA reduction assays described herein.

Proliferation Assay

The model proliferation assay used in the study requires a cell-plating density of 2,000-10,000 cells/well in 96-well plates and at least 2 cell doublings over a 5-day treatment period. Cells used in proliferation studies were either lung or ovarian cancer cells (A549 and SKOV-3 cells respectively). To calculate cell density for proliferation assays, the FIPS (fluoro-imaging processing system) method known in the art was used. This method allows for cell density measurements after nucleic acids are stained with CyQuant® dye, and has the advantage of accurately measuring cell densities over a very wide range 1,000-100,000 cells/well in 96-well format. Enzymatic nucleic acid molecules (50-200 nM) are delivered in the presence of cationic lipid at 2.5-5.0 μg/mL and inhibition of proliferation was determined on day 5 post-treatment.

RNA Assay

RNA is harvested 24 hours post-treatment using the Qiagen RNeasy® 96 procedure. Real time RT-PCR (TaqMan® assay) is performed on purified RNA samples using separate primer/probe sets specific for target REL-A RNA.

Indications

Particular degenerative and disease states that can be associated with REL-A expression modulation include but are not limited to cancerous and/or inflammatory diseases and conditions such as breast, lung, prostate, colorectal, brain, esophageal, bladder, pancreatic, cervical, head and neck, and ovarian cancer, melanoma, lymphoma, glioma, multidrug resistant cancers, rheumatoid arthritis, restenosis, asthma, Crohn's disease, diabetes, obesity, autoimmune disease, lupus, multiple sclerosis, transplant/graft rejection, gene therapy applications, ischemia/reperfusion injury (CNS and myocardial), glomerulonephritis, sepsis, allergic airway inflammation, inflammatory bowel disease, infection, and any other diseases or conditions that are related to or respond to the levels of REL-A in a cell or tissue. The present body of knowledge in REL-A research indicates the need for methods to assay REL-A activity and for compounds that can regulate REL-A expression for research, diagnostic, and therapeutic use.

The use of monoclonal antibodies, chemotherapy, radiation therapy, analgesics, and/or anti-inflammatory compounds, are all non-limiting examples of a methods that can be combined with or used in conjunction with the nucleic acid molecules (e.g. ribozymes and antisense molecules) of the instant invention. Common chemotherapies that can be combined with nucleic acid molecules of the instant invention include various combinations of cytotoxic drugs to kill cancer cells. These drugs include but are not limited to paclitaxel (Taxol), docetaxel, cisplatin, methotrexate, cyclophosphamide, doxorubin, fluorouracil carboplatin, edatrexate, gemcitabine, vinorelbine etc. Those skilled in the art will recognize that other drug compounds and therapies can be similarly be readily combined with the nucleic acid molecules of the instant invention (e.g. ribozymes and antisense molecules) are hence within the scope of the instant invention.

Diagnostic Uses

The nucleic acid molecules of this invention (e.g., enzymatic nucleic acid molecules) can be used as diagnostic tools to examine genetic drift and mutations within diseased cells or to detect the presence of REL-A RNA in a cell. The close relationship between enzymatic nucleic acid molecule activity and the structure of the target RNA allows the detection of mutations in any region of the molecule which alters the base-pairing and three-dimensional structure of the target RNA. By using multiple enzymatic nucleic acid molecules described in this invention, one can map nucleotide changes which are important to RNA structure and function in vitro, as well as in cells and tissues. Cleavage of target RNAs with enzymatic nucleic acid molecules can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple enzymatic nucleic acid molecules targeted to different genes, enzymatic nucleic acid molecules coupled with known small molecule inhibitors, or intermittent treatment with combinations of enzymatic nucleic acid molecules and/or other chemical or biological molecules). Other in vitro uses of enzymatic nucleic acid molecules of this invention are well known in the art, and include detection of the presence of mRNAs associated with REL-A-related condition. Such RNA is detected by determining the presence of a cleavage product after treatment with an enzymatic nucleic acid molecule using standard methodology.

In a specific example, enzymatic nucleic acid molecules which cleave only wild-type or mutant forms of the target RNA are used for the assay. The first enzymatic nucleic acid molecule is used to identify wild-type RNA present in the sample and the second enzymatic nucleic acid molecule is used to identify mutant RNA in the sample. As reaction controls, synthetic substrates of both wild-type and mutant RNA are cleaved by both enzymatic nucleic acid molecules to demonstrate the relative enzymatic nucleic acid molecule efficiencies in the reactions and the absence of cleavage of the “non-targeted” RNA species. The cleavage products from the synthetic substrates also serve to generate size markers for the analysis of wild-type and mutant RNAs in the sample population. Thus each analysis requires two enzymatic nucleic acid molecules, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., REL-A) is adequate to establish risk. If probes of comparable specific activity are used for both transcripts, then a qualitative comparison of RNA levels will be adequate and will decrease the cost of the initial diagnosis. Higher mutant form to wild-type ratios are correlated with higher risk whether RNA levels are compared qualitatively or quantitatively. The use of enzymatic nucleic acid molecules in diagnostic applications contemplated by the instant invention is more fully described in George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.

Additional Uses

Potential uses of sequence-specific enzymatic nucleic acid molecules of the instant invention can have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant has described the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably can be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiment are within the claims that follow.

TABLE I
Characteristics of naturally occurring ribozymes

Group I Introns
Size: ˜150 to >1000 nucleotides.
Requires a U in the target sequence immediately 5′ of the cleavage site.
Binds 4-6 nucleotides at the 5-side of the cleavage site.
Reaction mechanism: attack by the 3′-OH of guanosine to generate
cleavage products with 3′-OH and 5′-guanosine.
Additional protein cofactors required in some cases to help folding and
maintenance of the active structure.
Over 300 known members of this class. Found as an intervening sequence
in Tetrahymena thermophila rRNA, fungal mitochondria, chloroplasts,
phage T4, blue-green algae, and others.
Major structural features largely established through phylogenetic
comparisons, mutagenesis, and biochemical studies [^{i,ii}].
Complete kinetic framework established for one ribozyme [^{iii,iv,v,vi}].
Studies of ribozyme folding and substrate docking underway [^{vii,viii,ix}].
Chemical modification investigation of important residues well established
[^{x,xi}].
The small (4-6 nt) binding site may make this ribozyme too non-specific
for targeted RNA cleavage, however, the Tetrahymena group I intron has
been used to repair a “defective” β-galactosidase message by the
ligation of new galactosidase sequences onto the defective message [^{xii}].
RNAse P RNA (M1 RNA)
Size: ˜290 to 400 nucleotides.
RNA portion of a ubiquitous ribonucleoprotein enzyme.
Cleaves tRNA precursors to form mature tRNA [^{xiii}].
Reaction mechanism: possible attack by M²⁺ -OH to generate cleavage
products with 3′-OH and 5′-phosphate.
RNAse P is found throughout the prokaryotes and eukaryotes. The RNA
subunit has been sequenced from bacteria, yeast, rodents, and primates.
Recruitment of endogenous RNAse P for therapeutic applications is
possible through hybridization of an External Guide Sequence (EGS)
to the target RNA [^{xiv,xv}]
Important phosphate and 2′ OH contacts recently identified [^{xvi,xvii}]
Group II Introns
Size: >1000 nucleotides.
Trans cleavage of target RNAs recently demonstrated [^{xviii,xix}].
Sequence requirements not fully determined.
Reaction mechanism: 2′-OH of an internal adenosine generates cleavage
products with 3′-OH and a “lariat” RNA containing a 3′-5′ and a
2′-5′ branch point.
Only natural ribozyme with demonstrated participation in DNA cleavage
[^{xx,xxi}] in addition to RNA cleavage and ligation.
Major structural features largely established through phylogenetic
comparisons [^{xxii}].
Important 2′ OH contacts beginning to be identified [^{xxiii}]
Kinetic framework under development [^{xxiv}]
Neurospora VS RNA
Size: ˜144 nucleotides.
Trans cleavage of hairpin target RNAs recently demonstrated [^{xxv}].
Sequence requirements not fully determined.
Reaction mechanism: attack by 2′-OH 5′ to the scissile bond to
generate cleavage products with 2′,3′-cyclic phosphate and 5′-OH ends.
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to
generate cleavage products with 2′,3′-cyclic phosphate and 5′-OH ends.
14 known members of this class. Found in a number of plant pathogens
(virusoids) that use RNA as the infectious agent.
Essential structural features largely defined, including 2 crystal structures
[^{xxvi,xxvii}]
Minimal ligation activity demonstrated (for engineering through in vitro
selection) [^{xxviii}]
Complete kinetic framework established for two or more ribozymes [^{xxix}].
Chemical modification investigation of important residues well established
[^{xxx}].
Hairpin Ribozyme
Size: ˜50 nucleotides.
Requires the target sequence GUC immediately 3′ of the cleavage site.
Binds 4-6 nucleotides at the 5-side of the cleavage site and a variable
number to the 3′-side of the cleavage site.
Reaction mechanism: attack by 2′-OH 5′ to the scissile bond to generate
cleavage products with 2′,3′-cyclic phosphate and 5′-OH ends.
3 known members of this class. Found in three plant pathogen (satellite
RNAs of the tobacco ringspot virus, arabis mosaic virus and chicory
yellow mottle virus) which uses RNA as the infectious agent.
Essential structural features largely defined [^{xxxi,xxxii,xxxiii,xxxiv}]
Ligation activity (in addition to cleavage activity) makes ribozyme
amenable to engineering through in vitro selection [^{xxxv}]
Complete kinetic framework established for one ribozyme [^{xxxvi}].
Chemical modification investigation of important residues begun
[^{xxxvii,xxxviii}].
Hepatitis Delta Virus (HDV) Ribozyme
Size: ˜60 nucleotides.
Trans cleavage of target RNAs demonstrated [^{xxxix}].
Binding sites and structural requirements not fully determined, although no
sequences 5′ of cleavage site are required. Folded ribozyme contains a
pseudoknot structure [^{xl}]
Reaction mechanism: attack by 2′-OH 5′ to the scissile bond to generate
cleavage products with 2′,3′-cyclic phosphate and 5′-OH ends.
Only 2 known members of this class. Found in human HDV.
Circular form of HDV is active and shows increased nuclease stability [^{xli}]
[ⁱ], Michel, Francois; Westhof, Eric. Slippery substrates. Nat. Struct. Biol. (1994), 1(1), 5-7.
[ⁱⁱ], Lisacek, Frederique; Diaz, Yolande; Michel, Francois. Automatic identification of group I intron cores in genomic DNA sequences. J. Mol. Biol. (1994), 235(4), 1206-17.
[ⁱⁱⁱ], Herschlag, Daniel; Cech, Thomas R.. Catalysis of RNA cleavage by the Tetrahymena thermophila ribozyme. 1. Kinetic description of the reaction of an RNA substrate complementary to the active site. Biochemistry (1990), 29(44), 10159-71.
[^{iv}], Herschlag, Daniel; Cech, Thomas R.. Catalysis of RNA cleavage by the Tetrahymena thermophila ribozyme. 2. Kinetic description of the reaction of an RNA substrate that forms a mismatch at the active site. Biochemistry (1990), 29(44), 10172-80.
[^{v}], Knitt, Deborah S.; Herschlag, Daniel. pH Dependencies of the Tetrahymena Ribozyme Reveal an Unconventional Origin of an Apparent pKa. Biochemistry (1996), 35(5), 1560-70.
[^{vi}], Bevilacqua, Philip C.; Sugimoto, Naoki; Turner, Douglas H.. A mechanistic framework for the second step of splicing catalyzed by the Tetrahymena ribozyme. Biochemistry (1996), 35(2), 648-58.
[^{vii}], Li, Yi; Bevilacqua, Philip C.; Mathews, David; Turner, Douglas H.. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.
[^{viii}], Banerjee, Aloke Raj; Turner, Douglas H.. The time dependence of chemical modification reveals slow steps in the folding of a group I ribozyme. Biochemistry (1995), 34(19), 6504-12.
[^{ix}], Zarrinkar, Patrick P.; Williamson, James R.. The P9.1-P9.2 peripheral extension helps guide folding of the Tetrahymena ribozyme. Nucleic Acids Res. (1996), 24(5), 854-8.
[^{x}], Strobel, Scott A.; Cech, Thomas R.. Minor groove recognition of the conserved G.cntdot.U pair at the Tetrahymena ribozyme reaction site. Science (Washington, D. C.) (1995), 267(5198), 675-9.
[^{xi}], Strobel, Scott A.; Cech, Thomas R.. Exocydic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.
[^{xii}], Sullenger, Bruce A.; Cech, Thomas R.. Ribozyme-mediated repair of defective mRNA by targeted trans-splicing. Nature (London) (1994), 371(6498), 619-22.
[^{xiii}], Robertson, H. D.; Altman, S.; Smith, J. D. J. Biol. Chem., 247, 5243-5251 (1972).
[^{xiv}], Forster, Anthony C.; Altman, Sidney. External guide sequences for an RNA enzyme. Science (Washington, D. C., 1883-) (1990), 249(4970), 783-6.
[^{xv}], Yuan, Y.; Hwang, E. S.; Altman, S. Targeted cleavage of mRNA by human RNase P. Proc. Natl. Acad. Sci. USA (1992) 89, 8006-10.
[^{xvi}], Harris, Michael E.; Pace, Norman R.. Identification of phosphates involved in catalysis by the ribozyme RNase P RNA. RNA (1995), 1(2), 210-18.
[^{xvii}], Pan, Tao; Loria, Andrew; Zhong, Kun. Probing of tertiary interactions in RNA: 2-hydroxyl-base contacts between the RNase P RNA and pre-tRNA. Proc. Natl. Acad. Sci. U.S.A. (1995), 92(26), 12510-14.
[^{xviii}], Pyle, Anna Marie; Green, Justin B.. Building a Kinetic Framework for Group II Intron Ribozyme Activity: Quantitation of Interdomain Binding and Reaction Rate. Biochemistry (1994), 33(9), 2716-25.
[^{xix}], Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 296-77.
[^{xx}], Zimmerly, Steven; Guo, Huatao; Eskes, Robert; Yang, Jian; Perlman, Philip S.; Lambowitz, Alan M.. A group II intron RNA is a catalytic component of a DNA endonuclease involved in intron mobility. Cell (Cambridge, Mass.) (1995), 83(4), 529-38.
[^{xxi}], Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′-hydroxyl groups. Chem. Biol. (1995), 2(11), 761-70.
[^{xxii}], Michel, Francois; Ferat, Jean Luc. Structure and activities of group II introns. Annu. Rev. Biochem. (1995), 64, 435-61.
[^{xxiii}], Abramovitz, Dana L.; Friedman, Richard A.; Pyle, Anna Marie. Catalytic role of 2′-hydroxyl groups within a group II intron active site. Science (Washington, D. C.) (1996), 271(5254), 1410-13.
[^{xxiv}], Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.
[^{xxv}], Guo, Hans C. T.; Collins, Richard A.. Efficient trans-cleavage of a stem-loop RNA substrate by a ribozyme derived from Neurospora VS RNA. EMBO J. (1995), 14(2), 368-76.
[^{xxvi}], Scott, W. G., Finch, J. T., Aaron, K. The crystal structure of an all RNA hammerhead ribozyme: A proposed mechanism for RNA catalytic cleavage. Cell, (1995), 81, 991-1002.
[^{xxvii}], McKay, Structure and function of the hammerhead ribozyme: an unfinished story. RNA, (1996), 2, 395-403.
[^{xxviii}], Long, D., Uhlenbeck, O., Hertel, K. Ligation with hammerhead ribozymes. U.S. Pat. No. 5,633,133.
[^{xxix}], Hertel, K. J., Herschlag, D., Uhlenbeck, O. A kinetic and thermodynamic framework for the hammerhead ribozyme reaction. Biochemistry, (1994) 33, 3374-3385. Beigelman, L., et al., Chemical modifications of hammerhead ribozymes. J. Biol. Chem., (1995) 270, 25702-25708.
[^{xxx}], Beigelman, L., et al, Chemical modifications of hammerhead ribozymes. J. Biol. Chem., (1995) 270, 25702-25708.
[^{xxxi}], Hampel, Arnold; Tritz, Richard; Hicks, Margaret; Cruz, Philip. ′Hairpin′ catalytic RNA model: evidence for helixes and sequence requirement for substrate RNA. Nucleic Acids Res. (1990), 18(2), 299-304.
[^{xxxii}], Chowrira, Bharat M.; Berzal-Herranz, Alfredo; Burke, John M.. Novel guanosine requirement for catalysis by the hairpin ribozyme. Nature (London) (1991), 354(6351), 320-2.
[^{xxxiii}], Berzal-Herranz, Alfredo; Joseph, Simpson; Chowrira, Bharat M.; Butcher, Samuel E.; Burke, John M.. Essential nucleotide sequences and secondary structure elements of the hairpin ribozyme. EMBO J.(1993), 12(6), 2567-73.
[^{xxxiv}], Joseph, Simpson; Berzal-Herranz, Alfredo; Chowrira, Bharat M.; Butcher, Samuel E.. Substrate selection rules for the hairpin ribozyme determined by in vitro selection, mutation, and analysis of mismatched substrates. Genes Dev. (1993), 7(1), 130-8.
[^{xxxv}], Berzal-Herranz, Alfredo; Joseph, Simpson; Burke, John M.. In vitro selection of active hairpin ribozymes by sequential RNA-catalyzed cleavage and ligation reactions. Genes Dev. (1992), 6(1), 129-34.
[^{xxxvi}], Hegg, Lisa A.; Fedor, Martha J.. Kinetics and Thermodynamics of Intermolecular Catalysis by Hairpin Ribozymes. Biochemistry (1995), 34(48), 15813-28.
[^{xxxvii}], Grasby, Jane A.; Mersmann, Karin; Singh, Mohinder; Gait, Michael I.. Purine Functional Groups in Essential Residues of the Hairpin Ribozyme Required for Catalytic Cleavage of RNA. Biochemistry (1995), 34(12), 4068-76.
[^{xxxviii}], Schmidt, Sabine; Beigelman, Leonid; Karpeisky, Alexander; Usman, Nassim; Sorensen, Ulrik S.; Gait, Michael J.. Base and sugar requirements for RNA cleavage of essential nucleoside residues in internal loop B of the hairpin ribozyme: implications for secondary structure. Nucleic Acids Res. (1996), 24(4), 573-81.
[^{xxxix}], Perrotta, Anne T.; Been, Michael D.. Cleavage of oligoribonucleotides by a ribozyme derived from the hepatitis .delta. virus RNA sequence. Biochemistry (1992), 31(1), 16-21.
[^{xl}], Perrotta, Anne T.; Been, Michael D.. A pseudoknot-like structure required for efficient self-cleavage of hepatitis delta virus RNA. Nature (London) (1991), 350(6317), 434-6.
[^{xli}], Puttaraju, M.; Perrotta, Anne T.; Been, Michael D.. A circular trans-acting hepatitis delta virus ribozyme. Nucleic Acids Res. (1993), 21(18), 4253-8.

TABLE II
A. 2.5 μmol Synthesis Cycle ABI 394 Instrument

| | | | | | |
|---|---|---|---|---|---|
| Reagent | Equivalents | Amount | Wait Time* DNA | Wait Time* 2′-O-methyl | Wait Time*RNA |

| | | | | | | | | |
|---|---|---|---|---|---|---|---|---|
| Phosphoramidites | 6.5 | 163 | μL | 45 sec | 2.5 | min | 7.5 | min |
| S-Ethyl Tetrazole | 23.8 | 238 | μL | 45 sec | 2.5 | min | 7.5 | min |
| Acetic Anhydride | 100 | 233 | μL | 5 sec | 5 | sec | 5 | sec |
| N-Methyl | 186 | 233 | μL | 5 sec | 5 | sec | 5 | sec |
| Imidazole | | | | | | | | |
| TCA | 176 | 2.3 | mL | 21 sec | 21 | sec | 21 | sec |
| Iodine | 11.2 | 1.7 | mL | 45 sec | 45 | sec | 45 | sec |
| Beaucage | 12.9 | 645 | μL | 100 sec | 300 | sec | 300 | sec |

| | | | | | | |
|---|---|---|---|---|---|---|
| Acetonitrile | NA | 6.67 | mL | NA | NA | NA |

B. 0.2 μmol Synthesis Cycle ABI 394 Instrument

| | | | | | |
|---|---|---|---|---|---|
| Reagent | Equivalents | Amount | Wait Time* DNA | Wait Time* 2′-O-methyl | Wait Time*RNA |

| | | | | | | | |
|---|---|---|---|---|---|---|---|
| Phosphoramidites | 15 | 31 | μL | 45 sec | 233 | sec | 465 sec |
| S-Ethyl Tetrazole | 38.7 | 31 | μL | 45 sec | 233 | min | 465 sec |
| Acetic Anhydride | 655 | 124 | μL | 5 sec | 5 | sec | 5 sec |
| N-Methyl | 1245 | 124 | μL | 5 sec | 5 | sec | 5 sec |
| Imidazole | | | | | | | |
| TCA | 700 | 732 | μL | 10 sec | 10 | sec | 10 sec |
| Iodine | 20.6 | 244 | μL | 15 sec | 15 | sec | 15 sec |
| Beaucage | 7.7 | 232 | μL | 100 sec | 300 | sec | 300 sec |

| | | | | | | |
|---|---|---|---|---|---|---|
| Acetonitrile | NA | 2.64 | mL | NA | NA | NA |

C. 0.2 μmol Synthesis Cycle 96 well Instrument

| | | | | | |
|---|---|---|---|---|---|
| | Equivalents: | | | | |
| | DNA/2′-O- | Amount: DNA/2′-O- | | Wait Time* 2′-O- | |
| Reagent | methyl/Ribo | methyl/Ribo | Wait Time* DNA | methyl | Wait Time* Ribo |

| | | | | | | | |
|---|---|---|---|---|---|---|---|
| Phosphoramidites | 22/33/66 | 40/60/120 | μL | 60 sec | 180 | sec | 360 sec |
| S-Ethyl Tetrazole | 70/105/210 | 40/60/120 | μL | 60 sec | 180 | min | 360 sec |
| Acetic Anhydride | 265/265/265 | 50/50/50 | μL | 10 sec | 10 | sec | 10 sec |
| N-Methyl | 502/502/502 | 50/50/50 | μL | 10 sec | 10 | sec | 10 sec |
| Imidazole | | | | | | | |
| TCA | 238/475/475 | 250/500/500 | μL | 15 sec | 15 | sec | 15 sec |
| Iodine | 6.8/6.8/6.8 | 80/80/80 | μL | 30 sec | 30 | sec | 30 sec |
| Beaucage | 34/51/51 | 80/120/120 | | 100 sec | 200 | sec | 200 sec |
| Acetonitrile | NA | 1150/1150/1150 | μL | NA | NA | | NA |

*Wait time does not include contact time during delivery.

TABLE III
Human REL-A Inozyme and Substrate Sequence

| | | Seq | | Seq |
|---|---|---|---|---|
| Pos | Substrate | ID | Inozyme | ID |
| 16 | GGCGGGGC C GGGUCGCA | 1 | UGCGACCC CUGAUGAGGCCGUUAGGCCGAA ICCCCGCC | 711 |
| 24 | CGGGUCGC A GCUGGGCC | 2 | GGCCCAGC CUGAUGAGGCCGUUAGGCCGAA ICCACCCG | 712 |
| 27 | GUCCCAGC U GGGCCCGC | 3 | GCGGGCCC CUGAUGAGGCCGUUAGGCCGAA ICUGCGAC | 713 |
| 32 | AGCUGGGC C CGCGGCAU | 4 | AUGCCGCG CUGAUGAGGCCGUUAGGCCGAA ICCCAGCU | 714 |
| 33 | GCUGGGCC C GCGGCAUG | 5 | CAUGCCGC CUGAUGAGGCCGUUAGGCCGAA IGCCCAGC | 715 |
| 39 | CCCGCGGC A UGGACGAA | 6 | UUCGUCCA CUGAUGAGGCCGUUAGGCCGAA ICCGCGGG | 716 |
| 49 | GGACGAAC U GUUCCCCC | 7 | GGGGGAAC CUGAUGAGGCCGUUAGGCCGAA IUUCGUCC | 717 |
| 54 | AACUGUUC C CCCUCAUC | 8 | GAUGAGGG CUGAUGAGGCCGUUAGGCCGAA IAACAGUU | 718 |
| 55 | ACUGUUCC C CCUCAUCU | 9 | AGAUGAGG CUGAUGAGGCCGUUAGGCCGAA IGAACAGU | 719 |
| 56 | CUGUUCCC C CUCAUCUU | 10 | AAGAUGAG CUGAUGAGGCCGUUAGGCCGAA IGGAACAG | 720 |
| 57 | UGUUCCCC C UCAUCUUC | 11 | GAAGAUGA CUGAUGAGGCCGUUAGGCCGAA IGGGAACA | 721 |
| 58 | GUUCCCCC U CAUCUUCC | 12 | GGAAGAUG CUGAUGAGGCCGUUAGGCCGAA IGGGGAAC | 722 |
| 60 | UCCCCCUC A UCUUCCCG | 13 | CCGGAAGA CUGAUGAGGCCGUUAGGCCGAA IAGGGGGA | 723 |
| 63 | COCUCAUC U UCCCGCCA | 14 | UGCCGGGA CUGAUGAGGCCGUUAGGCCGAA IAUGAGGG | 724 |
| 66 | UCAUCUUC C CGGCAGAG | 15 | CUCUGCCG CUGAUGAGGCCGUUAGGCCGAA IAAGAUGA | 725 |
| 67 | CAUCUUCC C GGCAGAGC | 16 | GCUCUGCC CUGAUGAGGCCGUUAGGCCGAA IGAAGAUG | 726 |
| 71 | UUCCCGGC A GAGCAGCC | 17 | GGCUGCUC CUGAUGAGGCCGUUAGGCCGAA ICCGGGAA | 727 |
| 76 | GGCAGAGC A GCCCAAGC | 18 | GCUUGGGC CUGAUGAGGCCGUUAGGCCGAA ICUCUGCC | 728 |
| 79 | AGAGCAGC C CAAGCAGC | 19 | GCUGCUUG CUGAUGAGGCCGUUAGGCCGAA ICUGCUCU | 729 |
| 80 | GAGCAGCC C AAGCAGCG | 20 | CGCUGCUU CUGAUGAGGCCGUUAGGCCGAA IGCUGCUC | 730 |
| 81 | AGCAGCCC A AGCAGCGG | 21 | CCGCUGCU CUGAUGAGGCCGUUAGGCCGAA IGGCUGCU | 731 |
| 85 | GCCCAAGC A GCGGGGCA | 22 | UGCCCCGC CUGAUGAGGCCGUUAGGCCGAA ICUUGGGC | 732 |
| 93 | AGCGGGGC A UGCGCUUC | 23 | GAAGCGCA CUGAUGAGGCCGUUAGGCCGAA ICCCCGCU | 733 |
| 99 | GCAUGCGC U UCCGCUAC | 24 | GUAGCGGA CUGAUGAGGCCGUUAGGCCGAA ICGCAUGC | 734 |
| 102 | UGCGCUUC C GCUACAAG | 25 | CUUGUAGC CUGAUGAGGCCGUUAGGCCGAA IAAGCGCA | 735 |
| 105 | GCUUCCGC U ACAAGUGC | 26 | GCACUUGU CUGAUGAGGCCGUUAGGCCGAA ICGGAAGC | 736 |
| 108 | UCCGCUAC A AGUGCGAG | 27 | CUCGCACU CUGAUGAGGCCGUUAGGCCGAA IUAGCGGA | 737 |
| 123 | AGGGGCGC U CCGCGGGC | 28 | GCCCGCGG CUGAUGAGGCCGUUAGGCCGAA ICGCCCCU | 738 |
| 125 | GGGCGCUC C GCGGGCAG | 29 | CUGCCCGC CUGAUGAGGCCGUUAGGCCGAA IAGCGCCC | 739 |
| 132 | CCCCGGGC A GCAUCCCA | 30 | UGGGAUGC CUGAUGAGGCCGUUAGGCCGAA ICCCGCGG | 740 |
| 135 | CGGGCAGC A UCCCAGGC | 31 | GCCUGGGA CUGAUGAGGCCGUUAGGCCGAA ICUGCCCG | 741 |
| 138 | GCAGCAUC C CAGGCGAG | 32 | CUCGCCUG CUGAUGAGGCCGUUAGGCCGAA IAUGCUGC | 742 |
| 139 | CAGCAUCC C AGGCGAGA | 33 | UCUCGCCU CUGAUGAGGCCGUUAGGCCGAA IGAUGCUG | 743 |
| 140 | AGCAUCCC A GGCGAGAG | 34 | CUCUCGCC CUGAUGAGGCCGUUAGGCCGAA IGGAUGCU | 744 |
| 153 | AGAGGAGC A CAGAUACC | 35 | GGUAUCUG CUGAUGAGGCCGUUAGGCCGAA ICUCCUCU | 745 |
| 155 | AGGAGCAC A GAUACCAC | 36 | GUGGUAUC CUGAUGAGGCCGUUAGGCCGAA IUGCUCCU | 746 |
| 161 | ACAGAUAC C ACCAAGAC | 37 | GUCUUGGU CUGAUGAGGCCGUUAGGCCGAA IUAUCUGU | 747 |
| 162 | CAGAUACC A CCAAGACC | 38 | GGUCUUGG CUGAUCAGGCCGUUAGGCCGAA IGUAUCUG | 748 |
| 164 | GAUACCAC C AAGACCCA | 39 | UGCCUCUU CUGAUGAGGCCGUUAGGCCGAA IUGGUAUC | 749 |
| 165 | AUACCACC A AGACCCAC | 40 | GUGGGUCU CUGAUGAGGCCGUUAGGCCGAA IGUGGUAU | 750 |
| 170 | ACCAAGAC C CACCCCAC | 41 | GUGGGGUG CUGAUGAGGCCGUUAGGCCGAA IUCUUGGU | 751 |
| 171 | CCAAGACC C ACCCCACC | 42 | GGUGGGGU CUGAUGAGGCCGUUAGGCCGAA ICUCUUGG | 752 |
| 172 | CAAGACCC A CCCCACCA | 43 | UGGUGGGG CUGAUGAGGCCGUUAGGCCGAA IGGUCUUG | 753 |
| 174 | AGACCCAC C CCACCAUC | 44 | GAUGGUGG CUGAUGAGGCCGUUAGGCCGAA IUGGGUCU | 754 |
| 175 | GACCCACC C CACCAUCA | 45 | UGAUGGUG CUGAUGAGGCCGUUAGGCCGAA IGUGGGUC | 755 |
| 176 | ACCCACCC C ACCAUCAA | 46 | UUGAUGGU CUGAUGAGGCCGUUAGGCCGAA IGGUGGGU | 756 |
| 177 | CCCACCCC A CCAUCAAG | 47 | CUUGAUGG CUGAUGAGGCCGUUAGGCCGAA IGGGUGGG | 757 |
| 179 | CACCCCAC C AUCAAGAU | 48 | AUCUUGAU CUGAUGAGGCCGUUAGGCCGAA IUGGGGUG | 758 |
| 180 | ACCCCACC A UCAAGAUC | 49 | GAUCUUGA CUGAUGAGGCCGUUAGGCCGAA IGUGCGGU | 759 |
| 183 | CCACCAUC A AGAUCAAU | 50 | AUUGAUCU CUGAUGAGGCCGUUAGGCCGAA IAUGGUGG | 760 |
| 189 | UCAAGAUC A AUGGCUAC | 51 | GUAGCCAU CUGAUGAGGCCGUUAGGCCGAA IAUCUUGA | 761 |
| 195 | UCAAUGGC U ACACAGGA | 52 | UCCUGUGU CUGAUGAGGCCGUUAGGCCGAA ICCAUUGA | 762 |
| 198 | AUGGCUAC A CAGGACCA | 53 | UGGUCCUG CUGAUGAGGCCGUUAGGCCGAA IUAGCCAU | 763 |
| 200 | GGCUACAC A GGACCAGG | 54 | CCUGGUCC CUGAUGAGGCCGUUAGGCCGAA IUGUAGCC | 764 |
| 205 | CACAGGAC C AGGGACAG | 55 | CUGUCCCU CUGAUGAGGCCGUUAGGCCGAA IUCCUGUG | 765 |
| 206 | ACAGGACC A GGGACAGU | 56 | ACUGUCCC CUGAUGAGGCCGUUAGGCCGAA IGUCCUGU | 766 |
| 212 | CCAGGGAC A GUGCGCAU | 57 | AUGCGCAC CUGAUGAGGCCGUUAGGCCGAA IUCCCUGG | 767 |
| 219 | CAGUGCGC A UCUCCCUG | 58 | CAGGGAGA CUGAUGAGGCCGUUAGGCCGAA ICGCACUG | 768 |
| 222 | UGCGCAUC U CCCUGGUC | 59 | GACCAGGG CUGAUGAGGCCGUUAGGCCGAA IAUGCGCA | 769 |
| 224 | CGCAUCUC C CUGGUCAC | 60 | GUGACCAG CUGAUGAGGCCGUUAGGCCGAA IAGAUGCG | 770 |
| 225 | GCAUCUCC C UGGUCACC | 61 | GGUGACCA CUGAUGAGGCCGUUAGGCCGAA IGAGAUGC | 771 |
| 226 | CAUCUCCC U GGUCACCA | 62 | UGGUGACC CUGAUGAGGCCGUUAGGCCGAA IGGAGAUG | 772 |
| 231 | CCCUGGUC A CCAAGGAC | 63 | GUCCUUGG CUGAUGAGGCCGUUAGGCCGAA IACCAGGG | 773 |
| 233 | CUGGUCAC C AAGGACCC | 64 | GGGUCCUU CUGAUGAGGCCGUUAGGCCGAA IUGACCAG | 774 |
| 234 | UGGUCACC A AGGACCCU | 65 | AGGGUCCU CUGAUGAGGCCGUUAGGCCGAA IGUGACCA | 775 |
| 240 | CCAAGGAC C CUCCUCAC | 66 | GUGAGGAG CUGAUGAGGCCGUUAGGCCGAA IUCCUUGG | 776 |
| 241 | CAAGGACC C UCCUCACC | 67 | GGUGAGGA CUGAUGAGGCCGUUAGGCCGAA IGUCCUUG | 777 |
| 242 | AAGGACCC U CCUCACCG | 68 | CGGUGAGG CUGAUGAGGCCGUUAGGCCGAA IGGUCCUU | 778 |
| 244 | GGACCCUC C UCACCGGC | 69 | GCCGGUGA CUGAUGAGGCCGUUAGGCCGAA IAGGGUCC | 779 |
| 245 | GACCCUCC U CACCGGCC | 70 | GGCCGGUG CUGAUGAGGCCGUUAGGCCGAA IGAGGGUC | 780 |
| 247 | CCCUCCUC A CCGGCCUC | 71 | GAGGCCGG CUGAUGAGGCCGUUAGGCCGAA IAGGAGGG | 781 |
| 249 | CUCCUCAC C GGCCUCAC | 72 | GUGAGGCC CUGAUGAGGCCGUUAGGCCGAA IUGAGGAG | 782 |
| 253 | UCACCGGC C UCACCCCC | 73 | GGGGGUGA CUGAUGAGGCCGUUAGGCCGAA ICCUGUGA | 783 |
| 254 | CACCGGCC U CACCCCCA | 74 | UGGGGGUG CUGAUGAGGCCGUUAGGCCGAA IGOOGGUG | 784 |
| 256 | CCGGCCUC A CCCCCACG | 75 | CGUGGGGG CUGAUGAGGCCGUUAGGCCGAA IAGGCCGG | 785 |
| 258 | GGCCUCAC C CCCACGAG | 76 | CUCGUGGG CUGAUGAGGCCGUUAGGCCGAA IUGAGGCC | 786 |
| 259 | GCCUCACC C CCACGAGC | 77 | GCUCGUGG CUGAUGAGGCCGUUAGGCCGAA IGUGAGGC | 787 |
| 260 | CCUCACCC C CACGAGCU | 78 | AGCUCGUG CUGAUGAGGCCGUUAGGCCGAA IGGUGAGG | 788 |
| 261 | CUCACCCC C ACGAGCUU | 79 | AAGCUCGU CUGAUGAGGCCGUUAGGCCGAA IGGGUGAG | 789 |
| 262 | UCACCCCC A CGAGCUUG | 80 | CAAGCUCG CUGAUGAGGCCGUUAGGCCGAA IGGGGUGA | 790 |
| 268 | CCACGAGC U UGUAGGAA | 81 | UUCCUACA CUGAUGAGGCCGUUAGGCCGAA ICUCGUGG | 791 |
| 282 | GAAAGGAC U GCCGGGAU | 82 | AUCCCGGC CUGAUGAGGCCGUUAGGCCGAA IUCCUUUC | 792 |
| 285 | AGGACUGC C GGGAUGGC | 83 | GCCAUCCC CUGAUGAGGCCGUUAGGCCGAA ICAGUCCU | 793 |
| 294 | GGGAUGGC U UCUAUGAG | 84 | CUCAUAGA CUGAUGAGGCCGUUAGGCCGAA ICCAUCCC | 794 |
| 297 | AUGGCUUC U AUGAGGCU | 85 | AGCCUCAU CUGAUGAGGCCGUUAGGCCGAA IAAGCCAU | 795 |
| 305 | UAUGAGGC U GAGCUCUG | 86 | CAGAGCUC CUGAUGAGGCCGUUAGGCCGAA ICCUCAUA | 796 |
| 310 | GGCUGAGC U CUGCCCGG | 87 | CCGGGCAG CUGAUGAGGCCGUUAGGCCGAA ICUCAGCC | 797 |
| 312 | CUGAGCUC U GCCCGGAC | 88 | GUCCGGGC CUGAUGAGGCCGUUAGGCCGAA IAGCUCAG | 798 |
| 315 | AGCUCUGC C CGGACCGC | 89 | GCGGUCCG CUGAUGAGGCCGUUAGGCCGAA ICAGAGCU | 799 |
| 316 | GCUCUGCC C GGACCGCU | 90 | AGCGGUCC CUGAUGAGGCCGUUAGGCCGAA IGCAGAGC | 800 |
| 321 | GCCCGGAC C GCUGCAUC | 91 | GAUGCAGC CUGAUGAGGCCGUUAGGCCGAA IUCCGGGC | 801 |
| 324 | CGGACCGC U GCAUCCAC | 92 | GUGGAUGC CUGAUGAGGCCGUUAGGCCGAA ICGGUCCG | 802 |
| 327 | ACCGCUGC A UCCACAGU | 93 | ACUGUGGA CUGAUGAGGCCGUUAGGCCGAA ICAGCGGU | 803 |
| 330 | GCUGCAUC C ACAGUUUC | 94 | GAAACUGU CUGAUGAGGCCGUUAGGCCGAA IAUGCAGC | 804 |
| 331 | CUGCAUCC A CAGUUUCC | 95 | GGAAACUG CUGAUGAGGCCGUUAGGCCGAA IGAUGCAG | 805 |
| 333 | UCAUCCAC A GUUUCCAG | 96 | CUGGAAAC CUGAUGAGGCCGUUAGGCCGAA IUGGAUGC | 806 |
| 339 | ACAGUUUC C AGAACCUG | 97 | CAGGUUCU CUGAUGAGGCCGUUAGGCCGAA IAAACUGU | 807 |
| 340 | CAGUUUCC A GAACCUGG | 98 | CCAGGUUC CUGAUGAGGCCGUUAGGCCGAA IGAAACUG | 808 |
| 345 | UCCAGAAC C UGGGAAUC | 99 | GAUUCCCA CUGAUGAGGCCGUUAGGCCGAA IUUCUGGA | 809 |
| 346 | CCAGAACC U GGGAAUCC | 100 | GGAUUCCC CUGAUGAGGCCGUUAGGCCGAA IGUUCUGG | 810 |
| 354 | UGGGAAUC C AGUGUGUG | 101 | CACACACU CUGAUGAGGCCGUUAGGCCGAA IAUUCCCA | 811 |
| 355 | GGGAAUCC A GUGUGUGA | 102 | UCACACAC CUGAUGAGGCCGUUAGGCCGAA IGAUUCCC | 812 |
| 375 | AGCGGGAC C UGGAGCAG | 103 | CUGCUCCA CUGAUGAGGCCGUUAGGCCGAA IUCCCGCU | 813 |
| 376 | GCGGGACC U GGAGCAGG | 104 | CCUGCUCC CUGAUGAGGCCGUUAGGCCGAA IGUCCCGC | 814 |
| 382 | CCUGGAGC A GGCUAUCA | 105 | UGAUAGCC CUGAUGAGGCCGUUAGGCCGAA ICUCCAGG | 815 |
| 386 | GAGCAGGC U AUCAGUCA | 106 | UGACUGAU CUGAUGAGGCCGUUAGGCCGAA ICCUGCUC | 816 |
| 390 | AGGCUAUC A GUCAGCGC | 107 | GCGCUGAC CUGAUGAGGCCGUUAGGCCGAA IAUAGCCU | 817 |
| 394 | UAUCAGUC A GCGCAUCC | 108 | GGAUGCGC CUGAUGAGGCCGUUAGGCCGAA IACUGAUA | 818 |
| 399 | GUCAGCGC A UCCAGACC | 109 | GGUCUGGA CUGAUGAGGCCGUUAGGCCGAA ICGCUGAC | 819 |
| 402 | AGCGCAUC C AGACCAAC | 110 | GUUGGUCU CUGAUGAGGCCGUUAGGCCGAA IAUGCGCU | 820 |
| 403 | GCGCAUCC A GACCAACA | 111 | UGUUGGUC CUGAUGAGGCCGUUAGGCCGAA IGAUGCGC | 821 |
| 407 | AUCCAGAC C AACAACAA | 112 | UUGUUGUU CUGAUGAGGCCGUUAGGCCGAA IUCUGGAU | 822 |
| 408 | UCCAGACC A ACAACAAC | 113 | GUUGUUGU CUGAUGAGGCCGUUAGGCCGAA IGUCUGGA | 823 |
| 411 | AGACCAAC A ACAACCCC | 114 | GGGGUUGU CUGAUGAGGCCGUUAGGCCGAA IUUGGUCU | 824 |
| 414 | CCAACAAC A ACCCCUUC | 115 | GAAGGGGU CUGAUGAGGCCGUUAGGCCGAA IUUGUUGG | 825 |
| 417 | ACAACAAC C CCUUCCAA | 116 | UUGGAAGG CUGAUGAGGCCGUUAGGCCGAA IUUGUUGU | 826 |
| 418 | CAACAACC C CUUCCAAG | 117 | CUUGGAAG CUGAUGAGGCCGUUAGGCCGAA IGUUGUUG | 827 |
| 419 | AACAACCC C UUCCAAGU | 118 | ACUUGGAA CUGAUGAGGCCGUUAGGCCGAA IGGUUGUU | 828 |
| 420 | ACAACCCC U UCCAAGUU | 119 | AACUUGGA CUGAUGAGGCCGUUAGGCCGAA IGGGUUGU | 829 |
| 423 | ACCCCUUC C AAGUUCCU | 120 | AGGAACUU CUGAUGAGGCCGUUAGGCCGAA IAAGGGGU | 830 |
| 424 | CCCCUUCC A AGUUCCUA | 121 | UAGGAACU CUGAUGAGGCCGUUAGGCCGAA IGAAGGGG | 831 |
| 430 | CCAAGUUC C UAUAGAAG | 122 | CUUCUAUA CUGAUGAGGCCGUUAGGCCGAA IAACUUGG | 832 |
| 431 | CAAGUUCC U AUAGAAGA | 123 | UCUUCUAU CUGAUGAGGCCGUUAGGCCGAA IGAACUUG | 833 |
| 442 | AGAAGAGC A GCGUGGGG | 124 | CCCCACGC CUGAUGAGGCCGUUAGGCCGAA ICUCUUCU | 834 |
| 453 | GUGGGGAC U ACGACCUG | 125 | CAGGUCGU CUGAUGAGGCCGUUAGGCCGAA IUCCCCAC | 835 |
| 459 | ACUACGAC C UGAAUGCU | 126 | AGCAUUCA CUGAUGAGGCCGUUAGGCCGAA IUCGUAGU | 836 |
| 460 | CUACGACC U GAAUGCUG | 127 | CAGCAUUC CUGAUGAGGCCGUUAGGCCGAA IGUCGUAG | 837 |
| 467 | CUGAAUGC U GUGCGGCU | 128 | AGCCGCAC CUGAUGAGGCCGUUAGGCCGAA ICAUUCAG | 838 |
| 475 | UGUGCGGC U CUGCUUCC | 129 | GGAAGCAG CUGAUGAGGCCGUUAGGCCGAA ICCGCACA | 839 |
| 477 | UGCGGCUC U GCUUCCAG | 130 | CUGGAAGC CUGAUGAGGCCGUUAGGCCGAA IAGCCGCA | 840 |
| 480 | GGCUCUGC U UCCAGGUG | 131 | CACCUGGA CUGAUGAGGCCGUUAGGCCGAA ICAGAGCC | 841 |
| 483 | UCUGCUUC C AGGUGACA | 132 | UGUCACCU CUGAUGAGGCCGUUAGGCCGAA IAAGCAGA | 842 |
| 484 | CUGCUUCC A GGUGACAG | 133 | CUGUCACC CUGAUGAGGCCGUUAGGCCGAA IGAAGCAG | 843 |
| 491 | CAGGUGAC A GUGCGGGA | 134 | UCCCGCAC CUGAUGAGGCCGUUAGGCCGAA IUCACCUG | 844 |
| 501 | UGCGGGAC C CAUCAGGC | 135 | GCCUGAUG CUGAUGAGGCCGUUAGGCCGAA IUCCCGCA | 845 |
| 502 | GCGGGACC C AUCAGGCA | 136 | UGCCUGAU CUGAUGAGGCCGUUAGGCCGAA IGUCCCGC | 846 |
| 503 | CGGGACCC A UCAGGCAG | 137 | CUGCCUGA CUGAUGAGGCCGUUAGGCCGAA IGGUCCCG | 847 |
| 506 | GACCCAUC A GGCAGGCC | 138 | GGCCUGCC CUGAUGAGGCCGUUAGGCCGAA IAUGGGUC | 848 |
| 510 | CAUCAGGC A GGCCCCUC | 139 | GAGGGGCC CUGAUGAGGCCGUUAGGCCGAA ICCUGAUG | 849 |
| 514 | AGGCAGGC C CCUCCGCC | 140 | GGCGGAGG CUGAUGAGGCCGUUAGGCCGAA ICCUGCCU | 850 |
| 515 | GGCAGGCC C CUCCGCCU | 141 | AGGCGGAG CUGAUGAGGCCGUUAGGCCGAA IGCCUGCC | 851 |
| 516 | GCAGGCCC C UCCGCCUG | 142 | CAGGCGGA CUGAUGAGGCCGUUAGGCCGAA IGGCCUGC | 852 |
| 517 | CAGGCCCC U CCGCCUGC | 143 | GCAGGCGG CUGAUGAGGCCGUUAGGCCGAA IGGGCCUG | 853 |
| 519 | GGCCCCUC C GCCUGCCG | 144 | CGGCAGGC CUGAUGAGGCCGUUAGGCCGAA IAGGGGCC | 854 |
| 522 | CCCUCCGC C UGCCGCCU | 145 | AGGCGGCA CUGAUGAGGCCGUUAGGCCGAA ICGGAGGG | 855 |
| 523 | CCUCCGCC U GCCGCCUG | 146 | CAGGCGGC CUGAUGAGGCCGUUAGGCCGAA IGCGGAGG | 856 |
| 526 | CCGCCUGC C GCCUGUCC | 147 | GGACAGGC CUGAUGAGGCCGUUAGGCCGAA ICAGGCGG | 857 |
| 529 | CCUGCCGC C UGUCCUUU | 148 | AAAGGACA CUGAUGAGGCCGUUAGGCCGAA ICGGCAGG | 858 |
| 530 | CUGCCGCC U GUCCUUUC | 149 | GAAAGGAC CUGAUGAGGCCGUUAGGCCGAA IGCGGCAG | 859 |
| 534 | CGCCUGUC C UUUCUCAU | 150 | AUGAGAAA CUGAUGAGGCCGUUAGGCCGAA IACAGGCG | 860 |
| 535 | GCCUGUCC U UUCUCAUC | 151 | GAUGAGAA CUGAUGAGGCCGUUAGGCCGAA IGACAGGC | 861 |
| 539 | GUCCUUUC U CAUCCCAU | 152 | AUGGGAUG CUGAUGAGGCCGUUAGGCCGAA IAAAGGAC | 862 |
| 541 | CCUUUCUC A UCCCAUCU | 153 | AGAUGGGA CUGAUGAGGCCGUUAGGCCGAA IAGAAAGG | 863 |
| 544 | UUCUCAUC C CAUCUUUG | 154 | CAAAGAUG CUGAUGAGGCCGUUAGGCCGAA IAUGACAA | 864 |
| 545 | UCUCAUCC C AUCUUUGA | 155 | UCAAAGAU CUGAUGAGGCCGUUAGGCCGAP IGAUGAGA | 865 |
| 546 | CUCAUCCC A UCUUUGAC | 156 | GUCAAAGA CUGAUGAGGCCGUUAGGCCGAA IGGAUGAG | 866 |
| 549 | AUCCCAUC U UUGACAAU | 157 | AUUGUCAA CUGAUGAGGCCGUUAGGCCGAA IAUGGGAU | 867 |
| 555 | UCUUUGAC A AUCGUGCC | 158 | GGCACGAU CUGAUGAGGCCGUUAGGCCGAA IUCAAAGA | 868 |
| 563 | AAUCGUGC C CCCAACAC | 159 | GUGUUGGG CUGAUGAGGCCGUUAGGCCGAA ICACGAUU | 869 |
| 564 | AUCGUGCC C CCAACACU | 160 | AGUGUUGG CUGAUGAGGCCGUUAGGCCGAA IGCACGAU | 870 |
| 565 | UCGUGCCC C CAACACUG | 161 | CAGUGUUG CUGAUGAGGCCGUUAGGCCGAA IGGCACGA | 871 |
| 566 | CGUGCCCC C AACACUGC | 162 | GCAGUGUU CUGAUGAGGCCGUUAGGCCGAA IGGGCACG | 872 |
| 567 | GUGCCCCC A ACACUGCC | 163 | GGCAGUGU CUGAUGAGGCCGUUAGGCCGAA IGGGGCAC | 873 |
| 570 | CCCCCAAC A CUGCCGAG | 164 | CUCGGCAG CUGAUGAGGCCGUUAGGCCGAA IUUGGGGG | 874 |
| 572 | CCCAACAC U GCCGAGCU | 165 | AGCUCGGC CUGAUGAGGCCGUUAGGCCGAA IUGUUGGG | 875 |
| 575 | AACACUGC C GAGCUCAA | 166 | UUGAGCUC CUGAUGAGGCCGUUAGGCCGAA ICAGUGUU | 876 |
| 580 | UGCCGAGC U CAAGAUCU | 167 | AGAUCUUG CUGAUGAGGCCGUUAGGCCGAA ICUCGGCA | 877 |
| 582 | CCGAGCUC A AGAUCUGC | 168 | GCAGAUCU CUGAUGAGGCCGUUAGGCCGAA IAGCUCGG | 878 |
| 588 | UCAAGAUC U GCCGAGUG | 169 | CACUCGGC CUGAUGAGGCCGUUAGGCCGAA IAUCUUGA | 879 |
| 591 | AGAUCUGC C GAGUGAAC | 170 | GUUCACUC CUGAUGAGGCCGUUAGGCCGAA ICAGAUCU | 880 |
| 600 | GAGUGAAC C GAAACUCU | 171 | AGAGUUUC CUGAUGAGGCCGUUAGGCCGAA IUUCACUC | 881 |
| 606 | ACCGAAAC U CUGGCAGC | 172 | GCUGCCAG CUGAUGAGGCCGUUAGGCCGAA IUUUCGGU | 882 |
| 608 | CGAAACUC U GGCAGCUG | 173 | CAGCUGCC CUGAUGAGGCCGUUAGGCCGAA IAGUUUCG | 883 |
| 612 | ACUCUGGC A GCUGCCUC | 174 | GAGGCAGC CUGAUGAGGCCGUUAGGCCGAA ICCAGAGU | 884 |
| 615 | CUGGCAGC U GCCUCGGU | 175 | ACCGAGGC CUGAUGAGGCCGUUAGGCCGAA ICUGCCAG | 885 |
| 618 | GCAGCUGC C UCGGUGGG | 176 | CCCACCGA CUGAUGAGGCCGUUAGGCCGAA ICAGCUGC | 886 |
| 619 | CAGCUGCC U CGGUGGGG | 177 | CCCCACCG CUGAUGAGGCCGUUAGGCCGAA IGCAGCUG | 887 |
| 636 | AUGAGAUC U UCCUACUG | 178 | CAGUAGGA CUGAUGAGGCCGUUAGGCCGAA IAUCUCAU | 888 |
| 639 | AGAUCUUC C UACUGUGU | 179 | ACACAGUA CUGAUGAGGCCGUUAGGCCGAA IAAGAUCU | 889 |
| 640 | GAUCUUCC U ACUGUGUG | 180 | CACACAGU CUGAUGAGGCCGUUAGGCCGAA IGAAGAUC | 890 |
| 643 | CUUCCUAC U GUGUGACA | 181 | UGUCACAC CUGAUGAGGCCGUUAGGCCGAA IUAGGAAG | 891 |
| 651 | UGUGUGAC A AGGUGCAG | 182 | CUGCACCU CUGAUGAGGCCGUUAGGCCGAA IUCACACA | 892 |
| 658 | CAAGGUGC A GAAAGAGG | 183 | CCUCUUUC CUGAUGAGGCCGUUAGGCCGAA ICACCUUG | 893 |
| 669 | AAGAGGAC A UUGAGGUG | 184 | CACCUCAA CUGAUGAGGCCGUUAGGCCGAA IUCCUCUU | 894 |
| 684 | UGUAUUUC A CGGGACCA | 185 | UGGUCCCG CUGAUGAGGCCGUUAGGCCGAA IAAAUACA | 895 |
| 691 | CACGGGAC C AGGCUGGG | 186 | CCCAGCCU CUGAUGAGGCCGUUAGGCCGAA IUCCCGUG | 896 |
| 692 | ACGGGACC A GGCUGGGA | 187 | UCCCAGCC CUGAUGAGGCCGUUAGGCCGAA IGUCCCGU | 897 |
| 696 | GACCAGGC U GGGAGGCC | 188 | GGCCUCCC CUGAUGAGGCCGUUAGGCCGAA ICCUGGUC | 898 |
| 704 | UGGGAGGC C CGAGGCUC | 189 | GAGCCUCG CUGAUGAGGCCGUUAGGCCGAA ICCUCCCA | 899 |
| 705 | GGGAGGCC C GAGGCUCC | 190 | GGAGCCUC CUGAUGAGGCCGUUAGGCCGAA IGCCUCCC | 900 |
| 711 | CCCGAGGC U CCUUUUCG | 191 | CGAAAAGG CUGAUGAGGCCGUUAGGCCGAA ICCUCGGG | 901 |
| 713 | CGAGGCUC C UUUUCGCA | 192 | UGCGAAAA CUGAUGAGGCCGUUAGGCCGAA IAGCCUCG | 902 |
| 714 | GAGGCUCC U UUUCGCAA | 193 | UUGCGAAA CUGAUGAGGCCGUUAGGCCGAA IGAGCCUC | 903 |
| 721 | CUUUUCGC A AGCUGAUG | 194 | CAUCAGCU CUGAUGAGGCCGUUAGGCCGAA ICGAAAAG | 904 |
| 725 | UCGCAAGC U GAUGUGCA | 195 | UGCACAUC CUGAUGAGGCCGUUAGGCCGAA ICUUGCGA | 905 |
| 733 | UGAUGUOC A CCGACAAG | 196 | CUUGUCGG CUGAUGAGGCCGUUAGGCCGAA ICACAUCA | 906 |
| 735 | AUGUGCAC C GACAAGUG | 197 | CACUUGUC CUGAUGAGGCCGUUAGGCCGAA IUGCACAU | 907 |
| 739 | GCACCGAC A AGUGGCCA | 198 | UGGCCACU CUGAUGAGGCCGUUAGGCCGAA IUCGGUGC | 908 |
| 746 | CAAGUGGC C AUUGUGUU | 199 | AACACAAU CUGAUGAGGCCGUUAGGCCGAA ICCACUUG | 909 |
| 747 | AAGUGGCC A UUGUGUUC | 200 | GAACACAA CUGAUGAGGCCGUUAGGCCGAA IGCCACUU | 910 |
| 756 | UUGUGUUC C GGACCCCU | 201 | ACGGGUCC CUGAUGAGGCCGUUAGGCCGAA IAACACAA | 911 |
| 761 | UUCCGGAC C CCUCCCUA | 202 | UAGGGAGG CUGAUGAGGCCGUUAGGCCGAA IUCCGGAA | 912 |
| 762 | UCCGGACC C CUCCCUAC | 203 | GUAGGGAG CUGAUGAGGCCGUUAGGCCGAA IGUCCGGA | 913 |
| 763 | CCGGACCC C UCCCUACG | 204 | CGUAGGGA CUGAUGAGGCCGUUAGGCCGAA ICGUCCGG | 914 |
| 764 | CGGACCCC U CCCUACGC | 205 | GCGUAGGG CUGAUGAGGCCGUUAGGCCGAA IGGGLICCG | 915 |
| 766 | GACCCCUC C CUACGCAG | 206 | CUGCGUAG CUGAUGAGGCCGUUAGGCCGAA IAGGGGUC | 916 |
| 767 | ACCCCUCC C UACGCAGA | 207 | UCUGCGUA CUGAUGAGGCCGUUAGGCCGAA IGAGGGGU | 917 |
| 768 | CCCCUCCC U ACGCAGAC | 208 | GUCUGCGU CUGAUGAGGCCGUUAGGCCGAA IGGAGGGG | 918 |
| 773 | CCCUACGC A GACCCCAG | 209 | CUGGGGUC CUGAUGAGGCCGUUAGGCCGAA ICGUAGGG | 919 |
| 777 | ACGCAGAC C CCAGCCUG | 210 | CAGGCUGG CUGAUGAGGCCGUUAGGCCGAA IUCUGCGU | 920 |
| 778 | CGCAGACC C CAGCCUGC | 211 | GCAGGCUG CUGAUGAGGCCGUUAGGCCGAA IGUCUGCG | 921 |
| 779 | GCAGACCC C AGCCUGCA | 212 | UGCAGGCU CUGAUGAGGCCGUUAGGCCGAA IGGUCUGC | 922 |
| 780 | CAGACCCC A GCCUGCAG | 213 | CUGCAGGC CUGAUCAGGCCGUUAGGCCGAA IGGGUCUG | 923 |
| 783 | ACCCCAGC C UGCAGGCU | 214 | AGCCUGCA CUGAUGAGGCCGUUAGGCCGAA ICUGGGGU | 924 |
| 784 | CCCCAGCC U GCAGGCUC | 215 | GAGCCUGC CUGAUGAGGCCGUUAGGCCGAA IGCUGGGG | 925 |
| 787 | CAGCCUGC A GCCUCCUG | 216 | CAGGAGCC CUGAUGAGGCCGUUAGGCCGAA ICAGGCUG | 926 |
| 791 | CUGCACGC U CCUGUGCG | 217 | CGCACAGG CUGAUGAGGCCGUUAGGCCGAA ICCUGCAG | 927 |
| 793 | GCAGGCUC C UGUGCGUG | 218 | CACGCACA CUGAUGAGGCCGUUAGGCCGAA IAGCCUGC | 928 |
| 794 | CAGGCUCC U GUGCGUGU | 219 | ACACGCAC CUGAUGAGGCCGUUAGGCCGAA IGAGCCUG | 929 |
| 804 | UGCGUGUC U CCAUGCAG | 220 | CUGCAUGG CUGAUGAGGCCGUUAGGCCGAA IACACGCA | 930 |
| 806 | CGUGUCUC C AUGCAGCU | 221 | AGCUGCAU CUGAUGAGGCCGUUAGGCCGAA IAGACACG | 931 |
| 807 | GUGUCUCC A UGCAGCUG | 222 | CAGCUGCA CUGAUGAGGCCGUUAGGCCGAA IGAGACAC | 932 |
| 811 | CUCCAUGC A GCUGCGGC | 223 | GCCGCAGC CUGAUGAGGCCGUUAGGCCGAA ICAUGGAG | 933 |
| 814 | CAUGCAGC U GCGGCGGC | 224 | GCCGCCGC CUGAUGAGGCCGUUAGGCCGAA ICUGCAUG | 934 |
| 823 | GCGGCGGC C UUCCGACC | 225 | GGUCGGAA CUGAUGAGGCCGUUAGGCCGAA ICCGCCGC | 935 |
| 824 | CGGCGGCC U UCCGACCG | 226 | CGGUCGGA CUGAUGAGGCCGUUAGGCCGAA IGCCGCCG | 936 |
| 827 | CGGCCUUC C GACCGGGA | 227 | UCCCGGUC CUGAUGAGGCCGUUAGGCCGAA IAAGGCCG | 937 |
| 831 | CUUCCGAC C GGGAGCUC | 228 | GAGCUCCC CUGAUGAGGCCGUUAGGCCGAA IUCGGAAG | 938 |
| 838 | CCGGGAGC U CAGUGAGC | 229 | GCUCACUG CUGAUGAGGCCGUUAGGCCGAA ICUCCCGG | 939 |
| 840 | GGGAGCUC A GUGAGCCC | 230 | GGGCUCAC CUGAUGAGGCCGUUAGGCCGAA IAGCUCCC | 940 |
| 847 | CAGUGAGC C CAUGGAAU | 231 | AUUCCAUG CUGAUGAGGCCGUUAGGCCGAA ICUCACUG | 941 |
| 848 | AGUGAGCC C AUGGAAUU | 232 | AAUUCCAU CUGAUGAGGCCGUUAGGCCGAA IGCUCACU | 942 |
| 849 | GUGAGCCC A UGGAAUUC | 233 | GAAUUCCA CUGAUGAGGCCGUUAGGCCGAA IGGCUCAC | 943 |
| 858 | UGGAAUUC C AGUACCUG | 234 | CAGGUACU CUGAUGAGGCCGUUAGGCCGAA IAAUUCCA | 944 |
| 859 | GGAAUUCC A GUACCUGC | 235 | GCAGGUAC CUGAUGAGGCCGUUAGGCCGAA IGAAUUCC | 945 |
| 864 | UCCAGUAC C UGCCAGAU | 236 | AUCUGGCA CUGAUGAGGCCGUUAGGCCGAA IUACUGGA | 946 |
| 865 | CCAGUACC U GCCAGAUA | 237 | UAUCUGGC CUGAUGAGGCCGUUAGGCCGAA IGUACUGG | 947 |
| 868 | GUACCUGC C AGAUACAG | 238 | CUGUAUCU CUGAUGGAGCCGUUAGGCCGAA ICAGGUAC | 948 |
| 869 | UACCUGCC A GAUACAGA | 239 | UCUGUAUC CUGAUGAGGCCGUUAGGCCGAA IGCAGGUA | 949 |
| 875 | CCAGAUAC A GACGAUCG | 240 | CGAUCGUC CUGAUGAGGCCGUUAGGCCGAA IUAUCUGG | 950 |
| 886 | CGAUCGUC A CCGGAUUG | 241 | CAAUCCGG CUGAUGAGGCCGUUAGGCCGAA IACGAUCG | 951 |
| 888 | AUCGUCAC C GGAUUGAG | 242 | CUCAAUCC CUGAUGAGGCCGUUAGGCCGAA IUGACGAU | 952 |
| 914 | AAAAGGAC A UAUGAGAC | 243 | GUCUCAUA CUGAUGAGGCCGUUAGGCCGAA IUCCUUUU | 953 |
| 923 | UAUGAGAC C UUAAAGAG | 244 | CUCUUGAA CUGAUGAGGCCGUUAGGCCGAA IUCUCAUA | 954 |
| 924 | AUGAGACC U UCAAGAGC | 245 | GCUCUUGA CUGAUGAGGCCGUUAGGCCGAA IGUCUCAU | 955 |
| 927 | AGACCUUC A AGAGCAUC | 246 | GAUGCUCU CUGAUGAGGCCGUUAGGCCGAA IAAGGUCU | 956 |
| 933 | UCAAGAGC A UCAUGAAG | 247 | CUUCAUGA CUGAUGAGGCCGUUAGGCCGAA ICUCUUGA | 957 |
| 936 | AGAGCAUC A UGAAGAAG | 248 | CUUCUUCA CUGAUGAGGCCGUUAGGCCGAA IAUGCUCU | 958 |
| 949 | GAAGAGUC C UUUCAGCG | 249 | CGCUGAAA CUGAUGAGGCCGUUAGGCCGAA IACUCUUC | 959 |
| 950 | AAGAGUCC U UUCAGCGG | 250 | CCGCUGAA CUGAUGAGGCCGUUAGGCCGAA IGACUCUU | 960 |
| 954 | GUCCUUUC A GCGGACCC | 251 | GGGUCCGC CUGAUGAGGCCGUUAGGCCGAA IAAAGGAC | 961 |
| 961 | CAGCGGAC C CACCGACC | 252 | GGUCGGUG CUGAUGAGGCCGUUAGGCCGAA IUCCGCUG | 962 |
| 962 | AGCGGACC C ACCGACCC | 253 | GGGUCGGU CUGAUGAGGCCGUUAGGCCGAA IGUCCGCU | 963 |
| 963 | GCGGACCC A CCGACCCC | 254 | GGGGUCGG CUGAUGAGGCCGUUAGGCCGAA IGGUCCGC | 964 |
| 965 | GGACCCAC C GACCCCCG | 255 | CGGGGGUC CUGAUGAGGCCGUUAGGCCGAA IUGGGUCC | 965 |
| 969 | CCACCGAC C CCCGGCCU | 256 | AGGCCGGG CUGAUGAGGCCGUUAGGCCGAA IUCGGUGG | 966 |
| 970 | CACCGACC C CCGGCCUC | 257 | GAGGCCGG CUGAUGAGGCCGUUAGGCCGAA IGUCGGUG | 967 |
| 971 | ACCGACCC C CGGCCUCC | 258 | GGAGGCCG CUGAUGAGGCCGUUAGGCCGAA IGGUCGGU | 968 |
| 972 | CCGACCCC C GGCCUCCA | 259 | UGGAGGCC CUGAUGAGGCCGUUAGGCCGAA IGGGUCGG | 969 |
| 976 | CCCCCGGC C UCCACCUC | 260 | GAGGUGGA CUGAUGAGGCCGUUAGGCCGAA ICCGGGGG | 970 |
| 977 | CCCCGGCC U CCACCUCG | 261 | CGAGGUGG CUGAUGAGGCCGUUAGGCCGAA IGCCGGGG | 971 |
| 979 | CCGGCCUC C ACCUCGAC | 262 | GUCGAGGU CUGAUGAGGCCGUUAGGCCGAA IAGGCCGG | 972 |
| 980 | CGGCCUCC A CCUCGACG | 263 | CGUCGAGG CUGAUGAGGCCGUUAGGCCGAA IGAGGCCG | 973 |
| 982 | GCCUCCAC C UCGACGCA | 264 | UGCGUCGA CUGAUGAGGCCGUUAGGCCGAA IUGGAGGC | 974 |
| 983 | CCUCCACC U CGACGCAU | 265 | AUGCGUCG CUGAUGAGGCCGUUAGGCCGAA IGUGGAGG | 975 |
| 990 | CUCGACGC A UUGCUGUG | 266 | CACAGCAA CUGAUGAGGCCGUUAGGCCGAA ICGUCGAG | 976 |
| 995 | CGCAUUGC U GUGCCUUC | 267 | GAAGGCAC CUGAUGAGGCCGUUAGGCCGAA ICAAUGCG | 977 |
| 1000 | UGCUGUGC C UUCCCGCA | 268 | UGCGGGAA CUGAUGAGGCCGUUAGGCCGAA ICACAGCA | 978 |
| 1001 | GCUGUGCC U UCCCGCAG | 269 | CUGCGGGA CUGAUGAGGCCGUUAGGCCGAA IGCACAGC | 979 |
| 1004 | GUGCCUUC C CGCAGCUC | 270 | GAGCUGCG CUGAUGAGGCCGUUAGGCCGAA IAAGGCAC | 980 |
| 1005 | UGCCUUCC C GCAGCUCA | 271 | UGAGCUGC CUGAUGAGGCCGUUAGGCCGAA IGAAGGCA | 981 |
| 1008 | CUUCCCGC A GCUCAGCU | 272 | AGCUGAGC CUGAUGAGGCCGUUAGGCCGAA ICGGGAAG | 982 |
| 1011 | CCCGCAGC U CAGCUUCU | 273 | AGAAGCUG CUGAUGAGGCCGUUAGGCCGAA ICUGCGGG | 983 |
| 1013 | CGCAGCUC A GCUUCUGU | 274 | ACAGAAGC CUGAUGAGGCCGUUAGGCCGAA IAGCUGCG | 984 |
| 1016 | AGCUCAGC U UCUGUCCC | 275 | GGGACAGA CUGAUGAGGCCGUUAGGCCGAA ICUGAGCU | 985 |
| 1019 | UCAGCUUC U GUCCCCAA | 276 | UUGGGGAC CUGAUGAGGCCGUUAGGCCGAA IAAGCUGA | 986 |
| 1023 | CUUCUGUC C CCAAGCCA | 277 | UGGCUUGG CUGAUGAGGCCGUUAGGCCGAA IACAGAAG | 987 |
| 1024 | UUCUGUCC C CAAGCCAG | 278 | CUGGCUUG CUGAUGAGGCCGUUAGGCCGAA IGACAGAA | 988 |
| 1025 | UCUGUCCC C AAGCCAGC | 279 | GCUGGCUU CUGAUGAGGCCGUUAGGCCGAA IGGACAGA | 989 |
| 1026 | CUGUCCCC A AGCCAGCA | 280 | UGCUGGCU CUGAUGAGGCCGUUAGGCCGAA IGGGACAG | 990 |
| 1030 | CCCCAAGC C AGCACCCC | 281 | GGGGUGCU CUGAUGAGGCCGUUAGGCCGAA ICUUGGGG | 991 |
| 1031 | CCCAAGCC A GCACCCCA | 282 | UGGGGUGC CUGAUGAGGCCGUUAGGCCGAA IGCUUGGG | 992 |
| 1034 | AAGCCAGC A CCCCAGCC | 283 | GGCUGGGG CUGAUGAGGCCGUUAGGCCGAA ICUGGCUU | 993 |
| 1036 | GCCAGCAC C CCAGCCCU | 284 | AGGGCUGG CUGAUGAGGCCGUUAGGCCGAA IUGCUGGC | 994 |
| 1037 | CCAGCACC C CAGCCCUA | 285 | UAGGGCUG CUGAUGAGGCCGUUAGGCCGAA IGUGCUGG | 995 |
| 1038 | CAGCACCC C AGCCCUAU | 286 | AUAGGGCU CUGAUGAGGCCGUUAGGCCGAA IGGUGCUG | 996 |
| 1039 | AGCACCCC A GCCCUAUC | 287 | GAUAGGGC CUGAUGAGGCCGUUAGGCCGAA IGGGUGCU | 997 |
| 1042 | ACCCCAGC C CUAUCCCU | 288 | AGGGAUAG CUGAUGAGGCCGUUAGGCCGAA ICUGGGGU | 998 |
| 1043 | CCCCAGCC C UAUCCCUU | 289 | AAGGGAUA CUGAUGAGGCCGUUAGGCCGAA IGCUGGGG | 999 |
| 1044 | CCCAGCCC U AUCCCUUU | 290 | AAAGGGAU CUGAUGAGGCCGUUAGGCCGAA IGGCUGGG | 1000 |
| 1048 | GCCCUAUC C CUUUACGU | 291 | ACGUAAAG CUGAUGAGGCCGUUAGGCCGAA IAUAGGGC | 1001 |
| 1049 | CCCUAUCC C UUUACGUC | 292 | GACGUAAA CUGAUGAGGCCGUUAGGCCGAA IGAUAGGG | 1002 |
| 1050 | CCUAUCCC U UUACGUCA | 293 | UGACGUAA CUGAUGAGGCCGUUAGGCCGAA IGGAUAGG | 1003 |
| 1058 | UUUACGUC A UCCCUGAG | 294 | CUCAGGGA CUGAUGAGGCCGUUAGGCCGAA IACGUAAA | 1004 |
| 1061 | ACGUCAUC C CUGAGCAC | 295 | GUGCUCAG CUGAUGAGGCCGUUAGGCCGAA IAUGACGU | 1005 |
| 1062 | CGUCAUCC C UGAGCACC | 296 | GGUGCUCA CUGAUGAGGCCGUUAGGCCGAA IGAUGACG | 1006 |
| 1063 | GUCAUCCC U GAGCACCA | 297 | UGGUGCUC CUGAUGAGGCCGUUAGGCCGAA IGGAUGAC | 1007 |
| 1068 | CCCUGAGC A CCAUCAAC | 298 | GUUGAUGG CUGAUGAGGCCGUUAGGCCGAA ICUCAGGG | 1008 |
| 1070 | CUGAGCAC C AUCAACUA | 299 | UAGUUGAU CUGAUGAGGCCGUUAGGCCGAA IUGCUCAG | 1009 |
| 1071 | UGAGCACC A UCAACUAU | 300 | AUAGUUGA CUGAUGAGGCCGUUAGGCCGAA IGUGCUCA | 1010 |
| 1074 | GCACCAUC A ACUAUGAU | 301 | AUCAUAGU CUGAUGAGGCCGUUAGGCCGAA IAUGGUGC | 1011 |
| 1077 | CCAUCAAC U AUGAUGAG | 302 | CUCAUCAU CUGAUGAGGCCGUUAGGCCGAA IUUGAUGG | 1012 |
| 1090 | UGAGUUUC C CACCAUGG | 303 | CCAUGGUG CUGAUGAGGCCGUUAGGCCGAA IAAACUCA | 1013 |
| 1091 | GAGUUUCC C ACCAUGGU | 304 | ACCAUGGU CUGAUGAGGCCGUUAGGCCGAA IGAAACUC | 1014 |
| 1092 | AGUUUCCC A CCAUGGUG | 305 | CACCAUGG CUGAUGAGGCCGUUAGGCCGAA IGGAAACU | 1015 |
| 1094 | UUUCCCAC C AUGGUGUU | 306 | AACACCAU CUGAUGAGGCCGUUAGGCCGAA IUGGGAAA | 1016 |
| 1095 | UUCCCACC A UGGUGUUU | 307 | AAACACCA CUGAUGAGGCCGUUAGGCCGAA IGUGGGAA | 1017 |
| 1105 | GGUGUUUC C UUCUGGGC | 308 | GCCCAGAA CUGAUGAGGCCGUUAGGCCGAA IAAACACC | 1018 |
| 1106 | GUGUUUCC U UCUGGGCA | 309 | UGCCCAGA CUGAUGAGGCCGUUAGGCCGAA IGAAACAC | 1019 |
| 1109 | UUUCCUUC U GGGCAGAU | 310 | AUCUGCCC CUGAUGAGGCCGUUAGGCCGAA IAAGGAAA | 1020 |
| 1114 | UUCUGGGC A GAUCAGCC | 311 | GGCUGAUC CUGAUGAGGCCGUUAGGCCGAA ICCCAGAA | 1021 |
| 1119 | GGCAGAUC A GCCAGGCC | 312 | GGCCUGGC CUGAUGAGGCCGUUAGGCCGAA IAUCUGCC | 1022 |
| 1122 | AGAUCAGC C AGGCCUCG | 313 | CGAGGCCU CUGAUGAGGCCGUUAGGCCGAA ICUGAUCU | 1023 |
| 1123 | GAUCAGCC A GGCCUCGG | 314 | CCGAGGCC CUGAUGAGGCCGUUAGGCCGAA IGCUGAUC | 1024 |
| 1127 | AGCCAGGC C UCGGCCUU | 315 | AAGGCCGA CUGAUGAGGCCGUUAGGCCGAA ICCUGGCU | 1025 |
| 1128 | GCCAGGCC U CGGCCUUG | 316 | CAAGGCCG CUGAUGAGGCCGUUAGGCCGAA IGCCUGGC | 1026 |
| 1133 | GCCUCGGC C UUGGCCCC | 317 | GGGGCCAA CUGAUGAGGCCGUUAGGCCGAA ICCGAGGC | 1027 |
| 1134 | CCUCGGCC U UGGCCCCG | 318 | CGCGGCCA CUGAUGAGGCCGUUAGGCCGAA ICCCGAGG | 1028 |
| 1139 | GCCUUGGC C CCGGCCCC | 319 | GGGGCCGG CUGAUGAGGCCGUUAGGCCGAA ICCAAGGC | 1029 |
| 1140 | CCUUGGCC C CGGCCCCU | 320 | ACGGGCCG CUGAUGAGGCCGUUAGGCCGAA IGCCAAGG | 1030 |
| 1141 | CUUGGCCC C GGCCCCUC | 321 | GAGGGGCC CUGAUGAGGCCGUUAGGCCGAA IGGCCAAG | 1031 |
| 1145 | GCCCCGGC C CCUCCCCA | 322 | UGGGGAGG CUGAUGAGGCCGUUAGGCCGAA ICCGGGGC | 1032 |
| 1146 | CCCCGGCC C CUCCCCAA | 323 | UUGGGGAG CUGAUGAGGCCGUUAGGCCGAA IGCCGGGG | 1033 |
| 1147 | CCCGGCCC C UCCCCAAG | 324 | CUUGGGGA CUGAUGAGGCCGUUAGGCCGAA IGGCCGGG | 1034 |
| 1148 | CCGGCCCC U CCCCAAGU | 325 | ACUUCGGG CUGAUGAGGCCGUUAGGCCGAA IGGGCCGG | 1035 |
| 1150 | GGCCCCUC C CCAAGUCC | 326 | GGACUUGG CUGAUGAGGCCGUUAGGCCGAA IAGGGCCC | 1036 |
| 1151 | GCCCCUCC C CAAGUCCU | 327 | AGGACUUG CUGAUGAGGCCGUUAGGCCGAA IGAGGGGC | 1037 |
| 1152 | CCCCUCCC C AAGUCCUG | 328 | CAGGACUU CUGAUGAGGCCGUUAGGCCGAA IGGAGGGG | 1038 |
| 1153 | CCCUCCCC A ACUCCUGC | 329 | GCAGGACU CUGAUGAGGCCGUUAGGCCGAA IGGGAGGG | 1039 |
| 1158 | CCCAAGUC C UGCCCCAG | 330 | CUGGGGCA CUGAUGAGGCCGUUAGGCCGAA IACUUGGG | 1040 |
| 1159 | CCAAGUCC U GCCCCAGG | 331 | CCUGGGGC CUGAUGAGGCCGUUAGGCCGAA IGACUUGG | 1041 |
| 1162 | AGUCCUGC C CCAGGCUC | 332 | GAGCCUGG CUGAUGAGGCCGUUAGGCCGAA ICAGGACU | 1042 |
| 1163 | GUCCUCCC C CAGGCUCC | 333 | GGAGCCUG CUGAUGAGGCCGUUAGGCCGAA IGCAGGAC | 1043 |
| 1164 | UCCUGCCC C AGGCUCCA | 334 | UGGAGCCU CUGAUGAGGCCGUUAGGCCGAA IGGCAGGA | 1044 |
| 1165 | CCUGCCCC A GGCUCCAG | 335 | CUGGAGCC CUGAUGAGGCCGUUAGGCCGAA IGGGCAGG | 1045 |
| 1169 | CCCCAGGC U CCAGCCCC | 336 | GGGGCUGG CUGAUGAGGCCGUUAGGCCGAA ICCUGGGG | 1046 |
| 1171 | CCAGGCUC C AGCCCCUG | 337 | CAGGGGCU CUGAUGAGGCCGUUAGGCCGAA IAGCCUGG | 1047 |
| 1172 | CAGGCUCC A GCCCCUGC | 338 | GCAGGGGC CUGAUGAGGCCGUUAGGCCGAA IGAGCCUG | 1048 |
| 1175 | GCUCCAGC C CCUGCCCC | 339 | GGGGCAGG CUGAUGAGGCCGUUAGGCCGAA ICUGGAGC | 1049 |
| 1176 | CUCCAGCC C CUGCCCCU | 340 | AGGGGCAG CUGAUGAGGCCGUUAGGCCGAA IGCUGGAG | 1050 |
| 1177 | UCCAGCCC C UGCCCCUG | 341 | CAGGGGCA CUGAUGAGGCCGUUAGGCCGAA IGGCUGGA | 1051 |
| 1178 | CCAGCCCC U GCCCCUGC | 342 | GCAGGGGC CUGAUGAGGCCGUUAGGCCGAA IGGGCUGG | 1052 |
| 1181 | GCCCCUGC C CCUGCUCC | 343 | GGAGCAGG CUGAUGAGGCCGUUAGGCCGAA ICAGGGGC | 1053 |
| 1182 | CCCCUGCC C CUGCUCCA | 344 | UGGAGCAG CUGAUGAGGCCGUUAGGCCGAA IGCAGGGG | 1054 |
| 1183 | CCCUGCCC C UGCUCCAG | 345 | CUGGAGCA CUGAUGAGGCCGUUAGGCCGAA IGGCAGGG | 1055 |
| 1184 | CCUGCCCC U GCUCCAGC | 346 | GCUGGAGC CUGAUGAGGCCGUUAGGCCGAA IGGGCAGG | 1056 |
| 1187 | GCCCCUCC U CCAGCCAU | 347 | AUGGCUGG CUGAUGAGGCCGUUAGGCCGAA ICAGGGGC | 1057 |
| 1189 | CCCUGCUC C AGCCAUGG | 348 | CCAUGGCU CUGAUGAGGCCGUUAGGCCGAA IAGCAGGG | 1058 |
| 1190 | CCUGCUCC A GCCAUGGU | 349 | ACCAUGGC CUGAUGAGGCCGUUAGGCCGAA IGAGCAGG | 1059 |
| 1193 | GCUCCAGC C AUGGUAUC | 350 | GAUACCAU CUGAUGAGGCCOUUAGGCCGAA ICUGGAGC | 1060 |
| 1194 | CUCCAGCC A UGGUAUCA | 351 | UGAUACCA CUGAUGAGGCCGUUAGGCCGAA IGCUGGAG | 1061 |
| 1202 | AUGGUAUC A GCUCUGGC | 352 | GCCAGAGC CUGAUGAGGCCGUUAGGCCGAA IAUACCAU | 1062 |
| 1205 | GUAUCAGC U CUGGCCCA | 353 | UGGGCCAG CUGAUGAGGCCGUUAGGCCGAA ICUGAUAC | 1063 |
| 1207 | AUCAGOUC U GGCCCAGG | 354 | CCUGGGCC CUGAUGAGGCCGUUAGGCCGAA IAGCUGAU | 1064 |
| 1211 | GCUCUGGC C CAGGCCCC | 355 | GGGGCCUG CUGAUGAGGCCGUUAGGCCGAA ICCAGAGC | 1065 |
| 1212 | CUCUGGCC C AGGCCCCA | 356 | UGGGGCCU CUGAUGAGGCCGUUAGGCCGAA IGCCAGAG | 1066 |
| 1213 | UCUGGCCC A GGCCCCAG | 357 | CUGGGGCC CUGAUGAGGCCGUUAGGCCGAA IGGCCAGA | 1067 |
| 1217 | GCCCAGGC C CCAGCCCC | 358 | GGGGCUGG CUGAUGAGGCCGUUAGGCCGAA ICCUGGGC | 1068 |
| 1218 | CCCAGGCC C CAGCCCCU | 359 | AGGGGCUG CUGAUGAGGCCGUUAGGCCGAA IGCCUGGG | 1069 |
| 1219 | CCAGGCCC C AGCCCCUG | 360 | CAGGGGCU CUGAUGAGGCCGUUAGGCCGAA IGGCCUGG | 1070 |
| 1220 | CAGGCCCC A GCCCCUGU | 361 | ACAGGGGC CUGAUGAGGCCGUUAGGCCGAA IGGGCCUG | 1071 |
| 1223 | GCCCCAGC C CCUGUCCC | 362 | GGGACAGG CUGAUGAGGCCGUUAGGCCGAA ICUGGGGC | 1072 |
| 1224 | CCCCAGCC C CUGUCCCA | 363 | UGGGACAG CUGAUGAGGCCGUUAGGCCGAA IGCUGGGG | 1073 |
| 1225 | CCCAGCCC C UGUCCCAG | 364 | CUGGGACA CUGAUGAGGCCGUUAGGCCGAA IGGCUGGG | 1074 |
| 1226 | CCAGCCCC U GUCCCAGU | 365 | ACUGGGAC CUGAUGAGGCCGUUAGGCCGAA IGGGCUGG | 1075 |
| 1230 | CCCCUGUC C CAGUCCUA | 366 | UAGGACUG CUGAUGAGGCCGUUAGGCCGAA IACAGGGG | 1076 |
| 1231 | CCCUGUCC C AGUCCUAG | 367 | CUAGGACU CUGAUGAGGCCGUUAGGCCGAA IGACAGGG | 1077 |
| 1232 | CCUGUCCC A GUCCUAGC | 368 | GCUAGGAC CUGAUGAGGCCGUUAGGCCGAA IGGACAGG | 1078 |
| 1236 | UCCCAGUC C UAGCCCCA | 369 | UGGGGCUA CUGAUGAGGCCGUUAGGCCGAA IACUGGGA | 1079 |
| 1237 | CCCAGUCC U AGCCCCAG | 370 | CUGGGGCU CUGAUGAGGCCGUUAGGCCGAA IGACUGGG | 1080 |
| 1241 | GUCCUAGC C CCAGGCCC | 371 | GGGCCUGG CUGAUGAGGCCGUUAGGCCGAA ICUAGGAC | 1081 |
| 1242 | UCCUAGCC C CAGGCCCU | 372 | AGGGCCUG CUGAUGAGGCCGUUAGGCCGAA IGCUAGGA | 1082 |
| 1243 | CCUAGCCC C AGGCCCUC | 373 | GAGGGCCU CUGAUGAGGCCGUUAGGCCGAA IGGCUAGG | 1083 |
| 1244 | CUAGCCCC A GGCCCUCC | 374 | GGAGGGCC CUGAUGAGGCCGUUAGGCCGAA IGGGCUAG | 1084 |
| 1248 | CCCCAGGC C CUCCUCAG | 375 | CUGAGGAG CUGAUGAGGCCGUUAGGCCGAA ICCUGGGG | 1085 |
| 1249 | CCCAGGCC C UCCUCAGG | 376 | CCUGAGGA CUGAUGAGGCCGUUAGGCCGAA IGCCUGGG | 1086 |
| 1250 | CCAGGCCC U CCUCAGGC | 377 | GCCUGAGG CUGAUGAGGCCGUUAGGCCGAA IGGCCUGG | 1087 |
| 1252 | AGGCCCUC C UCAGGCUG | 378 | CAGCCUGA CUGAUGAGGCCGUUAGGCCGAA IAGGGCCU | 1088 |
| 1253 | GGCCCUCC U CAGGCUGU | 379 | ACAGCCUG CUGAUGAGGCCGUUAGGCCGAA IGAGGGCC | 1089 |
| 1255 | CCCUCCUC A GGCUGUGG | 380 | CCACAGCC CUGAUGAGGCCGUUAGGCCGAA IAGGAGGG | 1090 |
| 1259 | CCUCAGGC U GUGGCCCC | 381 | GGGGCCAC CUGAUGAGGCCGUUAGGCCGAA ICCUGAGG | 1091 |
| 1265 | GCUGUGGC C CCACCUGC | 382 | GCAGGUGG CUGAUGAGGCCGUUAGGCCGAA ICCACAGC | 1092 |
| 1266 | CUGUGGCC C CACCUGCC | 383 | GGCAGGUG CUGAUGAGGCCGUUAGGCCGAA IGCCACAG | 1093 |
| 1267 | UGUGGCCC C ACCUGCCC | 384 | GGGCAGGU CUGAUGAGGCCGUUAGGCCGAA IGOCCACA | 1094 |
| 1268 | GUGGCCCC A CCUGCCCC | 385 | GGGGCAGG CUGAUGAGGCCGUUAGGCCGAA IGGGCCAC | 1095 |
| 1270 | GGCCCCAC C UGCCCCCA | 386 | UGGGGGCA CUGAUGAGGCCGUUAGGCCGAA IUGGGGCC | 1096 |
| 1271 | GCCCCACC U GCCCCCAA | 387 | UUGGGGGC CUGAUGAGGCCGUUAGGCCGAA IGUGGGGC | 1097 |
| 1274 | CCACCUGC C CCCAAGCC | 388 | GGCUUGGG CUGAUGAGGCCGUUAGGCCGAA ICAGGUGC | 1098 |
| 1275 | CACCUGCC C CCAAGCCC | 389 | GGGCUUGG CUGAUGAGGCCGUUAGGCCGAA IGCAGGUG | 1099 |
| 1276 | ACCUGCCC C CAAGCCCA | 390 | UGGGCUUG CUGAUGAGGCCGUUAGGCCGAA IGGCAGGU | 1100 |
| 1277 | CCUGCCCC C AAGCCCAC | 391 | GUGGGCUU CUGAUGAGGCCGUUAGGCCGAA IGGGCAGG | 1101 |
| 1278 | CUGCCCCC A AGCCCACC | 392 | GGUGGGCU CUGAUGAGGCCGUUAGGCCGAA IGGGGCAG | 1102 |
| 1282 | CCCCAAGC C CACCCAGG | 393 | CCUGGGUG CUGAUGAGGCCGUUAGGCCGAA ICUUGGGG | 1103 |
| 1283 | CCCAAGCC C ACCCAGGC | 394 | GCCUGGGU CUGAUGAGGCCGUUAGGCCGAA IGCUUGGG | 1104 |
| 1284 | CCAAGCCC A CCCAGGCU | 395 | AGCCUGGG CUGAUGAGGCCGUUAGGCCGAA IGGCUUGG | 1105 |
| 1286 | AAGCCCAC C CAGGCUGG | 396 | CCAGCCUG CUGAUGAGGCCGUUAGGCCGAA IUGGGCUU | 1106 |
| 1287 | AGCCCACC C AGGCUGGG | 397 | CCCAGCCU CUGAUGAGGCCGUUAGGCCGAA IGUGGGCU | 1107 |
| 1288 | GCCCACCC A GGCUGGGG | 398 | CCCCAGCC CUGAUGAGGCCGUUAGGCCGAA IGGUGGGC | 1108 |
| 1292 | ACCCAGGC U GGGGAAGG | 399 | CCUUCCCC CUGAUGAGGCCGUUAGGCCGAA ICCUGGGU | 1109 |
| 1306 | AGGAACGC U GUCAGAGG | 400 | CCUCUGAC CUGAUGAGGCCGUUAGGCCGAA ICGUUCCU | 1110 |
| 1310 | ACGCUGUC A GAGGCCCU | 401 | AGGGCCUC CUGAUGAGGCCGUUAGGCCGAA IACAGCGU | 1111 |
| 1316 | UCAGAGGC C CUGCUGCA | 402 | UGCAGCAG CUGAUGAGGCCGUUAGGCCGAA ICCUCUGA | 1112 |
| 1317 | CAGAGGCC C UGCUGCAG | 403 | CUGCAGCA CUGAUGAGGCCGUUAGGCCGAA IGCCUCUG | 1113 |
| 1318 | AGAGGCCC U GCUGCAGC | 404 | GCUGCAGC CUGAUGAGGCCGUUAGGCCGAA IGGCCUCU | 1114 |
| 1321 | GGCCCUGC U GCAGCUGC | 405 | GCAGCUGC CUGAUGAGGCCGUUAGGCCGAA ICAGGGCC | 1115 |
| 1324 | CCUGCUGC A GCUGCAGU | 406 | ACUGCAGC CUGAUGAGGCCGUUAGGCCGAA ICAGCAGG | 1116 |
| 1327 | GCUGCAGC U GCAGUUUG | 407 | CAAACUGC CUGAUGAGGCCGUUAGGCCGAA ICUGCAGC | 1117 |
| 1330 | GCAGCUGC A GUUUGAUG | 408 | CAUCAAAC CUGAUGAGGCCGUUAGGCCGAA ICAGCUGC | 1118 |
| 1347 | AUGAAGAC C UGGGGGCC | 409 | GCCCCCCA CUGAUGAGGCCGUUAGGCCGAA IUCUUCAU | 1119 |
| 1348 | UGAAGACC U GGGGGCCU | 410 | ACGCCCCC CUGAUGAGGCCGUUAGGCCGAA IGUCUUCA | 1120 |
| 1355 | CUGGGGGC C UUGCUUGG | 411 | CCAAGCAA CUGAUGAGGCCGUUAGGCCGAA ICCCCCAG | 1121 |
| 1356 | UGGGGGCC U UGCUUGGC | 412 | GCCAAGCA CUGAUGAGGCCGUUAGGCCGAA IGCCCCCA | 1122 |
| 1360 | GGCCUUGC U UGGCAACA | 413 | UCUUGCCA CUGAUGAGGCCGUUAGGCCGAA ICAAGGCC | 1123 |
| 1365 | UGCUUGGC A ACAGCACA | 414 | UGUGCUGU CUGAUGAGGCCGUUAGGCCGAA ICCAACCA | 1124 |
| 1368 | UUGGCAAC A GCACAGAC | 415 | GUCUGUGC CUGAUGAGGCCGUUAGGCCGAA IUUGCCAA | 1125 |
| 1371 | GCAACAGC A CAGACCCA | 416 | UGGGUCUG CUGAUGAGGCCGUUAGGCCGAA ICUGUUGC | 1126 |
| 1373 | AACAGCAC A GACCCAGC | 417 | GCUGGGUC CUGAUGAGGCCGUUAGGCCGAA IUGCUGUU | 1127 |
| 1377 | GOACAGAC C CAGCUGUG | 418 | CACAGCUG CUGAUGAGGCCGUUAGGCCGAA IUCUGUGC | 1128 |
| 1378 | CACAGACC C AGCUGUGU | 419 | ACACAGCU CUGAUGAGGCCGUUAGGCCGAA IGUCUGUG | 1129 |
| 1379 | ACAGACCC A GCUGUGUU | 420 | AACACAGC CUGAUGAGGCCGUUAGGCCGAA IGGUCUGU | 1130 |
| 1382 | GACCCAGC U GUGUUCAC | 421 | GUGAACAC CUGAUGAGGCCGUUAGGCCGAA ICUGGGUC | 1131 |
| 1389 | CUGUGUUC A CAGACCUG | 422 | CAGGUCUG CUGAUGAGGCCGUUAGGCCGAA IAACACAG | 1132 |
| 1391 | GUGGUCAC A GACCUGGC | 423 | GCCAGGUC CUGAUGAGGCCGUUAGGCCGAA IUGAACAC | 1133 |
| 1395 | UCACAGAC C UGGCAUCC | 424 | GGAUGCCA CUGAUGAGGCCGUUAGGCCGAA IUCUGUGA | 1134 |
| 1396 | CACAGACC U GGCAUCCG | 425 | CGGAUGCC CUGAUGAGGCCGUUAGGCCGAA IGUCUGUG | 1135 |
| 1400 | GACCUGGC A UCCGUCGA | 426 | UCGACGGA CUGAUGAGGCCGUUAGGCCGAA ICCAGGUC | 1136 |
| 1403 | CUGGCAUC C GUCGACAA | 427 | UUGUCGAC CUGAUGAGGCCGUUAGGCCGAA IAUGCCAG | 1137 |
| 1410 | CCGUCGAC A ACUCCGAG | 428 | CUCGGAGU CUGAUGAGGCCGUUAGGCCGAA IUCGACGG | 1138 |
| 1413 | UCGACAAC U CCGAGUIJU | 429 | AAACUCGG CUGAUGAGGCCGUUAGGCCGAA IUUGUCGA | 1139 |
| 1415 | GACAACUC C GAGUUUCA | 430 | UGAAACUC CUGAUGAGGCCGUUAGGCCGAA IAGUUGUC | 1140 |
| 1423 | CGAGUUUC A GCAGCUGC | 431 | GCAGCUGC CUGAUGAGGCCGUUAGGCCGAA IAAACUCG | 1141 |
| 1426 | GUU1ICAGC A GCUGCUGA | 432 | UCAGCAGC CUGAUGAGGCCGUUAGGCCGAA ICUGAAAC | 1142 |
| 1429 | UCAGCAGC U GCUGAACC | 433 | GGUUCAGC CUGAUGAGGCCGUUAGGCCGAA ICUGCUGA | 1143 |
| 1432 | GCAGCUGC U GAACCAGG | 434 | CCUGGUUC CUGAUGAGGCCGUUAGGCCGAA ICAGCUGC | 1144 |
| 1437 | UGCUGAAC C AGGGCAUA | 435 | UAUGCCCU CUGAUGAGGCCGUUAGGCCGAA IUUCAGCA | 1145 |
| 1438 | GCUGAACC A GGGCAUAC | 436 | GUAUGCCC CUGAUGAGGCCGUUAGGCCGAA IGUUCAGC | 1146 |
| 1443 | ACCAGGGC A UACCUGUG | 437 | CACAGGUA CUGAUGAGGCCGUUAGGCCGAA ICCCUGGU | 1147 |
| 1447 | GGGCAUAC C UGUGGCCC | 438 | GGGCCACA CUGAUGAGGCCGUUAGGCCGAA IUAUGCCC | 1148 |
| 1448 | GGCAUACC U GUGGCCCC | 439 | GGGGCCAC CUGAUGAGGCCGUUAGGCCGAA IGUAUGCC | 1149 |
| 1454 | CCUGUGGC C CCCCACAC | 440 | GUGUGGGG CUGAUGAGGCCGUUAGGCCGAA ICCACAGG | 1150 |
| 1455 | CUGUGGCC C CCCACACA | 441 | UGUGUGGG CUGAUGAGGCCGUUAGGCCGAA IGCCACAG | 1151 |
| 1455 | UGUGGCCC C CCACACAA | 442 | UUGUGUGG CUGAUGAGGCCGUUAGGCCGAA IGGCCACA | 1152 |
| 1457 | GUGGCCCC C CACACAAC | 443 | GUUGUGUG CUGAUGAGGCCGUUAGGCCGAA IGGGCCAC | 1153 |
| 1458 | UGGCCCCC C ACACAACU | 444 | AGUUGUGU CUGAUGAGGCCGUUAGGCCGAA IGGGGCCA | 1154 |
| 1459 | GGCCCCCC A CACAACUG | 445 | CAGUUGUG CUGAUGAGGCCGUUAGGCCGAA IGGGGGCC | 1155 |
| 1461 | CCCCCCAC A CAACUGAG | 446 | CUCAGUUG CUGAUGAGGCCGUUAGGCCGAA IUGGGGGG | 1156 |
| 1463 | CCCCACAC A ACUGAGCC | 447 | GGCUCAGU CUGAUGAGGCCGUUAGGCCGAA IUGUGGGG | 1157 |
| 1466 | CACACAAC U GAGCCCAU | 448 | AUGGGCUC CUGAUGAGGCCGUUAGGCCGAA IUUGUGUG | 1158 |
| 1471 | AACUGAGC C CAUGCUGA | 449 | UCAGCAUG CUGAUGAGGCCGUUAGGCCGAA ICUCAGUU | 1159 |
| 1472 | ACUGAGCC C AUGCUGAU | 450 | AUCAGCAU CUGAUGAGGCCGUUAGGCCGAA IGCUCAGU | 1160 |
| 1473 | CUGAGCCC A UGCUGAUG | 451 | CAUCAGCA CUGAUGAGGCCGUUAGGCCGAA IGGCUCAG | 1161 |
| 1477 | GCCCAUGC U GAUGGAGU | 452 | ACUCCAUC CUGAUGAGGCCGUUAGGCCGAA ICAUGGGC | 1162 |
| 1488 | UGGAGUAC C CUGAGGCU | 453 | AGCCUCAG CUGAUGAGGCCGUUAGGCCGAA IUACUCCA | 1163 |
| 1489 | GGAGUACC C UGAGGCUA | 454 | UAGCCUCA CUGAUGAGGCCGUUAGGCCGAA IGUACUCC | 1164 |
| 1490 | GAGUACCC U GAGGCUAU | 455 | AUAGCCUC CUGAUGAGGCCGUUAGGCCGAA IGGUACUC | 1165 |
| 1496 | CCUGAGGC U AUAACUCG | 456 | CGAGUUAU CUGAUGAGGCCGUUAGGCCGAA ICCUCAGG | 1166 |
| 1502 | GCUAUAAC U CGCCUAGU | 457 | ACUAGGCG CUGAUGAGGCCGUUAGGCCGAA IUUAUAGC | 1167 |
| 1506 | UAACUCGC C UAGUGACA | 458 | UGUCACUA CUGAUGAGGCCGUUAGGCCGAA ICGAGUUA | 1168 |
| 1507 | AACUCGCC U AGUGACAG | 459 | CUGUCACU CUGAUGAGGCCGUUAGGCCGAA IGCGAGUU | 1169 |
| 1514 | CUAGUGAC A GCCCAGAG | 460 | CUCUGGGC CUGAUGAGGCCGUUAGGCCGAA IUCACUAG | 1170 |
| 1517 | GUGACAGC C CAGAGGCC | 461 | GGCCUCUG CUGAUGAGGCCGUUAGGCCGAA ICUGUCAC | 1171 |
| 1518 | UGACAGCC C AGAGGCCC | 462 | GGGCCUCU CUGAUGAGGCCGUUAGGCCGAA ICCUGUCA | 1172 |
| 1519 | GACAGCCC A GAGGCCCC | 463 | GGGGCCUC CUGAUGAGGCCGUUAGGCCGAA IGGCUGUC | 1173 |
| 1525 | CCAGAGGC C CCCCGACC | 464 | GGUCGGGG CUGAUGAGGCCGUUAGGCCGAA ICCUCUGG | 1174 |
| 1526 | CAGAGGCC C CCCGACCC | 465 | GGGUCGGG CUGAUGAGGCCGUUAGGCCGAA IGCCUCUG | 1175 |
| 1527 | AGAGGCCC C CCGACCCA | 466 | UGGGUCGG CUGAUGAGGCCGUUAGGCCGAA IGGCCUCU | 1176 |
| 1528 | GAGGCCCC C CGACCCAG | 467 | CUGGGUCG CUGAUGAGGCCGUUAGGCCGAA IGGGCCUC | 1177 |
| 1529 | AGGCCCCC C GACCCAGC | 468 | GCUGGGUC CUGAUGAGGCCGUUAGGCCGAA IGGGGCCU | 1178 |
| 1533 | CCCCCGAC C CAGCUCCU | 469 | AGGAGCUG CUGAUGAGGCCGUUAGGCCGAA IUCGGGGG | 1179 |
| 1534 | CCCCGACC C AGCUCCUG | 470 | CAGGAGCU CUGAUGAGGCCGUUAGGCCGAA IGUCGGGG | 1180 |
| 1535 | CCCGACCC A GCUCCUGC | 471 | GCAGGAGC CUGAUGAGGCCGUUAGGCCGAA IGGUCGGG | 1181 |
| 1538 | GACCCAGC U CCUGCUCC | 472 | GGAGCAGG CUGAUGAGGCCGUUAGGCCGAA ICUGGGUC | 1182 |
| 1540 | CCCAGCUC C UGCUCCAC | 473 | GUGGAGCA CUGAUGAGGCCGUUAGGCCGAA IAGCUGGG | 1183 |
| 1541 | CCAGCUCC U GCUCCACU | 474 | AGUGGAGC CUGAUGAGGCCGUUAGGCCGAA IGAGCUGG | 1184 |
| 1544 | GCUCCUGC U CCACUGGG | 475 | CCCAGUGG CUGAUGAGGCCGUUAGGCCGAA ICAGGAGC | 1185 |
| 1546 | UCCUGCUC C ACUGGGGG | 476 | CCCCCAGU CUGAUGAGGCCGUUAGGCCGAA IAGCAGGA | 1186 |
| 1547 | CCUGCUCC A CUGGGGGC | 477 | GCCCCCAG CUGAUGAGGCCGUUAGGCCGAA IGAGCAGG | 1187 |
| 1549 | UGCUCCAC U GGGGGCCC | 478 | GGGCCCCC CUGAUGAGGCCGUUAGGCCGAA IUGGAGCA | 1188 |
| 1556 | CUGGGGGC C CCGGGGCU | 479 | AGCCCCGG CUGAUGAGGCCGUUAGGCCGAA ICCCCCAG | 1189 |
| 1557 | UGGGGGCC C CGGGGCUC | 480 | GAGCCCCG CUGAUGAGGCCGUUAGGCCGAA IGCCCCCA | 1190 |
| 1558 | GGGGGCCC C GGGGCUCC | 481 | GGAGCCCC CUGAUGAGGCCGUUAGGCCGAA IGGCCCCC | 1191 |
| 1564 | CCCGGGGC U CCCCPAUG | 482 | CAUUGGGG CUGAUGAGGCCGUUAGGCCGAA ICCCCGGG | 1192 |
| 1566 | CGGGGCUC C CCAAUGGC | 483 | GCCAUUGG CUGAUGAGGCCGUUAGGCCGAA IAGCCCCG | 1193 |
| 1567 | GGGGCUCC C CAAUGGCC | 484 | GGCCAUUG CUGAUGAGGCCGUUAGGCCGAA IGAGCCCC | 1194 |
| 1568 | GGGCUCCC C AAUGGCCU | 485 | AGGCCAUU CUGAUGAGGCCGUUAGGCCGAA IGGAGCCC | 1195 |
| 1569 | GGCUCCCC A AUGGCCUC | 486 | GAGGCCAU CUGAUGAGGCCGUUAGGCCGAA IGGGAGCC | 1196 |
| 1575 | CCAAUGGC C UCCUUUCA | 487 | UGAAAGGA CUGAUGAGGCCGUUAGGCCGAA ICCAUUGG | 1197 |
| 1576 | CAAUGGCC U CCUUUCAG | 488 | CUGAAAGG CUGAUGAGGCCGUUAGGCCGAA IGCCAUUG | 1198 |
| 1578 | AUGOCCUC C UUUCAGGA | 489 | UCCUGAAA CUGAUGAGGCCGUUAGGCCGAA IAGGCCAU | 1199 |
| 1579 | UGGCCUCC U UUCAGGAG | 490 | CUCCUGAA CUGAUGAGGCCGUUAGGCCGAA IGAGCCCA | 1200 |
| 1583 | CUCCUUUC A GGAGAUGA | 491 | UCAUCUCC CUGAUGAGGCCGUUAGGCCGAA IAAAGGAG | 1201 |
| 1596 | AUGAAGAC U UCUCCUCC | 492 | GGAGGAGA CUGAUGAGGCCGUUAGGCCGAA IUCUUCAU | 1202 |
| 1599 | AAGACUUC U CCUCCAUU | 493 | AAUGGAGG CUGAUGAGGCCGUUAGGCCGAA IAAGUCUU | 1203 |
| 1601 | GACUUCUC C UCCAUUGC | 494 | GCAAUGGA CUGAUGAGGCCGUUAGGCCGAA IAGAAGUC | 1204 |
| 1602 | ACUUCUCC U CCAUUGCG | 495 | CGCAAUGG CUGAUGAGGCCGUUAGGCCGAA IGAGAAGU | 1205 |
| 1604 | UUCUCCUC C AUUGCGGA | 496 | UCCGCAAU CUGAUGAGGCCGUUAGGCCGAA IAGGAGAA | 1206 |
| 1605 | UCUCCUCC A UUGCGGAC | 497 | GUCCGCAA CUGAUGAGGCCGUUAGGCCGAA IGAGGAGA | 1207 |
| 1614 | UUGCGGAC A UGGACUUC | 498 | GAAGUCCA CUGAUGAGGCCGUUAGGCCGAA IUCCGCAA | 1208 |
| 1620 | ACAUGGAC U UCUCAGCC | 499 | GGCUGAGA CUGAUGAGGCCGUUAGGCCGAA IUCCAUGU | 1209 |
| 1623 | UGGACUUC U CAGCCCUG | 500 | CAGGGCUG CUGAUGAGGCCGUUAGGCCGAA IAAGUCCA | 1210 |
| 1625 | GACUUCUC A GCCCUGCU | 501 | AGCAGGGC CUGAUGAGGCCGUUAGGCCGAA IAGAAGUC | 1211 |
| 1628 | UUCUCAGC C CUGCUGAG | 502 | CUCAGCAG CUGAUGAGGCCGUUAGGCCGAA ICUGAGAA | 1212 |
| 1629 | UCUCAGCC C UGCUGAGU | 503 | ACUCAGCA CUGAUGAGGCCGUUAGGCCGAA IGCUGAGA | 1213 |
| 1630 | CUCAGCCC U GCUGAGUC | 504 | GACUCAGC CUGAUGAGGCCGUUAGGCCGAA IGGCUGAG | 1214 |
| 1633 | AGCCCUGC U GAGUCAGA | 505 | UCUGACUC CUGAUGAGGCCGUUAGGCCGAA ICAGGGCU | 1215 |
| 1639 | GCUGAGUC A GAUCAGCU | 506 | AGCUGAUC CUGAUGAGGCCGUUAGGCCGAA IACUCAGC | 1216 |
| 1644 | GUCAGAUC A GCUCCUAA | 507 | UUAGGAGC CUGAUGAGGCCGUUAGGCCGAA IAUCUGAC | 1217 |
| 1647 | AGAUCAGC U CCUAAGGG | 508 | CCCUUAGG CUGAUGAGGCCGUUAGGCCGAA ICUGAUCU | 1218 |
| 1649 | AUCAGCUC C UAAGGGGG | 509 | CCCCCUUA CUGAUGAGGCCGUUAGGCCGAA IAGCUGAU | 1219 |
| 1650 | UCAGCUCC U AAGGGGGU | 510 | ACCCCCUU CUGAUGAGGCCGUUAGGCCGAA IGAGCUGA | 1220 |
| 1664 | GGUGACGC C UGCCCUCC | 511 | GGAGGGCA CUGAUGAGGCCGUUAGGCCGAA ICGUCACC | 1221 |
| 1665 | GUGACGCC U GCCCUCCC | 512 | GGGAGGGC CUGAUGAGGCCGUUAGGCCGAA IGCGUCAC | 1222 |
| 1668 | ACGCCUGC C CUCCCCAG | 513 | CUGGGGAG CUGAUGAGGCCGUUAGGCCGAA ICAGGCGU | 1223 |
| 1669 | CGCCUGCC C UCCCCAGA | 514 | UCUGGGGA CUGAUGAGGCCGUUAGGCCGAA IGCAGGCG | 1224 |
| 1670 | GCCUGCCC U CCCCAGAG | 515 | CUCUGGGG CUGAUGAGGCCGUUAGGCCGAA IGGCAGGC | 1225 |
| 1672 | CUGCCCUC C CCAGAGCA | 516 | UGCUCUGG CUGAUGAGGCCGUUAGGCCGAA IAGGGCAG | 1226 |
| 1673 | UGCCCUCC C CAGAGCAC | 517 | GUGCUCUG CUGAUGAGGCCGUUAGGCCGAA IGAGGGCA | 1227 |
| 1674 | GCCCUCCC C AGAGCACU | 518 | AGUGCUCU CUGAUGAGGCCGUUAGGCCGAA IGGAGGGC | 1228 |
| 1675 | CCCUCCCC A GAGCACUG | 519 | CAGUGCUC CUGAUGAGGCCGUUAGGCCGAA IGGGAGGG | 1229 |
| 1680 | CCCAGAGC A CUGGUUGC | 520 | GCAACCAG CUGAUGAGGCCGUUAGGCCGAA ICUCUGGG | 1230 |
| 1682 | CAGAGCAC U GGUUGCAG | 521 | CUGCAACC CUGAUGAGGCCGUUAGGCCGAA IUGCUCUG | 1231 |
| 1689 | CUGGUUGC A GGGGAUUG | 522 | CAAUCCCC CUGAUGAGGCCGUUAGGCCGAA ICAACCAG | 1232 |
| 1702 | AUUGAAGC C CUCCAAAA | 523 | UUUUGGAG CUGAUGAGGCCGUUAGGCCGAA ICUUCAAU | 1233 |
| 1703 | UUGAAGCC C UCCAAAAG | 524 | CUUUUGGA CUGAUGAGGCCGUUAGGCCGAA IGCUUCAA | 1234 |
| 1704 | UGAAGCCC U CCAAAAGC | 525 | GCUUUUGG CUGAUGAGGCCGUUAGGCCGAA IGGCUUCA | 1235 |
| 1706 | AAGCCCUC C AAAAGCAC | 526 | GUGCUUUU CUGAUGAGGCCGUUAGGCCGAA IAGGGCUU | 1236 |
| 1707 | AGCCCUCC A AAAGCACU | 527 | AGUGCUUU CUGAUGAGGCCGUUAGGCCGAA IGAGGGCU | 1237 |
| 1713 | CCAAAAGC A CUUACGGA | 528 | UCCGUAAG CUGAUGAGGCCGUUAGGCCGAA ICUUUUGG | 1238 |
| 1715 | AAAAGCAC U UACGGAUU | 529 | AAUCCGUA CUGAUGAGGCCGUUAGGCCGAA IUGCUUUU | 1239 |
| 1725 | ACGGAUUC U GGUGGGGU | 530 | ACCCCACC CUGAUGAGGCCGUUAGGCCGAA IAAUCCGU | 1240 |
| 1740 | GUGUGUUC C AACUGCCC | 531 | GGGCAGUU CUGAUGAGGCCGUUAGGCCGAA IAACACAC | 1241 |
| 1741 | UGUGUUCC A ACUGCCCC | 532 | GGGGCAGU CUGAUGAGGCCGUUAGGCCGAA IGAACACA | 1242 |
| 1744 | GUUCCAAC U GCCCCCAA | 533 | UUGGGGGC CUGAUGAGGCCGUUAGGCCGAA IUUGGAAC | 1243 |
| 1747 | CCAACUGC C CCCAACUU | 534 | AAGUUGGG CUGAUGAGGCCGUUAGGCCGAA ICAGUUGG | 1244 |
| 1748 | CAACUGCC C CCAACUUU | 535 | AAAGUUGG CUGAUGAGGCCGUUAGGCCGAA IGCAGUUG | 1245 |
| 1749 | AACUGCCC C CAACUUUG | 536 | CAAAGUUG CUGAUGAGGCCGUUAGGCCGAA IGGCAGUU | 1246 |
| 1750 | ACUGCCCC C AACUUUGU | 537 | ACAAAGUU CUGAUGAGGCCGUUAGGCCGAA IGGGCAGU | 1247 |
| 1751 | CUGCCCCC A ACUUUGUG | 538 | CACAAAGU CUGAUGAGGCCGUUAGGCCGAA IGGGGCAG | 1248 |
| 1754 | CCCCCAAC U UUGUGGAU | 539 | AUCCACAA CUGAUGAGGCCGUUAGGCCGAA IUUGGGGG | 1249 |
| 1766 | UGGAUGUC U UCCUUGGA | 540 | UCCAAGGA CUGAUGAGGCCGUUAGGCCGAA IACAUCCA | 1250 |
| 1769 | AUGUCUUC C UUGGAGGG | 541 | CCCUCCAA CUGAUGAGGCCGUUAGGCCGAA IAAGACAU | 1251 |
| 1770 | UGUCUUCC U UGGAGGGG | 542 | CCCCUCCA CUGAUGAGGCCGUUAGGCCGAA IGAAGACA | 1252 |
| 1784 | GGGGGAGC C AUAUUUUA | 543 | UAAAAUAU CUGAUGAGGCCGUUAGGCCGAA ICUCCCCC | 1253 |
| 1785 | GGGGAGCC A UAUUUUAU | 544 | AUAAAAUA CUGAUGAGGCCGUUAGGCCGAA IGCUCCCC | 1254 |
| 1796 | UUUUAUUC U UUUAUUGU | 545 | ACAAUAAA CUGAUGAGGCCGUUAGGCCGAA IAAUAAAA | 1255 |
| 1806 | UUAUUGUC A GUAUCUGU | 546 | ACAGAUAC CUGAUGAGGCCGUUAGGCCGAA IACAAUAA | 1256 |
| 1812 | UCAGUAUC U GUAUCUCU | 547 | AGAGAUAC CUGAUGAGGCCGUUAGGCCGAA IAUACUGA | 1257 |
| 1818 | UCUGUAUC U CUCUCUCU | 548 | AGAGAGAG CUGAUGAGGCCGUUAGGCCGAA IAUACAGA | 1258 |
| 1820 | UGUAUCUC U CUCUCUUU | 549 | AAAGAGAG CUGAUGAGGCCGUUAGGCCGAA IAGAUACA | 1259 |
| 1822 | UAUCUCUC U CUCUUUUU | 550 | AAAAAGAG CUGAUGAGGCCGUUAGGCCGAA IAGAGAUA | 1260 |
| 1824 | UCUCUCUC U CUUUUUGG | 551 | CCAAAAAG CUGAUGAGGCCGUUAGGCCGAA IAGAGAGA | 1261 |
| 1826 | UCUCUCUC U UUUUGGAG | 552 | CUCCAAAA CUGAUGAGGCCGUUAGGCCGAA IAGAGAGA | 1262 |
| 1839 | GGAGGUGC U UAAGCAGA | 553 | UCUGCUUA CUGAUGAGGCCGUUAGGCCGAA ICACCUCC | 1263 |
| 1845 | GCUUAAGC A GAAGCAUU | 554 | AAUGCUUC CUGAUGAGGCCGUUAGGCCGAA ICUUAAGC | 1264 |
| 1851 | GCAGAAGC A UUAACUUC | 555 | GAAGUUAA CUGAUGAGGCCGUUAGGCCGAA ICUUCUGC | 1265 |
| 1857 | GCAUUAAC U UCUCUGGA | 556 | UCCAGAGA CUGAUGAGGCCGUUAGGCCGAA IUUAAUGC | 1266 |
| 1860 | UUAACUUC U CUGGAAAG | 557 | CUUUCCAG CUGAUGAGGCCGUUAGGCCGAA IAAGUUAA | 1267 |
| 1862 | AACUUCUC U GGAAAGGG | 558 | CCCUUUCC CUGAUGAGGCCGUUAGGCCGAA IAGAAGUU | 1268 |
| 1877 | GGGGGAGC U GGGGAAAC | 559 | GUUUCCCC CUGAUGAGGCCGUUAGGCCGAA ICUCCCCC | 1269 |
| 1886 | GGGGAAAC U CAAACUUU | 560 | AAAGUUUG CUGAUGAGGCCGUUAGGCCGAA IUUUCCCC | 1270 |
| 1888 | GGAAACUC A AACUUUUC | 561 | GAAAAGUU CUGAUGAGGCCGUUAGGCCGAA IAGUUUCC | 1271 |
| 1892 | ACUCAAAC U UUUCCCCU | 562 | AGGGGAAA CUGAUGAGGCCGUUAGGCCGAA IUUUGAGU | 1272 |
| 1897 | AACUUUUC C CCUGUCCU | 563 | AGGACAGG CUGAUGAGGCCGUUAGGCCGAA IAAAAGUU | 1273 |
| 1898 | ACUUUUCC C CUGUCCUG | 564 | CAGGACAG CUGAUGAGGCCGUUAGGCCGAA IGAAAAGU | 1274 |
| 1899 | CUUUUCCC C UGUCCUGA | 565 | UCAGGACA CUGAUGAGGCCGUUAGGCCGAA IGGAAAAG | 1275 |
| 1900 | UUUUCCCC U GUCCUGAU | 566 | AUCAGGAC CUGAUGAGGCCGUUAGGCCGAA IGGGAAAA | 1276 |
| 1904 | CCCCUGUC C UGAUGGUC | 567 | GACCAUCA CUGAUGAGGCCGUUAGGCCGAA IACAGGGG | 1277 |
| 1905 | CCCUGUCC U GAUGGUCA | 568 | UGACCAUC CUGAUGAGGCCGUUAGGCCGAA IGACAGGG | 1278 |
| 1913 | UGAUGGUC A GCUCCCUU | 569 | AAGGGAGC CUGAUGAGGCCGUUAGGCCGAA IACCAUCA | 1279 |
| 1916 | UGGUCAUC U CCCUUCUC | 570 | GAGAAGGG CUGAUGAGGCCGUUAGGCCGAA ICUGACCA | 1280 |
| 1918 | GUCAGCUC C CUUCUCUG | 571 | CAGAGAAG CUGAUGAGGCCGUUAGGCCGAA IAGCUGAC | 1281 |
| 1919 | UCAGCUCC C UUCUCUGU | 572 | ACAGAGAA CUGAUGAGGCCGUUAGGCCGAA IGAGCUGA | 1282 |
| 1920 | CAGCUCCC U UCUCUGUA | 573 | UACAGAGA CUGAUGAGGCCGUUAGGCCGAA IGGAGCUG | 1283 |
| 1923 | CUCCCUUC U CUGUAGGG | 574 | CCCUACAG CUGAUGAGGCCGUUAGGCCGAA IAAGGGAG | 1284 |
| 1925 | CCCUUCUC U GUAGGGAA | 575 | UUCCCUAC CUGAUGAGGCCGUUAGGCCGAA IAGAAGGG | 1285 |
| 1935 | UAGGGAAC U GUGGGGUC | 576 | GACCCCAC CUGAUGAGGCCGUUAGGCCGAA IUUCCCUA | 1286 |
| 1944 | GUGGGGUC C CCCAUCCC | 577 | GGGAUGGG CUGAUGAGGCCGUUAGGCCGAA IACCCCAC | 1287 |
| 1945 | UGGGGUCC C CCAUCCCC | 578 | GGGGAUGG CUGAUGAGGCCGUUAGGCCGAA IGACCCCA | 1288 |
| 1946 | GGGGUCCC C CAUCCCCA | 579 | UGGGGAUG CUGAUGAGGCCGUUAGGCCGAA IGGACCCC | 1289 |
| 1947 | GGGUCCCC C AUCCCCAU | 580 | AUGGGGAU CUGAUGAGGCCGUUAGGCCGAA IGGGACCC | 1290 |
| 1948 | GGUCCCCC A UCCCCAUC | 581 | GAUGGGGA CUGAUGAGGCCGUUAGGCCGAA IGGGGACC | 1291 |
| 1951 | CCCCCAUC C CCAUCCUC | 582 | GAGGAUGG CUGAUGAGGCCGUUAGGCCGAA IAUGGGGG | 1292 |
| 1952 | CCCCAUCC C CAUCCUCC | 583 | GGAGGAUG CUGAUGAGGCCGUUAGGCCGAA IGAUGGGG | 1293 |
| 1953 | CCCAUCCC C AUCCUCCA | 584 | UGGAGGAU CUGAUGAGGCCGUUAGGCCGAA IGGAUGGG | 1294 |
| 1954 | CCAUCCCC A UCCUCCAG | 585 | CUGGAGGA CUGAUGAGGCCGUUAGGCCGAA IGGGAUGG | 1295 |
| 1957 | UCCCCAUC C UCCAGCUU | 586 | AGCUGGAA CUGAUGAGGCCGUUAGGCCGAA IAUGGGGA | 1296 |
| 1958 | CCCCAUCC U CCAGCUUC | 587 | GAAGCUGG CUGAUGAGGCCGUUAGGCCGAA IGAUGGGG | 1297 |
| 1960 | CCAUCCUC C AGCUUCUG | 588 | CAGAAGCU CUGAUGAGGCCGUUAGGCCGAA IAGGAUGG | 1298 |
| 1961 | CAUCCUCC A GCUUCUGG | 589 | CCAGAAGC CUGAUGAGGCCGUUAGGCCGAA IGAGGAUG | 1299 |
| 1964 | CCUCCAGC U UCUGGUAC | 590 | GUACCAGA CUGAUGAGGCCGUUAGGCCGAA ICUGGAGG | 1300 |
| 1967 | CCAGCUUC U GGUACUCU | 591 | AGAGUACC CUGAUGAGGCCGUUAGGCCGAA IAAGCUGG | 1301 |
| 1973 | UCUGGUAC U CUCCUAGA | 592 | UCUAGGAG CUGAUGAGGCCGUUAGGCCGAA IUACCAGA | 1302 |
| 1975 | UGGUACUC U CCUAGAGA | 593 | UCUCUAGG CUGAUGAGGCCGUUAGGCCGAA IAGUACCA | 1303 |
| 1977 | GUACUCUC C UAGAGACA | 594 | UGUCUCUA CUGAUGAGGCCGUUAGGCCGAA IAGAGUAC | 1304 |
| 1978 | UACUCUCC U AGAGACAG | 595 | CUGUCUCU CUGAUGAGGCCGUUAGGCCGAA IGAGAGUA | 1305 |
| 1985 | CUAGAGAC A GAAGCAGG | 596 | CCUGCUUC CUGAUGAGGCCGUUAGGCCGAA IUCUCUAG | 1306 |
| 1991 | ACAGAAGC A GGCUGGAG | 597 | CUCCAGCC CUGAUGAGGCCGUUAGGCCGAA ICUUCUGU | 1307 |
| 1995 | AAGCAGGC U GGAGGUAA | 598 | UUACCUCC CUGAUGAGGCCGUUAGGCCGAA ICCUGCUU | 1308 |
| 2007 | GGUAAGGC C UUUGAGCC | 599 | GGCUCAAA CUGAUGAGGCCGUUAGGCCGAA ICCUUACC | 1309 |
| 2008 | GUAAGGCC U UUGAGCCC | 600 | GGGCUCAA CUGAUGAGGCCGUUAGGCCGAA IGCCUUAC | 1310 |
| 2015 | CUUUGAGC C CACAAAGC | 601 | GCUUUGUG CUGAUGAGGCCGUUAGGCCGAA ICUCAAAG | 1311 |
| 2016 | UUUGAGCC C ACAAAGCC | 602 | GGCUUUGU CUGAUGAGGCCGUUAGGCCGAA IGCUCAAA | 1312 |
| 2017 | UUGAGCCC A CAAAGCCU | 603 | AGGCUUUG CUGAUGAGGCCGUUAGGCCGAA IGGCUCAA | 1313 |
| 2019 | GAGCCCAC A AAGCCUUA | 604 | UAAGGCUU CUGAUGAGGCCGUUAGGCCGAA IUGGGCUC | 1314 |
| 2024 | CACAAAGC C UUAUCAAG | 605 | CUUGAUAA CUGAUGAGGCCGUUAGGCCGAA ICUCUGUG | 1315 |
| 2025 | ACAAAGCC U UAUCAAGU | 606 | ACUUGAUA CUGAUGAGGCCGUUAGGCCGAA IGCUUUGU | 1316 |
| 2030 | GCCUUAUC A AGUGUCUU | 607 | AAGACACU CUGAUGAGGCCGUUAGGCCGAA IAUAAGGC | 1317 |
| 2037 | CAAGUGUC U UCCAUCAU | 608 | AUGAUGGA CUGAUGAGGCCGUUAGGCCGAA IACACUUG | 1318 |
| 2040 | GUGUCUUC C AUCAUGGA | 609 | UCCAUGAU CUGAUGAGGCCGUUAGGCCGAA IAAGACAC | 1319 |
| 2041 | UGUCUUCC A UCAUGGAU | 610 | AUCCAUGA CUGAUGAGGCCGUUAGGCCGAA IGAAGACA | 1320 |
| 2044 | CUUCCAUC A UGGAUUCA | 611 | UGAAUCCA CUGAUGAGGCCGUUAGGCCGAA IAUGGAAG | 1321 |
| 2052 | AUGGAUUC A UUACAGCU | 612 | AGCUGUAA CUGAUGAGGCCGUUAGGCCGAA IAAUCCAU | 1322 |
| 2057 | UUCAUUAC A GCUUAAUC | 613 | GAUUAAGC CUGAUGAGGCCGUUAGGCCGAA IUAAUGAA | 1323 |
| 2060 | AUUACAGC U UAAUCAAA | 614 | UUUGAUUA CUGAUGAGGCCGUUAGGCCGAA ICUGUAAU | 1324 |
| 2066 | GCUUAAUC A AAAUAACG | 615 | CGUUAUUU CUGAUGAGGCCGUUAGGCCGAA IAUUAAGC | 1325 |
| 2076 | AAUAACGC C CCAGAUAC | 616 | GUAUCUGG CUGAUGAGGCCGUUAGGCCGAA ICGUUAUU | 1326 |
| 2077 | AUAACGCC C CAGAUACC | 617 | GGUAUCUG CUGAUGAGGCCGUUAGGCCGAA IGCGUUAU | 1327 |
| 2078 | UAACGCCC C AGAUACCA | 618 | UGGUAUCU CUGAUGAGGCCGUUAGGCCGAA IGGCGUUA | 1328 |
| 2079 | AACGCCCC A GAUACCAG | 619 | CUGGUAUC CUGAUGAGGCCGUUAGGCCGAA IGGGCGUU | 1329 |
| 2085 | CCAGAUAC C AGCCCCUG | 620 | CAGGGGCU CUGAUGAGGCCGUUAGGCCGAA IUAUCUGG | 1330 |
| 2086 | CAGAUACC A GCCCCUGU | 621 | ACAGGGGC CUGAUGAGGCCGUUAGGCCGAA IGUAUCUG | 1331 |
| 2089 | AUACCAGC C CCUGUAUG | 622 | CAUACAGG CUGAUGAGGCCGUUAGGCCGAA ICUGGUAU | 1332 |
| 2090 | UACCAGCC C CUGUAUGG | 623 | CCAUACAG CUGAUGAGGCCGUUAGGCCGAA IGCUGGUA | 1333 |
| 2091 | ACCAGCCC C UGUAUGGC | 624 | GCCAUACA CUGAUGAGGCCGUUAGGCCGAA IGGCUGGU | 1334 |
| 2092 | CCAGCCCC U GUAUGGCA | 625 | UGCCAUAC CUGAUGAGGCCGUUAGGCCGAA IGGGCUGG | 1335 |
| 2100 | UGUAUGGC A CUGGCAUU | 626 | AAUGCCAG CUGAUGAGGCCGUUAGGCCGAA ICCAUACA | 1336 |
| 2102 | UAUGGCAC U GGCAUUGU | 627 | ACAAUGCC CUGAUGAGGCCGUUAGGCCGAA IUGCCAUA | 1337 |
| 2106 | GCACUGGC A UUGUCCCU | 628 | AGGGACAA CUGAUGAGGCCGUUAGGCCGAA ICCAGUGC | 1338 |
| 2112 | GCAUUGUC C CUGUGCCU | 629 | AGGCACAG CUGAUGAGGCCGUUAGGCCGAA IACAAUGC | 1339 |
| 2113 | CAUUGUCC C UGUGCCUA | 630 | UAGGCACA CUGAUGAGGCCGUUAGGCCGAA IGACAAUG | 1340 |
| 2114 | AUUGUCCC U GUGCCUAA | 631 | UUAGGCAC CUGAUGAGGCCGUUAGGCCGAA IGGACAAU | 1341 |
| 2119 | CCCUGUGC C UAACACCA | 632 | UGGUGUUA CUGAUGAGGCCGUUAGGCCGAA ICACAGGG | 1342 |
| 2120 | CCUGUGCC U AACACCAG | 633 | CUGGUGUU CUGAUGAGGCCGUUAGGCCGAA IGCACAGG | 1343 |
| 2124 | UGCCUAAC A CCAGCGUU | 634 | AACGCUGG CUGAUGAGGCCGUUAGGCCGAA IUUAGGCA | 1344 |
| 2126 | CCUAACAC C AGCGUUUG | 635 | CAAACGCU CUGAUGAGGCCGUUAGGCCGAA IUGUUAGG | 1345 |
| 2127 | CUAACACC A GCGUUUGA | 636 | UCAAACGC CUGAUGAGGCCGUUAGGCCGAA IGUGUUAG | 1346 |
| 2141 | UGAGGGGC U GCCUUCCU | 637 | AGGAAGGC CUGAUGAGGCCGUUAGGCCGAA ICCCCUCA | 1347 |
| 2144 | GGGGCUGC C UUCCUGCC | 638 | GGCAGGAA CUGAUGAGGCCGUUAGGCCGAA ICAGCCCC | 1348 |
| 2145 | GGGCUGCC U UCCUGCCC | 639 | GGGCAGGA CUGAUGAGGCCGUUAGGCCGAA IGCAGCCC | 1349 |
| 2148 | CUGCCUUC C UGCCCUAC | 640 | GUAGGGCA CUGAUGAGGCCGUUAGGCCGAA IAAGGCAG | 1350 |
| 2149 | UGCCUUCC U GCCCUACA | 641 | UGUAGGGC CUGAUGAGGCCGUUAGGCCGAA IGAAGGCA | 1351 |
| 2152 | CUUCCUGC C CUACAGAG | 642 | CUCUGUAG CUGAUGAGGCCGUUAGGCCGAA ICAGGAAG | 1352 |
| 2153 | UUCCUGCC C UACAGAGG | 643 | CCUCUGUA CUGAUGAGGCCGUUAGGCCGAA IGCAGGAA | 1353 |
| 2154 | UCCUGCCC U ACAGAGGU | 644 | ACCUCUGU CUGAUGAGGCCGUUAGGCCGAA IGGCAGGA | 1354 |
| 2157 | UGCCCUAC A GAGGUCUC | 645 | GAGACCUC CUGAUGAGGCCGUUAGGCCGAA IUAGGGCA | 1355 |
| 2164 | CAGAGGUC U CUGCCGGC | 646 | GCCGGCAG CUGAUGAGGCCGUUAGGCCGAA IACCUCUG | 1356 |
| 2166 | GAGGUCUC U GCCGGCUC | 647 | GAGCCGGC CUGAUGAGGCCGUUAGGCCGAA IAGACCUC | 1357 |
| 2169 | GUCUCUGC C GGCUCUUU | 648 | AAAGAGCC CUGAUGAGGCCGUUAGGCCGAA ICAGAGAC | 1358 |
| 2173 | CUGCCGGC U CUUUCCUU | 649 | AAGGAAAG CUGAUGAGGCCGUUAGGCCGAA ICCGGCAG | 1359 |
| 2175 | GCCGGCUC U UUCCUUGC | 650 | GCAAGGAA CUGAUGAGGCCGUUAGGCCGAA IAGCCGGC | 1360 |
| 2179 | GCUCUUUC C UUGCUCAA | 651 | UUGAGCAA CUGAUGAGGCCGUUAGGCCGAA IAAAGAGC | 1361 |
| 2180 | CUCUUUCC U UGCUCAAC | 652 | GUUGAGCA CUGAUGAGGCCGUUAGGCCGAA IGAAAGAG | 1362 |
| 2184 | UUCCUUGC U CAACCAUG | 653 | CAUGGUUG CUGAUGAGGCCGUUAGGCCGAA ICAAGGAA | 1363 |
| 2186 | CCUUGCUC A ACCAUGGC | 654 | GCCAUGGU CUGAUGAGGCCGUUAGGCCGAA IAGCAAGG | 1364 |
| 2189 | UGCUCAAC C AUGGCUGA | 655 | UCAGCCAU CUGAUGAGGCCGUUAGGCCGAA IUUGAGCA | 1365 |
| 2190 | GCUCAACC A UGGCUGAA | 656 | UUCAGCCA CUGAUGAGGCCGUUAGGCCGAA IGUUGAGC | 1366 |
| 2195 | ACCAUGGC U GAAGGAAA | 657 | UUUCCUUC CUGAUGAGGCCGUUAGGCCGAA ICCAUGGU | 1367 |
| 2205 | AAGGAAAC A GUGCAACA | 658 | UGUUGCAC CUGAUGAGGCCGUUAGGCCGAA IUUUCCUU | 1368 |
| 2210 | AACAGUGC A ACAGCACU | 659 | AGUGCUGU CUGAUGAGGCCGUUAGGCCGAA ICACUGUU | 1369 |
| 2213 | AGUGCAAC A GCACUGGC | 660 | GCCAGUGC CUGAUGAGGCCGUUAGGCCGAA IUUGCACU | 1370 |
| 2216 | GCAACAGC A CUGGCUCU | 661 | AGAGCCAG CUCAUGAGGCCGUUAGGCCGAA ICUGUUGC | 1371 |
| 2218 | AACAGCAC U GGCUCUCU | 662 | AGAGAGCC CUGAUGAGGCCGUUAGGCCGAA IUGCUGUU | 1372 |
| 2222 | GCACUGGC U CUCUCCAG | 663 | CUGGAGAG CUGAUGAGGCCGUUAGGCCGAA ICCAGUOC | 1373 |
| 2224 | ACUGGCUC U CUCCAGGA | 664 | UCCUGGAG CUGAUGAGGCCGUUAGGCCGAA IAGCCAGU | 1374 |
| 2226 | UGGCUCUC U CCAGGAUC | 663 | GAUCCUGG CUGAUGAGGCCGUUAGGCCGAA IAGAGCCA | 1375 |
| 2228 | GCUCUCUC C AGGAUCCA | 666 | UGGAUCCU CUGAUGAGGCCGUUAGGCCGAA IAGAGAGC | 1376 |
| 2229 | CUCUCUCC A GGAUCCAG | 667 | CUGGAUCC CUGAUGAGGCCGUUAGGCCGAA IGAGAGAG | 1377 |
| 2235 | CCAGGAUC C AGAAGGGG | 668 | CCCCUUCU CUGAUGAGGCCGUUAGGCCGAA IAUCCUGG | 1378 |
| 2236 | CAGGAUCC A GAAGGGGU | 669 | ACCCCUUC CUGAUGAGGCCGUUAGGCCGAA IGAUCCUG | 1379 |
| 2251 | GUUUGGUC U GGACUUCC | 670 | GGAAGUCC CUGAUGAGGCCGUUAGGCCGAA IACCAAAC | 1380 |
| 2256 | GUCUGGAC U UCCUUGCU | 671 | AGCAAGGA CUGAUGAGGCCGUUAGGCCGAA IUCCAGAC | 1381 |
| 2259 | UGGACUUC C UUGCUCUC | 672 | GAGAGCAA CUGAUGAGGCCGUUAGGCCGAA IAAGUCCA | 1382 |
| 2260 | GGACUUCC U UGCUCUCC | 673 | GGAGAGCA CUGAUGAGGCCGUUAGGCCGAA IGAAGUCC | 1383 |
| 2264 | UUCCUUGC U CUCCCCUC | 674 | GAGGGGAC CUGAUGAGGCCGUUAGGCCGAA ICAAGGAA | 1384 |
| 2266 | CCUUGCUC U CCCCUCUU | 675 | AAGAGGGG CUGAUGAGGCCGUUAGGCCGAA IAGCAAGG | 1385 |
| 2268 | UUGCUCUC C CCUCUUCU | 676 | AGAAGAGG CUGAUGAGGCCGUUAGGCCGAA IAGAGCAA | 1386 |
| 2269 | UGCUCUCC C CUCUUCUC | 677 | GAGAAGAG CUGAUGAGGCCGUUAGGCCGAA IGAGAGCA | 1387 |
| 2270 | GCUCUCCC C UCUUCUCA | 678 | UGAGAAGA CUGAUGAGGCCGUUAGGCCGAA IGGAGAGC | 1388 |
| 2271 | CUCUCCCC U CUUCUCAA | 679 | UUGAGAAG CUGAUGAGGCCGUUAGGCCGAA IGGGAGAG | 1389 |
| 2273 | CUCCCCUC U UCUCAAGU | 680 | ACUUGAGA CUGAUGAGGCCGUUAGGCCGAA IAGGGGAG | 1390 |
| 2276 | CCCUCUUC U CAAGUGCC | 681 | GGCACUUG CUGAUGAGGCCGUUAGGCCGAA IAAGAGGG | 1391 |
| 2278 | CUCUUCUC A AGUGCCUU | 682 | AACGCACU CUGAUGAGGCCGUUAGGCCGAA IAGAAGAG | 1392 |
| 2284 | UCAAGUGC C UUAAUAGU | 683 | ACUAUUAA CUGAUGAGGCCGUUAGGCCGAA ICACUUGA | 1393 |
| 2285 | CAAGUGCC U UAAUAGUA | 684 | UACUAUUA CUGAUGAGGCCGUUAGGCCGAA IGCACUUG | 1394 |
| 2322 | GGGAGAGC A GGCUGGCA | 685 | UGCCAGCC CUGAUGAGGCCGUUAGGCCGAA ICUCUCCC | 1395 |
| 2326 | GAGCAGGC U GGCAGCUC | 686 | GAGCUGCC CUGAUGAGGCCGUUAGGCCGAA ICCUGCUC | 1396 |
| 2330 | AGGCUGGC A GCUCUCCA | 687 | UGGAGAGC CUGAUGAGGCCGUUAGGCCGAA ICCAGCCU | 1397 |
| 2333 | CUGGCAGC U CUCCAGUC | 688 | GACUGGAG CUGAUGAGGCCGUUAGGCCGAA ICUGCCAG | 1398 |
| 2335 | GGCAGCUC U CCAGUCAG | 689 | CUGACUGO CUGAUGAGGCCGUUAGGCCGAA IAGCUGCC | 1399 |
| 2337 | CAGCUCUC C AGUCAGGA | 690 | UCCUGACU CUGAUGAGGCCGUUAGGCCGAA IAGAGCUG | 1400 |
| 2338 | AGCUCUCC A GUCACGAG | 691 | CUCCUGAC CUGAUGAGGCCGUUAGGCCGAA IGAGAGCU | 1401 |
| 2342 | CUCCAGUC A GGAGGCAU | 692 | AUGCCUCC CUGAUGAGGCCGUUAGGCCGAA IACUGGAG | 1402 |
| 2349 | CAGGAGCC A UAGUUUUU | 693 | AAAAACUA CUGAUGAGGCCGUUAGGCCGAA ICCUCCUG | 1403 |
| 2365 | UAGUGAAC A AUCAAAGC | 694 | GCUUUGAU CUGAUGAGGCCGUUAGGCCGAA IUUCACUA | 1404 |
| 2369 | GAACAAUC A AAGCACUU | 695 | AAGUGCUU CUGAUGAGGCCGUUAGGCCGAA IAUUGUUC | 1405 |
| 2374 | AUCAAAGC A CUUGGACU | 696 | AGUCCAAG CUGAUGAGGCCGUUAGGCCGAA ICUUUGAU | 1406 |
| 2376 | CAAAGCAC U UGGACUCU | 697 | AGAGUCCA CUGAUGAGGCCGUUAGGCCGAA IUGCUUUG | 1407 |
| 2382 | ACUUGGAC U CUUGCUCU | 698 | AGAGCAAG CUGAUGAGGCCGUUAGGCCGAA IUCCAAGU | 1408 |
| 2384 | UUGGACUC U UGCUCUUU | 699 | AAAGAGCA CUGAUGAGGCCGUUAGGCCGAA IAGUCCAA | 1409 |
| 2388 | ACUCUUGC U CUUUCUAC | 700 | GUAGAAAG CUGAUGAGGCCGUUAGGCCGAA ICAAGAGU | 1410 |
| 2390 | UCUUGCUC U UUCUACUC | 701 | GAGUAGAA CUGAUGAGGCCGUUAGGCCGAA IAGCAAGA | 1411 |
| 2394 | GCUCUUUC U ACUCUGAA | 702 | UUCAGAGU CUGAUGAGGCCGUUAGGCCGAA IAAAGAGC | 1412 |
| 2397 | CUUUCUAC U CUGAACUA | 703 | UAGUUCAG CUGAUGAGGCCGUUAGGCCGAA IUAGAAAG | 1413 |
| 2399 | UUCUACUC U GAACUAAU | 704 | AUUAGUUC CUGAUGAGGCCGUUAGGCCGAA IAGUAGAA | 1414 |
| 2404 | CUCUGAAC U AAUAAAGC | 705 | GCUUUAUU CUGAUGAGGCCGUUAGGCCGAA IUUCAGAG | 1415 |
| 2413 | AAUAAAGC U GUUGCCAA | 706 | UUGGCAAC CUGAUGAGGCCGUUAGGCCGAA ICUUUAUU | 1416 |
| 2419 | GCUGUUGC C AAGCUGGA | 707 | UCCAGCUU CUGAUGAGGCCGUUAGGCCGAA ICAACAGC | 1417 |
| 2420 | CUGUUGCC A AGCUGGAC | 708 | GUCCAGCU CUGAUGAGGCCGUUAGGCCGAA IGCAACAG | 1418 |
| 2424 | UGCCAAGC U GGACGGCA | 709 | UGCCGUCC CUGAUGAGGCCGUUAGGCCGAA ICUUGGCA | 1419 |
| 2432 | UGGACGGC A CGAGCUCG | 710 | CGAGCUCG CUGAUGAGGCCGUUAGGCCGAA ICCGUCCA | 1420 |

Input Sequence = NM_021975. Cut Site = CH/.
Arm Length = 8. Core Sequence = CUGAUGAG GCCGUUAGGC CGAA
NM_021975 (Homo sapiens p65 RelA (NFKB), mRNA; 2444 bp)

TABLE IV
Human REL-A Zinzyme and Substrate Sequence

| | | Seq | | Seq |
|---|---|---|---|---|
| Pos | Substrate | ID | Zinzyme | ID |
| 9 | GGCACGAG G CGGGGCCG | 1421 | CGGCCCCG GCCGAAAGGCGAGUGAGGUCU CUCGUGCC | 1717 |
| 14 | GAGGCGGG G CCGGGUCG | 1422 | CGACCCGG GCCGAAAGGCGAGUGAGGUCU CCCGCCUC | 1718 |
| 19 | GGGGCCGG G UCGCAGCU | 1423 | AGCUGCGA GCCGAAAGGCGAGUGAGGUCU CCGGCCCC | 1719 |
| 22 | GCCGGGUC G CAGCUGGG | 1424 | CCCAGCUG GCCGAAAGGCGAGUGAGGUCU GACCCGGC | 1720 |
| 25 | GGGUCGCA G CUGGGCCC | 1425 | GGCCCCAG GCCGAAAGGCGAGUGAGGUCU UGCGACCC | 1721 |
| 30 | GCAGCUGG G CCCGCGGC | 1426 | GCCGCGGG GCCGAAAGGCGAGUGAGGUCU CCAGCUGC | 1722 |
| 34 | CUGGGCCC G CGGCAUGG | 1427 | CCAUGCCG GCCGAAAGGCGAGUGAGGUCU GGGCCCAG | 1723 |
| 37 | GGCCCGCG G CAUGGACG | 1428 | CGUCCAUG GCCGAAAGGCGAGUGAGGUCU CGCGGGCC | 1724 |
| 50 | GACGAACU G UUCCCCCU | 1429 | AGGGGGAA GCCGAAAGGCGAGUGAGGUCU AGUUCGUC | 1725 |
| 69 | UCUUCCCG G CACAGCAG | 1430 | CUGCUCUG GCCGAAAGGCGAGUGAGCUCU CGGGAAGA | 1726 |
| 74 | CCGGCAGA G CAGCCCAA | 1431 | UUGGGCUG GCCGAAAGGCGAGUGAGGUCU UCUGCCGG | 1727 |
| 77 | GCAGAGCA G CCCAAGCA | 1432 | UGCUUGGG GCCGAAAGGCGAGUGAGGUCU UGCUCUGC | 1728 |
| 83 | CAGCCCAA G CAGCGGGG | 1433 | CCCCGCUG GCCGAAAGGCGAGUGAGGUCU UUGGGCUG | 1729 |
| 86 | CCCAAGCA G CGGGGCAU | 1434 | AUGCCCCG GCCGAAAGGCGAGUGAGGUCU UGCUUGGG | 1730 |
| 91 | GCAGCGGG G CAUGCGCU | 1435 | AGCGCAUG GCCGAAAGGCGAGUGAGGUCU CCCGCUGC | 1731 |
| 95 | CGGGGCAU G CGCUUCCG | 1436 | CGGAAGCG GCCGAAAGGCGAGUGAGGUCU AUGCCCCG | 1732 |
| 97 | GGGCAUGC G CUUCCGCU | 1437 | AGCGGAAG GCCGAAAGGCGAGUGAGGUCU GCAUGCCC | 1733 |
| 103 | GCGCUUCC G CUACAAGU | 1438 | ACUUGUAG GCCGAAAGGCGAGUGAGGUCU GGAAGCGC | 1734 |
| 110 | CGCUACAA G UGCGAGGG | 1439 | CCCUCGCA GCCGAAAGGCGAGUGAGGUCU UUGUAGCG | 1735 |
| 112 | CUACAAGU G CGAGGGGC | 1440 | GCCCCUCG GCCGAAAGGCGAGUGACGUCU ACUUGUAG | 1736 |
| 119 | UGCGAGGG G CGCUCCGC | 1441 | GCGGAGCG GCCGAAAGGCGAGUGAGGUCU CCCUCGCA | 1737 |
| 121 | CGAGGGGC G CUCCGCGG | 1442 | CCGCGGAG GCCGAAAGGCCAGUGAGGUCU GCCCCUCG | 1738 |
| 126 | GGCGCUCC G CGGGCAGC | 1443 | GCUGCCCG GCCGAAAGGCGAGUGAGGUCU GGAGCGCC | 1739 |
| 130 | CUCCGCGG C CACCAUCC | 1444 | GGAUGCUG CCCGAAAGGCGAGUGAGGUCU CCGCGGAG | 1740 |
| 133 | CGCGGGCA G CAUCCCAG | 1445 | CUGGGAUG GCCGAAAGGCGAGUGAGGUCU UGCCCGCG | 1741 |
| 142 | CAUCCCAG G CGAGAGGA | 1446 | UCCUCUCG GCCGAAACGCGAGUGAGGUCU CUGGCAUG | 1742 |
| 151 | OGAGAGGA C CACAGAUA | 1447 | UAUCUGUG GCCGAAAGGCGAGUGAGGUCU UCCUCUCG | 1743 |
| 193 | GAUCAAUG C CUACACAG | 1448 | CUGUGUAG GCCGAAAGGCGAGUGAGGUCU CAUUGAUC | 1744 |
| 213 | CAGGGACA C UGCGCAUC | 1449 | GAUGCGCA GCCGAAAGGCGAGUGAGGUCU UGUCCCUG | 1745 |
| 215 | GGGACAGU G CGCAUCUC | 1450 | GAGAUGCG GCCGAAAGGCGAGUGAGGUCU ACUGUCCC | 1746 |
| 217 | GACAGUGC C CAUCUCCC | 1451 | GGGAGAUG GCCGAAAGGCGAGUGAGGUCU GCACUGUC | 1747 |
| 228 | UCUCCCUG G UCACCAAG | 1452 | CUUGGUGA GCCGAAAGGCGAGUGAGGUCU CAGGGAGA | 1748 |
| 251 | CCUCACCG C CCUCACCC | 1453 | GGGUGAGG GCCGAAAGGCGAGUGAGGUCU CGGUGAGG | 1749 |
| 266 | CCCCACGA G CUUGUAGG | 1454 | CCUACAAG GCCGAAAGGCGAGUGAGGUCU UCCUCGGG | 1750 |
| 270 | ACGAGCUU C UAGGAAAG | 1455 | CUUUCCUA GCCGAAAGGCGAGUGAGGUCU AAGCUCGU | 1751 |
| 283 | AAAGGACU G CCGGGAUG | 1456 | CAUCCCGG GCCGAAAGGCGAGUGAGGUCU AGUCCUUU | 1752 |
| 292 | CCGCGAUG G CUUCUAUG | 1457 | CAUAGAAG GCCCAAAGGCGAGUGAGGUCU CAUCCCGG | 1753 |
| 303 | UCUAUGAG C CUGAGCUC | 1458 | GACCUCAG CCCGAAACGCGAGUGAGGUCU CUCAUAGA | 1754 |
| 308 | GAGGCUGA C CUCUGCCC | 1459 | GCCCAGAG GCCGAAAGGCGAGUCACGUCU UCAGCCUC | 1755 |
| 313 | UCAGCUCU G CCCCGACC | 1460 | GGUCCGGG GCCGAAAGGCGAGUCAGGUCU AGACCUCA | 1756 |
| 322 | CCCGGACC G CUGCAUCC | 1461 | GGAUGCAC GCCGAAAGGCGAGUGAGGUCU GGUCCGGG | 1757 |
| 325 | GGACCGCU G CAUCCACA | 1462 | UGUGGAUG GCCGAAAGGCGAGUGAGGUCU AGCGGUCC | 1758 |
| 334 | CAUCCACA C UUUCCACA | 1463 | UCUCCAAA GCCCAAAGGCGACUCAGGUCU UGUGGAUG | 1759 |
| 356 | GGAAUCCA G UGUGUGAA | 1464 | UUCACACA GCCGAAAGGCGAGUGAGGUCU UGGAUUCC | 1760 |
| 358 | AAUCCACU C UCUCAAGA | 1465 | UCUUCACA GCCCAAAGGCGAGUGAGGUCU ACUCCAUU | 1761 |
| 360 | UCCAGUGU C UGAAGAAG | 1466 | CUUCUUCA GCCGAAAGGCGAGUCAGGUCU ACACUCCA | 1762 |
| 368 | GUGAACAA G CGGGACCU | 1467 | AGGUCCCC CCCGAAAGGCGAGUCACCUCU UUCUUCAC | 1763 |
| 380 | CACCUGGA G CAGGCUAU | 1468 | AUAGCCUG GCCGAAAGGCGAGUGAGGUCU UCCAGGUC | 1764 |
| 384 | UGGACCAG G CUAUCAGU | 1469 | ACUGAUAG GCCGAAAGGCGAGUGAGGUCU CUGCUCCA | 1765 |
| 391 | GGCUAUCA G UCAGCGCA | 1470 | UGCGCUGA GCCGAAAGGCGAGUGAGGUCU UGAUAGCC | 1766 |
| 395 | AUCAGUCA G CGCAUCCA | 1471 | UGGAUGCG GCCGAAAGGCCAGUGAGCUCU UGACUGAU | 1767 |
| 397 | CAGUCAGC G CAUCCAGA | 1472 | UCUGGAUG GCCGAAAGGCGAGUGAGGUCU GCUGACUG | 1768 |
| 426 | CCUUCCAA G UUCCUAUA | 1473 | UAUAGGAA GCCGAAAGGCGAGUGAGGUCU UUCGAAGG | 1769 |
| 440 | AUAGAACA C CAGCGUGG | 1474 | CCACGCUG GCCGAAAGGCGAGUGAGGUCU UCUUCUAU | 1770 |
| 443 | CAACACCA C COUGGOGA | 1475 | UCCCCACG GCCCAAAGGCGAGUGAGGUCU UGCUCUUC | 1771 |
| 445 | AGAGCAGC G UGGGGACU | 1476 | ACUCCCCA GCCGAAAGGCGAGUGAGGUCU GCUGCUCU | 1772 |
| 465 | ACCUCAAU C CUGUCCCG | 1477 | CCGCACAG GCCGPAAGGCGAGUGAGGUCU AUUCAGGU | 1773 |
| 468 | UGAAUGCU C UGCGGCUC | 1478 | GAGCCGCA GCCCAAAGGCGAGUGAGGUCU AGCAUUCA | 1774 |
| 470 | AAUGCUGU C CGGCUCUG | 1479 | CAGAGCCG CCCGAAAGGCGAGUGAGCUCU ACAGCAUU | 1775 |
| 473 | GCUGUGCG G CUCUGCUU | 1480 | AAGCAGAG GCCGAAAGGCGAGUGAGGUCU CGCACAGC | 1776 |
| 478 | GCGGCUCU G CUUCCAGC | 1481 | CCUGGAAG GCCGAAAGGCGACUGAGGUCU AGAGCCGC | 1777 |
| 486 | GCUUCCAG G UCACAGUG | 1482 | CACUGUCA GCCCAAAGGCGAGUGAGCUCU CUGGAAGC | 1778 |
| 492 | AGGUGACA C UGCGCGAC | 1483 | CUCCCGCA GCCGAAAGGCGAGUGAGGUCU UGUCACCU | 1779 |
| 494 | GUGACAGU G CCGCACCC | 1484 | CCCUCCCG CCCCAAAGGCCAGUGAGGUCU ACUGUCAC | 1780 |
| 508 | CCCAUCAG G CAGGCCCC | 1485 | CCCCCCUG GCCGAAAGGCCAGUGACCUCU CUCAUGGG | 1781 |
| 512 | UCAGCCAG G CCCCUCCC | 1486 | CCGAGCGG GCCGAAAGGCCACUGACGUCU CUGCCUGA | 1782 |
| 520 | GCCCCUCC G CCUGCCCC | 1487 | GCGCCAGG GCCGAAAGGCGAGUGAGGUCU CCACGCCC | 1783 |
| 524 | CUCCGCCU C CCGCCUGU | 1488 | ACACGCCG GCCGAAAGGCGACUGAGCUCU AGCCGGAG | 1784 |
| 527 | CGCCUGCC C CCUGUCCU | 1489 | ACGACACC CCCGAAAGGCGAGUGAGGUCU GGCAGCCC | 1785 |
| 531 | UGCCGCCU G UCCUUUCU | 1490 | AGAAAGGA CCCGAAAGGCCAGUGAGGUCU ACGCGGCA | 1786 |
| 559 | UGACAAUC G UGCCCCCA | 1491 | UCCGGGCA GCCGAAAGGCGAGUGAGGUCU CAUUGUCA | 1787 |
| 561 | ACAAUCGU C CCCCCAAC | 1492 | GUUCGCGG GCCGAAAGGCGAGUCACGUCU ACGAUUGU | 1788 |
| 573 | CCAACACU C CCGAGCUC | 1493 | GAGCUCGC CCCGAAAGGCGAGUGAGGUCU AGUGUUCG | 1789 |
| 578 | ACUGCCCA C CUCAAGAU | 1494 | AUCUUGAG GCCCAAAGCCGAGUCACCUCU UCCGCAGU | 1790 |
| 589 | CAAGAUCU C CCCACUGA | 1495 | UCACUCCC CCCGAAAGGCGACUGAGGUCU AGAUCUUG | 1791 |
| 594 | UCUCCCGA C UCAACCGA | 1496 | UCGGUUCA CCCGAAAGGCCACUGAGGUCU UCGGCACA | 1792 |
| 610 | AAACUCUG C CAGCUGCC | 1497 | GGCAGCUC GCCCAAAGGCGAGUGACGUCU CAGAGUUU | 1793 |
| 613 | CUCUGCCA C CUGCCUCC | 1498 | CCACGCAC GCCGAAAGCCCACUGAGGUCU UGCCACAC | 1794 |
| 616 | UGGCAGCU C CCUCGGUG | 1499 | CACCGAGG GCCCAAAGGCCAGUCACCUCU AGCUGCCA | 1795 |
| 622 | CUCCCUCC C UCGCCAUC | 1500 | CAUCCCCA GCCCAAAGCCCACUCACGUCU CGAGCCAC | 1796 |
| 644 | UUCCUACU C UCUGACAA | 1501 | UUGUCACA GCCGAAAGGCGACUCACCUCU ACUACCAA | 1797 |
| 646 | CCUACUCU G UCACAAGC | 1502 | CCUUGUCA CCCCAAAGCCGACUGACCUCU ACAGUAGC | 1798 |
| 654 | GUCACAAG G UGCAGAAA | 1503 | UUUCUGCA GCCGAAAGGCGAGUGAGGUCU CUUCUCAC | 1799 |
| 656 | GACAACGU C CACAAACA | 1504 | UCUUUCUC CCCCAAAGCCCAGUCACGUCU ACCUUGUC | 1800 |
| 675 | ACAUUGAG C UCUAUUUC | 1505 | CAAAUACA GCCCUAAGGCCAGUGAGGUCU CUCAAUGU | 1801 |
| 677 | AUUGAGGU C UAUUUCAC | 1506 | GUCAAAUA CCCCAAACCCCACUCACCUCU ACCUCAAU | 1802 |
| 694 | CCCACCAG C CUGGCAGG | 1507 | CCUCCCAG CCCCAAAGCCCAGUGACCUCU CUGGUCCC | 1803 |
| 702 | GCUCCGAG C CCCGACCC | 1508 | GCCUCGGC GCCGAAAGGCCAGUGAGGUCU CUCCCACC | 1804 |
| 709 | GGCCCCAG C CUCCUUUU | 1509 | AAAAGCAC CCCGAAAGGCGACUGACCUCU CUCCCCCC | 1805 |
| 719 | UCCUUUUC C CAACCUGA | 1510 | UCAGCUUG GCCCAAAGCCGACUGACCUCU GAAAAGCA | 1806 |
| 723 | UUUCCCAA C CUGAUGUC | 1511 | CACAUCAC CCCCAAACCCCACUGAGGUCU UUGCGAAA | 1807 |
| 729 | AACCUGAU C UCCACCCA | 1512 | UCCCUCCA CCCGAAAGCCCACUCACCUCU AUCAGCUU | 1808 |
| 731 | GCUCAUCU C CACCCACA | 1513 | UGUCGGUG CCCCAAACCCCACUCACCUCU ACAUCACC | 1809 |
| 741 | ACCCACAA C UCGCCAUU | 1514 | AAUCGCCA CCCCAAAGGCCACUCACGUCU UUCUCGGU | 1810 |
| 744 | CACAACUG C CCAUUCUC | 1515 | CACAAUCC CCCCAAAGCCCACUCACCUCU CACUUGUC | 1811 |
| 750 | UCGCCAUU C UCUUCCCC | 1516 | CCCCAACA GCCGAAAGGCCAGUGACCUCU AAUGCCCA | 1812 |
| 752 | GCCAUUCU C UUCCCCAC | 1517 | CUCCGCAA CCCCAAAGCCGACUCACCUCU ACAAUGCC | 1813 |
| 771 | CUCCCUAC C CAGACCCC | 1518 | CCCCUCUC CCCCAAAGGCCACUCACCUCU CUAGCGAC | 1814 |
| 781 | AGACCCCA G CCUGCAGG | 1519 | CCUGCAGG GCCGAAAGGCGAGUGAGGUCU UGGGGUCU | 1815 |
| 785 | CCCAGCCU G CAGGCUCC | 1520 | GGAGCCUG GCCGAAAGGCGAGUGAGGUCU AGGCUGGG | 1816 |
| 789 | GCCUGCAG G CUCCUGUG | 1521 | CACAGGAG GCCGAAAGGCGAGUGAGGUCU CUGCAGGC | 1817 |
| 795 | AGGCUCCU G UGCGUGUC | 1522 | GACACGCA GCCGAAAGGCGAGUGAGGUCU AGGAGCCU | 1818 |
| 797 | GCUCCUGU G CGUGUCUC | 1523 | GAGACACG GCCGAAAGGCGAGUGAGGUCU ACAGGAGC | 1819 |
| 799 | UCCUGUGC G UGUCUCCA | 1524 | UGGAGACA GCCGAAAGGCGAGUGAGGUCU GCACAGGA | 1820 |
| 801 | CUGUGCGU G UCUCCAUG | 1525 | CAUGGAGA GCCGAAAGGCGAGUGAGGUCU ACGCACAG | 1821 |
| 809 | GUCUCCAU G CAGCUGCG | 1526 | CGCAGCUG GCCGAAAGGCGAGUGAGGUCU AUGGAGAC | 1822 |
| 812 | UCCAUGCA G CUGCGGCG | 1527 | CGCCGCAG GCCGAAAGGCGAGUGAGGUCU UGCAUGGA | 1823 |
| 815 | AUGCAGCU G CGGCGGCC | 1528 | GGCCGCCG GCCGAAAGGCGAGUGAGGUCU AGCUGCAU | 1824 |
| 818 | CAGCUGCG G CGGCCUUC | 1529 | GAAGGCCG GCCGAAAGGCGAGUGAGGUCU CGCAGCUG | 1825 |
| 821 | CUGCGGCG G CCUUCCGA | 1530 | UCGGAAGG GCCGAAAGGCGAGUGAGGUCU CGCCGCAG | 1826 |
| 836 | GACCGGGA G CUCAGUGA | 1531 | UCACUGAG GCCGAAAGGCGAGUGAGGUCU UCCCGGUC | 1827 |
| 841 | GGAGCUCA G UGAGCCCA | 1532 | UGGGCUCA GCCGAAAGGCGAGUGAGGUCU UGAGCUCC | 1828 |
| 845 | CUCAGUGA G CCCAUGGA | 1533 | UCCAUGGG GCCGAAAGGCGAGUGAGGUCU UCACUGAG | 1829 |
| 860 | GAAUUCCA G UACCUGCC | 1534 | GGCAGGUA GCCGAAAGGCGAGUGAGGUCU UGGAAUUC | 1830 |
| 866 | CAGUACCU G CCAGAUAC | 1535 | GUAUCUGG GCCGAAAGGCGAGUGAGGUCU AGGUACUG | 1831 |
| 883 | AGACGAUC G UCACOGGA | 1536 | UCCGGUGA GCCGAAAGGCGAGUGAGGUCU GAUCGUCU | 1832 |
| 904 | GGAGAAAC G UAAAAGGA | 1537 | UCCUUUUA GCCGAAAGGCGAGUGAGGUCU GUUUCUCC | 1833 |
| 931 | CUUCAAGA G CAUCAUGA | 1538 | UCAUGAUG GCCGAAAGGCGAGUGAGGUCU UCUUGAAG | 1834 |
| 946 | GAAGAAGA G UCCUUUCA | 1539 | UGAAAGGA GCCGAAAGGCGAGUGAGGUCU UCUUCUUC | 1835 |
| 955 | UCCUUUCA G CGGACCCA | 1540 | UGGGUCCG GCCGAAAGGCGAGUGAGGUCU UGAAAGGA | 1836 |
| 974 | GACCCCCG G CCUCCACC | 1541 | GGUGGAGG GCCGAAAGGCGAGUGAGGUCU CGGGGGUC | 1837 |
| 988 | ACCUCGAC G CAUUGCUG | 1542 | CAGCAAUG GCCGAAAGGCGAGUGAGGUCU GUCGAGGU | 1838 |
| 993 | GACGCAUU G CUGUGCCU | 1543 | AGGCACAG GCCGAAAGGCGAGUGAGGUCU AAUGCGUC | 1839 |
| 996 | GCAUUGCU G UGCCUUCC | 1544 | GGAAGGCA GCCGAAAGGCGAGUGAGGUCU AGCAAUGC | 1840 |
| 998 | AUUGCUGU G CCUUCCCG | 1545 | CGGGAAGG GCCGAAAGGCGAGUGAGGUCU ACAGCAAU | 1841 |
| 1006 | GCCUUCCC G CAGCUCAG | 1546 | CUGAGCUG GCCGAAAGGCGAGUGAGGUCU GGGAAGGC | 1842 |
| 1009 | UUCCCGCA G CUCAGCUU | 1547 | AAGCUGAG GCCGAAAGGCGAGUGAGGUCU UGCGGGAA | 1843 |
| 1014 | GCAGCUCA G CUUCUGUC | 1548 | GACAGAAG GCCGAAAGGCGAGUGAGGUCU UGAGCUGC | 1844 |
| 1020 | CAGCUUCU G UCCCCAAG | 1549 | CUUGGGGA GCCGAAAGGCGAGUGAGGUCU AGAAGCUG | 1845 |
| 1028 | GUCCCCAA G CCAGCACC | 1550 | GGUGCUGG GCCGAAAGGCGAGUGAGGUCU UUGGGGAC | 1846 |
| 1032 | CCAAGCCA G CACCCCAG | 1551 | CUGGGGUG GCCGAAAGGCGAGUGAGGUCU UGGCUUGG | 1847 |
| 1040 | GCACCCCA G CCCUAUCC | 1552 | GGAUAGGG GCCGAAAGGCGAGUGAGGUCU UGGGGUGC | 1848 |
| 1055 | CCCUUUAC G UCAUCCCU | 1553 | AGGGAUGA GCCGAAAGGCGAGUGAGGUCU GUAAAGGG | 1849 |
| 1066 | AUCCCUGA G CACCAUCA | 1554 | UGAUGGUG GCCGAAAGGCGAGUGAGGUCU UCAGGGAU | 1850 |
| 1085 | UAUGAUGA G UUUCCCAC | 1555 | GUGGGAAA GCCGAAAGGCGAGUGAGGUCU UCAUCAUA | 1851 |
| 1098 | CCACCAUG G UGUUUCCU | 1556 | AGGAAACA GCCGAAAGGCGAGUGAGGUCU CAUGGUGG | 1852 |
| 1100 | ACCAUGGU G UUUCCUUC | 1557 | GAAGGAAA GCCGAAAGGCGAGUGAGGUCU ACCAUGGU | 1853 |
| 1112 | CCUUCUGG G CAGAUCAG | 1558 | CUGAUCUG GCCGAAAGGCGAGUGAGGUCU CCAGAAGG | 1854 |
| 1120 | GCAGAUCA G CCAGGCCU | 1559 | AGGCCUGG GCCGAAAGGCGAGUGAGGUCU UGAUCUGC | 1855 |
| 1125 | UCAUCCAG G CCUCGGCC | 1560 | GGCCGAGG GCCGAAAGGCGAGUGAGGUCU CUGGCUGA | 1856 |
| 1131 | AGGCCUCG G CCUUGGCC | 1561 | GGCCAAGG GCCGAAAGGCGAGUGAGGUCU CGAGGCCU | 1857 |
| 1137 | CGGCCUUG G CCCCGGCC | 1562 | GGCCGGGG GCCGAAAGGCGAGUGAGGUCU CAAGGCCG | 1858 |
| 1143 | UGGCCCCG G CCCCUCCC | 1563 | GGGAGGGG GCCGAAAGGCGAGUGAGGUCU CGGGGCCA | 1859 |
| 1155 | CUCCCCAA G UCCUGCCC | 1564 | GGGCAGGA GCCGAAAGGCGAGUGAGGUCU UUGGGGAG | 1860 |
| 1160 | CAAGUCCU G CCCCAGGC | 1565 | GCCUGGGG GCCGAAAGGCGAGUGAGGUCU AGGACUUG | 1861 |
| 1167 | UGCCCCAG G CUCCAGCC | 1566 | GGCUGGAG GCCGAAAGGCGAGUGAGGUCU CUGGGGCA | 1862 |
| 1173 | AGGCUCCA G CCCCUGCC | 1567 | GGCAGGGG GCCGAAAGGCGAGUGAGGUCU UGGAGCCU | 1863 |
| 1179 | CAGCCCCU G CCCCUGCU | 1568 | AGCAGGGG GCCGAAAGGCGAGUGAGGUCU AGGGGCUG | 1864 |
| 1185 | CUGCCCCU G CUCCAGCC | 1569 | GGCUGGAG GCCGAAAGGCGAGUGAGGUCU AGGGGCAG | 1865 |
| 1191 | CUCCUCCA G CCAUGGUA | 1570 | UACCAUGG GCCGAAAGGCGAGUGAGGUCU UGGAGCAG | 1866 |
| 1197 | CAGCCAUG G UAUCAGCU | 1571 | AGCUGAUA GCCGAAAGGCGAGUGACGUCU CAUGCCUG | 1867 |
| 1203 | UGGUAUCA G CUCUGGCC | 1572 | CGCCAGAG GCCGAAAGGCGAGUGAGGUCU UGAUACCA | 1868 |
| 1209 | CAGCUCUG G CCCAAGCC | 1573 | GGCCUGGG GCCGAAAGGCGAAUGAGGUCU CAGAGCUG | 1869 |
| 1215 | UGGCCCAG G CCCCAGCC | 1574 | GGCUGGGG GCCGAAAGGCGAGUGAGGUCU CUGGGCCA | 1870 |
| 1221 | AGGCCCCA C CCCCUGUC | 1575 | GACAGCGG CCCGAAAGGCGAGUCAGGUCU UGGGGCCU | 1871 |
| 1227 | CACCCCCU G UCCCAGUC | 1576 | GACUGGGA GCCGAAAGGCGAGUGAGGUCU AGGGGCUG | 1872 |
| 1233 | CUGUCCCA G UCCUAGCC | 1577 | CGCUAGGA GCCGAAAGGCGAGUGAGGUCU UGGGACAG | 1873 |
| 1239 | CAGUCCUA C CCCCAGGC | 1578 | GCCUGGCG CCCGAAACGCGACUGAGGUCU UAGCACUG | 1874 |
| 1246 | AGCCCCAG G CCCUCCUC | 1579 | CAGGACGG CCCGAAAGGCGACUGAGGUCU CUGGGGCU | 1875 |
| 1257 | CUCCUCAG G CUGUCGCC | 1580 | GGCCACAG GCCGAAAGGCGAGUGAGGUCU CUGAGGAG | 1876 |
| 1260 | CUCAGGCU G UGGCCCCA | 1581 | UGGGGCCA GCCGAAAGGCGAGUGAGGUCU AGCCUGAG | 1877 |
| 1263 | AGGCUGUG G CCCCACCU | 1582 | AGGUGGGG GCCGAAAGGCGAGUGAGGUCU CACAGCCU | 1878 |
| 1272 | CCCCACCU G CCCCCAAC | 1583 | CUUCGGGG GCCGAAAGGCGAGUGAGGUCU AGGUGGGG | 1879 |
| 1280 | GCCCCCAA G CCCACCCA | 1584 | UGGCUGGG GCCGAAAGGCGAGUGAGGUCU UUGGGGGC | 1880 |
| 1290 | CCACCCAG G CUGGGGAA | 1585 | UUCCCCAG GCCGAAAGGCGAGUGAGCUCU CUGOGUOG | 1881 |
| 1304 | GAAGGAAC G CUGUCAGA | 1586 | UCUGACAG GCCGAAAGGCGAGUGAGGUCU GUUCCUUC | 1882 |
| 1307 | GGAACGCU G UCAGAGGC | 1587 | GCCUCUGA GCCGAAAGGCGAGUGAGGUCU AGCGUUCC | 1883 |
| 1314 | UGUCAGAG C CCCUGCUG | 1588 | CAGCAGGG GCCGAAAGGCGAGUGAGGUCU CUCUGACA | 1884 |
| 1319 | GAGGCCCU G CUGCAGCU | 1589 | AGCUGCAG GCCGAAAGGCCAGUGAGGUCU AGGGCCUC | 1885 |
| 1322 | CCCCUGCU G CACCUCCA | 1590 | UGCAGCUG GCCGAAAGGCGAGUGAGGUCU AGCAGGGC | 1886 |
| 1325 | CUGCUGCA G CUGCAGUU | 1591 | AACUGCAG GCCGAAACGCGAGUGAGGUCU UGCAGCAG | 1887 |
| 1328 | CUGCAGCU G CAGUUUGA | 1592 | UCAAACUG GCCGAAAGGCGAGUGAGGUCU AGCUCCAG | 1888 |
| 1331 | CAGCUGCA G UUUGAUGA | 1593 | UCAUCAAA GCCGAAACGCCAGUGAGGUCU UGCAGCUG | 1889 |
| 1353 | ACCUGGGG C CCUUGCUU | 1594 | AAGCAAGG CCCGAAAGGCGAGUGAGGUCU CCCCAGGU | 1890 |
| 1358 | GGGGCCUU C CUUGGCAA | 1595 | UUGCCAAG GCCGAAAGGCGAGUGAGGUCU AAGGCCCC | 1891 |
| 1363 | CUUGCUUG C CAACAGCA | 1596 | UGCUGUUG GCCGAAAGGCGAGUGAGGUCU CAAGCAAG | 1892 |
| 1369 | UGGCAACA C CACAGACC | 1597 | GCUCUGUC GCCGAAACGCGAGUGAGGUCU UGUUGCCA | 1893 |
| 1380 | CAGACCCA G CUGUGGUC | 1598 | GAACACAG GCCGAAACGCGAGUGAGGUCU UGGGUCUG | 1894 |
| 1383 | ACCCAGCU G UGUUCACA | 1599 | UGUGAACA GCCGAAAGGCGAGUGAGGUCU AGCUGGGU | 1895 |
| 1385 | CCAGCUGU C UUCACAGA | 1600 | UCUGUGAA GCCGAAAGGCGAGUGAGCUCU ACAGCUCG | 1896 |
| 1398 | CAGACCUG C CAUCCCUC | 1601 | GACGGAUG GCCGAAAGGCGAGUGAGGUCU CAGGUCUG | 1897 |
| 1404 | UGGCAUCC C UCCACAAC | 1602 | GUUGUCCA GCCCAAAGCCGAGUGAGGUCU GCAUCCCA | 1898 |
| 1418 | AACUCCGA C UUUCAGCA | 1603 | UGCUGAAA GCCGAAAGGCGAGUGAGGUCU UCGGAGUU | 1899 |
| 1424 | GAGUUUCA C CAGCUGCU | 1604 | ACCACCUG CCCGAAACGCGAGUGAGGUCU UGAAACUC | 1900 |
| 1427 | UUUCAGCA C CUGCUGAA | 1605 | UUCAGCAG GCCCAAAGGCGACUGAGGUCU UCCUCAAA | 1901 |
| 1430 | CAGCAGCU C CUGAACCA | 1606 | UGGUUCAG GCCGAAAGGCGAGUGACGUCU ACCUCCUG | 1902 |
| 1441 | CAACCAGG C CAUACCUG | 1607 | CAGGUAUG GCCGAAAGGCGAGUGAGGUCU CCUGGUUC | 1903 |
| 1449 | GCAUACCU C UCGCCCCC | 1608 | GGGGGCCA GCCGAAAGGCGAGUGAGGUCU AGCUAUGC | 1904 |
| 1452 | UACCUCUG C CCCCCCAC | 1609 | CUGCGGCG GCCGAAAGGCGAGUGAGGUCU CACAGGUA | 1905 |
| 1469 | ACAACUCA C CCCAUGCU | 1610 | AGCAUGGG GCCGAAAGGCGAGUGAGGUCU UCAGUUCU | 1906 |
| 1475 | GAGCCCAU C CUGAUGGA | 1611 | UCCAUCAC GCCCAAACGCCAGUGAGCUCU AUCGGCUC | 1907 |
| 1484 | CUGAUGGA C UACCCUGA | 1612 | UCAGGCUA GCCGAAACCCGAGUGACGUCU UCCAUCAC | 1908 |
| 1494 | ACCCUCAG C CUAUAACU | 1613 | AGUUAUAG GCCGAAAGGCGAGUGAGGUCU CUCAGGGU | 1909 |
| 1504 | UAUAACUC C CCUACUGA | 1614 | UCACUAGC CCCCAAACGCGACUGACGUCU GAGUUAUA | 1910 |
| 1509 | CUCGCCUA C UGACAGCC | 1615 | CGCUGUCA GCCGAAAGGCGAGUGAGGUCU UAGGCGAG | 1911 |
| 1515 | UACUGACA C CCCACACC | 1616 | CCUCUCCG GCCCAAACGCGACUGACCUCU UGUCACUA | 1912 |
| 1523 | CCCCACAG C CCCCCCGA | 1617 | UCGGCGCC CCCGAAAGGCGAGUGAGGUCU CUCUCGCC | 1913 |
| 1536 | CCGACCCA C CUCCUGCU | 1618 | ACCAGGAG CCCCAAACCCCAGUCACGUCU UCCGUCGG | 1914 |
| 1542 | CAGCUCCU C CUCCACUG | 1619 | CAGUGGAG GCCGAAAGCCGAGUGACGUCU AGGACCUC | 1915 |
| 1554 | CACUGGGC C CCCCCGGG | 1620 | CCCCGGCG GCCGAAAGGCGAGUGAGCUCU CCCCAGUC | 1916 |
| 1562 | GCCCCGGG G CUCCCCAA | 1621 | UUGGGGAG GCCGAAAGGCGAGUGAGGUCU CCCGGGAAC | 1917 |
| 1573 | CCCCAAUG G CCUCCUUU | 1622 | AAAGGAGG GCCGAAAGGCGAGUGAGGUCU CAUUGGGG | 1918 |
| 1608 | CCUCCAUU G OGGACAUG | 1623 | CAUGUCCG GCCGAAAGGCGAGUGAGGUCU AAUGGAGG | 1919 |
| 1626 | ACUUCUCA G CCCUGCUG | 1624 | CAGCAGGG GCCGAAAGGCGAGUGAGGUCU UGAGAAGU | 1920 |
| 1631 | UCAGCCCU G CUGAGUCA | 1625 | UGACUCAG GCCGAAAGGCGAGUGAGGUCU AGGGCUGA | 1921 |
| 1636 | CCUGCUGA G UCAGAUCA | 1626 | UGAUCUGA GCCGAAAGGCGAGUGAGGUCU UCAGCAGG | 1922 |
| 1645 | UCAGAUCA C CUCCUAAG | 1627 | CUCAGGAG GCCGAAAGGCGAGUGAGGUCU UGAUCUGA | 1923 |
| 1657 | CUAAGGGG G UGACGCCU | 1628 | AGGCGUCA GCCGAAAGGCGAGUGAGGUCU CCCCUUAG | 1924 |
| 1662 | GGGGUGAC G CCUGCCCU | 1629 | AGGGCAGG GCCGAAAGGCGAGUGAGGUCU GUCACCOC | 1925 |
| 1666 | UGACGCCU G CCCUCCCC | 1630 | GGGGAGGG GCCGAAAGGCGAGUGAGGUCU AGGCGUCA | 1926 |
| 1678 | UCCCCAGA G CACUGGUC | 1631 | AACCAGUG GCCGAAAGGCGAGUGAGGUCU UCUGGGGA | 1927 |
| 1684 | GACCACUG G UUGCAGGG | 1632 | CCCUGCAA GCCGAAAGGCGAGUGAGGUCU CAGUGCUC | 1928 |
| 1687 | CACUGGUC G CAGGGGAU | 1633 | AUCCCCUG GCCGAAAGGCGAGUGAGGUCU AACCAGUG | 1929 |
| 1700 | GGAUUGAA G CCCUCCAA | 1634 | UUGGAGGG GCCGAAAGGCGAGUGAGGUCU UUCAAUCC | 1930 |
| 1711 | CUCCAAAA C CACUUACG | 1635 | CGUAAGUG GCCGAAAGGCGAGUGAGGUCU UUUUGGAG | 1931 |
| 1727 | GGAUUCUG G UGGGGUGU | 1636 | ACACCCCA GCCGAAAGGCGAGUGAGGUCU CAGAAUCC | 1932 |
| 1732 | CUGGUGGG G UGUGUUCC | 1637 | GGAACACA GCCGAAAGGCGAGUGAGGUCU CCCACCAG | 1933 |
| 1734 | GGUGGGGU G UGUUCCAA | 1638 | UUGGAACA GCCGAAAGGCGAGUGAGGUCU ACCCCACC | 1934 |
| 1736 | UGGGGUGU C UUCCAACU | 1639 | AGUUGGAA GCCGAAAGGCGAGUGAGGUCU ACACCCCA | 1935 |
| 1745 | UUCCAACU G CCCCCAAC | 1640 | GUUGGGGG GCCGAAAGGCGAGUGAGGUCU AGUUGGAA | 1936 |
| 1757 | CCAACUUU G UGGAUGUC | 1641 | GACAUCCA GCCGAAAGGCGAGUGAGGUCU AAAGUUGG | 1937 |
| 1763 | UUGUGGAU G UCUUCCUC | 1642 | AAGGAAGA GCCGAAAGGCGAGUGAGGUCU AUCCACAA | 1938 |
| 1782 | AGGGGGGA G CCAUAUUU | 1643 | AAAUAUGG GCCGAAAGGCGAGUGAGGUCU UCCCCCCU | 1939 |
| 1803 | CUUUUAUU G UCAGUAUC | 1644 | GAUACUGA GCCGAAAGGCGAGUGAGGUCU AAUAAAAG | 1940 |
| 1807 | UAUUGUCA C UAUCUGUA | 1645 | UACAGAUA GCCGAAAGGCGAGUGAGGUCU UGACAAUA | 1941 |
| 1813 | CAGUAUCU C UAUCUCUC | 1646 | GAGAGAUA GCCGAAAGGCGAGUGAGGUCU AGAUACUG | 1942 |
| 1835 | UUUUGGAG C UGCUUAAG | 1647 | CUUAAGCA GCCGAAAGGCGAGUGAGGUCU CUCCAAAA | 1943 |
| 1837 | UUGGAGGU C CUUAAGCA | 1648 | UGCUUAAG GCCGAAAGGCGAGUGAGGUCU ACCUCCAA | 1944 |
| 1843 | GUGCUUAA C CAGAAGCA | 1649 | UGCUUCUG GCCGAAAGGCGAGUGAGGUCU UUAAGCAC | 1945 |
| 1849 | AAGCAGAA C CAUUAACU | 1650 | AGUUAAUG GCCGAAAGGCGAGUGAGGUCU UUCUGCUU | 1946 |
| 1875 | AGCGCCCA C CUGGGGAA | 1651 | UUCCCCAG CCCGAAAGGCGAGUGAGGUCU UCCCCCCU | 1947 |
| 1901 | UUUCCCCU C UCCUGAUG | 1652 | CAUCAGGA GCCGAAAGGCGAGUGAGGUCU AGGGGAAA | 1948 |
| 1910 | UCCUCAUC C UCACCUCC | 1653 | COACCUCA GCCGAAAGGCGAGUGAGGUCU CAUCACCA | 1949 |
| 1914 | GAUCCUCA C CUCCCUUC | 1654 | GAAGGGAG GCCGAAAGGCGAGUGAGGUCU UCACCAUC | 1950 |
| 1926 | CCUUCUCU C UAGGGAAC | 1655 | GUUCCCUA GCCGAAAGGCGAGUGAGGUCU AGAGAAGG | 1951 |
| 1936 | AGGGAACU C UCGGGUCC | 1656 | CCACCCCA GCCGAAAGGCGAGUGAGGUCU AGUUCCCU | 1952 |
| 1941 | ACUGUGGG C UCCCCCAU | 1657 | AUGGGGCA GCCGAAACGCGAGUGAGGUCU CCCACACU | 1953 |
| 1962 | AUCCUCCA C CUUCUGGU | 1658 | ACCAGAAG GCCGAAAGGCGAGUGAGGUCU UGCACGAU | 1954 |
| 1969 | AGCUUCUG C UACUCUCC | 1659 | GGAGAGUA GCCGAAAGGCGAGUGAGGUCU CAGAAGCU | 1955 |
| 1989 | AGACAGAA C CACCCUCC | 1660 | CCACCCUC GCCGAAACGCGACUGAGGUCU UUCUGUCU | 1956 |
| 1993 | AGAAGCAG C CUCCAGCU | 1661 | ACCUCCAG GCCGAAAGGCGAGUGAGGUCU CUGCUUCU | 1957 |
| 2000 | GGCUCGAC C UAAGGCCU | 1662 | AGGCCUUA GCCGAAAGGCGAGUGAGCUCU CUCCAGCC | 1958 |
| 2005 | GAGGUAAG C CCUUUGAG | 1663 | CUCAAAGG CCCGAAACCCGAGUCAGCUCU CUCACCUC | 1959 |
| 2013 | GCCUUUGA C CCCACAAA | 1664 | UUUGUGGG GCCCAAAGCCCACUGACCUCU UCAAAGGC | 1960 |
| 2022 | CCCACAAA C CCUUAUCA | 1665 | UCAUAAGC CCCGAAACGCGACUGAGGUCU UUUCUGGG | 1961 |
| 2032 | CUUAUCAA C UCUCUUCC | 1666 | GGAAGACA GCCGAAAGGCGAGUGAGGUCU UUGAUAAG | 1962 |
| 2034 | UAUCAAGU C UCUUCCAU | 1667 | AUCCAAGA CCCGAAACCCGACUCACCUCU ACUUCAUA | 1963 |
| 2058 | UCAUUACA C CUUAAUCA | 1668 | UCAUUAAC CCCGAAAGGCGACUGACGUCU UGUAAUGA | 1964 |
| 2074 | AAAAUAAC C CCCCACAU | 1669 | AUCUCCGC CCCCAAACCCCAGUCACCUCU CUUAUUUU | 1965 |
| 2087 | AGAUACCA C CCCCUGUA | 1670 | UACAGCGG CCCGAAAGGCGAGUGAGGUCU UCGUAUCU | 1966 |
| 2093 | CAGCCCCU C UAUGGCAC | 1671 | GUCCCAUA CCCGAAAGCCGACUCACCUCU ACCCCCUC | 1967 |
| 2098 | CCUGUAUG G CACUGGCA | 1672 | UGCCAGUG GCCGAAAGGCGAGUGAGGUCU CAUACAGG | 1968 |
| 2104 | UGGCACUG G CAUUGUCC | 1673 | GGACAAUG GCCGAAAGGCGAGUGAGGUCU CAGUGCCA | 1969 |
| 2109 | CUGGCAUU G UCCCUGUG | 1674 | CACAGGGA GCCGAAAGGCGAGUGAGGUCU AAUCCCAG | 1970 |
| 2115 | UUGUCCCU G UCCCUAAC | 1675 | GUUAGGCA GCCGAAAGGCGAGUGAGCUCU AGCGACAA | 1971 |
| 2117 | GUCCCUGU C CCUAACAC | 1676 | GUGUUAGG GCCGAAAGGCGAGUGAGGUCU ACAGGGAC | 1972 |
| 2128 | UAACACCA G CGUUUGAG | 1677 | CUCAAACG GCCGAAAGGCGAGUGAGGUCU UGGUGUUA | 1973 |
| 2130 | ACACCAGC G UUUGAGGG | 1678 | CCCUCAAA GCCGAAAGGCGAGUGAGGUCU GCUGGUGU | 1974 |
| 2139 | UUUGAGCG G CUGCCUUC | 1679 | GAAGGCAG GCCGAAAGGCGAGUGAGGUCU CCCUCAAA | 1975 |
| 2142 | GAGGGGCU G CCUUCCUG | 1680 | CAGGAAGG GCCGAAAGGCGAGUGAGGUCU AGCCCCUC | 1976 |
| 2150 | GCCUUCCU G CCCUACAG | 1681 | CUGUAGGG GCCGAAAGGCCACUGAGGUCU AGGAAGGC | 1977 |
| 2161 | CUACAGAG C UCUCUGCC | 1682 | GCCAGACA GCCGAAAGGCGAGUGAGGUCU CUCUGUAG | 1978 |
| 2167 | AGGUCUCU G CCGGCUCU | 1683 | AGAGCCGG GCCGAAAGGCGAGUGAGGUCU AGAGACCU | 1979 |
| 2171 | CUCUGCCG G CUCUUUCC | 1684 | GGAAAGAG GCCGAAAGGCGAGUGAGGUCU CGGCAGAG | 1980 |
| 2182 | CUUUCCUU G CUCAACCA | 1685 | UGGUUGAG GCCGAAAGGCGAGUGAGGUCU AAGGAAAG | 1981 |
| 2193 | CAACCAUG G CUGAAGGA | 1686 | UCCUUCAG GCCGAAAGGCGAGUGAGGUCU CAUGGUUG | 1982 |
| 2206 | AGGAAACA G UCCAACAG | 1687 | CUGUUGCA GCCGAAAGGCCAGUGAGGUCU UGUUUCCU | 1983 |
| 2208 | GAAACAGU G CAACAGCA | 1688 | UGCUGUUG GCCGAAAGGCGAGUGAGGUCU ACUGUUUC | 1984 |
| 2214 | GUGCAACA G CACUGGCU | 1689 | AGCCAGUG GCCGAAAGGCGAGUGAGGUCU UGUUGCAC | 1985 |
| 2220 | CACCACUG G CUCUCUCC | 1690 | GGAGACAG GCCGAAACCCCAGUGAGGUCU CAGUGCUC | 1986 |
| 2243 | CAGAAGGG G UUUGGUCU | 1691 | AGACCAAA GCCGAAAGGCGAGUGAGGUCU CCCUUCUG | 1987 |
| 2248 | GGGGUUUG G UCUGGACU | 1692 | AGUCCAGA GCCGAAAGCCGAGUGAGGUCU CAAACCCC | 1988 |
| 2262 | ACUUCCUU C CUCUCCCC | 1693 | GGGGAGAG GCCGAAAGGCGAGUGAGGUCU AAGGAAGU | 1989 |
| 2280 | CUUCUCAA C UGCCUUAA | 1694 | UUAAGGCA GCCGAAAGGCGAGUGAGCUCU UUGAGAAG | 1990 |
| 2282 | UCUCAAGU G CCUUAAUA | 1695 | UAUUAAGG CCCGAAAGGCGACUGAGGUCU ACUGGAGA | 1991 |
| 2291 | CCUUAAUA G UACGGUAA | 1696 | UUACCCUA GCCGAAAGGCGAGUGAGGUCU UAUUAAGG | 1992 |
| 2296 | AUAGUAGG C UAAGUUGU | 1697 | ACAACUUA GCCGAAAGGCGAGUCAGGUCU CCUACUAU | 1993 |
| 2300 | UAGGGUAA G UUGUUAAG | 1698 | CUUAACAA GCCGAAAGGCGAGUGAGGUCU UUACCCUA | 1994 |
| 2303 | GGUAAGUU C UUAAGAGU | 1699 | ACUCUUAA GCCGAAACGCGAGUGAGCUCU AACUUACC | 1995 |
| 2310 | UGUUAAGA G UGGGGGAG | 1700 | CUCCCCCA GCCGAAAGGCGAGUGAGGUCU UCUUAACA | 1996 |
| 2320 | GGGGGAGA G CAGCCUGG | 1701 | CCAGCCUG GCCGAAAGGCGAGUGAGGUCU UCUCCCCC | 1997 |
| 2324 | GAGACCAC C CUGGCAGC | 1702 | GCUGCCAC GCCGAAAGGCCACUGAGGUCU CUGCUCUC | 1998 |
| 2328 | GCAGGCUG C CAGCUCUC | 1703 | GAGAGCUG GCCGAAAGGCGAGUGAGGUCU CAGCCUGC | 1999 |
| 2331 | CGCUGGCA C CUCUCCAG | 1704 | CUGGAGAG GCCCAAAGGCGAGUGAGGUCU UGCCAGCC | 2000 |
| 2339 | GCUCUCCA G UCAGGAGG | 1705 | CCUCCUGA GCCGAAAGGCGAGUGAGGUCU UGGAGAGC | 2001 |
| 2347 | GUCAGGAG C CAUAGUUU | 1706 | AAACUAUG GCCGAAAGGCGAGUGAGGUCU CUCCUGAC | 2002 |
| 2352 | GAGGCAUA G UUUUUAGU | 1707 | ACUAAAAA GCCGAAAGGCGAGUGAGGUCU UAUGCCUC | 2003 |
| 2359 | AGUUUUUA G UGAACAAU | 1708 | AUUGUUCA GCCGAAACGCGAGUGAGGUCU UAAAAACU | 2004 |
| 2372 | CAAUCAAA C CACUUGGA | 1709 | UCCAACUG GCCGAAAGGCGAGUGAGGUCU UUUGAUUG | 2005 |
| 2386 | GGACUCUU C CUCUUUCU | 1710 | ACAAACAG GCCGAAAGGCGAGUGACGUCU AAGAGUCC | 2006 |
| 2411 | CUAAUAAA C CUGUUCCC | 1711 | GCCAACAC GCCGAAAGGCGAGUGACCUCU UUUAUUAG | 2007 |
| 2414 | AUAAAGCU G UUCCCAAG | 1712 | CUUGGCAA CCCGAAAGGCGAGUGACCUCU AGCUUUAU | 2008 |
| 2417 | AAGCUGUU C CCAAGCUG | 1713 | CAGCUUGG GCCGAAAGGCGAGUGAGCUCU AACAGCUU | 2009 |
| 2422 | CUUGCCAA G CUGGACGG | 1714 | CCGUCCAC GCCCAAAGGCGACUGAGGUCU UUGGCAAC | 2010 |
| 2430 | GCUGGACG C CACGAGCU | 1715 | AGCUCCUC CCCGAAAGCCCACUGAGGUCU CCUCCACC | 2011 |
| 2436 | CGCCACCA C CUCCUGCC | 1716 | GCCACGAG GCCGAAAGGCCAGUCAGGUCU UCCUGCCG | 2012 |

Input Sequence = NM_021975. Cut Site = G/Y
Arm Length = 8. Core Sequence = GCcgaaagGCGaGUCaaGGUCU
NM_021975 (Homo sapiens p65 RelA (NFKB), mRNA; 2444 bp)

TABLE V
Human REL-A DNAzyme and Substrate Sequence

| | | Seq | | Seq |
|---|---|---|---|---|
| Pos | Substrate | ID | DNAzyzme | ID |
| 9 | GGCACGAG G CGGGGCCG | 1421 | CGGCCCCG GGCTAGCTACAACGA CTCGTGCC | 2151 |
| 14 | GAGGCGGG G CCGGGUCG | 1422 | CGACCCGG GGCTAGCTACAACGA CCCGCCTC | 2152 |
| 19 | GGGGCCGG G UCGCAGCU | 1423 | AGCTGCGA GGCTAGCTACAACGA CCGGCCCC | 2153 |
| 22 | GCCGGGUC G CAGCUGGG | 1424 | CCCAGCTG GGCTAGCTACAACGA GACCCGGC | 2154 |
| 25 | GGGUCGCA G CUGGGCCC | 1425 | GGGCCCAG GGCTAGCTACAACGA TGCGACCC | 2155 |
| 30 | GCAGCUGG G CCCGCGGC | 1426 | GCCGCGGG GGCTAGCTACAACGA CCAGCTGC | 2156 |
| 34 | CUGGGCCC G CGGCAUGG | 1427 | CCATGCCG GGCTAGCTACAACGA GGGCCCAG | 2157 |
| 37 | GGCCCGCG G CAUGGACG | 1428 | CGTCCATG GGCTAGCTACAACGA CGCGGGCC | 2158 |
| 39 | CCCGCGGC A UGGACGAA | 6 | TTCGTCCA GGCTAGCTACAACGA GCCGCGGG | 2159 |
| 43 | CGGCAUGG A CGAACUGU | 2013 | ACAGTTCG GGCTAGCTACAACGA CCATGCCG | 2160 |
| 47 | AUGGACGA A CUGUUCCC | 2014 | GGGAACAG GGCTAGCTACAACGA TCGTCCAT | 2161 |
| 50 | GACGAACU G UUCCCCCU | 1429 | AGGGGGAA GGCTAGCTACAACGA AGTTCGTC | 2162 |
| 60 | UCCCCCUC A UCUUCCCG | 13 | CGGGAAGA GGCTAGCTACAACGA GAGGGGGA | 2163 |
| 69 | UCUUCCCG G CAGAGCAG | 1430 | CTGCTCTG GGCTAGCTACAACGA CGGGAAGA | 2164 |
| 74 | CCGGCAGA G CAGCCCAA | 1431 | TTGGGCTG GGCTAGCTACAACGA TCTGCCGG | 2165 |
| 77 | GCAGAGCA G CCCAAGCA | 1432 | TGCTTGGG GGCTAGCTACAACGA TGCTCTGC | 2166 |
| 83 | CAGCCCAA G CAGCGGGG | 1433 | CCCCGCTG GGCTAGCTACAACGA TTGGGCTG | 2167 |
| 86 | CCCAAGCA G CGGGGCAU | 1434 | ATGCCCCG GGCTAGCTACAACGA TGCTTGGG | 2168 |
| 91 | GCAGCGGG G CAUGCGCU | 1435 | AGCGCATG GGCTAGCTACAACGA CCCGCTGC | 2169 |
| 93 | AGCGGGGC A UGCGCUUC | 23 | GAAGCGCA GGCTAGCTACAACGA GCCCCGCT | 2170 |
| 95 | CGGGGCAU G CGCUUCCG | 1436 | CGGAAGCG GGCTAGCTACAACGA ATGCCCCG | 2171 |
| 97 | GGGCAUGC G CUUCCGCU | 1437 | AGCGGAAG GGCTAGCTACAACGA GCATGCCC | 2172 |
| 103 | GCGCUUCC G CUACAAGU | 1438 | ACTTGTAG GGCTAGCTACAACGA GGAAGCGC | 2173 |
| 106 | CUUCCGCU A CAAGUGCG | 2015 | CGCACTTG GGCTAGCTACAACGA AGCGGAAG | 2174 |
| 110 | CGCUACAA G UGCGAGGG | 1439 | CCCTCGCA GGCTAGCTACAACGA TTGTAGCG | 2175 |
| 112 | CUACAAGU G CGAGGGGC | 1440 | GCCCCTCG GGCTAGCTACAACGA ACTTGTAG | 2176 |
| 119 | UGCGAGGG G CGCUCCGC | 1441 | GCGGAGCG GGCTAGCTACAACGA CCCTCGCA | 2177 |
| 121 | CGAGGCGC G CUCCGCGG | 1442 | CCGCGGAG GGCTAGCTACAACGA GCCCCTCG | 2178 |
| 126 | GGCGCUCC G CGGGCAGC | 1443 | GCTGCCCG GGCTAGCTACAACGA GGAGCGCC | 2179 |
| 130 | CUCCGCGG G CAGCAUCC | 1444 | GGATGCTG GGCTAGCTACAACGA CCGCGGAG | 2180 |
| 133 | CGCGGGCA G CAUCCCAG | 1445 | CTGGGATG GGCTAGCTACAACGA TGCCCGCG | 2181 |
| 135 | CGGGCAGC A UCCCAGGC | 31 | GCCTGGGA GGCTAGCTACAACGA GCTGCCCG | 2182 |
| 142 | CAUCCCAG G CGAGAGGA | 1446 | TCCTCTCG GGCTAGCTACAACGA CTGGGATG | 2183 |
| 151 | CGAGAGGA G CACAGAUA | 1447 | TATCTGTG GGCTAGCTACAACGA TCCTCTCG | 2184 |
| 153 | AGAGGAGC A CAGAUACC | 35 | GGTATCTG GGCTAGCTACAACGA GCTCCTCT | 2185 |
| 157 | GAGCACAG A UACCACCA | 2016 | TGGTGGTA GGCTAGCTACAACGA CTGTGCTC | 2186 |
| 159 | GCACAGAU A CCACCAAG | 2017 | CTTGGTGG GGCTAGCTACAACGA ATCTGTGC | 2187 |
| 162 | CAGAUACC A CCAAGACC | 38 | GGTCTTGG GGCTAGCTACAACGA GGTATCTG | 2188 |
| 168 | CCACCAAG A CCCACCCC | 2018 | GGGGTGGG GGCTAGCTACAACGA CTTGGTGG | 2189 |
| 172 | CAAGACCC A CCCCACCA | 43 | TGGTGGGG GGCTAGCTACAACGA GGGTCTTG | 2190 |
| 177 | CCCACCCC A CCAUCAAG | 47 | CTTGATGG GGCTAGCTACAACGA GGGGTGGG | 2191 |
| 180 | ACCCCACC A UCAAGAUC | 49 | GATCTTGA GGCTAGCTACAACGA GGTGGGGT | 2192 |
| 186 | CCAUCAAG A UCAAUGGC | 2019 | GCCATTGA GGCTAGCTACAACGA CTTGATGG | 2193 |
| 190 | CAAGAUCA A UGGCUACA | 2020 | TGTAGCCA GGCTAGCTACAACGA TGATCTTG | 2194 |
| 193 | GAUCAAUG G CUACACAG | 1448 | CTGTGTAG GGCTAGCTACAACGA CATTGATC | 2195 |
| 196 | CAAUGGCU A CACAGGAC | 2021 | GTCCTGTG GGCTAGCTACAACGA AGCCATTG | 2196 |
| 198 | AUGGCUAC A CAGGACCA | 53 | TGGTCCTG GGCTAGCTACAACGA GTAGCCAT | 2197 |
| 203 | UACACAGG A CCAGGGAC | 2022 | GTCCCTGG GGCTAGCTACAACGA CCTGTGTA | 2198 |
| 210 | GACCAGGG A CAGUGCGC | 2023 | GCGCACTG GGCTAGCTACAACGA CCCTGGTC | 2199 |
| 213 | CAGGGACA G UGCGCAUC | 1449 | GATGCGCA GGCTAGCTACAACGA TGTCCCTG | 2200 |
| 215 | GGGACAGU G CGCAUCUC | 1450 | GAGATGCG GGCTAGCTACAACGA ACTGTCCC | 2201 |
| 217 | GACAGUGC G CAUCUCCC | 1451 | GGGAGATG GGCTAGCTACAACGA GCACTGTC | 2202 |
| 219 | CAGUGCGC A UCUCCCUG | 58 | CAGGGAGA GGCTAGCTACAACGA GCGCACTG | 2203 |
| 228 | UCUCCCUG G UCACCAAG | 1452 | CTTGGTGA GGCTAGCTACAACGA CAGGGAGA | 2204 |
| 231 | CCCUGGUC A CCAAGGAC | 63 | GTCCTTGG GGCTAGCTACAACGA GACCAGGG | 2205 |
| 238 | CACCAAGG A CCCUCCUC | 2024 | GAGGAGGG GGCTAGCTACAACGA CCTTGGTG | 2206 |
| 247 | CCCUCCUC A CCGGCCUC | 71 | GAGGCCGG GGCTAGCTACAACGA GAGGAGGG | 2207 |
| 251 | CCUCACCG G CCUCACCC | 1453 | GGGTGAGG GGCTAGCTACAACGA CGGTGAGG | 2208 |
| 256 | CCGGCCUC A CCCCCACG | 75 | CGTGGGGG GGCTAGCTACAACGA GAGGCCGG | 2209 |
| 262 | UCACCCCC A CGAGCUUG | 80 | CAAGCTCG GGCTAGCTACAACGA GGGGGTGA | 2210 |
| 266 | CCCCACGA G CUUGUAGG | 1454 | CCTACAAG GGCTAGCTACAACGA TCGTGGGG | 2211 |
| 270 | ACGAGCUU G UAGGAAAG | 1455 | CTTTCCTA GGCTAGCTACAACGA AAGCTCGT | 2212 |
| 280 | AGCAAAGG A CUGCCGGG | 2025 | CCCGGCAG GGCTAGCTACAACGA CCTTTCCT | 2213 |
| 283 | AAAGGACU G CCGGGAUG | 1456 | CATCCCGG GGCTAGCTACAACGA AGTCCTTT | 2214 |
| 289 | CUGCCGGG A UGGCUUCU | 2026 | AGAAGCCA GGCTAGCTACAACGA CCCGGCAG | 2215 |
| 292 | CCGGGAUG G CUUCUAUG | 1457 | CATAGAAG GGCTAGCTACAACGA CATCCCGG | 2216 |
| 298 | UGGCUUCU A UGAGGCUG | 2027 | CAGCCTCA GGCTAGCTACAACGA AGAAGCCA | 2217 |
| 303 | UCUAUGAG G CUGAGCUC | 1458 | GAGCTCAG GGCTAGCTACAACGA CTCATAGA | 2218 |
| 308 | GAGGCUGA G CUCUGCCC | 1459 | GGGCAGAG GGCTAGCTACAACGA TCAGCCTC | 2219 |
| 313 | UGAGCUCU G CCCGGACC | 1460 | GGTCCGGG GGCTAGCTACAACGA AGAGCTCA | 2220 |
| 319 | CUGCCCGG A CCGCUGCA | 2028 | TGCAGCGG GGCTAGCTACAACGA CCGGGCAG | 2221 |
| 322 | CCCGGACC G CUGCAUCC | 1461 | GGATGCAG GGCTAGCTACAACGA GGTCCGGG | 2222 |
| 325 | GGACCGCU G CAUCCACA | 1462 | TGTGGATG GGCTAGCTACAACGA AGCGGTCC | 2223 |
| 327 | ACCGCUGC A UCCACAGU | 93 | ACTGTGGA GGCTAGCTACAACGA GCAGCGGT | 2224 |
| 331 | CUGCAUCC A CAGUUUCC | 95 | GGAAACTG GGCTAGCTACAACGA GGATGCAG | 2225 |
| 334 | CAUCCACA G UUUCCAGA | 1463 | TCTGGAAA GGCTAGCTACAACGA TGTGGATG | 2226 |
| 343 | UUUCCAGA A CCUGGGAA | 2029 | TTCCCAGG GGCTAGCTACAACGA TCTGGAAA | 2227 |
| 351 | ACCUGGGA A UCCAGUGU | 2030 | ACACTGGA GGCTAGCTACAACGA TCCCAGGT | 2228 |
| 356 | GGAAUCCA G UGUGUGAA | 1464 | TTCACACA GGCTAGCTACAACGA TGGATTCC | 2229 |
| 358 | AAUCCAGU G UGUGAAGA | 1465 | TCTTCACA GGCTAGCTACAACGA ACTGGATT | 2230 |
| 360 | UCCAGUGU G UGAAGAAG | 1466 | CTTCTTCA GGCTAGCTACAACGA ACACTGGA | 2231 |
| 368 | GUGAAGAA G CGGGACCU | 1467 | AGGTCCCG GGCTAGCTACAACGA TTCTTCAC | 2232 |
| 373 | GAAGCGGG A CCUGGAGC | 2031 | GCTCCAGG GGCTAGCTACAACGA CCCGCTTC | 2233 |
| 380 | GACCUGGA G CAGGCUAU | 1468 | ATAGCCTG GGCTAGCTACAACGA TCCAGGTC | 2234 |
| 384 | UGGAGCAG G CUAUCAGU | 1469 | ACTGATAG GGCTAGCTACAACGA CTGCTCCA | 2235 |
| 387 | AGCAGGCU A UCAGUCAG | 2032 | CTGACTGA GGCTAGCTACAACGA AGCCTGCT | 2236 |
| 391 | GGCUAUCA G UCAGCGCA | 1470 | TGCGCTGA GGCTAGCTACAACGA TGATAGCC | 2237 |
| 395 | AUCAGUCA G CGCAUCCA | 1471 | TGGATGCG GGCTAGCTACAACGA TGACTGAT | 2238 |
| 397 | CAGUCAGC G CAUCCAGA | 1472 | TCTGGATG GGCTAGCTACAACGA GCTGACTG | 2239 |
| 399 | GUCAGCGC A UCCAGACC | 109 | GGTCTGGA GGCTAGCTACAACGA GCGCTGAC | 2240 |
| 405 | GCAUCCAG A CCAACAAC | 2033 | GTTGTTGG GGCTAGCTACAACGA CTGGATGC | 2241 |
| 409 | CCAGACCA A CAACAACC | 2034 | GGTTGTTG GGCTAGCTACAACGA TGGTCTGG | 2242 |
| 412 | GACCAACA A CAACCCCU | 2035 | AGGGGTTG GGCTAGCTACAACGA TGTTGGTC | 2243 |
| 415 | CAACAACA A CCCCUUCC | 2036 | GGAAGGGG GGCTAGCTACAACGA TGTTGTTG | 2244 |
| 426 | CCUUCCAA G UUCCUAUA | 1473 | TATAGGAA GGCTAGCTACAACGA TTGGAAGG | 2245 |
| 432 | AAGUUCCU A UAGAAGAG | 2037 | CTCTTCTA GGCTAGCTACAACGA AGGAACTT | 2246 |
| 440 | AUAGAAGA G CAGCGUGG | 1474 | CCACGCTG GGCTAGCTACAACGA TCTTCTAT | 2247 |
| 443 | GAAGAGCA G CGUGGGGA | 1475 | TCCCCACG GGCTAGCTACAACGA TGCTCTTC | 2248 |
| 445 | AGAGCAGC G UGGGGACU | 1476 | AGTCCCCA GGCTAGCTACAACGA GCTGCTCT | 2249 |
| 451 | GCGUGGGG A CUACGACC | 2038 | GGTCGTAG GGCTAGCTACAACGA CCCCACGC | 2250 |
| 454 | UGGGGACU A CGACCUGA | 2039 | TCAGGTCG GGCTAGCTACAACGA AGTCCCCA | 2251 |
| 457 | GGACUACG A CCUGAAUG | 2040 | CATTCAGG GGCTAGCTACAACGA CGTAGTCC | 2252 |
| 463 | CGACCUGA A UGCUGUGC | 2041 | GCACAGCA GGCTAGCTACAACGA TCAGGTCG | 2253 |
| 465 | ACCUCAAU G CUGUGCGG | 1477 | CCGCACAG GGCTAGCTACAACGA ATTCAGGT | 2254 |
| 468 | UGAAUGCU G UGCGGCUC | 1478 | GAGCCGCA GGCTAGCTACAACGA AGCATTCA | 2255 |
| 470 | AAUGCUGU G CGGCUCUG | 1479 | CAGAGCCG GGCTAGCTACAACGA ACAGCATT | 2256 |
| 473 | GCUGUGCG G CUCUGCUU | 1480 | AAGCAGAG GGCTAGCTACAACGA CGCACAGC | 2257 |
| 478 | GCGGCUCU G CUUCCAGG | 1481 | CCTGGAAG GGCTAGCTACAACGA AGAGCCGC | 2258 |
| 486 | GCUUCCAG G UGACAGUG | 1482 | CACTGTCA GGCTAGCTACAACGA CTGGAAGC | 2259 |
| 489 | UCCAGGUG A CAGUGCGG | 2042 | CCGCACTG GGCTAGCTACAACGA CACCTGGA | 2260 |
| 492 | AGGUGACA G UGCGGGAC | 1483 | GTCCCGCA GGCTAGCTACAACGA TGTCACCT | 2261 |
| 494 | GUGACAGU G CGGGACCC | 1484 | GGGTCCCG GGCTAGCTACAACGA ACTGTCAC | 2262 |
| 499 | AGUGCGGG A CCCAUCAG | 2043 | CTGATGGG GGCTAGCTACAACGA CCCGCACT | 2263 |
| 503 | CGGGACCC A UCAGGCAG | 137 | CTGCCTGA GGCTAGCTACAACGA GGGTCCCG | 2264 |
| 508 | CCCAUCAG G CAGGCCCC | 1485 | GGGGCCTG GGCTAGCTACAACGA CTGATGGG | 2265 |
| 512 | UCAGGCAG G CCCCUCCG | 1486 | CGGAGGGG GGCTAGCTACAACGA CTGCCTGA | 2266 |
| 520 | GCCCCUCC G CCUGCCGC | 1487 | GCGGCAGG GGCTAGCTACAACGA GGAGGGGC | 2267 |
| 524 | CUCCGCCU G CCGCCUGU | 1488 | ACAGGCGG GGCTAGCTACAACGA AGGCGGAG | 2268 |
| 527 | CGCCUGCC G CCUGUCCU | 1489 | AGGACAGG GGCTAGCTACAACGA GGCAGGCG | 2269 |
| 531 | UGCCGCCU G UCCUUUCU | 1490 | AGAAAGGA GGCTAGCTACAACGA AGGCGGCA | 2270 |
| 541 | CCUUUCUC A UCCCAUCU | 153 | AGATGGGA GGCTAGCTACAACGA GAGAAAGG | 2271 |
| 546 | CUCAUCCC A UCUUUGAC | 156 | GTCAAAGA GGCTAGCTACAACGA GGGATGAG | 2272 |
| 553 | CAUCUUUG A CAAUCGUG | 2044 | CACGATTG GGCTAGCTACAACGA CAAAGATG | 2273 |
| 556 | CUUUGACA A UCGUGCCC | 2045 | GGGCACGA GGCTAGCTACAACGA TGTCAAAG | 2274 |
| 559 | UGACAAUC G UGCCCCCA | 1491 | TGGGGGCA GGCTAGCTACAACGA GATTGTCA | 2275 |
| 561 | ACAAUCGU G CCCCCAAC | 1492 | GTTGGGGG GGCTAGCTACAACGA ACGATTGT | 2276 |
| 568 | UGCCCCCA A CACUGCCG | 2046 | CGGCAGTG GGCTAGCTACAACGA TGGGGGCA | 2277 |
| 570 | CCCCCAAC A CUGCCGAG | 164 | CTCGGCAG GGCTAGCTACAACGA GTTGGGGG | 2278 |
| 573 | CCAACACU G CCGAGCUC | 1493 | GAGCTCGG GGCTAGCTACAACGA AGTGTTGG | 2279 |
| 578 | ACUGCCGA G CUCAAGAU | 1494 | ATCTTGAG GGCTAGCTACAACGA TCGGCAGT | 2280 |
| 585 | AGCUCAAG A UCUGCCGA | 2047 | TCGGCAGA GGCTAGCTACAACGA CTTGAGCT | 2281 |
| 589 | CAAGAUCU G CCGAGUGA | 1495 | TCACTCGG GGCTAGCTACAACGA AGATCTTG | 2282 |
| 594 | UCUGCCGA G UGAACCGA | 1496 | TCGGTTCA GGCTAGCTACAACGA TCGGCAGA | 2283 |
| 598 | CCGAGUGA A CCGAAACU | 2048 | AGTTTCGG GGCTAGCTACAACGA TCACTCGG | 2284 |
| 604 | GAACCGAA A CUCUGGCA | 2049 | TGCCAGAG GGCTAGCTACAACGA TTCGGTTC | 2285 |
| 610 | AAACUCUG G CAGCUGCC | 1497 | GGCAGCTG GGCTAGCTACAACGA CAGAGTTT | 2286 |
| 613 | CUCUGGCA G CUGCCUCG | 1498 | CGAGGCAG GGCTAGCTACAACGA TGCCAGAG | 2287 |
| 616 | UGGCAGCU G CCUCGGUG | 1499 | CACCGAGG GGCTAGCTACAACGA AGCTGCCA | 2288 |
| 622 | CUGCCUCG G UGGGGAUG | 1500 | CATCCCCA GGCTAGCTACAACGA CGAGGCAG | 2289 |
| 628 | CGGUGGGG A UGAGAUCU | 2050 | AGATCTCA GGCTAGCTACAACGA CCCCACCG | 2290 |
| 633 | GGGAUGAG A UCUUCCUA | 2051 | TAGGAAGA GGCTAGCTACAACGA CTCATCCC | 2291 |
| 641 | AUCUUCCU A CUGUGUGA | 2052 | TCACACAG GGCTAGCTACAACGA AGGAAGAT | 2292 |
| 644 | UUCCUACU G UGUGACAA | 1501 | TTGTCACA GGCTAGCTACAACGA AGTAGGAA | 2293 |
| 646 | CCUACUGU G UGACAAGG | 1502 | CCTTGTCA GGCTAGCTACAACGA ACAGTAGG | 2294 |
| 649 | ACUGUGUG A CAAGGUGC | 2053 | GCACCTTG GGCTAGCTACAACGA CACACAGT | 2295 |
| 654 | GUGACAAG G UGCAGAAA | 1503 | TTTCTGCA GGCTAGCTACAACGA CTTGTCAC | 2296 |
| 656 | GACAAGGU G CAGAAAGA | 1504 | TCTTTCTG GGCTAGCTACAACGA ACCTTGTC | 2297 |
| 667 | GAAAGAGG A CAUUGAGG | 2054 | CCTCAATG GGCTAGCTACAACGA CCTCTTTC | 2298 |
| 669 | AAGAGGAC A UUGAGGUG | 184 | CACCTCAA GGCTAGCTACAACGA GTCCTCTT | 2299 |
| 675 | ACAUUGAG G UGUAUUUC | 1505 | GAAATACA GGCTAGCTACAACGA CTCAATGT | 2300 |
| 677 | AUUGAGGU G UAUUUCAC | 1506 | GTGAAATA GGCTAGCTACAACGA ACCTCAAT | 2301 |
| 679 | UGAGGUGU A UUUCACGG | 2055 | CCGTGAAA GGCTAGCTACAACGA ACACCTCA | 2302 |
| 684 | UGUAUUUC A CGGGACCA | 185 | TGGTCCCG GGCTAGCTACAACGA GAAATACA | 2303 |
| 689 | UUCACGGG A CCAGGCUG | 2056 | CAGCCTGG GGCTAGCTACAACGA CCCGTGAA | 2304 |
| 694 | GGGACCAG G CUGGGAGG | 1507 | CCTCCCAG GGCTAGCTACAACGA CTGGTCCC | 2305 |
| 702 | GCUGGGAG G CCCGAGGC | 1508 | GCCTCGGG GGCTAGCTACAACGA CTCCCAGC | 2306 |
| 709 | GGCCCGAG G CUCCUUUU | 1509 | AAAAGGAG GGCTAGCTACAACGA CTCGGGCC | 2307 |
| 719 | UCCUUUUC G CAAGCUGA | 1510 | TCAGCTTG GGCTAGCTACAACGA GAAAAGGA | 2308 |
| 723 | UUUCGCAA G CUGAUGUG | 1511 | CACATCAG GGCTAGCTACAACGA TTGCGAAA | 2309 |
| 727 | GCAAGCUG A UGUGCACC | 2057 | GGTGCACA GGCTAGCTACAACGA CAGCTTGC | 2310 |
| 729 | AAGCUGAU G UGCACCGA | 1512 | TCGGTGCA GGCTAGCTACAACGA ATCAGCTT | 2311 |
| 731 | GCUGAUGU G CACCGACA | 1513 | TGTCGGTG GGCTAGCTACAACGA ACATCAGC | 2312 |
| 733 | UGAUGUGC A CCGACAAG | 196 | CTTGTCGG GGCTAGCTACAACGA GCACATCA | 2313 |
| 737 | GUGCACCG A CAAGUGGC | 2058 | GCCACTTG GGCTAGCTACAACGA CGGTGCAC | 2314 |
| 741 | ACCGACAA G UGGCCAUU | 1514 | AATGGCCA GGCTAGCTACAACGA TTGTCGGT | 2315 |
| 744 | GACAAGUG G CCAUUGUG | 1515 | CACAATGG GGCTAGCTACAACGA CACTTGTC | 2316 |
| 747 | AAGUGGCC A UUGUGUUC | 200 | GAACACAA GGCTAGCTACAACGA GGCCACTT | 2317 |
| 750 | UGGCCAUU G UGUUCCGG | 1516 | CCGGAACA GGCTAGCTACAACGA AATGGCCA | 2318 |
| 752 | GCCAUUGU G UUCCGGAC | 1517 | GTCCGGAA GGCTAGCTACAACGA ACAATGGC | 2319 |
| 759 | UGUUCCGG A CCCCUCCC | 2059 | GGGAGGGG GGCTAGCTACAACGA CCGGAACA | 2320 |
| 769 | CCCUCCCU A CGCAGACC | 2060 | GGTCTGCG GGCTAGCTACAACGA AGGGAGGG | 2321 |
| 771 | CUCCCUAC G CAGACCCC | 1518 | GGGGTCTG GGCTAGCTACAACGA GTAGGGAG | 2322 |
| 775 | CUACGCAG A CCCCAGCC | 2061 | GGCTGGGG GGCTAGCTACAACGA CTGCGTAG | 2323 |
| 781 | AGACCCCA G CCUGCAGG | 1519 | CCTGCAGG GGCTAGCTACAACGA TGGGGTCT | 2324 |
| 785 | CCCAGCCU G CAGGCUCC | 1520 | GGAGCCTG GGCTAGCTACAACGA AGGCTGGG | 2325 |
| 789 | GCCUGCAG G CUCCUGUG | 1521 | CACAGGAG GGCTAGCTACAACGA CTGCAGGC | 2326 |
| 795 | AGGCUCCU G UGCGUGUC | 1522 | GACACGCA GGCTAGCTACAACGA AGGAGCCT | 2327 |
| 797 | GCUCCUGU G CGUGUCUC | 1523 | GAGACACG GGCTAGCTACAACGA ACAGGAGC | 2328 |
| 799 | UCCUGUGC G UGUCUCCA | 1524 | TGGAGACA GGCTAGCTACAACGA GCACAGGA | 2329 |
| 801 | CUGUGCGU G UCUCCAUG | 1525 | CATGGAGA GGCTAGCTACAACGA ACGCACAG | 2330 |
| 807 | GUGUCUCC A UGCAGCUG | 222 | CAGCTGCA GGCTAGCTACAACGA GGAGACAC | 2331 |
| 809 | GUCUCCAU G CAGCUGCG | 1526 | CGCAGCTG GGCTAGCTACAACGA ATGGAGAC | 2332 |
| 812 | UCCAUGCA G CUGCGGCG | 1527 | CGCCGCAG GGCTAGCTACAACGA TGCATGGA | 2333 |
| 815 | AUGCAGCU G CCGCGGCC | 1528 | GGCCGCCG GGCTAGCTACAACGA AGCTGCAT | 2334 |
| 818 | CAGCUGCG G CGGCCUUC | 1529 | GAAGGCCG GGCTAGCTACAACGA CGCAGCTG | 2335 |
| 821 | CUGCGGCG G CCUUCCGA | 1530 | TCGGAAGG GGCTAGCTACAACGA CGCCGCAG | 2336 |
| 829 | GCCUUCCG A CCGGGAGC | 2062 | GCTCCCGG GGCTAGCTACAACGA CGGAAGGC | 2337 |
| 836 | GACCGGGA G CUCAGUGA | 1531 | TCACTGAG GGCTAGCTACAACGA TCCCGGTC | 2338 |
| 841 | GGAGCUCA G UGAGCCCA | 1532 | TGGGCTCA GGCTAGCTACAACGA TGAGCTCC | 2339 |
| 845 | CUCAGUGA G CCCAUGGA | 1533 | TCCATGGG GGCTAGCTACAACGA TCACTGAG | 2340 |
| 849 | GUGAGCCC A UGGAAUUC | 233 | GAATTCCA GGCTAGCTACAACGA GGGCTCAC | 2341 |
| 854 | CCCAUGGA A UUCCAGUA | 2063 | TACTGGAA GGCTAGCTACAACGA TCCATGGG | 2342 |
| 860 | GAAUUCCA G UACCUGCC | 1534 | GGCAGGTA GGCTAGCTACAACGA TGGAATTC | 2343 |
| 862 | AUUCCAGU A CCUGCCAG | 2064 | CTGGCAGG GGCTAGCTACAACGA ACTGGAAT | 2344 |
| 866 | CAGUACCU G CCAGAUAC | 1535 | GTATCTGG GGCTAGCTACAACGA AGGTACTG | 2345 |
| 871 | CCUGCCAG A UACAGACG | 2065 | CGTCTGTA GGCTAGCTACAACGA CTGGCAGG | 2346 |
| 873 | UGCCAGAU A CAGACGAU | 2066 | ATCGTCTG GGCTAGCTACAACGA ATCTGGCA | 2347 |
| 877 | AGAUACAG A CGAUCGUC | 2067 | GACGATCG GGCTAGCTACAACGA CTGTATCT | 2348 |
| 880 | UACAGACG A UCGUCACC | 2068 | GGTGACGA GGCTAGCTACAACGA CGTCTGTA | 2349 |
| 883 | AGACCAUC G UCACCGGA | 1536 | TCCGGTGA GGCTAGCTACAACGA GATCCTCT | 2350 |
| 886 | CGAUCGUC A CCGGAUUG | 241 | CAATCCGG GGCTAGCTACAACGA GACGATCG | 2351 |
| 891 | GUCACCGG A UUGAGGAG | 2069 | CTCCTCAA GGCTAGCTACAACGA CCGGTGAC | 2352 |
| 902 | GAGGAGAA A CGUAAAAG | 2070 | CTTTTACG GGCTAGCTACAACGA TTCTCCTC | 2353 |
| 904 | GGAGAAAC G UAAAAGGA | 1537 | TCCTTTTA GGCTAGCTACAACGA GTTTCTCC | 2354 |
| 912 | GUAAAAGG A CAUAUGAG | 2071 | CTCATATG GGCTAGCTACAACGA CCTTTTAC | 2355 |
| 914 | AAAAGGAC A UAUGAGAC | 243 | GTCTCATA GGCTAGCTACAACGA GTCCTTTT | 2356 |
| 916 | AAGGACAU A UGAGACCU | 2072 | AGGTCTCA GGCTAGCTACAACGA ATGTCCTT | 2357 |
| 921 | CAUAUGAG A CCUUCAAG | 2073 | CTTGAAGG GGCTAGCTACAACGA CTCATATG | 2358 |
| 931 | CUUCAAGA G CAUCAUGA | 1538 | TCATGATG GGCTAGCTACAACGA TCTTGAAG | 2359 |
| 933 | UCAAGAGC A UCAUGAAG | 247 | CTTCATGA GGCTAGCTACAACGA GCTCTTGA | 2360 |
| 936 | AGAGCAUC A UGAAGAAG | 248 | CTTCTTCA GGCTAGCTACAACGA GATGCTCT | 2361 |
| 946 | GAAGAAGA G UCCUUUCA | 1539 | TGAAAGGA GGCTAGCTACAACGA TCTTCTTC | 2362 |
| 955 | UCCUUUCA G CGGACCCA | 1540 | TGGGTCCG GGCTAGCTACAACGA TGAAAGGA | 2363 |
| 959 | UUCAGCGG A CCCACCGA | 2074 | TCGGTGGG GGCTAGCTACAACGA CCGCTGAA | 2364 |
| 963 | GCGGACCC A CCGACCCC | 254 | GGGGTCGG GGCTAGCTACAACGA GGGTCCGC | 2365 |
| 967 | ACCCACCG A CCCCCGGC | 2075 | GCCGGGGG GGCTAGCTACAACGA CGGTGGGT | 2366 |
| 974 | GACCCCCG G CCUCCACC | 1541 | GGTGGAGG GGCTAGCTACAACGA CGGGGGTC | 2367 |
| 980 | CGGCCUCC A CCUCGACG | 263 | CGTCGAGG GGCTAGCTACAACGA GGAGGCCG | 2368 |
| 986 | CCACCUCG A CGCAUUGC | 2076 | GCAATGCG GGCTAGCTACAACGA CGAGGTGG | 2369 |
| 988 | ACCUCGAC G CAUUGCUG | 1542 | CAGCAATG GGCTAGCTACAACGA GTCGAGGT | 2370 |
| 990 | CUCGACGC A UUGCUGUG | 266 | CACAGCAA GGCTAGCTACAACGA GCGTCGAG | 2371 |
| 993 | GACGCAUU G CUGUGCCU | 1543 | AGGCACAG GGCTAGCTACAACGA AATGCGTC | 2372 |
| 996 | GCAUUGCU G UGCCUUCC | 1544 | GGAAGGCA GGCTAGCTACAACGA AGCAATGC | 2373 |
| 998 | AUUGCUGU G CCUUCCCG | 1545 | CGGGAAGG GGCTAGCTACAACGA ACAGCAAT | 2374 |
| 1006 | GCCUUCCC G CAGCUCAG | 1546 | CTGAGCTG GGCTAGCTACAACGA GGGAAGGC | 2375 |
| 1009 | UUCCCGCA G CUCAGCUU | 1547 | AAGCTGAG GGCTAGCTACAACGA TGCGGGAA | 2376 |
| 1014 | GCAGCUCA G CUUCUGUC | 1548 | GACAGAAG GGCTAGCTACAACGA TGAGCTGC | 2377 |
| 1020 | CAGCUUCU G UCCCCAAG | 1549 | CTTGGGGA GGCTAGCTACAACGA AGAAGCTG | 2378 |
| 1028 | GUCCCCAA G CCAGCACC | 1550 | GGTGCTGG GGCTAGCTACAACGA TTGGGGAC | 2379 |
| 1032 | CCAAGCCA G CACCCCAG | 1551 | CTGGGGTG GGCTAGCTACAACGA TGGCTTGG | 2380 |
| 1034 | AAGCCAGC A CCCCAGCC | 283 | GGCTGGGG GGCTAGCTACAACGA GCTGGCTT | 2381 |
| 1040 | GCACCCCA G CCCUAUCC | 1552 | GGATAGGG GGCTAGCTACAACGA TGGGGTGC | 2382 |
| 1045 | CCAGCCCU A UCCCUUUA | 2077 | TAAAGGGA GGCTAGCTACAACGA AGGGCTGG | 2383 |
| 1053 | AUCCCUUU A CGUCAUCC | 2078 | GGATGACG GGCTAGCTACAACGA AAAGGGAT | 2384 |
| 1055 | CCCUUUAC G UCAUCCCU | 1553 | AGGGATGA GGCTAGCTACAACGA GTAAAGGG | 2385 |
| 1058 | UUUACGUC A UCCCUGAG | 294 | CTCAGGGA GGCTAGCTACAACGA GACGTAAA | 2386 |
| 1066 | AUCCCUGA G CACCAUCA | 1554 | TGATGGTG GGCTAGCTACAACGA TCAGGGAT | 2387 |
| 1068 | CCCUGAGC A CCAUCAAC | 298 | GTTGATGG GGCTAGCTACAACGA GCTCAGGG | 2388 |
| 1071 | UGAGCACC A UCAACUAU | 300 | ATAGTTGA GGCTAGCTACAACGA GGTGCTCA | 2389 |
| 1075 | CACCAUCA A CUAUGAUG | 2079 | CATCATAG GGCTAGCTACAACGA TGATGGTG | 2390 |
| 1078 | CAUCAACU A UGAUGAGU | 2080 | ACTCATCA GGCTAGCTACAACGA AGTTGATG | 2391 |
| 1081 | CAACUAUG A UGAGUUUC | 2081 | GAAACTCA GGCTAGCTACAACGA CATAGTTG | 2392 |
| 1085 | UAUGAUGA G UUUCCCAC | 1555 | GTGGGAAA GGCTAGCTACAACGA TCATCATA | 2393 |
| 1092 | AGUUUCCC A CCAUGGUG | 305 | CACCATGG GGCTAGCTACAACGA GGGAAACT | 2394 |
| 1095 | UUCCCACC A UGGUGUUU | 307 | AAACACCA GGCTAGCTACAACGA GGTGGGAA | 2395 |
| 1098 | CCACCAUG G UGUUUCCU | 1556 | AGGAAACA GGCTAGCTACAACGA CATGGTGG | 2396 |
| 1100 | ACCAUGGU G UUUCCUUC | 1557 | GAAGGAAA GGCTAGCTACAACGA ACCATGGT | 2397 |
| 1112 | CCUUCUGG G CAGAUCAG | 1558 | CTGATCTG GGCTAGCTACAACGA CCAGAAGG | 2398 |
| 1116 | CUGGGCAG A UCAGCCAG | 2082 | CTGGCTGA GGCTAGCTACAACGA CTGCCCAG | 2399 |
| 1120 | GCAGAUCA G CCAGGCCU | 1559 | AGGCCTGG GGCTAGCTACAACGA TGATCTGC | 2400 |
| 1125 | UCAGCCAG G CCUCGGCC | 1560 | GGCCGAGG GGCTAGCTACAACGA CTGGCTGA | 2401 |
| 1131 | AGGCCUCG G CCUUGGCC | 1561 | GGCCAAGG GGCTAGCTACAACGA CGAGGCCT | 2402 |
| 1137 | CGGCCUUG G CCCCGGCC | 1562 | GGCCGGGG GGCTAGCTACAACGA CAAGGCCG | 2403 |
| 1143 | UGGCCCCG G CCCCUCCC | 1563 | GGGAGGGG GGCTAGCTACAACGA CGGGGCCA | 2404 |
| 1155 | CUCCCCAA G UCCUGCCC | 1564 | GGGCAGGA GGCTAGCTACAACGA TTGGGGAG | 2405 |
| 1160 | CAAGUCCU G CCCCAGGC | 1565 | GCCTGGGG GGCTAGCTACAACGA AGGACTTG | 2406 |
| 1167 | UGCCCCAG G CUCCAGCC | 1566 | GGCTGGAG GGCTAGCTACAACGA CTGGGGCA | 2407 |
| 1173 | AGGCUCCA G CCCCUGCC | 1567 | GGCAGGGG GGCTAGCTACAACGA TGGAGCCT | 2408 |
| 1179 | CAGCCCCU G CCCCUGCU | 1568 | AGCAGGGG GGCTAGCTACAACGA AGGGGCTG | 2409 |
| 1185 | CUGCCCCU G CUCCAGCC | 1569 | GGCTGGAG GGCTAGCTACAACGA AGGCGCAG | 2410 |
| 1191 | CUGCUCCA G CCAUGGUA | 1570 | TACCATGG GGCTAGCTACAACGA TGGAGCAG | 2411 |
| 1194 | CUCCAGCC A UGGUAUCA | 351 | TGATACCA GGCTAGCTACAACGA GGCTGGAG | 2412 |
| 1197 | CAGCCAUG G UAUCAGCU | 1571 | AGCTGATA GGCTAGCTACAACGA CATGGCTG | 2413 |
| 1199 | GCCAUGGU A UCAGCUCU | 2083 | AGAGCTGA GGCTAGCTACAACGA ACCATGGC | 2414 |
| 1203 | UGGUAUCA G CUCUGGCC | 1572 | GGCCAGAG GGCTAGCTACAACGA TGATACCA | 2415 |
| 1209 | CAGCUCUG G CCCAGGCC | 1573 | GGCCTGGG GGCTAGCTACAACGA CAGAGCTG | 2416 |
| 1215 | UGGCCCAG G CCCCAGCC | 1574 | GGCTGGGG GGCTAGCTACAACGA CTGGGCCA | 2417 |
| 1221 | AGGCCCCA G CCCCUGUC | 1575 | GACAGGGG GGCTAGCTACAACGA TGGGGCCT | 2418 |
| 1227 | CAGCCCCU G UCCCAGUC | 1576 | GACTGGGA GGCTAGCTACAACGA AGGGGCTG | 2419 |
| 1233 | CUGUCCCA G UCCUAGCC | 1577 | GGCTAGGA GGCTAGCTACAACGA TGGGACAG | 2420 |
| 1239 | CAGUCCUA G CCCCAGGC | 1578 | GCCTGGGG GGCTAGCTACAACGA TAGGACTG | 2421 |
| 1246 | AGCCCCAG G CCCUCCUC | 1579 | CAGGAGGG GGCTAGCTACAACGA CTGCGGCT | 2422 |
| 1257 | CUCCUCAG G CUGUGGCC | 1580 | GGCCACAG GGCTAGCTACAACGA CTGAGGAG | 2423 |
| 1260 | CUCAGGCU G UGGCCCCA | 1581 | TGGGGCCA GGCTAGCTACAACGA AGCCTGAG | 2424 |
| 1263 | AGGCUGUG G CCCCACCU | 1582 | AGGTGGGG GGCTAGCTACAACGA CACAGCCT | 2425 |
| 1268 | GUGGCCCC A CCUGCCCC | 385 | GGGGCAGG GGCTAGCTACAACGA GGGGCCAC | 2426 |
| 1272 | CCCCACCU G CCCCCAAG | 1583 | CTTGGGGG GGCTAGCTACAACGA AGGTGGGG | 2427 |
| 1280 | GCCCCCAA G CCCACCCA | 1584 | TGGGTGGG GGCTAGCTACAACGA TTGGGGGC | 2428 |
| 1284 | CCAAGCCC A CCCAGGCU | 395 | AGCCTGGG GGCTAGCTACAACGA GGGCTTGG | 2429 |
| 1290 | CCACCCAG G CUGGGGAA | 1585 | TTCCCCAG GGCTAGCTACAACGA CTGGGTGG | 2430 |
| 1302 | GGGAAGGA A CGCUGUCA | 2084 | TGACAGCG GGCTAGCTACAACGA TCCTTCCC | 2431 |
| 1304 | GAAGGAAC G CUGUCAGA | 1586 | TCTGACAG GGCTAGCTACAACGA GTTCCTTC | 2432 |
| 1307 | GGAACGCU G UCAGAGGC | 1587 | GCCTCTGA GGCTAGCTACAACGA AGCGTTCC | 2433 |
| 1314 | UGUCAGAG G CCCUGCUG | 1588 | CAGCAGGG GGCTAGCTACAACGA CTCTGACA | 2434 |
| 1319 | GAGGCCCU G CUGCAGCU | 1589 | AGCTGCAG GGCTAGCTACAACGA AGGGCCTC | 2435 |
| 1322 | GCCCUGCU G CAGCUGCA | 1590 | TGCAGCTG GGCTAGCTACAACGA AGCAGGGC | 2436 |
| 1325 | CUGCUGCA G CUGCAGUU | 1591 | AACTGCAG GGCTAGCTACAACGA TGCAGCAG | 2437 |
| 1328 | CUGCAGCU G CAGUUUGA | 1592 | TCAAACTG GGCTAGCTACAACGA AGCTGCAG | 2438 |
| 1331 | CAGCUGCA G UUUGAUGA | 1593 | TCATCAAA GGCTAGCTACAACGA TGCAGCTG | 2439 |
| 1336 | GCAGUUUG A UGAUGAAG | 2085 | CTTCATCA GGCTAGCTACAACGA CAAACTGC | 2440 |
| 1339 | GUUUGAUG A UGAAGACC | 2086 | GGTCTTCA GGCTAGCTACAACGA CATCAAAC | 2441 |
| 1345 | UGAUGAAG A CCUGGGGG | 2087 | CCCCCAGG GGCTAGCTACAACGA CTTCATCA | 2442 |
| 1353 | ACCUGGGG G CCUUGCUU | 1594 | AAGCAAGG GGCTAGCTACAACGA CCCCAGGT | 2443 |
| 1358 | GGGGCCUU G CUUGGCAA | 1595 | TTGCCAAG GGCTAGCTACAACGA AAGGCCCC | 2444 |
| 1363 | CUUGCUUG G CAACAGCA | 1596 | TGCTGTTG GGCTAGCTACAACGA CAAGCAAG | 2445 |
| 1366 | GCUUGGCA A CAGCACAG | 2088 | CTGTGCTG GGCTAGCTACAACGA TGCCAAGC | 2446 |
| 1369 | UGGCAACA G CACAGACC | 1597 | GGTCTGTG GGCTAGCTACAACGA TGTTGCCA | 2447 |
| 1371 | GCAACAGC A CAGACCCA | 416 | TGGGTCTG GGCTAGCTACAACGA GCTGTTGC | 2448 |
| 1375 | CAGCACAG A CCCAGCUG | 2089 | CAGCTGGG GGCTAGCTACAACGA CTGTGCTG | 2449 |
| 1380 | CAGACCCA G CUGUGUUC | 1598 | GAACACAG GGCTAGCTACAACGA TGGGTCTG | 2450 |
| 1383 | ACCCAGCU G UGUUCACA | 1599 | TGTGAACA GGCTAGCTACAACGA AGCTGGGT | 2451 |
| 1385 | CCAGCUGU G UUCACAGA | 1600 | TCTGTGAA GGCTAGCTACAACGA ACAGCTGG | 2452 |
| 1389 | CUGUGUUC A CAGACCUG | 422 | CAGGTCTG GGCTAGCTACAACGA GAACACAG | 2453 |
| 1393 | GUUCACAG A CCUGGCAU | 2090 | ATGCCAGG GGCTAGCTACAACGA CTGTGAAC | 2454 |
| 1398 | CAGACCUG G CAUCCGUC | 1601 | GACGGATG GGCTAGCTACAACGA CAGGTCTG | 2455 |
| 1400 | GACCUGGC A UCCGUCGA | 426 | TCGACGGA GGCTAGCTACAACGA GCCAGGTC | 2456 |
| 1404 | UGGCAUCC G UCGACAAC | 1602 | GTTGTCGA GGCTAGCTACAACGA GGATGCCA | 2457 |
| 1408 | AUCCGUCG A CAACUCCG | 2091 | CGGAGTTG GGCTAGCTACAACGA CGACGGAT | 2458 |
| 1411 | CGUCGACA A CUCCGAGU | 2092 | ACTCGGAG GGCTAGCTACAACGA TGTCGACG | 2459 |
| 1418 | AACUCCGA G UUUCAGCA | 1603 | TGCTGAAA GGCTAGCTACAACGA TCGGAGTT | 2460 |
| 1424 | GAGUUUCA G CAGCUGCU | 1604 | AGCAGCTG GGCTAGCTACAACGA TGAAACTC | 2461 |
| 1427 | UUUCAGCA G CUGCUGAA | 1605 | TTCAGCAG GGCTAGCTACAACGA TGCTGAAA | 2462 |
| 1430 | CAGCAGCU G CUGAACCA | 1606 | TGGTTCAG GGCTAGCTACAACGA AGCTGCTG | 2463 |
| 1435 | GCUGCUGA A CCAGGGCA | 2093 | TGCCCTGG GGCTAGCTACAACGA TCAGCAGC | 2464 |
| 1441 | GAACCAGG G CAUACCUG | 1607 | CAGGTATG GGCTAGCTACAACGA CCTGGTTC | 2465 |
| 1443 | ACCAGGGC A UACCUGUG | 437 | CACAGGTA GGCTAGCTACAACGA GCCCTGGT | 2466 |
| 1445 | CAGGGCAU A CCUGUGGC | 2094 | GCCACAGG GGCTAGCTACAACGA ATGCCCTG | 2467 |
| 1449 | GCAUACCU G UGGCCCCC | 1608 | GGGGGCCA GGCTAGCTACAACGA AGGTATGC | 2468 |
| 1452 | UACCUGUG G CCCCCCAC | 1609 | GTGGGGGG GGCTAGCTACAACGA CACAGGTA | 2469 |
| 1459 | GGCCCCCC A CACAACUG | 445 | CAGTTGTG GGCTAGCTACAACGA GGGGGGCC | 2470 |
| 1461 | CCCCCCAC A CAACUGAG | 446 | CTCAGTTG GGCTAGCTACAACGA GTGGGGGG | 2471 |
| 1464 | CCCACACA A CUGAGCCC | 2095 | GGGCTCAG GGCTAGCTACAACGA TGTGTGGG | 2472 |
| 1469 | ACAACUGA G CCCAUGCU | 1610 | AGCATGGG GGCTAGCTACAACGA TCAGTTGT | 2473 |
| 1473 | CUGAGCCC A UGCUGAUG | 451 | CATCAGCA GGCTAGCTACAACGA GGGCTCAG | 2474 |
| 1475 | GAGCCCAU G CUGAUGGA | 1611 | TCCATCAG GGCTAGCTACAACGA ATGGGCTC | 2475 |
| 1479 | CCAUGCUG A UGGAGUAC | 2096 | GTACTCCA GGCTAGCTACAACGA CAGCATGG | 2476 |
| 1484 | CUGAUGGA G UACCCUGA | 1612 | TCAGGGTA GGCTAGCTACAACGA TCCATCAG | 2477 |
| 1486 | GAUGGAGU A CCCUGAGG | 2097 | CCTCAGGG GGCTAGCTACAACGA ACTCCATC | 2478 |
| 1494 | ACCCUGAG G CUAUAACU | 1613 | AGTTATAG GGCTAGCTACAACGA CTCAGGGT | 2479 |
| 1497 | CUGAGGCU A UAACUCGC | 2098 | GCGAGTTA GGCTAGCTACAACGA AGCCTCAG | 2480 |
| 1500 | AGGCUAUA A CUCGCCUA | 2099 | TAGGCGAG GGCTAGCTACAACGA TATAGCCT | 2481 |
| 1504 | UAUAACUC G CCUAGUGA | 1614 | TCACTAGG GGCTAGCTACAACGA GAGTTATA | 2482 |
| 1509 | CUCGCCUA G UGACAGCC | 1615 | GGCTGTCA GGCTAGCTACAACGA TAGGCGAG | 2483 |
| 1512 | GCCUAGUG A CAGCCCAG | 2100 | CTGGGCTG GGCTAGCTACAACGA CACTAGGC | 2484 |
| 1515 | UAGUGACA G CCCAGAGG | 1616 | CCTCTGGG GGCTAGCTACAACGA TGTCACTA | 2485 |
| 1523 | GCCCAGAG G CCCCCCGA | 1617 | TCGGGGGG GGCTAGCTACAACGA CTCTGGGC | 2486 |
| 1531 | GCCCCCCG A CCCAGCUC | 2101 | GAGCTGGG GGCTAGCTACAACGA CGGGGGGC | 2487 |
| 1536 | CCGACCCA G CUCCUGCU | 1618 | AGCAGGAG GGCTAGCTACAACGA TGGGTCGG | 2488 |
| 1542 | CAGCUCCU G CUCCACUG | 1619 | CAGTGGAG GGCTAGCTACAACGA AGGAGCTG | 2489 |
| 1547 | CCUGCUCC A CUGGGGGC | 477 | GCCCCCAG GGCTAGCTACAACGA GGAGCAGG | 2490 |
| 1554 | CACUGGGG G CCCCGGGG | 1620 | CCCCGGGG GGCTAGCTACAACGA CCCCAGTG | 2491 |
| 1562 | GCCCCCGG G CUCCCCAA | 1621 | TTCGGGAG GGCTAGCTACAACGA CCCGGGGC | 2492 |
| 1570 | CCUCCCCA A UGGCCUCC | 2102 | GGAGGCCA GGCTAGCTACAACGA TGGGGAGC | 2493 |
| 1573 | CCCCAAUG G CCUCCUUU | 1622 | AAAGGAGG GGCTAGCTACAACGA CATTGGGG | 2494 |
| 1588 | UUCAGGAG A UGAAGACU | 2103 | AGTCTTCA GGCTAGCTACAACGA CTCCTGAA | 2495 |
| 1594 | AGAUGAAG A CUUCUCCU | 2104 | AGGAGAAG GGCTAGCTACAACGA CTTCATCT | 2496 |
| 1605 | UCUCCUCC A UUGCGGAC | 497 | GTCCGCAA GGCTAGCTACAACGA GGAGGAGA | 2497 |
| 1608 | CCUCCAUU G CGGACAUG | 1623 | CATGTCCG GGCTAGCTACAACGA AATGGAGG | 2498 |
| 1612 | CAUUGCGG A CAUGGACU | 2105 | AGTCCATG GGCTAGCTACAACGA CCGCAATG | 2499 |
| 1614 | UUGCGGAC A UGGACUUC | 498 | GAAGTCCA GGCTAGCTACAACGA GTCCGCAA | 2500 |
| 1618 | CGACAUGG A CUUCUCAG | 2106 | CTGAGAAG GGCTAGCTACAACGA CCATGTCC | 2501 |
| 1626 | ACUUCUCA G CCCUGCUG | 1624 | CAGCAGGG GGCTAGCTACAACGA TGAGAAGT | 2502 |
| 1631 | UCAGCCCU G CUGAGUCA | 1625 | TGACTCAG GGCTAGCTACAACGA AGGGCTGA | 2503 |
| 1636 | CCUGCUGA G UCAGAUCA | 1626 | TGATCTGA GGCTAGCTACAACGA TCAGCAGG | 2504 |
| 1641 | UGAGUCAG A UCAGCUCC | 2107 | GGAGCTGA GGCTAGCTACAACGA CTGACTCA | 2505 |
| 1645 | UCAGAUCA G CUCCUAAG | 1627 | CTTAGGAG GGCTAGCTACAACGA TGATCTGA | 2506 |
| 1657 | CUAAGGGG G UGACGCCU | 1628 | AGGCGTCA GGCTAGCTACAACGA CCCCTTAG | 2507 |
| 1660 | AGGGGGUG A CGCCUGCC | 2108 | GGCAGGCG GGCTAGCTACAACGA CACCCCCT | 2508 |
| 1662 | GGGGUGAC G CCUGCCCU | 1629 | AGGGCAGG GGCTAGCTACAACGA GTCACCCC | 2509 |
| 1666 | UGACGCCU G CCCUCCCC | 1630 | GGGGAGGG GGCTAGCTACAACGA AGGCGTCA | 2510 |
| 1678 | UCCCCAGA G CACUGGUU | 1631 | AACCAGTG GGCTAGCTACAACGA TCTGGGGA | 2511 |
| 1680 | CCCAGAGC A CUGGUUGC | 520 | GCAACCAG GGCTAGCTACAACGA GCTCTGGG | 2512 |
| 1684 | GAGCACUG G UUGCAGGG | 1632 | CCCTGCAA GGCTAGCTACAACGA CAGTGCTC | 2513 |
| 1687 | CACUGGUU G CAGGGGAU | 1633 | ATCCCCTG GGCTAGCTACAACGA AACCAGTG | 2514 |
| 1694 | UGCAGGGG A UUGAAGCC | 2109 | GGCTTCAA GGCTAGCTACAACGA CCCCTGCA | 2515 |
| 1700 | GGAUUGAA G CCCUCCAA | 1634 | TTGGAGGG GGCTAGCTACAACGA TTCAATCC | 2516 |
| 1711 | CUCCAAAA G CACUUACG | 1635 | CGTAAGTG GGCTAGCTACAACGA TTTTGGAG | 2517 |
| 1713 | CCAAAAGC A CUUACGGA | 528 | TCCGTAAG GGCTAGCTACAACGA GCTTTTGG | 2518 |
| 1717 | AAGCACUU A CGGAUUCU | 2110 | AGAATCCG GGCTAGCTACAACGA AAGTGCTT | 2519 |
| 1721 | ACUUACGG A UUCUGGUG | 2111 | CACCAGAA GGCTAGCTACAACGA CCGTAAGT | 2520 |
| 1727 | GGAUUCUG G UGGGGUGU | 1636 | ACACCCCA GGCTAGCTACAACGA CAGAATCC | 2521 |
| 1732 | CUGGUGGG G UGUGUUCC | 1637 | GGAACACA GGCTAGCTACAACGA CCCACCAG | 2522 |
| 1734 | GGUGGGGU G UGUUCCAA | 1638 | TTGGAACA GGCTAGCTACAACGA ACCCCACC | 2523 |
| 1736 | UGGGGUGU G UUCCAACU | 1639 | AGTTGGAA GGCTAGCTACAACGA ACACCCCA | 2524 |
| 1742 | GUGUUCCA A CUGCCCCC | 2112 | GGGGGCAG GGCTAGCTACAACGA TGGAACAC | 2525 |
| 1745 | UUCCAACU G CCCCCAAC | 1640 | GTTGGGGG GGCTAGCTACAACGA AGTTGGAA | 2526 |
| 1752 | UGCCCCCA A CUUUGUGG | 2113 | CCACAAAG GGCTAGCTACAACGA TGGGGGCA | 2527 |
| 1757 | CCAACUUU G UGGAUGUC | 1641 | GACATCCA GGCTAGCTACAACGA AAAGTTGG | 2528 |
| 1761 | CUUUGUGG A UGUCUUCC | 2114 | GGAAGACA GGCTAGCTACAACGA CCACAAAG | 2529 |
| 1763 | UUGUGGAU G UCUUCCUU | 1642 | AAGGAAGA GGCTAGCTACAACGA ATCCACAA | 2530 |
| 1782 | AGGGGGGA G CCAUAUUU | 1643 | AAATATGG GGCTAGCTACAACGA TCCCCCCT | 2531 |
| 1785 | GGGGAGCC A UAUUUUAU | 544 | ATAAAATA GGCTAGCTACAACGA GGCTCCCC | 2532 |
| 1787 | GGAGCCAU A UUUUAUUC | 2115 | GAATAAAA GGCTAGCTACAACGA ATGGCTCC | 2533 |
| 1792 | CAUAUUUU A UUCUUUUA | 2116 | TAAAAGAA GGCTAGCTACAACGA AAAATATG | 2534 |
| 1800 | AUUCUUUU A UUGUCAGU | 2117 | ACTGACAA GGCTAGCTACAACGA AAAAGAAT | 2535 |
| 1803 | CUUUUAUU G UCAGUAUC | 1644 | GATACTGA GGCTAGCTACAACGA AATAAAAG | 2536 |
| 1807 | UAUUGUCA G UAUCUGUA | 1645 | TACAGATA GGCTAGCTACAACGA TGACAATA | 2537 |
| 1809 | UUGUCAGU A UCUGUAUC | 2118 | GATACAGA GGCTAGCTACAACGA ACTGACAA | 2538 |
| 1813 | CAGUAUCU G UAUCUCUC | 1646 | GAGAGATA GGCTAGCTACAACGA AGATACTG | 2539 |
| 1815 | GUAUCUGU A UCUCUCUC | 2119 | GAGAGAGA GGCTAGCTACAACGA ACAGATAC | 2540 |
| 1835 | UUUUGGAG G UGCUUAAG | 1647 | CTTAAGCA GGCTAGCTACAACGA CTCCAAAA | 2541 |
| 1837 | UUGGAGGU G CUUAAGCA | 1648 | TGCTTAAG GGCTAGCTACAACGA ACCTCCAA | 2542 |
| 1843 | GUGCUUAA G CAGAAGCA | 1649 | TGCTTCTG GGCTAGCTACAACGA TTAAGCAC | 2543 |
| 1849 | AAGCAGAA G CAUUAACU | 1650 | AGTTAATG GGCTAGCTACAACGA TTCTGCTT | 2544 |
| 1851 | GCAGAAGC A UUAACUUC | 555 | GAAGTTAA GGCTAGCTACAACGA GCTTCTGC | 2545 |
| 1855 | AAGCAUUA A CUUCUCUG | 2120 | CAGAGAAG GGCTAGCTACAACGA TAATGCTT | 2546 |
| 1875 | AGGGGGGA G CUGGGGAA | 1651 | TTCCCCAG GGCTAGCTACAACGA TCCCCCCT | 2547 |
| 1884 | CUGGGGAA A CUCAAACU | 2121 | AGTTTGAG GGCTAGCTACAACGA TTCCCCAG | 2548 |
| 1890 | AAACUCAA A CUUUUCCC | 2122 | GGGAAAAG GGCTAGCTACAACGA TTGAGTTT | 2549 |
| 1901 | UUUCCCCU G UCCUGAUG | 1652 | CATCAGGA GGCTAGCTACAACGA AGGGGAAA | 2550 |
| 1907 | CUGUCCUG A UGGUCAGC | 2123 | GCTGACCA GGCTAGCTACAACGA CAGGACAG | 2551 |
| 1910 | UCCUGAUG G UCAGCUCC | 1653 | GGAGCTGA GGCTAGCTACAACGA CATCAGGA | 2552 |
| 1914 | GAUGGUCA G CUCCCUUC | 1654 | GAAGGGAG GGCTAGCTACAACGA TGACCATC | 2553 |
| 1926 | CCUUCUCU G UAGGGAAC | 1655 | GTTCCCTA GGCTAGCTACAACGA AGAGAAGG | 2554 |
| 1933 | UGUAGGGA A CUGUGGGG | 2124 | CCCCACAG GGCTAGCTACAACGA TCCCTACA | 2555 |
| 1936 | AGGGAACU G UGGGGUCC | 1656 | GGACCCCA GGCTAGCTACAACGA AGTTCCCT | 2556 |
| 1941 | ACUGUGGG G UCCCCCAU | 1657 | ATGGGGGA GGCTAGCTACAACGA CCCACAGT | 2557 |
| 1948 | GGUCCCCC A UCCCCAUC | 581 | GATGGGGA GGCTAGCTACAACGA GGGGGACC | 2558 |
| 1954 | CCAUCCCC A UCCUCCAG | 585 | CTGGAGGA GGCTAGCTACAACGA GGGGATGG | 2559 |
| 1962 | AUCCUCCA G CUUCUGGU | 1658 | ACCAGAAG GGCTAGCTACAACGA TGGAGGAT | 2560 |
| 1969 | AGCUUCUG G UACUCUCC | 1659 | GGAGAGTA GGCTAGCTACAACGA CAGAAGCT | 2561 |
| 1971 | CUUCUGGU A CUCUCCUA | 2125 | TAGGAGAG GGCTAGCTACAACGA ACCAGAAG | 2562 |
| 1983 | UCCUAGAG A CAGAAGCA | 2126 | TGCTTCTG GGCTAGCTACAACGA CTCTAGGA | 2563 |
| 1989 | AGACAGAA G CAGGCUGG | 1660 | CCAGCCTG GGCTAGCTACAACGA TTCTGTCT | 2564 |
| 1993 | AGAAGCAG G CUGGAGGU | 1661 | ACCTCCAG GGCTAGCTACAACGA CTGCTTCT | 2565 |
| 2000 | GGCUGGAG G UAAGGCCU | 1662 | AGGCCTTA GGCTAGCTACAACGA CTCCAGCC | 2566 |
| 2005 | GAGGUAAG G CCUUUGAG | 1663 | CTCAAAGG GGCTAGCTACAACGA CTTACCTC | 2567 |
| 2013 | GCCUUUGA G CCCACAAA | 1664 | TTTGTGGG GGCTAGCTACAACGA TCAAAGGC | 2568 |
| 2017 | UUGAGCCC A CAAAGCCU | 603 | AGGCTTTG GGCTAGCTACAACGA GGGCTCAA | 2569 |
| 2022 | CCCACAAA G CCUUAUCA | 1665 | TGATAAGG GGCTAGCTACAACGA TTTGTGGG | 2570 |
| 2027 | AAAGCCUU A UCAAGUGU | 2127 | ACACTTGA GGCTAGCTACAACGA AAGGCTTT | 2571 |
| 2032 | CUUAUCAA G UGUCUUCC | 1666 | GGAAGACA GGCTAGCTACAACGA TTGATAAG | 2572 |
| 2034 | UAUCAAGU G UCUUCCAU | 1667 | ATGGAAGA GGCTAGCTACAACGA ACTTGATA | 2573 |
| 2041 | UGUCUUCC A UCAUGGAU | 610 | ATCCATGA GGCTAGCTACAACGA GGAAGACA | 2574 |
| 2044 | CUUCCAUC A UGGAUUCA | 611 | TGAATCCA GGCTAGCTACAACGA GATGGAAG | 2575 |
| 2048 | CAUCAUGG A UUCAUUAC | 2128 | GTAATGAA GGCTAGCTACAACGA CCATGATG | 2576 |
| 2052 | AUGGAUUC A UUACAGCU | 612 | AGCTGTAA GGCTAGCTACAACGA GAATCCAT | 2577 |
| 2055 | GAUUCAUU A CAGCUUAA | 2129 | TTAAGCTG GGCTAGCTACAACGA AATGAATC | 2578 |
| 2058 | UCAUUACA G CUUAAUCA | 1668 | TGATTAAG GGCTAGCTACAACGA TGTAATGA | 2579 |
| 2063 | ACAGCUUA A UCAAAAUA | 2130 | TATTTTGA GGCTAGCTACAACGA TAAGCTGT | 2580 |
| 2069 | UAAUCAAA A UAACGCCC | 2131 | GGGCGTTA GGCTAGCTACAACGA TTTGATTA | 2581 |
| 2072 | UCAAAAUA A CGCCCCAG | 2132 | CTGGGGCG GGCTAGCTACAACGA TATTTTGA | 2582 |
| 2074 | AAAAUAAC G CCCCAGAU | 1669 | ATCTGGGG GGCTAGCTACAACGA GTTATTTT | 2583 |
| 2081 | CGCCCCAG A UACCAGCC | 2133 | GGCTGGTA GGCTAGCTACAACGA CTGGGGCG | 2584 |
| 2083 | CCCCAGAU A CCAGCCCC | 2134 | GGGGCTGG GGCTAGCTACAACGA ATCTGGGG | 2585 |
| 2087 | AGAUACCA G CCCCUGUA | 1670 | TACAGGGG GGCTAGCTACAACGA TGGTATCT | 2586 |
| 2093 | CAGCCCCU G UAUGGCAC | 1671 | GTGCCATA GGCTAGCTACAACGA AGGGGCTG | 2587 |
| 2095 | GCCCCUGU A UGGCACUG | 2135 | CAGTGCCA GGCTAGCTACAACGA ACAGGGGC | 2588 |
| 2098 | CCUGUAUG G CACUGGCA | 1672 | TGCCAGTG GGCTAGCTACAACGA CATACAGG | 2589 |
| 2100 | UGUAUGGC A CUGGCAUU | 626 | AATGCCAG GGCTAGCTACAACGA GCCATACA | 2590 |
| 2104 | UGGCACUG G CAUUGUCC | 1673 | GGACAATG GGCTAGCTACAACGA CAGTGCCA | 2591 |
| 2106 | GCACUGGC A UUGUCCCU | 628 | AGGGACAA GGCTAGCTACAACGA GCCAGTGC | 2592 |
| 2109 | CUGGCAUU G UCCCUGUG | 1674 | CACAGGGA GGCTAGCTACAACGA AATGCCAG | 2593 |
| 2115 | UUGUCCCU G UGCCUAAC | 1675 | GTTAGGCA GGCTAGCTACAACGA AGGGACAA | 2594 |
| 2117 | GUCCCUGU G CCUAACAC | 1676 | GTGTTAGG GGCTAGCTACAACGA ACAGGGAC | 2595 |
| 2122 | UGUGCCUA A CACCAGCG | 2136 | CGCTGGTG GGCTAGCTACAACGA TAGGCACA | 2596 |
| 2124 | UGCCUAAC A CCAGCGUU | 634 | AACGCTGG GGCTAGCTACAACGA GTTAGGCA | 2597 |
| 2128 | UAACACCA G CGUUUGAG | 1677 | CTCAAACG GGCTAGCTACAACGA TGGTGTTA | 2598 |
| 2130 | ACACCAGC G UUUGAGGG | 1678 | CCCTCAAA GGCTAGCTACAACGA GCTGGTGT | 2599 |
| 2139 | UUUGAGGG G CUGCCUUC | 1679 | GAAGGCAG GGCTAGCTACAACGA CCCTCAAA | 2600 |
| 2142 | GAGGGGCU G CCUUCCUG | 1680 | CAGGAAGG GGCTAGCTACAACGA AGCCCCTC | 2601 |
| 2150 | GCCUUCCU G CCCUACAG | 1681 | CTGTAGGG GGCTAGCTACAACGA AGGAAGGC | 2602 |
| 2155 | CCUGCCCU A CAGAGGUC | 2137 | GACCTCTG GGCTAGCTACAACGA AGGGCAGG | 2603 |
| 2161 | CUACAGAG G UCUCUGCC | 1682 | GGCAGAGA GGCTAGCTACAACGA CTCTGTAG | 2604 |
| 2167 | AGGUCUCU G CCGGCUCU | 1683 | AGAGCCGG GGCTAGCTACAACGA AGAGACCT | 2605 |
| 2171 | CUCUGCCG G CUCUUUCC | 1684 | GGAAAGAG GGCTAGCTACAACGA CGGCAGAG | 2606 |
| 2182 | CUUUCCUU G CUCAACCA | 1685 | TGGTTGAG GGCTAGCTACAACGA AAGGAAAG | 2607 |
| 2187 | CUUGCUCA A CCAUGGCU | 2138 | AGCCATGG GGCTAGCTACAACGA TGAGCAAG | 2608 |
| 2190 | GCUCAACC A UGGCUGAA | 656 | TTCAGCCA GGCTAGCTACAACGA GGTTGAGC | 2609 |
| 2193 | CAACCAUG G CUGAAGGA | 1686 | TCCTTCAG GGCTAGCTACAACGA CATGGTTG | 2610 |
| 2203 | UGAAGGAA A CAGUGCAA | 2139 | TTGCACTG GGCTAGCTACAACGA TTCCTTCA | 2611 |
| 2206 | AGGAAACA G UGCAACAG | 1687 | CTGTTGCA GGCTAGCTACAACGA TGTTTCCT | 2612 |
| 2208 | GAAACAGU G CAACAGCA | 1688 | TGCTGTTG GGCTAGCTACAACGA ACTGTTTC | 2613 |
| 2211 | ACAGUGCA A CAGCACUG | 2140 | CAGTGCTG GGCTAGCTACAACGA TGCACTGT | 2614 |
| 2214 | GUGCAACA G CACUGGCU | 1689 | AGCCAGTG GGCTAGCTACAACGA TGTTGCAC | 2615 |
| 2216 | GCAACAGC A CUGGCUCU | 661 | AGAGCCAG GGCTAGCTACAACGA GCTGTTGC | 2616 |
| 2220 | CAGCACUG G CUCUCUCC | 1690 | GGAGAGAG GGCTAGCTACAACGA CAGTGCTG | 2617 |
| 2232 | UCUCCAGG A UCCAGAAG | 2141 | CTTCTGGA GGCTAGCTACAACGA CCTGGAGA | 2618 |
| 2243 | CAGAAGGG G UUUGGUCU | 1691 | AGACCAAA GGCTAGCTACAACGA CCCTTCTG | 2619 |
| 2248 | GGGGUUUG G UCUGGACU | 1692 | AGTCCAGA GGCTAGCTACAACGA CAAACCCC | 2620 |
| 2254 | UGGUCUGG A CUUCCUUG | 2142 | CAAGGAAG GGCTAGCTACAACGA CCAGACCA | 2621 |
| 2262 | ACUUCCUU G CUCUCCCC | 1693 | GUGGAGAG GGCTAGCTACAACGA AAGGAAGT | 2622 |
| 2280 | CUUCUCAA G UGCCUUAA | 1694 | TTAAGGCA GGCTAGCTACAACGA TTGAGAAG | 2623 |
| 2282 | UCUCAAGU G CCUUAAUA | 1695 | TATTAAGG GGCTAGCTACAACGA ACTTGAGA | 2624 |
| 2288 | GUGCCUUA A UAGUAGGG | 2143 | CCCTACTA GGCTAGCTACAACGA TAAGGCAC | 2625 |
| 2291 | CCUUAAUA G UAGGGUAA | 1696 | TTACCCTA GGCTAGCTACAACGA TATTAAGG | 2626 |
| 2296 | AUAGUAGG G UAAGUUGU | 1697 | ACAACTTA GGCTAGCTACAACGA CCTACTAT | 2627 |
| 2300 | UAGGGUAA G UUGUUAAG | 1698 | CTTAACAA GGCTAGCTACAACGA TTACCCTA | 2628 |
| 2303 | GGUAAGUU G UUAAGAGU | 1699 | ACTCTTAA GGCTAGCTACAACGA AACTTACC | 2629 |
| 2310 | UGUUAAGA G UGGGGGAG | 1700 | CTCCCCCA GGCTAGCTACAACGA TCTTAACA | 2630 |
| 2320 | GGGGGAGA G CAGGCUGG | 1701 | CCAGCCTG GGCTAGCTACAACGA TCTCCCCC | 2631 |
| 2324 | GAGAGCAG G CUGGCAGC | 1702 | GCTGCCAG GGCTAGCTACAACGA CTGCTCTC | 2632 |
| 2328 | GCAGGCUG G CAGCUCUC | 1703 | GAGAGCTG GGCTAGCTACAACGA CAGCCTGC | 2633 |
| 2331 | GGCUGGCA G CUCUCCAG | 1704 | CTGGAGAG GGCTAGCTACAACGA TGCCAGCC | 2634 |
| 2339 | GCUCUCCA G UCAGGAGG | 1705 | CCTCCTGA GGCTAGCTACAACGA TGGAGAGC | 2635 |
| 2347 | GUCAGGAG G CAUAGUUU | 1706 | AAACTATG GGCTAGCTACAACGA CTCCTGAC | 2636 |
| 2349 | CAGGAGGC A UAGUUUUU | 693 | AAAAACTA GGCTAGCTACAACGA GCCTCCTG | 2637 |
| 2352 | GAGGCAUA G UUUUUAGU | 1707 | ACTAAAAA GGCTAGCTACAACGA TATGCCTC | 2638 |
| 2359 | AGUUUUUA G UGAACAAU | 1708 | ATTGTTCA GGCTAGCTACAACGA TAAAAACT | 2639 |
| 2363 | UUUAGUGA A CAAUCAAA | 2144 | TTTGATTG GGCTAGCTACAACGA TCACTAAA | 2640 |
| 2366 | AGUGAACA A UCAAAGCA | 2145 | TGCTTTGA GGCTAGCTACAACGA TGTTCACT | 2641 |
| 2372 | CAAUCAAA G CACUUGGA | 1709 | TCCAAGTG GGCTAGCTACAACGA TTTGATTG | 2642 |
| 2374 | AUCAAAGC A CUUGGACU | 696 | AGTCCAAG GGCTAGCTACAACGA GCTTTGAT | 2643 |
| 2380 | GCACUUGG A CUCUUGCU | 2146 | AGCAAGAG GGCTAGCTACAACGA CCAAGTGC | 2644 |
| 2386 | GGACUCUU G CUCUUUCU | 1710 | AGAAAGAG GGCTAGCTACAACGA AAGAGTCC | 2645 |
| 2395 | CUCUUUCU A CUCUGAAC | 2147 | GTTCAGAG GGCTAGCTACAACGA AGAAAGAC | 2646 |
| 2402 | UACUCUGA A CUAAUAAA | 2148 | TTTATTAG GGCTAGCTACAACGA TCAGAGTA | 2647 |
| 2406 | CUGAACUA A UAAAGCUG | 2149 | CAGCTTTA GGCTAGCTACAACGA TAGTTCAG | 2648 |
| 2411 | CUAAUAAA G CUGUUGCC | 1711 | GGCAACAG GGCTAGCTACAACGA TTTATTAG | 2649 |
| 2414 | AUAAAGCU G UUGCCAAG | 1712 | CTTGGCAA GGCTAGCTACAACGA AGCTTTAT | 2650 |
| 2417 | AAGCUGUU G CCAAGCUG | 1713 | CAGCTTGG GGCTAGCTACAACGA AACAGCTT | 2651 |
| 2422 | GUUGCCAA G CUGGACGG | 1714 | CCGTCCAG GGCTAGCTACAACGA TTGGCAAC | 2652 |
| 2427 | CAAGCUGG A CGGCACGA | 2150 | TCGTGCCG GGCTAGCTACAACGA CCAGCTTG | 2653 |
| 2430 | GCUGGACG G CACGAGCU | 1715 | AGCTCGTG GGCTAGCTACAACGA CGTCCAGC | 2654 |
| 2432 | UGGACGGC A CGAGCUCG | 710 | CGAGCTCG GGCTAGCTACAACGA GCCGTCCA | 2655 |
| 2436 | CGGCACGA G CUCGUGCC | 1716 | GGCACGAG GGCTAGCTACAACGA TCGTGCCG | 2656 |

Input Sequence = NM_021975. Cut Site = R/Y Arm Length = 8. Core Sequence = GGCTAGCTACAACGA NM_021975 (Homo sapiens p65 RelA (NFKB), mRNA; 2444 bp)

TABLE VI
Human REL-A Amberzyme and Substrate Sequence

| | | Seq | | Seq |
|---|---|---|---|---|
| Pos | Substrate | ID | Amberzyme | ID |
| 9 | GGCACGAG G CGGGGCCG | 1421 | CGGCCCCG GGAGGAAACUCC CU UCAAGGACAUCGUCCGGG CUCGUGCC | 2994 |
| 11 | CACGAGGC G GGGCCGGG | 2657 | CCCGGCCC GGAGGAAACUCC CU UCAAGGACAUCGUCCGGG GCCUCGUG | 2995 |
| 12 | ACGAGGCG G GGCCGGGU | 2658 | ACCCGGCC GGAGGAAACUCC CU UCAAGGACAUCGUCCGGG CGCCUCGU | 2996 |
| 13 | CGAGGCGG G GCCGGGUC | 2659 | GACCCGGC GGAGGAAACUCC CU UCAAGGACAUCGUCCGGG CCGCCUCG | 2997 |
| 14 | GAGGCGGG G CCGGGUCG | 1422 | CGACCCGG GGAGGAAACUCC CU UCAAGGACAUCGUCCGGG CCCGCCUC | 2998 |
| 17 | GCGGGGCC G GGUCGCAG | 2660 | CUGCGACC GGAGGAAACUCC CU UCAAGGACAUCGUCCGGG GGCCCCGC | 2999 |
| 18 | CGGGGCCG G GUCGCAGC | 2661 | GCUGCGAC GGAGGAAACUCC CU UCAAGGACAUCGUCCGGG CGGCCCCG | 3000 |
| 19 | GGGGCCGG G UCGCAGCU | 1423 | AGCUGCGA GGAGGAAACUCC CU UCAAGGACAUCGUCCGGG CCGGCCCC | 3001 |
| 22 | GCCGGGUC G CAGCUGGG | 1424 | CCCAGCUG GGAGGAAACUCC CU UCAAGGACAUCGUCCGGG GACCCGGC | 3002 |
| 25 | GGGUCGCA G CUGGGCCC | 1425 | GGGCCCAG GGAGGAAACUCC CU UCAAGGACAUCGUCCGGG UGCGACCC | 3003 |
| 28 | UCGCAGCU G GGCCCGCG | 2662 | CGCGGGCC GGAGGAAACUCC CU UCAAGGACAUCGUCCGGG AGCUGCGA | 3004 |
| 29 | CGCAGCUG G GCCCGCGG | 2663 | CCGCGGGC GGAGGAAACUCC CU UCAAGGACAUCGUCCGGG CAGCUGCG | 3005 |
| 30 | GCAGCUGG G CCCGCGGC | 1426 | GCCGCGGG GGAGGAAACUCC CU UCAAGGACAUCGUCCGGG CCAGCUGC | 3006 |
| 34 | CUGGGCCC G CGGCAUGG | 1427 | CCAUGCCG GGAGGAAACUCC CU UCAAGGACAUCGUCCGGG GGGCCCAG | 3007 |
| 36 | GGGCCCGC G GCAUGGAC | 2664 | GUCCAUGC GGAGGAAACUCC CU UCAAGGACAUCGUCCGGG GCGGGCCC | 3008 |
| 37 | GGCCCGCG G CAUGGACG | 1428 | CGUCCAUG GGAGGAAACUCC CU UCAAGGACAUCGUCCGGG CGCGGGCC | 3009 |
| 41 | CGCGGCAU G GACGAACU | 2665 | AGUUCGUC GGAGGAAACUCC CU UCAAGGACAUCGUCCGGG AUGCCGCG | 3010 |
| 42 | GCGGCAUG G ACGAACUG | 2666 | CAGUUCGU GGAGGAAACUCC CU UCAAGGACAUCGUCCGGG CAUGCCGC | 3011 |
| 45 | GCAUCGAC G AACUGUUC | 2667 | GAACAGUU GGAGGAAACUCC CU UCAAGGACAUCGUCCGGG CUCCAUGC | 3012 |
| 50 | GACGAACU G UUCCCCCU | 1429 | AGGGGGAA GGAGGAAACUCC CU UCAAGGACAUCGUCCGGG AGUUCGUC | 3013 |
| 68 | AUCUUCCC G GCAGAGCA | 2668 | UGCUCUGC GGAGGAAACUCC CU UCAAGGACAUCGUCCGGG GGGAAGAU | 3014 |
| 69 | UCUUCCCG G CAGAGCAG | 1430 | CUGCUCUG GGAGGAAACUCC CU UCAAGGACAUCGUCCGGG CGGGAAGA | 3015 |
| 72 | UCCCCGCA G AGCAGCCC | 2669 | GGGCUGCU GGAGGAAACUCC CU UCAAGGACAUCGUCCGGG UGCCGCGA | 3016 |
| 74 | CCGGCAGA G CAGCCCAA | 1431 | UUGGGCUG GGAGGAAACUCC CU UCAAGGACAUCGUCCGGG UCUGCCGG | 3017 |
| 77 | GCAGACCA G CCCAAGCA | 1432 | UGCUUGGG GGAGGAAACUCC CU UCAAGGACAUCGUCCGGG UGCUCUGC | 3018 |
| 83 | CAGCCCAA G CAGCGGGG | 1433 | CCCCGCUG GGAGGAAACUCC CU UCAAGGACAUCGUCCGGG UUGGGCUG | 3019 |
| 86 | CCCAAGCA G CGGGGCAU | 1434 | AUGCCCCG GGAGGAAACUCC CU UCAAGGACAUCGUCCGGG UGCUUGGG | 3020 |
| 88 | CAAGCAGC G GGGCAUGC | 2670 | GCAUGCCC GGAGGAAACUCC CU UCAAGGACAUCGUCCGGG GCUGCUUG | 3021 |
| 89 | AAGCAGCG G GGCAUGCG | 2671 | CGCAUGCC GGAGGAAACUCC CU UCAAGGACAUCGUCCGGG CGCUGCUU | 3022 |
| 90 | AGCAGCGG G GCAUGCGC | 2672 | GCGCAUGC GGAGGAAACUCC CU UCAAGGACAUCGUCCGGG CCGCUGCU | 3023 |
| 91 | GCAGCGGG G CAUGCGCU | 1435 | AGCGCAUG GGAGGAAACUCC CU UCAAGGACAUCGUCCGGG CCCGCUGC | 3024 |
| 95 | CGGGGCAU G CGCUUCCG | 1436 | CGGAAGCG GGAGGAAACUCC CU UCAAGGACAUCGUCCGGG AUGCCCCG | 3025 |
| 97 | GGGCAUGC G CUUCCGCU | 1437 | GCAUGCCC GGAGGAAACUCC CU UCAAGGACAUCGUCCGGG GCAUGCCC | 3026 |
| 103 | GCGCUUCC G CUACAAGU | 1438 | GGAAGCGC GGAGGAAACUCC CU UCAAGGACAUCGUCCGGG GGAAGCGC | 3027 |
| 110 | CGCUACAA G UGCGAGGG | 1439 | UUGUAGCG GGAGGAAACUCC CU UCAAGGACAUCGUCCGGG UUGUAGCG | 3028 |
| 112 | CUACAAGU G CGAGGGGC | 1440 | ACUUGUAG GGAGGAAACUCC CU UCAAGGACAUCGUCCGGG ACUUGUAG | 3029 |
| 114 | ACAAGUGC G AGGGGCGC | 2673 | GCACUUGU GGAGGAAACUCC CU UCAAGGACAUCGUCCGGG GCACUUGU | 3030 |
| 116 | AAGUGCGA G GGGCGCUC | 2674 | UCGCACUU GGAGGAAACUCC CU UCAAGGACAUCGUCCGGG UCGCACUU | 3031 |
| 117 | AGUGCGAG G GGCGCUCC | 2675 | CUCGCACU GGAGGAAACUCC CU UCAAGGACAUCGUCCGGG CUCGCACU | 3032 |
| 118 | GUGCGAGG G GCGCUCCG | 2676 | CCUCGCAC GGAGGAAACUCC CU UCAAGGACAUCGUCCGGG CCUCGCAC | 3033 |
| 119 | UGCGAGGG G CGCUCCGC | 1441 | CCCUCGCA GGAGGAAACUCC CU UCAAGGACAUCGUCCGGG CCCUCGCA | 3034 |
| 121 | CGAGGGGC G CUCCGCGG | 1442 | GCCCCUCG GGAGGAAACUCC CU UCAAGGACAUCGUCCGGG GCCCCUCG | 3035 |
| 126 | GGCGCUCC G CGGGCAGC | 1443 | GGAGCGCC GGAGGAAACUCC CU UCAAGGACAUCGUCCGGG GGAGCGCC | 3036 |
| 128 | CGCUCCGC G GGCAGCAU | 2677 | GCGGAGCG GGAGGAAACUCC CU UCAAGGACAUCGUCCGGG GCGGAGCG | 3037 |
| 129 | GCUCCGCG G GCAGCAUC | 2678 | CGCGCAGC GGAGGAAACUCC CU UCAAGGACAUCGUCCGGG CGCGGAGC | 3038 |
| 130 | CUCCGCGG G CAGCAUCC | 1444 | GGAUGCUG GGAGGAAACUCC CU UCAAGGACAUCGUCCGGG CCGCGGAG | 3039 |
| 133 | CGCGGGCA G CAUCCCAG | 1445 | CUGGGAUG GGAGGAAACUCC CU UCAAGGACAUCGUCCGGG UGCCCGCG | 3040 |
| 141 | GCAUCCCA G GCGAGAGG | 2679 | CCUCUCGC GGAGGAAACUCC CU UCAAGGACAUCGUCCGGG UGGGAUGC | 3041 |
| 142 | CAUCCCAG G CGAGAGGA | 1446 | UCCUCUCG GGAGGAAACUCC CU UCAAGGACAUCGUCCGGG CUGGGAUG | 3042 |
| 144 | UCCCAGGC G AGAGGAGC | 2680 | GCUCCUCU GGAGGAAACUCC CU UCAAGGACAUCGUCCGGG GCCUGGGA | 3043 |
| 146 | CCAGGCGA G AGGAGCAC | 2681 | GUGCUCCU GGAGGAAACUCC CU UCAAGGACAUCGUCCGGG UCGCCUGG | 3044 |
| 148 | AGGCGAGA G GAGCACAG | 2682 | CUGUGCUC GGAGGAAACUCC CU UCAAGGACAUCGUCCGGG UCUCGCCU | 3045 |
| 149 | GGCGAGAG G AGCACAGA | 2683 | UCUGUGCU GGAGGAAACUCC CU UCAAGGACAUCGUCCGGG CUCUCGCC | 3046 |
| 151 | CGAGAGGA G CACAGAUA | 1447 | UAUCUGUG GGAGGAAACUCC CU UCAAGGACAUCGUCCGGG UCCUCUCG | 3047 |
| 156 | GGAGCACA G AUACCACC | 2684 | GGUGGUAU GGAGGAAACUCC CU UCAAGGACAUCGUCCGGG UGUGCUCC | 3048 |
| 167 | ACCACCAA G ACCCACCC | 2685 | GGGUGGGU GGAGGAAACUCC CU UCAAGGACAUCGUCCGGG UUGGUGGU | 3049 |
| 185 | ACCAUCAA G AUCAAUGG | 2686 | CCAUUGAU GGAGGAAACUCC CU UCAAGGACAUCGUCCGGG UUGAUGGU | 3050 |
| 192 | AGAUCAAU G GCUACACA | 2687 | UGUGUAGC GGAGGAAACUCC CU UCAAGGACAUCGUCCGGG AUUGAUCU | 3051 |
| 193 | GAUCAAUG G CUACACAG | 1448 | CUGUGUAG GGAGGAAACUCC CU UCAAGGACAUCGUCCGGG CAUUGAUC | 3052 |
| 201 | GCUACACA G GACCAGGG | 2688 | CCCUGGUC GGAGGAAACUCC CU UCAAGGACAUCGUCCGGG UGUGUAGC | 3053 |
| 202 | CUACACAG G ACCAGGGA | 2689 | UCCCUGGU GGAGGAAACUCC CU UCAAGGACAUCGUCCGGG CUGUGUAG | 3054 |
| 207 | CAGGACCA G GGACAGUG | 2690 | CACUGUCC GGAGGAAACUCC CU UCAAGGACAUCGUCCGGG UGGUCCUG | 3055 |
| 208 | AGGACCAG G GACAGUGC | 2691 | GCACUGUC GGAGGAAACUCC CU UCAAGGACAUCGUCCGGG CUGGUCCU | 3056 |
| 209 | GGACCAGG G ACAGUGCG | 2692 | CGCACUGU GGAGGAAACUCC CU UCAAGGACAUCGUCCGGG CCUGGUCC | 3057 |
| 213 | CAGGGACA G UGCGCAUC | 1449 | GAUGCGCA GGAGGAAACUCC CU UCAAGGACAUCGUCCGGG UGUCCCUG | 3058 |
| 215 | GGGACAGU G GGCAUCUC | 1450 | GAGAUGCG GGAGGAAACUCC CU UCAAGGACAUCGUCCGGG ACUGUCCC | 3059 |
| 217 | GACAGUGC G CAUCUCCC | 1451 | GGGAGAUG GGAGGAAACUCC CU UCAAGGACAUCGUCCGGG GCACUGUC | 3060 |
| 227 | AUCUCCCU G CUCACCAA | 2693 | UUGGUGAC GGAGGAAACUCC CU UCAAGGACAUCGUCCGGG AGGGAGAU | 3061 |
| 228 | UCUCCCUG G UCACCAAG | 1452 | CUUGGUGA GGAGGAAACUCC CU UCAAGGACAUCGUCCGGG CAGGGAGA | 3062 |
| 236 | GUCACCAA G GACCCUCC | 2694 | GGAGGGUC GGAGGAAACUCC CU UCAAGGACAUCGUCCGGG UUGGUGAC | 3063 |
| 237 | UCACCAAG G ACCCUCCU | 2695 | AGGAGGGU GGAGGAAACUCC CU UCAAGGACAUCGUCCGGG CUUGGUGA | 3064 |
| 250 | UCCUCACC G GCCUCACC | 2696 | GGUCAGGC GGAGGAAACUCC CU UCAAGGACAUCGUCCGGG GGUGAGGA | 3065 |
| 251 | CCUCACCG G CCUCACCC | 1453 | GGGUGAGG GGAGGAAACUCC CU UCAAGGACAUCGUCCGGG CGGUGAGG | 3066 |
| 264 | ACCCCCAC G AGCUUGUA | 2697 | UACAAGCU GGAGGAAACUCC CU UCAAGGACAUCGUCCGGG GUGGGGGU | 3067 |
| 266 | CCCCACGA G CUUGUAGG | 1454 | CCUACAAG GGAGGAAACUCC CU UCAAGGACAUCGUCCGGG UCGUGGGG | 3068 |
| 270 | ACGAGCUU G UAGGAAAG | 1455 | CUUUCCUA GGAGGAAACUCC CU UCAAGGACAUCGUCCGGG AAGCUCGU | 3069 |
| 273 | AGCUUGUA G GAAAGGAC | 2698 | GUCCUUUC GGAGGAAACUCC CU UCAAGGACAUCGUCCGGG UACAAGCU | 3070 |
| 274 | GCUUGUAG G AAAGGACU | 2699 | AGUCCUUU GGAGGAAACUCC CU UCAAGGACAUCGUCCGGG CUACAAGC | 3071 |
| 278 | GUAGGAAA G GACUGCCG | 2700 | CGGCAGUC GGAGGAAACUCC CU UCAAGGACAUCGUCCGGG UUUCCUAC | 3072 |
| 279 | UAGGAAAG G ACUGCCGG | 2701 | CCGGCAGU GGAGGAAACUCC CU UCAAGGACAUCGUCCGGG CUUUCCUA | 3073 |
| 283 | AAAGGACU G CCGGGAUG | 1456 | CAUCCCGG GGAGGAAACUCC CU UCAAGGACAUCGUCCGGG AGUCCUUU | 3074 |
| 286 | GGACUGCC G GGAUGGCU | 2702 | AGCCAUCC GGAGGAAACUCC CU UCAAGGACAUCGUCCGGG GGCAGUCC | 3075 |
| 287 | GACUGCCG G GAUGGCUU | 2703 | AAGCCAUC GGAGGAAACUCC CU UCAAGGACAUCGUCCGGG CGGCAGUC | 3076 |
| 288 | ACUGCCGG G AUGGCUUC | 2704 | GAAGCCAU GGAGGAAACUCC CU UCAAGGACAUCGUCCGGG CCGGCAGU | 3077 |
| 291 | GCCGGGAU G GCUUCUAU | 2705 | AUAGAAGC GGAGGAAACUCC CU UCAAGGACAUCGUCCGGG AUCCCGGC | 3078 |
| 292 | CCGGGAUG G CUUCUAUG | 1457 | CAUAGAAG GGAGGAAACUCC CU UCAAGGACAUCGUCCGGG CAUCCCGG | 3079 |
| 300 | GCUUCUAU G AGGCUGAG | 2706 | CUCAGCCU GGAGGAAACUCC CU UCAAGGACAUCGUCCGGG AUAGAAGC | 3080 |
| 302 | UUCUAUGA G GCUGAGCU | 2707 | AGCUCAGC GGAGGAAACUCC CU UCAAGGACAUCGUCCGGG UCAUAGAA | 3081 |
| 303 | UCUAUGAG G CUGAGCUC | 1458 | GAGCUCAG GGAGGAAACUCC CU UCAAGGACAUCGUCCGGG CUCAUAGA | 3082 |
| 306 | AUGAGGCU G AGCUCUGC | 2708 | GCAGAGCU GGAGGAAACUCC CU UCAAGGACAUCGUCCGGG AGCCUCAU | 3083 |
| 308 | GAGGCUGA G CUCUGCCC | 1459 | GGGCAGAG GGAGGAAACUCC CU UCAAGGACAUCGUCCGGG UCAGCCUC | 3084 |
| 313 | UGAGCUCU G CCCGGACC | 1460 | GGUCCGGG GGAGGAAACUCC CU UCAAGGACAUCGUCCGGG AGAGCUCA | 3085 |
| 317 | CUCUGCCC G GACCGCUG | 2709 | CAGCGGUC GGAGGAAACUCC CU UCAAGGACAUCGUCCGGG GGGCAGAG | 3086 |
| 318 | UCUGCCCG G ACCGCUGC | 2710 | GCAGCGGU GGAGGAAACUCC CU UCAAGGACAUCGUCCGGG CGGGCAGA | 3087 |
| 322 | CCCGGACC G CUGCAUCC | 1461 | GGAUGCAG GGAGGAAACUCC CU UCAAGGACAUCGUCCGGG GGUCCGGG | 3088 |
| 325 | GGACCGCU G CAUCCACA | 1462 | UGUGGAUG GGAGGAAACUCC CU UCAAGGACAUCGUCCGGG AGCGGUCC | 3089 |
| 334 | CAUCCACA G UUUCCAGA | 1463 | UCUGGAAA GGAGGAAACUCC CU UCAAGGACAUCGUCCGGG UGUGGAUG | 3090 |
| 341 | AGUUUCCA G AACCUGGG | 2711 | CCCAGGUU GGAGGAAACUCC CU UCAAGGACAUCGUCCGGG UGGAAACU | 3091 |
| 347 | CAGAACCU G GGAAUCCA | 2712 | UGGAUUCC GGAGGAAACUCC CU UCAAGGACAUCGUCCGGG AGGUUCUG | 3092 |
| 348 | AGAACCUG G GAAUCCAG | 2713 | CUGGAUUC GGAGGAAACUCC CU UCAAGGACAUCGUCCGGG CAGGUUCU | 3093 |
| 349 | GAACCUGG G AAUCCAGU | 2714 | ACUGGAUU GGAGGAAACUCC CU UCAAGGACAUCGUCCGGG CCAGGUUC | 3094 |
| 356 | GGAAUCCA G UGUGUGAA | 1464 | UUCACACA GGAGGAAACUCC CU UCAAGGACAUCGUCCGGG UGGAUUCC | 3095 |
| 358 | AAUCCAGU G UGUGAAGA | 1465 | UCUUCACA GGAGGAAACUCC CU UCAAGGACAUCGUCCGGG ACUGGAUU | 3096 |
| 360 | UCCAGUGU G UGAAGAAG | 1466 | CUUCUUCA GGAGGAAACUCC CU UCAAGGACAUCGUCCGGG ACACUGGA | 3097 |
| 362 | CAGUGUGU G AAGAAGCG | 2715 | CGCUUCUU GGAGGAAACUCC CU UCAAGGACAUCGUCCGGG ACACACUG | 3098 |
| 365 | UGUGUGAA G AAGCGGGA | 2716 | UCCCGCUU GGAGGAAACUCC CU UCAAGGACAUCGUCCGGG UUCACACA | 3099 |
| 368 | GUGAAGAA G CGGGACCU | 1467 | AGGUCCCG GGAGGAAACUCC CU UCAAGGACAUCGUCCGGG UUCUUCAC | 3100 |
| 370 | GAAGAAGC G GGACCUGG | 2717 | CCAGGUCC GGAGGAAACUCC CU UCAAGGACAUCGUCCGGG GCUUCUUC | 3101 |
| 371 | AAGAAGCG G GACCUGGA | 2718 | UCCAGGUC GGAGGAAACUCC CU UCAAGGACAUCGUCCGGG CGCUUCUU | 3102 |
| 372 | AGAAGCGG G ACCUGGAG | 2719 | CUCCAGGU GGAGGAAACUCC CU UCAAGGACAUCGUCCGGG CCGCUUCU | 3103 |
| 377 | CGGGACCU G GAGCAGGC | 2720 | GCCUGCUC GGAGGAAACUCC CU UCAAGGACAUCGUCCGGG AGGUCCCG | 3104 |
| 378 | GGGACCUG G AGCAGGCU | 2721 | AGCCUGCU GGAGGAAACUCC CU UCAAGGACAUCGUCCGGG CAGGUCCC | 3105 |
| 380 | GACCUGGA G CAGGCUAU | 1468 | AUAGCCUG GGAGGAAACUCC CU UCAAGGACAUCGUCCGGG UCCAGGUC | 3106 |
| 383 | CUGGAGCA G GCUAUCAG | 2722 | CUGAUAGC GGAGGAAACUCC CU UCAAGGACAUCGUCCGGG UGCUCCAG | 3107 |
| 384 | UGGAGCAG G CUAUCAGU | 1469 | ACUGAUAG GGAGGAAACUCC CU UCAAGGACAUCGUCCGGG CUGCUCCA | 3108 |
| 391 | GGCUAUCA G UCAGCGCA | 1470 | UGCGCUGA GGAGGAAACUCC CU UCAAGGACAUCGUCCGGG UGAUAGCC | 3109 |
| 395 | AUCAGUCA G CGCAUCCA | 1471 | UGGAUGCG GGAGGAAACUCC CU UCAAGGACAUCGUCCGGG UGACUGAU | 3110 |
| 397 | CAGUCAGC G CAUCCAGA | 1472 | UCUGGAUG GGAGGAAACUCC CU UCAAGGACAUCGUCCGGG GCUGACUG | 3111 |
| 404 | CGCAUCCA G ACCAACAA | 2723 | UUGUUGGU GGAGGAAACUCC CU UCAAGGACAUCGUCCGGG UGGAUGCG | 3112 |
| 426 | CCUUCCAA G UUCCUAUA | 1473 | UAUAGGAA GGAGGAAACUCC CU UCAAGGACAUCGUCCGGG UUGGAAGG | 3113 |
| 435 | UUCCUAUA G AAGAGCAG | 2724 | CUGCUCUU GGAGGAAACUCC CU UCAAGGACAUCGUCCGGG UAUAGGAA | 3114 |
| 438 | CUAUAGAA G AGCAGCGU | 2725 | ACGCUGCU GGAGGAAACUCC CU UCAAGGACAUCGUCCGGG UUCUAUAG | 3115 |
| 440 | AUAGAAGA G CAGCGUGG | 1474 | CCACGCUG GGAGGAAACUCC CU UCAAGGACAUCGUCCGGG UCUUCUAU | 3116 |
| 443 | GAAGAGCA G CGUGGGGA | 1475 | UCCCCACG GGAGGAAACUCC CU UCAAGGACAUCGUCCGGG UGCUCUUC | 3117 |
| 445 | AGAGCAGC G UGGGGACU | 1476 | AGUCCCCA GGAGGAAACUCC CU UCAAGGACAUCGUCCGGG GCUGCUCU | 3118 |
| 447 | AGCAGCGU G GGGACUAC | 2726 | GUAGUCCC GGAGGAAACUCC CU UCAAGGACAUCGUCCGGG ACGCUGCU | 3119 |
| 448 | GCAGCGUG G GGACUACG | 2727 | CGUAGUCC GGAGGAAACUCC CU UCAAGGACAUCGUCCGGG CACGCUGC | 3120 |
| 449 | CAGCGUGG G GACUACGA | 2728 | UCGUAGUC GGAGGAAACUCC CU UCAAGGACAUCGUCCGGG CCACGCUG | 3121 |
| 450 | AGCGUGGG G ACUACGAC | 2729 | GUCGUAGU GGAGGAAACUCC CU UCAAGGACAUCGUCCGGG CCCACGCU | 3122 |
| 456 | GGGACUAC G ACCUGAAU | 2730 | AUUCAGGU GGAGGAAACUCC CU UCAAGGACAUCGUCCGGG GUAGUCCC | 3123 |
| 461 | UACGACCU G AAUGCUGU | 2731 | ACAGCAUU GGAGGAAACUCC CU UCAAGGACAUCGUCCGGG AGGUCGUA | 3124 |
| 465 | ACCUGAAU G CUGUGCGG | 1477 | CCGCACAG GGAGGAAACUCC CU UCAAGGACAUCGUCCGGG AUUCAGGU | 3125 |
| 468 | UGAAUGCU G UGCGGCUC | 1478 | GAGCCGCA GGAGGAAACUCC CU UCAAGGACAUCGUCCGGG AGCAUUCA | 3126 |
| 470 | AAUGCUGU G CGGCUCUG | 1479 | CAGAGCCG GGAGGAAACUCC CU UCAAGGACAUCGUCCGGG ACAGCAUU | 3127 |
| 472 | UGCUGUGC G GCUCUGCU | 2732 | AGCAGAGC GGAGGAAACUCC CU UCAAGGACAUCGUCCGGG GCACAGCA | 3128 |
| 473 | GCUGUGCG G CUCUGCUU | 1480 | AAGCAGAG GGAGGAAACUCC CU UCAAGGACAUCGUCCGGG CGCACAGC | 3129 |
| 478 | GCGGCUCU G CUUCCAGG | 1481 | CCUGGAAG GGAGGAAACUCC CU UCAAGGACAUCGUCCGGG AGAGCCGC | 3130 |
| 485 | UGCUUCCA G GUGACAGU | 2733 | ACUGUCAC GGAGGAAACUCC CU UCAAGGACAUCGUCCGGG UGGAAGCA | 3131 |
| 486 | GCUUCCAG G UGACAGUG | 1482 | CACUGUCA GGAGGAAACUCC CU UCAAGGACAUCGUCCGGG CUGGAAGC | 3132 |
| 488 | UUCCAGGU G ACAGUGCG | 2734 | CGCACUGU GGAGGAAACUCC CU UCAAGGACAUCGUCCGGG ACCUGGAA | 3133 |
| 492 | AGGUGACA G UGCGGGAC | 1483 | GUCCCGCA GGAGGAAACUCC CU UCAAGGACAUCGUCCGGG UGUCACCU | 3134 |
| 494 | GUGACAGU G CGGGACCC | 1484 | GGGUCCCG GGAGGAAACUCC CU UCAAGGACAUCGUCCGGG ACUGUCAC | 3135 |
| 496 | GACAGUGC G GGACCCAU | 2735 | AUGGGUCC GGAGGAAACUCC CU UCAAGGACAUCGUCCGGG GCACUGUC | 3136 |
| 497 | ACAGUGCG G GACCCAUC | 2736 | GAUGGGUC GGAGGAAACUCC CU UCAAGGACAUCGUCCGGG CGCACUGU | 3137 |
| 498 | CAGUGCGG G ACCCAUCA | 2737 | UGAUGGGU GGAGGAAACUCC CU UCAAGGACAUCGUCCGGG CCGCACUG | 3138 |
| 507 | ACCCAUCA G GCAGGCCC | 2738 | CGGCCUGC GGAGGAAACUCC CU UCAAGGACAUCGUCCGGG UGAUGCCU | 3139 |
| 508 | CCCAUCAG G CAGGCCCC | 1485 | CGGGCCUG GGAGGAAACUCC CU UCAAGGACAUCGUCCGGG CUGAUGGG | 3140 |
| 511 | AUCAGGCA G GCCCCUCC | 2739 | GGAGGGGC GGAGGAAACUCC CU UCAAGGACAUCGUCCGGG UGCCUGAU | 3141 |
| 512 | UCAGGCAG G CCCCUCCG | 1486 | CCGAGGGG GGAGGAAACUCC CU UCAAGGACAUCGUCCGGG CUGCCUGA | 3142 |
| 520 | GCCCCUCC G CCUGCCGC | 1487 | GCGGCAGG GGAGGAAACUCC CU UCAAGGACAUCGUCCGGG GGAGGGGC | 3143 |
| 524 | CUCCGCCU G CCGCCUGU | 1488 | ACAGGCGG GGAGGAAACUCC CU UCAAGGACAUCGUCCGGG AGGCGGAG | 3144 |
| 527 | CGCCUGCC G CCUGUCCU | 1489 | AGGACAGG GGAGGAAACUCC CU UCAAGGACAUCGUCCGGG GGCAGGCG | 3145 |
| 531 | UGCCGCCU G UCCUUUCU | 1490 | AGAAAGGA GGAGGAAACUCC CU UCAAGGACAUCGUCCGGG AGGCGGCA | 3146 |
| 552 | CCAUCUUU G ACAAUCGU | 2740 | ACGAUUGU GGAGGAAACUCC CU UCAAGGACAUCGUCCGGG AAAGAUGG | 3147 |
| 559 | UGACAAUC G UGCCCCCA | 1491 | UGGGGGCA GGAGGAAACUCC CU UCAAGGACAUCGUCCGGG GAUUGUCA | 3148 |
| 561 | ACAAUCGU G CCCCCAAC | 1492 | GUUGGGGG GGAGGAAACUCC CU UCAAGGACAUCGUCCGGG ACGAUUGU | 3149 |
| 573 | CCAACACU G CCGAGCUC | 1493 | GAGCUCGG GGAGGAAACUCC CU UCAAGGACAUCGUCCGGG AGUGUUGG | 3150 |
| 576 | ACACUGCC G AGCUCAAG | 2741 | CUUGAGCU GGAGGAAACUCC CU UCAAGGACAUCGUCCGGG GGCAGUGU | 3151 |
| 578 | ACUGCCGA G CUCAAGAU | 1494 | AUCUUGAG GGAGGAAACUCC CU UCAAGGACAUCGUCCGGG UCGGCAGU | 3152 |
| 584 | GAGCUCAA G AUCUGCCG | 2742 | CGGCAGAU GGAGGAAACUCC CU UCAAGGACAUCGUCCGGG UUGAGCUC | 3153 |
| 589 | CAAGAUCU G CCGAGUGA | 1495 | UCACUCGG GGAGGAAACUCC CU UCAAGGACAUCGUCCGGG AGAUCUUG | 3154 |
| 592 | GAUCUGCC G AGUGAACC | 2743 | GGUUCACU GGAGGAAACUCC CU UCAAGGACAUCGUCCGGG GGCAGAUC | 3155 |
| 594 | UCUGCCGA G UGAACCGA | 1496 | UCGGUUCA GGAGGAAACUCC CU UCAAGGACAUCGUCCGGG UCGGCAGA | 3156 |
| 596 | UGCCGAGU G AACCCAAA | 2744 | UUUCGGUU GGAGGAAACUCC CU UCAAGGACAUCGUCCGGG ACUCGGCA | 3157 |
| 601 | AGUGAACC G AAACUCUG | 2745 | CAGAGUUU GGAGGAAACUCC CU UCAAGGACAUCGUCCGGG GGUUCACU | 3158 |
| 609 | GAAACUCU G CCAGCUGC | 2746 | GCAGCUGC GGAGGAAACUCC CU UCAAGGACAUCGUCCGGG AGAGUUUC | 3159 |
| 610 | AAACUCUG G CAGCUGCC | 1497 | GGCAGCUG GGAGGAAACUCC CU UCAAGGACAUCGUCCGGG CAGAGUUU | 3160 |
| 613 | CUCUGGCA G CUGCCUCG | 1498 | CGAGGCAG GGAGGAAACUCC CU UCAAGGACAUCGUCCGGG UGCCAGAG | 3161 |
| 616 | UGGCAGCU G CCUCGGUG | 1499 | CACCGAGG GGAGGAAACUCC CU UCAAGGACAUCGUCCGGG AGCUGCCA | 3162 |
| 621 | GCUGCCUC G GUGGGGAU | 2747 | AUCCCCAC GGAGGAAACUCC CU UCAAGGACAUCGUCCGGG GAGGCAGC | 3163 |
| 622 | CUGCCUCG G UGGGGAUG | 1500 | CAUCCCCA GGAGGAAACUCC CU UCAAGGACAUCGUCCGGG CGAGGCAG | 3164 |
| 624 | GCCUCGGU G GGGAUGAG | 2748 | CUCAUCCC GGAGGAAACUCC CU UCAAGGACAUCGUCCGGG ACCGAGGC | 3165 |
| 625 | CCUCGGUG G GGAUGAGA | 2749 | UCUCAUCC GGAGGAAACUCC CU UCAAGGACAUCGUCCGGG CACCGAGG | 3166 |
| 626 | CUCGGUGG G GAUGAGAU | 2750 | AUCUCAUC GGAGGAAACUCC CU UCAAGGACAUCGUCCGGG CCACCGAG | 3167 |
| 627 | UCGGUGGG G AUGAGAUC | 2751 | GAUCUCAU GGAGGAAACUCC CU UCAAGGACAUCGUCCGGG CCCACCGA | 3168 |
| 630 | GUGGGGAU G AGAUCUUC | 2752 | GAAGAUCU GGAGGAAACUCC CU UCAAGGACAUCGUCCGGG AUCCCCAC | 3169 |
| 632 | GGGGAUGA G AUCUUCCU | 2753 | AGGAAGAU GGAGGAAACUCC CU UCAAGGACAUCGUCCGGG UCAUCCCC | 3170 |
| 644 | UUCCUACU G UGUGACAA | 1501 | UUGUCACA GGAGGAAACUCC CU UCAAGGACAUCGUCCGGG AGUAGGAA | 3171 |
| 646 | CCUACUGU G UGACAAGG | 1502 | CCUUGUCA GGAGGAAACUCC CU UCAAGGACAUCGUCCGGG ACAGUAGG | 3172 |
| 648 | UACUGUGU C ACAAGGUG | 2754 | CACCUUGU GGAGGAAACUCC CU UCAAGGACAUCGUCCGGG ACACAGUA | 3173 |
| 653 | UGUGACAA G GUGCAGAA | 2755 | UUCUGCAC GGAGGAAACUCC CU UCAAGGACAUCGUCCGGG UUGUCACA | 3174 |
| 654 | GUGACAAG G UGCAGAAA | 1503 | UUUCUGCA GGAGGAAACUCC CU UCAAGGACAUCGUCCGGG CUUGUCAC | 3175 |
| 656 | GACAAGGU G CAGAAAGA | 1504 | UCUUUCUG GGAGGAAACUCC CU UCAAGGACAUCGUCCGGG ACCUUGUC | 3176 |
| 659 | AAGGUGCA G AAAGAGGA | 2756 | UCCUCUUU GGAGGAAACUCC CU UCAAGGACAUCGUCCGGG UGCACCUU | 3177 |
| 663 | UGCAGAAA G AGGACAUU | 2757 | AAUGUCCU GGAGGAAACUCC CU UCAAGGACAUCGUCCGGG UUUCUGCA | 3178 |
| 665 | CAGAAAGA G GACAUUGA | 2758 | UCAAUGUC GGAGGAAACUCC CU UCAAGGACAUCGUCCGGG UCUUUCUG | 3179 |
| 666 | AGAAAGAG G ACAUUGAG | 2759 | CUCAAUGU GGAGGAAACUCC CU UCAAGGACAUCGUCCGGG CUCUUUCU | 3180 |
| 672 | AGGACAUU G AGGUGUAU | 2760 | AUACACCU GGAGGAAACUCC CU UCAAGGACAUCGUCCGGG AAUGUCCU | 3181 |
| 674 | GACAUUGA G GUGUAUUU | 2761 | AAAUACAC GGAGGAAACUCC CU UCAAGGACAUCGUCCGGG UCAAUGUC | 3182 |
| 675 | ACAUUGAG G UGUAUUUC | 1505 | GAAAUACA GGAGGAAACUCC CU UCAAGGACAUCGUCCGGG CUCAAUGU | 3183 |
| 677 | AUUGAGGU G UAUUUCAC | 1506 | GUGAAAUA GGAGGAAACUCC CU UCAAGGACAUCGUCCGGG ACCUCAAU | 3184 |
| 686 | UAUUUCAC G GGACCAGG | 2762 | CCUGGUCC GGAGGAAACUCC CU UCAAGGACAUCGUCCGGG GUGAAAUA | 3185 |
| 687 | AUUUCACG G GACCAGGC | 2763 | GCCUGGUC GGAGGAAACUCC CU UCAAGGACAUCGUCCGGG CGUGAAAU | 3186 |
| 688 | UUUCACGG G ACCAGGCU | 2764 | AGCCUGGU GGAGGAAACUCC CU UCAAGGACAUCGUCCGGG CCGUGAAA | 3187 |
| 693 | CGGGACCA G GCUGGGAG | 2765 | CUCCCAGC GGAGGAAACUCC CU UCAAGGACAUCGUCCGGG UGGUCCCG | 3188 |
| 694 | GGGACCAG G CUGGGAGG | 1507 | CCUCCCAG GGAGGAAACUCC CU UCAAGGACAUCGUCCGGG CUGGUCCC | 3189 |
| 697 | ACCAGGCU G GGAGGCCC | 2766 | GGGCCUCC GGAGGAAACUCC CU UCAAGGACAUCGUCCGGG AGCCUGGU | 3190 |
| 698 | CCAGGCUG G GAGGCCCG | 2767 | CGGGCCUC GGAGGAAACUCC CU UCAAGGACAUCGUCCGGG CAGCCUGG | 3191 |
| 699 | CAGGCUGG G AGGCCCGA | 2768 | UCGGGCCU GGAGGAAACUCC CU UCAAGGACAUCGUCCGGG CCAGCCUG | 3192 |
| 701 | GGCUGGGA G GCCCGAGG | 2769 | CCUCGGGC GGAGGAAACUCC CU UCAAGGACAUCGUCCGGG UCCCAGCC | 3193 |
| 702 | GCUGGGAG G CCCGAGGC | 1508 | GCCUCGGG GGAGGAAACUCC CU UCAAGGACAUCGUCCGGG CUCCCAGC | 3194 |
| 706 | GGAGGCCC G AGGCUCCU | 2770 | AGGAGCCU GGAGGAAACUCC CU UCAAGGACAUCGUCCGGG GGGCCUCC | 3195 |
| 708 | AGGCCCGA G GCUCCUUU | 2771 | AAAGGAGC GGAGGAAACUCC CU UCAAGGACAUCGUCCGGG UCGGGCCU | 3196 |
| 709 | GGCCCGAG G CUCCUUUU | 1509 | AAAAGGAG GGAGGAAACUCC CU UCAAGGACAUCGUCCGGG CUCGGGCC | 3197 |
| 719 | UCCUUUUC G CAAGCUGA | 1510 | UCAGCUUG GGAGGAAACUCC CU UCAAGGACAUCGUCCGGG GAAAAGGA | 3198 |
| 723 | UUUCGCAA G CUGAUGUG | 1511 | CACAUCAG GGAGGAAACUCC CU UCAAGGACAUCGUCCGGG UUGCGAAA | 3199 |
| 726 | CGCAAGCU G AUGUGCAC | 2772 | GUGCACAU GGAGGAAACUCC CU UCAAGGACAUCGUCCGGG AGUUUGCG | 3200 |
| 729 | AAGCUGAU G UGCACCGA | 1512 | UCGGUGCA GGAGGAAACUCC CU UCAAGGACAUCGUCCGGG AUCAGCUU | 3201 |
| 731 | GCUGAUGU G CACCGACA | 1513 | UGUCGGUG GGAGGAAACUCC CU UCAAGGACAUCGUCCGGG ACAUCAGC | 3202 |
| 736 | UGUGCACC G ACAAGUGG | 2773 | CCACUUGU GGAGGAAACUCC CU UCAAGGACAUCGUCCGGG GGUGCACA | 3203 |
| 741 | ACCGACAA G UGGCCAUU | 1514 | AAUGGCCA GGAGGAAACUCC CU UCAAGGACAUCGUCCGGG UUGUCGGU | 3204 |
| 743 | CGACAAGU G GCCAUUGU | 2774 | ACAAUGGC GGAGGAAACUCC CU UCAAGGACAUCGUCCGGG ACUUGUCG | 3205 |
| 744 | GACAAGUG G CCAUUGUG | 1515 | CACAAUGG GGAGGAAACUCC CU UCAAGGACAUCGUCCGGG CACUUGUC | 3206 |
| 750 | UGGCCAUU G UGUUCCGG | 1516 | CCGGAACA GGAGGAAACUCC CU UCAAGGACAUCGUCCGGG AAUGGCCA | 3207 |
| 752 | GCCAUUGU G UUCCGGAC | 1517 | GUCCGGAA GGAGGAAACUCC CU UCAAGGACAUCGUCCGGG ACAAUGGC | 3208 |
| 757 | UGUGUUCC G GACCCCUC | 2775 | GAGGGGUC GGAGGAAACUCC CU UCAAGGACAUCGUCCGGG GGAACACA | 3209 |
| 758 | GUGUUCCG G ACCCCUCC | 2776 | GGAGGGGU GGAGGAAACUCC CU UCAAGGACAUCGUCCGGG CCGAACAC | 3210 |
| 771 | CUCCCUAC G CAGACCCC | 1518 | GGGGUCUG GGAGGAAACUCC CU UCAAGGACAUCGUCCGGG GUAGGGAG | 3211 |
| 774 | CCUACGCA G ACCCCAGC | 2777 | GCUGGGGU GGAGGAAACUCC CU UCAAGGACAUCGUCCGGG UGCGUAGG | 3212 |
| 781 | AGACCCCA G CCUGCAGG | 1519 | CCUGCAGG GGAGGAAACUCC CU UCAAGGACAUCGUCCGGG UGGGGUCU | 3213 |
| 785 | CCCAGCCU G CAGGCUCC | 1520 | GGAGCCUG GGAGGAAACUCC CU UCAAGGACAUCGUCCGGG AGGCUGGG | 3214 |
| 788 | AGCCUGCA G GCUCCUGU | 2778 | ACAGGAGC GGAGGAAACUCC CU UCAAGGACAUCGUCCGGG UGCAGGCU | 3215 |
| 789 | GCCUGCAG G CUCCUGUG | 1521 | CACAGGAG GGAGGAAACUCC CU UCAAGGACAUCGUCCGGG CUGCAGGC | 3216 |
| 795 | AGGCUCCU G UGCGUGUC | 1522 | GACACGCA GGAGGAAACUCC CU UCAAGGACAUCGUCCGGG AGGAGCCU | 3217 |
| 797 | GCUCCUGU G CGUGUCUC | 1523 | GAGACACG GGAGGAAACUCC CU UCAAGGACAUCGUCCGGG ACAGGAGC | 3218 |
| 799 | UCCUGUGC G UGUCUCCA | 1524 | UGGAGACA GGAGGAAACUCC CU UCAAGGACAUCGUCCGGG GCACAGGA | 3219 |
| 801 | CUGUGCGU G UCUCCAUG | 1525 | CAUGGAGA GGAGGAAACUCC CU UCAAGGACAUCGUCCGGG ACGCACAG | 3220 |
| 809 | GUCUCCAU G CAGCUGCG | 1526 | CGCAGCUG GGAGGAAACUCC CU UCAAGGACAUCGUCCGGG AUGGAGAC | 3221 |
| 812 | UCCAUGCA G CUGCGGCG | 1527 | CGCCGCAG GGAGGAAACUCC CU UCAAGGACAUCGUCCGGG UGCAUGGA | 3222 |
| 815 | AUGCAGCU G CGGCGGCC | 1528 | GGCCGCCG GGAGGAAACUCC CU UCAAGGACAUCGUCCGGG AGCUGCAU | 3223 |
| 817 | GCAGCUGC G GCGGCCUU | 2779 | AAGGCCGC GGAGGAAACUCC CU UCAAGGACAUCGUCCGGG GCAGCUGC | 3224 |
| 818 | CAGCUGCG G CGGCCUUC | 1529 | GAAGGCCG GGAGGAAACUCC CU UCAAGGACAUCGUCCGGG CGCAGCUG | 3225 |
| 820 | GCUGCGGC G GCCUUCCG | 2780 | CGGAAGGC GGAGGAAACUCC CU UCAAGGACAUCGUCCGGG GCCGCAGC | 3226 |
| 821 | CUGCGGCG G CCUUCCGA | 1530 | UCGGAAGG GGAGGAAACUCC CU UCAAGGACAUCGUCCGGG CGCCGCAG | 3227 |
| 828 | GGCCUUCC G ACCGGGAG | 2781 | CUCCCGGU GGAGGAAACUCC CU UCAAGGACAUCGUCCGGG GGAAGGCC | 3228 |
| 832 | UUCCGACC G GGAGCUCA | 2782 | UGAGCUCC GGAGGAAACUCC CU UCAAGGACAUCGUCCGGG GGUCGGAA | 3229 |
| 833 | UCCGACCG G GAGCUCAG | 2783 | CUGAGCUC GGAGGAAACUCC CU UCAAGGACAUCGUCCGGG CGGUCGGA | 3230 |
| 834 | CCGACCGG G AGCUCAGU | 2784 | ACUGAGCU GGAGGAAACUCC CU UCAAGGACAUCGUCCGGG CCGGUCGG | 3231 |
| 836 | GACCGGGA G CUCAGUGA | 1531 | UCACUGAG GGAGGAAACUCC CU UCAAGGACAUCGUCCGGG UCCCGGUC | 3232 |
| 841 | GGAGCUCA G UGAGCCCA | 1532 | UGGGCUCA GGAGGAAACUCC CU UCAAGGACAUCGUCCGGG UGAGCUCC | 3233 |
| 843 | AGCUCAGU G AGCCCAUG | 2785 | CAUGGGCU GGAGGAAACUCC CU UCAAGGACAUCGUCCGGG ACUGAGCU | 3234 |
| 845 | CUCAGUGA G CCCAUGGA | 1533 | UCCAUGGG GGAGGAAACUCC CU UCAAGGACAUCGUCCGGG UCACUGAG | 3235 |
| 851 | GAGCCCAU G GAAUUCCA | 2786 | UGGAAUUC GGAGGAAACUCC CU UCAAGGACAUCGUCCGGG AUGGGCUC | 3236 |
| 852 | AGCCCAUG G AAUUCCAG | 2787 | CUGGAAUU GGAGGAAACUCC CU UCAAGGACAUCGUCCGGG CAUGGGCU | 3237 |
| 860 | GAAUUCCA G UACCUGCC | 1534 | GGCAGGUA GGAGGAAACUCC CU UCAAGGACAUCGUCCGGG UGGAAUUC | 3238 |
| 866 | CAGUACCU G CCAGAUAC | 1535 | GUAUCUGG GGAGGAAACUCC CU UCAAGGACAUCGUCCGGG AGGUACUG | 3239 |
| 870 | ACCUGCCA G AUACAGAC | 2788 | GUCUGUAU GGAGGAAACUCC CU UCAAGGACAUCGUCCGGG UGGCAGGU | 3240 |
| 876 | CAGAUACA G ACGAUCGU | 2789 | ACGAUCGU GGAGGAAACUCC CU UCAAGGACAUCGUCCGGG UGUAUCUG | 3241 |
| 879 | AUACAGAC G AUCGUCAC | 2790 | GUGACGAU GGAGGAAACUCC CU UCAAGGACAUCGUCCGGG GUCUGUAU | 3242 |
| 883 | AGACGAUC G UCACCGGA | 1536 | UCCGGUGA GGAGGAAACUCC CU UCAAGGACAUCGUCCGGG GAUCGUCU | 3243 |
| 889 | UCGUCACC G GAUUGAGG | 2791 | CCUCAAUC GGAGGAAACUCC CU UCAAGGACAUCGUCCGGG GGUGACGA | 3244 |
| 890 | CGUCACCG G AUUGAGGA | 2792 | UCCUCAAU GGAGGAAACUCC CU UCAAGGACAUCGUCCGGG CGGUGACG | 3245 |
| 894 | ACCGGAUU G AGGAGAAA | 2793 | UUUCUCCU GGAGGAAACUCC CU UCAAGGACAUCGUCCGGG AAUCCGGU | 3246 |
| 896 | CGGAUUGA G GAGAAACG | 2794 | CGUUUCUC GGAGGAAACUCC CU UCAAGGACAUCGUCCGGG UCAAUCCG | 3247 |
| 897 | GGAUUGAG G AGAAACGU | 2795 | ACGUUUCU GGAGGAAACUCC CU UCAAGGACAUCGUCCGGG CUCAAUCC | 3248 |
| 899 | AUUGAGGA G AAACGUAA | 2796 | UUACGUUU GGAGGAAACUCC CU UCAAGGACAUCGUCCGGG UCCUCAAU | 3249 |
| 904 | GGAGAAAC G UAAAAGGA | 1537 | UCCUUUUA GGAGGAAACUCC CU UCAAGGACAUCGUCCGGG GUUUCUCC | 3250 |
| 910 | ACGUAAAA G GACAUAUG | 2797 | CAUAUGUC GGAGGAAACUCC CU UCAAGGACAUCGUCCGGG UUUUACGU | 3251 |
| 911 | CGUAAAAG G ACAUAUGA | 2798 | UCAUAUGU GGAGGAAACUCC CU UCAAGGACAUCGUCCGGG CUUUUACG | 3252 |
| 918 | GGACAUAU G AGACCUUC | 2799 | GAAGGUCU GGAGGAAACUCC CU UCAAGGACAUCGUCCGGG AUAUGUCC | 3253 |
| 920 | ACAUAUGA G ACCUUCAA | 2800 | UUGAAGGU GGAGGAAACUCC CU UCAAGGACAUCGUCCGGG UCAUAUGU | 3254 |
| 929 | ACCUUCAA G AGCAUCAU | 2801 | AUGAUGCU GGAGGAAACUCC CU UCAAGGACAUCGUCCGGG UUGAAGGU | 3255 |
| 931 | CUUCAAGA G CAUCAUGA | 1538 | UCAUGAUG GGAGGAAACUCC CU UCAAGGACAUCGUCCGGG UCUUGAAG | 3256 |
| 938 | AGCAUCAU G AAGAAGAG | 2802 | CUCUUCUU GGAGGAAACUCC CU UCAAGGACAUCGUCCGGG AUGAUGCU | 3257 |
| 941 | AUCAUGAA G AAGAGUCC | 2803 | GGACUCUU GGAGGAAACUCC CU UCAAGGACAUCGUCCGGG UUCAUGAU | 3258 |
| 944 | AUGAAGAA G AGUCCUUU | 2804 | AAAGGACU GGAGGAAACUCC CU UCAAGGACAUCGUCCGGG UUCUUCAU | 3259 |
| 946 | GAAGAAGA G UCCUUUCA | 1539 | UGAAAGGA GGAGGAAACUCC CU UCAAGGACAUCGUCCGGG UCUUCUUC | 3260 |
| 955 | UCCUUUCA G CGGACCCA | 1540 | UGGGUCCG GGAGGAAACUCC CU UCAAGGACAUCGUCCGGG UGAAAGGA | 3261 |
| 957 | CUUUCAGC G GACCCACC | 2805 | GGUGGGUC GGAGGAAACUCC CU UCAAGGACAUCGUCCGGG GCUGAAAG | 3262 |
| 958 | UUUCAGCG G ACCCACCG | 2806 | CGGUGGGU GGAGGAAACUCC CU UCAAGGACAUCGUCCGGG CGCUGAAA | 3263 |
| 966 | GACCCACC G ACCCCCGG | 2807 | CCGGGGGU GGAGGAAACUCC CU UCAAGGACAUCGUCCGGG GGUGGGUC | 3264 |
| 973 | CGACCCCC G GCCUCCAC | 2808 | GUGGAGGC GGAGGAAACUCC CU UCAAGGACAUCGUCCGGG GGGGGUCG | 3265 |
| 974 | GACCCCCG G CCUCCACC | 1541 | GGUGGAGG GGAGGAAACUCC CU UCAAGGACAUCGUCCGGG CGGGGGUC | 3266 |
| 985 | UCCACCUC G ACGCAUUG | 2809 | CAAUGCGU GGAGGAAACUCC CU UCAAGGACAUCGUCCGGG GAGGUGGA | 3267 |
| 988 | ACCUCGAC G CAUUGCUG | 1542 | CAGCAAUG GGAGGAAACUCC CU UCAAGGACAUCGUCCGGG GUCGAGGU | 3268 |
| 993 | GACGCAUU G CUGUGCCU | 1543 | AGGCACAG GGAGGAAACUCC CU UCAAGGACAUCGUCCGGG AAUGCGUC | 3269 |
| 996 | GCAUUGCU G UGCCUUCC | 1544 | GGAAGGCA GGAGGAAACUCC CU UCAAGGACAUCGUCCGGG AGCAAUGC | 3270 |
| 998 | AUUGCUGU G CCUUCCCG | 1545 | CGGGAAGG GGAGGAAACUCC CU UCAAGGACAUCGUCCGGG ACAGCAAU | 3271 |
| 1006 | GCCUUCCC G CAGCUCAG | 1546 | CUGAGCUG GGAGGAAACUCC CU UCAAGGACAUCGUCCGGG GGGAAGGC | 3272 |
| 1009 | UUCCCGCA G CUCAGCUU | 1547 | AAGCUGAG GGAGGAAACUCC CU UCAAGGACAUCGUCCGGG UGCGGGAA | 3273 |
| 1014 | GCAGCUCA G CUUCUGUC | 1548 | GACAGAAG GGAGGAAACUCC CU UCAAGGACAUCGUCCGGG UGAGCUGC | 3274 |
| 1020 | CAGCUUCU G UCCCCAAG | 1549 | CUUGGGGA GGAGGAAACUCC CU UCAAGGACAUCGUCCGGG AGAAGCUG | 3275 |
| 1028 | GUCCCCAA G CCAGCACC | 1550 | GGUGCUGG GGAGGAAACUCC CU UCAAGGACAUCGUCCGGG UUGGGGAC | 3276 |
| 1032 | CCAAGCCA G CACCCCAG | 1551 | CUGGGGUG GGAGGAAACUCC CU UCAAGGACAUCGUCCGGG UGGCUUGG | 3277 |
| 1040 | GCACCCCA G CCCUAUCC | 1552 | GGAUAGGG GGAGGAAACUCC CU UCAAGGACAUCGUCCGGG UGGGGUGC | 3278 |
| 1055 | CCCUUUAC G UCAUCCCU | 1553 | AGGGAUGA GGAGGAAACUCC CU UCAAGGACAUCGUCCGGG GUAAAGGG | 3279 |
| 1064 | UCAUCCCU G AGCACCAU | 2810 | AUGGUGCU GGAGGAAACUCC CU UCAAGGACAUCGUCCGGG AGGGAUGA | 3280 |
| 1066 | AUCCCUGA G CACCAUCA | 1554 | UGAUGGUG GGAGGAAACUCC CU UCAAGGACAUCGUCCGGG UCAGGGAU | 3281 |
| 1080 | UCAACUAU G AUGAGUUU | 2811 | AAACUCAU GGAGGAAACUCC CU UCAAGGACAUCGUCCGGG AUAGUUGA | 3282 |
| 1083 | ACUAUGAU G AGUUUCCC | 2812 | GGGAAACU GGAGGAAACUCC CU UCAAGGACAUCGUCCGGG AUCAUAGU | 3283 |
| 1085 | UAUGAUGA G UUUCCCAC | 1555 | GUGGGAAA GGAGGAAACUCC CU UCAAGGACAUCGUCCGGG UCAUCAUA | 3284 |
| 1097 | CCCACCAU G GUGUUUCC | 2813 | GGAAACAC GGAGGAAACUCC CU UCAAGGACAUCGUCCGGG AUGGUGGG | 3285 |
| 1098 | CCACCAUG G UGUUUCCU | 1556 | AGGAAACA GGAGGAAACUCC CU UCAAGGACAUCGUCCGGG CAUGGUGG | 3286 |
| 1100 | ACCAUGGU G UUUCCUUC | 1557 | GAAGGAAA GGAGGAAACUCC CU UCAAGGACAUCGUCCGGG ACCAUGGU | 3287 |
| 1110 | UUCCUUCU G GGCAGAUC | 2814 | GAUCUGCC GGAGGAAACUCC CU UCAAGGACAUCGUCCGGG AGAAGGAA | 3288 |
| 1111 | UCCUUCUG G GCAGAUCA | 2815 | UGAUCUGC GGAGGAAACUCC CU UCAAGGACAUCGUCCGGG CAGAAGGA | 3289 |
| 1112 | CCUUCUGG G CAGAUCAG | 1558 | CUGAUCUG GGAGGAAACUCC CU UCAAGGACAUCGUCCGGG CCAGAAGG | 3290 |
| 1115 | UCUGGGCA G AUCAGCCA | 2816 | UGGCUGAU GGAGGAAACUCC CU UCAAGGACAUCGUCCGGG UGCCCAGA | 3291 |
| 1120 | GCAGAUCA G CCAGGCCU | 1559 | AGGCCUGG GGAGGAAACUCC CU UCAAGGACAUCGUCCGGG UGAUCUGC | 3292 |
| 1124 | AUCAGCCA G GCCUCGGC | 2817 | GCCGAGGC GGAGGAAACUCC CU UCAAGGACAUCGUCCGGG UGGCUGAU | 3293 |
| 1125 | UCAGCCAG G CCUCGGCC | 1560 | GGCCGAGG GGAGGAAACUCC CU UCAAGGACAUCGUCCGGG CUGGCUGA | 3294 |
| 1130 | CAGGCCUC G GCCUUGGC | 2818 | GCCAAGGC GGAGGAAACUCC CU UCAAGGACAUCGUCCGGG GAGGCCUG | 3295 |
| 1131 | AGGCCUCG G CCUUGGCC | 1561 | GGCCAAGG GGAGGAAACUCC CU UCAAGGACAUCGUCCGGG CGAGGCCU | 3296 |
| 1136 | UCGGCCUU G GCCCCGGC | 2819 | GCCGGGGC GGAGGAAACUCC CU UCAAGGACAUCGUCCGGG AAGGCCGA | 3297 |
| 1137 | CGGCCUUG G CCCCGGCC | 1562 | GGCCGGGG GGAGGAAACUCC CU UCAAGGACAUCGUCCGGG CAAGGCCG | 3298 |
| 1142 | UUGGCCCC G GCCCCUCC | 2820 | GGAGGGGC GGAGGAAACUCC CU UCAAGGACAUCGUCCGGG GGGGCCAA | 3299 |
| 1143 | UGGCCCCG G CCCCUCCC | 1563 | GGGAGGGG GGAGGAAACUCC CU UCAAGGACAUCGUCCGGG CGGGGCCA | 3300 |
| 1155 | CUCCCCAA G UCCUGCCC | 1564 | GGGCAGGA GGAGGAAACUCC CU UCAAGGACAUCGUCCGGG UUGGGGAG | 3301 |
| 1160 | CAAGUCCU G CCCCAGGC | 1565 | GCCUGGGG GGAGGAAACUCC CU UCAAGGACAUCGUCCGGG AGGACUUG | 3302 |
| 1166 | CUGCCCCA G GCUCCAGC | 2821 | GCUGGAGC GGAGGAAACUCC CU UCAAGGACAUCGUCCGGG UGGGGCAG | 3303 |
| 1167 | UGCCCCAG G CUCCAGCC | 1566 | GGCUGGAG GGAGGAAACUCC CU UCAAGGACAUCGUCCGGG CUGGGGCA | 3304 |
| 1173 | AGGCUCCA G CCCCUGCC | 1567 | GGCAGGGG GGAGGAAACUCC CU UCAAGGACAUCGUCCGGG UGGAGCCU | 3305 |
| 1179 | CAGCCCCU G CCCCUGCU | 1568 | AGCAGGGG GGAGGAAACUCC CU UCAAGGACAUCGUCCGGG AGGGGCUG | 3306 |
| 1185 | CUGCCCCU G CUCCAGCC | 1569 | GUCUGGAG GGAGGAAACUCC CU UCAAGGACAUCGUCCGGG AGGGGCAG | 3307 |
| 1191 | CUGCUCCA G CCAUGGUA | 1570 | UACCAUGG GGAGGAAACUCC CU UCAAGGACAUCGUCCGGG UGGAGCAG | 3308 |
| 1196 | CCAGCCAU G GUAUCAGC | 2822 | GCUGAUAC GGAGGAAACUCC CU UCAAGGACAUCGUCCGGG AUGGCUGG | 3309 |
| 1197 | CAGCCAUG G UAUCAGCU | 1571 | AGCUGAUA GGAGGAAACUCC CU UCAAGGACAUCGUCCGGG CAUGGCUG | 3310 |
| 1203 | UGGUAUCA G CUCUGGCC | 1572 | GUCCAGAG GGAGGAAACUCC CU UCAAGGACAUCGUCCGGG UGAUACCA | 3311 |
| 1208 | UCAGCUCU G GCCCAGGC | 2823 | UCCUUUGC GGAGGAAACUCC CU UCAAGGACAUCGUCCGGG AGAGCUGA | 3312 |
| 1209 | CAUCUCUG G CCCAGGCC | 1573 | GGCCUGGG GGAGGAAACUCC CU UCAAGGACAUCGUCCGGG CAGAGCUG | 3313 |
| 1214 | CUGGCCCA G GCCCCAGC | 2824 | UCUGGUUC GGAGGAAACUCC CU UCAAGGACAUCGUCCGGG UGGGCCAG | 3314 |
| 1215 | UGGCCCAG G CCCCAGCC | 1574 | GGCUGGGU GGAGGAAACUCC CU UCAAGGACAUCGUCCGGG CUGGGCCA | 3315 |
| 1221 | AUGCCCCA G CCCCUGUC | 1575 | GACAUGUG GGAGGAAACUCC CU UCAAGGACAUCGUCCGGG UGGGGCCU | 3316 |
| 1227 | CAGCCCCU G UCCCAGUC | 1576 | GACUGGGA GGAGGAAACUCC CU UCAAGGACAUCGUCCGGG AGGGGCUG | 3317 |
| 1233 | CUGUCCCA G UCCUAGCC | 1577 | GUCUAGGA GGAGGAAACUCC CU UCAAGGACAUCGUCCGGG UGGGACAG | 3318 |
| 1239 | CAGUCCUA G CCCCAGGC | 1578 | GCCUGGGG GGAGGAAACUCC CU UCAAGGACAUCGUCCGGG UAGGACUG | 3319 |
| 1245 | UAGCCCCA G GCCCUCCU | 2825 | AUGAUGUC GGAGGAAACUCC CU UCAAGGACAUCGUCCGGG UGGGGCUA | 3320 |
| 1246 | AGCCCCAG G CCCUCCUC | 1579 | GAUGAGGU GGAGGAAACUCC CU UCAAGGACAUCGUCCGGG CUGGGGCU | 3321 |
| 1256 | CCUCCUCA G GCUGUGGC | 2826 | GCCACAGC GGAGGAAACUCC CU UCAAGGACAUCGUCCGGG UGAGGAGG | 3322 |
| 1257 | CUCCUCAG G CUGUGGCC | 1380 | GGCCACAG GGAGGAAACUCC CU UCAAGGACAUCGUCCGGG CUGAGGAG | 3323 |
| 1260 | CUCAGGCU G UGGCCCCA | 1581 | UGGGGCCA GGAGGAAACUCC CU UCAAGGACAUCGUCCGGG AGCCUGAG | 3324 |
| 1262 | CAGGCUGU G GCCCCACC | 2827 | GGUGGGGC GGAGGAAACUCC CU UCAAGGACAUCGUCCGGG ACAUCCUG | 3325 |
| 1263 | AGGCUGUG G CCCCACCU | 1582 | AGGUGGGG GGAGGAAACUCC CU UCAAGGACAUCGUCCGGG CACAGCCU | 3326 |
| 1272 | CCCCACCU G CCCCCAAG | 1583 | CUUGGGGG GGAGGAAACUCC CU UCAAGGACAUCGUCCGGG AGGUGGGG | 3327 |
| 1280 | GCCCCCAA G CCCACCCA | 1584 | UGGGUGGG GGAGGAAACUCC CU UCAAGGACAUCGUCCGGG UUGGGGGC | 3328 |
| 1289 | CCCACCCA G GCUGGGGA | 2828 | UCCCCAGC GGAGGAAACUCC CU UCAAGGACAUCGUCCGGG UGGGUGGG | 3329 |
| 1290 | CCACCCAG G CUGGGGAA | 1585 | UUCCCCAG GGAGGAAACUCC CU UCAAGGACAUCGUCCGGG CUGGGUGG | 3330 |
| 1293 | CCCAGGCU G GGGAAGGA | 2829 | UCCUUCCC GGAGGAAACUCC CU UCAAGGACAUCGUCCGGG AGCCUGGG | 3331 |
| 1294 | CCAGGCUG G GGAAGGAA | 2830 | UUCCUUCC GGAGGAAACUCC CU UCAAGGACAUCGUCCGGG CAGCCUGG | 3332 |
| 1295 | CAGGCUGG G GAAGGAAC | 2831 | GUUCCUUC GGAGGAAACUCC CU UCAAGGACAUCGUCCGGG CCAGCCUG | 3333 |
| 1296 | AGGCUGGG G AAGGAACG | 2832 | CGUUCCUU GGAGGAAACUCC CU UCAAGGACAUCGUCCGGG CCCAGCCU | 3334 |
| 1299 | CUGGGGAA G GAACGCUG | 2833 | CAGCGUUC GGAGGAAACUCC CU UCAAGGACAUCGUCCGGG UUCCCCAG | 3335 |
| 1300 | UGGGGAAG G AACGCUGU | 2834 | ACAGCGUU GGAGGAAACUCC CU UCAAGGACAUCGUCCGGG CUUCCCCA | 3336 |
| 1304 | GAAGGAAC G CUGUCAGA | 1586 | UCUGACAG GGAGGAAACUCC CU UCAAGGACAUCGUCCGGG GUUCCUUC | 3337 |
| 1307 | GGAACGCU G UCAGAGGC | 1587 | GCCUCUGA GGAGGAAACUCC CU UCAAGGACAUCGUCCGGG AGCGUUCC | 3338 |
| 1311 | CGCUGUCA G AGGCCCUG | 2835 | CAGGGCCU GGAGGAAACUCC CU UCAAGGACAUCGUCCGGG UGACAGCG | 3339 |
| 1313 | CUGUCAGA G GCCCUGCU | 2836 | AGCAGGGC GGAGGAAACUCC CU UCAAGGACAUCGUCCGGG UCUGACAG | 3340 |
| 1314 | UGUCAGAG G CCCUGCUG | 1588 | CAGCAGGG GGAGGAAACUCC CU UCAAGGACAUCGUCCGGG CUCUGACA | 3341 |
| 1319 | GAGGCCCU G CUGCAGCU | 1589 | AGCUGCAG GGAGGAAACUCC CU UCAAGGACAUCGUCCGGG AGGGCCUC | 3342 |
| 1322 | GCCCUGCU G CAGCUGCA | 1590 | UGCAGCUG GGAGGAAACUCC CU UCAAGGACAUCGUCCGGG AGCAGGGC | 3343 |
| 1325 | CUGCUGCA G CUGCAGUU | 1591 | AACUGCAG GGAGGAAACUCC CU UCAAGGACAUCGUCCGGG UGCAGCAG | 3344 |
| 1328 | CUGCAGCU G CAGUUUGA | 1592 | UCAAACUG GGAGGAAACUCC CU UCAAGGACAUCGUCCGGG AGCUGCAG | 3345 |
| 1331 | CAGCUGCA G UUUGAUGA | 1593 | UCAUCAAA GGAGGAAACUCC CU UCAAGGACAUCGUCCGGG UGCAGCUG | 3346 |
| 1335 | UGCAGUUU G AUGAUGAA | 2837 | UUCAUCAU GGAGGAAACUCC CU UCAAGGACAUCGUCCGGG AAACUGCA | 3347 |
| 1338 | AGUUUGAU G AUGAAGAC | 2838 | GUCUUCAU GGAGGAAACUCC CU UCAAGGACAUCGUCCGGG AUCAAACU | 3348 |
| 1341 | UUGAUGAU G AAGACCUG | 2839 | CAGGUCUU GGAGGAAACUCC CU UCAAGGACAUCGUCCGGG AUCAUCAA | 3349 |
| 1344 | AUGAUGAA G ACCUGGGG | 2840 | CCCCAGGU GGAGGAAACUCC CU UCAAGGACAUCGUCCGGG UUCAUCAU | 3350 |
| 1349 | GAAGACCU G GGGGCCUU | 2841 | AAGGCCCC GGAGGAAACUCC CU UCAAGGACAUCGUCCGGG AGGUCUUC | 3351 |
| 1350 | AAGACCUG G GGGCCUUG | 2842 | CAAGGCCC GGAGGAAACUCC CU UCAAGGACAUCGUCCGGG CAGGUCUU | 3352 |
| 1351 | AGACCUGG G GGCCUUGC | 2843 | GCAAGGCC GGAGGAAACUCC CU UCAAGGACAUCGUCCGGG CCAGGUCU | 3353 |
| 1352 | GACCUGGG G GCCUUGCU | 2844 | AGCAAGGC GGAGGAAACUCC CU UCAAGGACAUCGUCCGGG CCCAGGUC | 3354 |
| 1353 | ACCUGGGG G CCUUGCUU | 1594 | AAGCAAGG GGAGGAAACUCC CU UCAAGGACAUCGUCCGGG CCCCAGGU | 3355 |
| 1358 | GGGGCCUU G CUUGGCAA | 1595 | UUGCCAAG GGAGGAAACUCC CU UCAAGGACAUCGUCCGGG AAGGCCCC | 3356 |
| 1362 | CCUUGCUU G GCAACAGC | 2845 | GCUGUUGC GGAGGAAACUCC CU UCAAGGACAUCGUCCGGG AAGCAAGG | 3357 |
| 1363 | CUUGCUUG G CAACAGCA | 1596 | UGCUGUUG GGAGGAAACUCC CU UCAAGGACAUCGUCCGGG CAAGCAAG | 3358 |
| 1369 | UGGCAACA G CACAGACC | 1597 | GGUCUGUG GGAGGAAACUCC CU UCAAGGACAUCGUCCGGG UGUUGCCA | 3359 |
| 1374 | ACAGCACA G ACCCAGCU | 2846 | AGCUGGGU GGAGGAAACUCC CU UCAAGGACAUCGUCCGGG UGUGCUGU | 3360 |
| 1380 | CAGACCCA G CUGUGUUC | 1598 | GAACACAG GGAGGAAACUCC CU UCAAGGACAUCGUCCGGG UGGGUCUG | 3361 |
| 1383 | ACCCAGCU G UGUUCACA | 1599 | UGUGAACA GGAGGAAACUCC CU UCAAGGACAUCGUCCGGG AGCUGGGU | 3362 |
| 1385 | CCAGCUGU G UUCACAGA | 1600 | UCUGUGAA GGAGGAAACUCC CU UCAAGGACAUCGUCCGGG ACAGCUGG | 3363 |
| 1392 | UGUUCACA G ACCUGGCA | 2847 | UGCCAGGU GGAGGAAACUCC CU UCAAGGACAUCGUCCGGG UGUGAACA | 3364 |
| 1397 | ACAGACCU G GCAUCCGU | 2848 | ACGGAUGC GGAGGAAACUCC CU UCAAGGACAUCGUCCGGG AGGUCUGU | 3365 |
| 1398 | CAGACCUG G CAUCCGUC | 1601 | GACGGAUG GGAGGAAACUCC CU UCAAGGACAUCGUCCGGG CAGGUCUG | 3366 |
| 1404 | UGGCAUCC G UCGACAAC | 1602 | GUUGUCGA GGAGGAAACUCC CU UCAAGGACAUCGUCCGGG GGAUGCCA | 3367 |
| 1407 | CAUCCGUC G ACAACUCC | 2849 | GGAGUUGU GGAGGAAACUCC CU UCAAGGACAUCGUCCGGG GACGGAUG | 3368 |
| 1416 | ACAACUCC G AGUUUCAG | 2850 | CUGAAACU GGAGGAAACUCC CU UCAAGGACAUCGUCCGGG GGAGUUGU | 3369 |
| 1418 | AACUCCGA G UUUCAGCA | 1603 | UGCUGAAA GGAGGAAACUCC CU UCAAGGACAUCGUCCGGG UCGGAGUU | 3370 |
| 1424 | GAGUUUCA G CAGCUGCU | 1604 | AGCAGCUG GGAGGAAACUCC CU UCAAGGACAUCGUCCGGG UGAAACUC | 3371 |
| 1427 | UUUCAGCA G CUGCUGAA | 1605 | UUCAGCAG GGAGGAAACUCC CU UCAAGGACAUCGUCCGGG UGCUGAAA | 3372 |
| 1430 | CAGCAGCU G CUGAACCA | 1606 | UGGUUCAG GGAGGAAACUCC CU UCAAGGACAUCGUCCGGG AGCUGCUG | 3373 |
| 1433 | CAGCUGCU G AACCAGGG | 2851 | CCCUGGUU GGAGGAAACUCC CU UCAAGGACAUCGUCCGGG AGCAGCUG | 3374 |
| 1439 | CUGAACCA G GGCAUACC | 2852 | GGUAUGCC GGAGGAAACUCC CU UCAAGGACAUCGUCCGGG UGGUUCAG | 3375 |
| 1440 | UGAACCAG G GCAUACCU | 2853 | AGGUAUGC GGAGGAAACUCC CU UCAAGGACAUCGUCCGGG CUGGUUCA | 3376 |
| 1441 | GAACCAGG G CAUACCUG | 1607 | CAGGUAUG GGAGGAAACUCC CU UCAAGGACAUCGUCCGGG CCUGGUUC | 3377 |
| 1449 | GCAUACCU G UGGCCCCC | 1608 | GGGGGCCA GGAGGAAACUCC CU UCAAGGACAUCGUCCGGG AGGUAUGC | 3378 |
| 1451 | AUACCUGU G GCCCCCCA | 2854 | UGGGGGGC GGAGGAAACUCC CU UCAAGGACAUCGUCCGGG ACAGGUAU | 3379 |
| 1452 | UACCUGUG G CCCCCCAC | 1609 | GUGGGGGG GGAGGAAACUCC CU UCAAGGACAUCGUCCGGG CACAGGUA | 3380 |
| 1467 | ACACAACU G AGCCCAUG | 2855 | CAUGGGCU GGAGGAAACUCC CU UCAAGGACAUCGUCCGGG AGUUGUGU | 3381 |
| 1469 | ACAACUGA G CCCAUGCU | 1610 | AGCAUGGG GGAGGAAACUCC CU UCAAGGACAUCGUCCGGG UCAGUUGU | 3382 |
| 1475 | GAGCCCAU G CUGAUGGA | 1611 | UCCAUCAG GGAGGAAACUCC CU UCAAGGACAUCGUCCGGG AUGGGCUC | 3383 |
| 1478 | CCCAUGCU G AUGGAGUA | 2856 | UACUCCAU GGAGGAAACUCC CU UCAAGGACAUCGUCCGGG AGCAUGGG | 3384 |
| 1481 | AUGCUGAU G GAGUACCC | 2857 | GGGUACUC GGAGGAAACUCC CU UCAAGGACAUCGUCCGGG AUCAGCAU | 3385 |
| 1482 | UGCUGAUG G AGUACCCU | 2858 | AGGGUACU GGAGGAAACUCC CU UCAAGGACAUCGUCCGGG CAUCAGCA | 3386 |
| 1484 | CUGAUGGA G UACCCUGA | 1612 | UCAGGGUA GGAGGAAACUCC CU UCAAGGACAUCGUCCGGG UCCAUCAG | 3387 |
| 1491 | AGUACCCU G AGGCUAUA | 2859 | UAUAGCCU GGAGGAAACUCC CU UCAAGGACAUCGUCCGGG AGGGUACU | 3388 |
| 1493 | UACCCUGA G GCUAUAAC | 2860 | GUUAUAGC GGAGGAAACUCC CU UCAAGGACAUCGUCCGGG UCAGGUUA | 3389 |
| 1494 | ACCCUGAG G CUAUAACU | 1613 | AGUUAUAG GGAGGAAACUCC CU UCAAGGACAUCGUCCGGG CUCAGGGU | 3390 |
| 1504 | UAUAACUC G CCUAGUGA | 1614 | UCACUAGG GGAGGAAACUCC CU UCAAGGACAUCGUCCGGG GAGUUAUA | 3391 |
| 1509 | CUCGCCUA G UGACAGCC | 1615 | GGCUGUCA GGAGGAAACUCC CU UCAAGGACAUCGUCCGGG UAGGCGAG | 3392 |
| 1511 | CGCCUAGU G ACAGCCCA | 2861 | UGGGCUGU GGAGGAAACUCC CU UCAAGGACAUCGUCCGGG ACUAUGCG | 3393 |
| 1515 | UAGUGACA G CCCAGAGG | 1616 | CCUCUGGG GGAGGAAACUCC CU UCAAGGACAUCGUCCGGG UGUCACUA | 3394 |
| 1520 | ACAGCCCA G AGGCCCCC | 2862 | GGGGGCCU GGAGGAAACUCC CU UCAAGGACAUCGUCCGGG UGGGCUGU | 3395 |
| 1522 | AGCCCAGA G GCCCCCCG | 2863 | CGGGGGGC GGAGGAAACUCC CU UCAAGGACAUCGUCCGGG UCUGGGCU | 3396 |
| 1523 | GCCCAGAG G CCCCCCGA | 1617 | UCGGGGGG GGAGGAAACUCC CU UCAAGGACAUCGUCCGGG CUCUGGGC | 3397 |
| 1530 | GGCCCCCC G ACCCAGCU | 2864 | AGCUGGGU GGAGGAAACUCC CU UCAAGGACAUCGUCCGGG GGGGGGCC | 3398 |
| 1536 | CCGACCCA G CUCCUGCU | 1618 | AGCAGGAG GGAGGAAACUCC CU UCAAGGACAUCGUCCGGG UGGGUCGG | 3399 |
| 1542 | CAGCUCCU G CUCCACUG | 1619 | CAGUGGAG GGAGGAAACUCC CU UCAAGGACAUCGUCCGGG AGGAGCUG | 3400 |
| 1550 | GCUCCACU G GGGGCCCC | 2865 | GGGGCCCC GGAGGAAACUCC CU UCAAGGACAUCGUCCGGG AGUGGAGC | 3401 |
| 1551 | CUCCACUG G GGGCCCCG | 2866 | CGGGGCCC GGAGGAAACUCC CU UCAAGGACAUCGUCCGGG CAGUGGAG | 3402 |
| 1552 | UCCACUGG G GGCCCCGG | 2867 | CCGGGGCC GGAGGAAACUCC CU UCAAGGACAUCGUCCGGG CCAGUGGA | 3403 |
| 1553 | CCACUGGG G GCCCCGGG | 2868 | CCCGGGGC GGAGGAAACUCC CU UCAAGGACAUCGUCCGGG CCCAGUGG | 3404 |
| 1554 | CACUGGGG G CCCCGGGG | 1620 | CCCCGGGG GGAGGAAACUCC CU UCAAGGACAUCGUCCGGG CCCCAGUG | 3405 |
| 1559 | GGGGCCCC G GGGCUCCC | 2869 | GGGAGCCC GGAGGAAACUCC CU UCAAGGACAUCGUCCGGG GGGGCCCC | 3406 |
| 1560 | GGGCCCCG G GGCUCCCC | 2870 | GGGGAGCC GGAGGAAACUCC CU UCAAGGACAUCGUCCGGG CGGGGCCC | 3407 |
| 1561 | GGCCCCGG G GCUCCCCA | 2871 | UGGGGAGC GGAGGAAACUCC CU UCAAGGACAUCGUCCGGG CCGGGGCC | 3408 |
| 1562 | GCCCCCGG G CUCCCCAA | 1621 | UUGGGGAG GGAGGAAACUCC CU UCAAGGACAUCGUCCGGG CCCGGGGC | 3409 |
| 1572 | UCCCCAAU G GCCUCCUU | 2872 | AAGGAGGC GGAGGAAACUCC CU UCAAGGACAUCGUCCGGG AUUGGGGA | 3410 |
| 1573 | CCCCAAUG G CCUCCUUU | 1622 | AAAGGAGG GGAGGAAACUCC CU UCAAGGACAUCGUCCGGG CAUUGGGG | 3411 |
| 1584 | UCCUUUCA G GAGAUGAA | 2873 | UUCAUCUC GGAGGAAACUCC CU UCAAGGACAUCGUCCGGG UGAAAGGA | 3412 |
| 1585 | CCUUUCAG G AGAUGAAG | 2874 | CUUCAUCU GGAGGAAACUCC CU UCAAGGACAUCGUCCGGG CUGAAAGG | 3413 |
| 1587 | UGUCAGGA G AUGAAGAC | 2875 | GUCUUCAU GGAGGAAACUCC CU UCAAGGACAUCGUCCGGG UCCUGAAA | 3414 |
| 1590 | CAGGAGAU G AAGACUUC | 2876 | GAAGUCUU GGAGGAAACUCC CU UCAAGGACAUCGUCCGGG AUCUCCUG | 3415 |
| 1593 | GAGAUGAA G ACUUCUCC | 2877 | GGAGAAGU GGAGGAAACUCC CU UCAAGGACAUCGUCCGGG UUCAUCUC | 3416 |
| 1608 | CCUCCAUU G CGGACAUG | 1623 | CAUGUCCG GGAGGAAACUCC CU UCAAGGACAUCGUCCGGG AAUGGAGG | 3417 |
| 1610 | UCCAUUGC G GACAUGGA | 2878 | UCCAUGUC GGAGGAAACUCC CU UCAAGGACAUCGUCCGGG GCAAUGGA | 3418 |
| 1611 | CCAUUGCG G ACAUGGAC | 2879 | GUCCAUGU GGAGGAAACUCC CU UCAAGGACAUCGUCCGGG CGCAAUGG | 3419 |
| 1616 | GCGGACAU G GACUUCUC | 2880 | GAGAAGUC GGAGGAAACUCC CU UCAAGGACAUCGUCCGGG AUGUCCGC | 3420 |
| 1617 | CGGACAUG G ACUUCUCA | 2881 | UGAGAAGU GGAGGAAACUCC CU UCAAGGACAUCGUCCGGG CAUGUCCG | 3421 |
| 1626 | ACUUCUCA G CCCUGCUG | 1624 | CAGCAGGG GGAGGAAACUCC CU UCAAGGACAUCGUCCGGG UGACAAGU | 3422 |
| 1631 | UCAGCCCU G CUGAGUCA | 1625 | UGACUCAG GGAGGAAACUCC CU UCAAGGACAUCGUCCGGG AGGGCUGA | 3423 |
| 1634 | GCCCUGCU G AGUCAGAU | 2882 | AUCUGACU GGAGGAAACUCC CU UCAAGGACAUCGUCCGGG AGCAGGGC | 3424 |
| 1636 | CCUGCUGA G UCAGAUCA | 1626 | UGAUCUGA GGAGGAAACUCC CU UCAAGGACAUCGUCCGGG UCAGCAGG | 3425 |
| 1640 | CUGAGUCA G AUCAGCUC | 2883 | GAGCUGAU GGAGGAAACUCC CU UCAAGGACAUCGUCCGGG UGACUCAG | 3426 |
| 1645 | UCAGAUCA G CUCCUAAG | 1627 | CUUAGGAG GGAGGAAACUCC CU UCAAGGACAUCGUCCGGG UCAUCUGA | 3427 |
| 1653 | GCUCCUAA G GGGGUGAC | 2884 | GUCACCCC GGAGGAAACUCC CU UCAAGGACAUCGUCCGGG UUAGGAGC | 3428 |
| 1654 | CUCCUAAG G GGGUGACG | 2885 | CGUCACCC GGAGGAAACUCC CU UCAAGGACAUCGUCCGGG CUUAGGAG | 3429 |
| 1655 | UCCUAAGG G GGUGACGC | 2886 | GCGUCACC GGAGGAAACUCC CU UCAAGGACAUCGUCCGGG CCUUAGGA | 3430 |
| 1656 | CCUAAGGG G GUGACGCC | 2887 | GGCCUCAC GGAGGAAACUCC CU UCAAGGACAUCGUCCGGG CCCUUAGG | 3431 |
| 1657 | CUAAGGGG G UGACGCCU | 1628 | AGGCGUCA GGAGGAAACUCC CU UCAAGGACAUCGUCCGGG CCCCUUAG | 3432 |
| 1659 | AAGGGCGU G ACGCCUGC | 2888 | GCAGGCGU GGAGGAAACUCC CU UCAAGGACAUCGUCCGGG ACCCCCUU | 3433 |
| 1662 | GGGGUGAC G CCUGCCCU | 1629 | AGGGCAGG GGAGGAAACUCC CU UCAAGGACAUCGUCCGGG GUCACCCC | 3434 |
| 1666 | UGACGCCU G CCCUCCCC | 1630 | GGGGAGGG GGAGGAAACUCC CU UCAAGGACAUCGUCCGGG AGGCGUCA | 3435 |
| 1676 | CCUCCCCA G AGCACUGG | 2889 | CCAGUGCU GGAGGAAACUCC CU UCAAGGACAUCGUCCGGG UCGGCAGG | 3436 |
| 1678 | UCCCCAGA G CACUGGUU | 1631 | AACCAGUG GGAGGAAACUCC CU UCAAGGACAUCGUCCGGG UCUGGGGA | 3437 |
| 1683 | AGAGCACU G GUUGCAGG | 2890 | CCUGCAAC GGAGGAAACUCC CU UCAAGGACAUCGUCCGGG AGUGCUCU | 3438 |
| 1684 | GAGCACUG G UUGCAGGG | 1632 | CCCUGCAA GGAGGAAACUCC CU UCAAGGACAUCGUCCGGG CAGUGCUC | 3439 |
| 1687 | CACUGGUU G CAGGGGAU | 1633 | AUCCCCUG GGAGGAAACUCC CU UCAAGGACAUCGUCCGGG AACCAGUG | 3440 |
| 1690 | UGGUUGCA G GGGAUUGA | 2891 | UCAAUCCC GGAGGAAACUCC CU UCAAGGACAUCGUCCGGG UGCAACCA | 3441 |
| 1691 | GGUUGCAG G GGAUUGAA | 2892 | UUCAAUCC GGAGGAAACUCC CU UCAAGGACAUCGUCCGGG CUGCAACC | 3442 |
| 1692 | GUUGCAGG G GAUUGAAG | 2893 | CUUCAAUC GGAGGAAACUCC CU UCAAGGACAUCGUCCGGG CCUGCAAC | 3443 |
| 1693 | UUGCAGGG G AUUGAAGC | 2894 | GCUUCAAU GGAGGAAACUCC CU UCAAGGACAUCGUCCGGG CCCUGCAA | 3444 |
| 1697 | AGGGGAUU G AAGCCCUC | 2895 | GAGGGCUU GGAGGAAACUCC CU UCAAGGACAUCGUCCGGG AAUCCCCU | 3445 |
| 1700 | GGAUUGAA G CCCUCCAA | 1634 | UUGGAGGG GGAGGAAACUCC CU UCAAGGACAUCGUCCGGG UUCAAUCC | 3446 |
| 1711 | CUCCAAAA G CACUUACG | 1635 | CGUAAGUG GGAGGAAACUCC CU UCAAGGACAUCGUCCGGG UUUUGGAG | 3447 |
| 1719 | GCACUUAC G GAUUCUGG | 2896 | CCAGAAUC GGAGGAAACUCC CU UCAAGGACAUCGUCCGGG GUAAGUGC | 3448 |
| 1720 | CACUUACG G AUUCUGGU | 2897 | ACCAGAAU GGAGGAAACUCC CU UCAAGGACAUCGUCCGGG CGUAAGUG | 3449 |
| 1726 | CGGAUUCU G CUGGGGUG | 2898 | CACCCCAC GGAGGAAACUCC CU UCAAGGACAUCGUCCGGG AGAAUCCG | 3450 |
| 1727 | GGAUUCUG G UGGGGUGU | 1636 | ACACCCCA GGAGGAAACUCC CU UCAAGGACAUCGUCCGGG CAGAAUCC | 3451 |
| 1729 | AUUCUGGU G GGGUGUGU | 2899 | ACACACCC GGAGGAAACUCC CU UCAAGGACAUCGUCCGGG ACCAGAAU | 3452 |
| 1730 | UUCUGGUG G GGUGUGUU | 2900 | AACACACC GGAGGAAACUCC CU UCAAGGACAUCGUCCGGG CACCAGAA | 3453 |
| 1731 | UCUGGUGG G GUGUGUUC | 2901 | GAACACAC GGAGGAAACUCC CU UCAAGGACAUCGUCCGGG CCACCAGA | 3454 |
| 1732 | CUGGUGGG G UGUGUUCC | 1637 | GGAACACA GGAGGAAACUCC CU UCAAGGACAUCGUCCGGG CCCACCAG | 3455 |
| 1734 | GGUGGGGU G UGUUCCAA | 1638 | UUGGAACA GGAGGAAACUCC CU UCAAGGACAUCGUCCGGG ACCCCACC | 3456 |
| 1736 | UGGGGUGU G UUCCAACU | 1639 | AGUUGGAA GGAGGAAACUCC CU UCAAGGACAUCGUCCGGG ACACCCCA | 3457 |
| 1745 | UUCCAACU G CCCCCAAC | 1640 | GUUGGGGG GGAGGAAACUCC CU UCAAGGACAUCGUCCGGG AGUUGGAA | 3458 |
| 1757 | CCAACUUU G UGGAUCUC | 1641 | GACAUCCA GGAGGAAACUCC CU UCAAGGACAUCGUCCGGG AAAGUUGG | 3459 |
| 1759 | AACUUUGU G GAUGUCUU | 2902 | AAGACAUC GGAGGAAACUCC CU UCAAGGACAUCGUCCGGG ACAAAGUU | 3460 |
| 1760 | ACUUUGUG G AUGUCUUC | 2903 | GAAGACAU GGAGGAAACUCC CU UCAAGGACAUCGUCCGGG CACAAAGU | 3461 |
| 1763 | UUGUGGAU G UCUUCCUU | 1642 | AAGGAAGA GGAGGAAACUCC CU UCAAGGACAUCGUCCGGG AUCCACAA | 3462 |
| 1772 | UCUUCCUU G GAGGGGGG | 2904 | CCCCCCUC GGAGGAAACUCC CU UCAAGGACAUCGUCCGGG AAGGAAGA | 3463 |
| 1773 | CUUCCUUG G AGGGGGGA | 2905 | UCCCCCCU GGAGGAAACUCC CU UCAAGGACAUCGUCCGGG CAAGGAAG | 3464 |
| 1775 | UCCUUGGA G GGGGGAGC | 2906 | GCUCCCCC GGAGGAAACUCC CU UCAAGGACAUCGUCCGGG UCCAAGGA | 3465 |
| 1776 | CCUUCGAG G GGGGAGCC | 2907 | GGCUCCCC GGAGGAAACUCC CU UCAAGGACAUCGUCCGGG CUCCAAGG | 3466 |
| 1777 | CUUGGAGG G GGGAGCCA | 2908 | UGGCUCCC GGAGGAAACUCC CU UCAAGGACAUCGUCCGGG CCUCCAAG | 3467 |
| 1778 | UUGGAGGG G GGAGCCAU | 2909 | AUGGCUCC GGAGGAAACUCC CU UCAAGGACAUCGUCCGGG CCCUCCAA | 3468 |
| 1779 | UGGAGGGG G GAGCCAUA | 2910 | UAUGGCUC GGAGGAAACUCC CU UCAAGGACAUCGUCCGGG CCCCUCCA | 3469 |
| 1780 | GGAGGGGG G AGCCAUAU | 2911 | AUAUGGCU GGAGGAAACUCC CU UCAAGGACAUCGUCCGGG CCCCCUCC | 3470 |
| 1782 | AGGGGGGA G CCAUAUUU | 1643 | AAAUAUGG GGAGGAAACUCC CU UCAAGGACAUCGUCCGGG UCCCCCCU | 3471 |
| 1803 | CUUUUAUU G UCAGUAUC | 1644 | GAUACUGA GGAGGAAACUCC CU UCAAGGACAUCGUCCGGG AAUAAAAG | 3472 |
| 1807 | UAUUGUCA G UAUCUGUA | 1645 | UACAGAUA GGAGGAAACUCC CU UCAAGGACAUCGUCCGGG UGACAAUA | 3473 |
| 1813 | CAGUAUCU G UAUCUCUC | 1646 | GAGAGAUA GGAGGAAACUCC CU UCAAGGACAUCGUCCGGG AGAUACUG | 3474 |
| 1831 | CUCUUUUU G GAGGUGCU | 2912 | AGCACCUC GGAGGAAACUCC CU UCAAGGACAUCGUCCGGG AAAAAGAG | 3475 |
| 1832 | UCUUUUUG G AGGUGCUU | 2913 | AAGCACCU GGAGGAAACUCC CU UCAAGGACAUCGUCCGGG CAAAAAGA | 3476 |
| 1834 | UUUUUGGA G GUGCUUAA | 2914 | UUAAGCAC GGAGGAAACUCC CU UCAAGGACAUCGUCCGGG UCCAAAAA | 3477 |
| 1835 | UUUUGGAG G UGCUUAAG | 1647 | CUUAAGCA GGAGGAAACUCC CU UCAAGGACAUCGUCCGGG CUCCAAAA | 3478 |
| 1837 | UUGGAGGU G CUUAAGCA | 1648 | UGCUUAAG GGAGGAAACUCC CU UCAAGGACAUCGUCCGGG ACCUCCAA | 3479 |
| 1843 | GUGCUUAA G CAGAAGCA | 1649 | UGCUUCUG GGAGGAAACUCC CU UCAAGGACAUCGUCCGGG UUAAGCAC | 3480 |
| 1846 | CUUAAGCA G AAGCAUUA | 2915 | UAAUGCUU GGAGGAAACUCC CU UCAAGGACAUCGUCCGGG UGCUUAAG | 3481 |
| 1849 | AAGCAGAA G CAUUAACU | 1650 | AGUUAAUG GGAGGAAACUCC CU UCAAGGACAUCGUCCGGG UUCUGCUU | 3482 |
| 1863 | ACUUCUCU G GAAAGGGG | 2916 | CCCCUUUC GGAGGAAACUCC CU UCAAGGACAUCGUCCGGG AGAGAAGU | 3483 |
| 1864 | CUUCUCUG G AAAGGGGG | 2917 | CCCCCUUU GGAGGAAACUCC CU UCAAGGACAUCGUCCGGG CAGAGAAG | 3484 |
| 1868 | UCUGGAAA G GGGGGAGC | 2918 | GCUCCCCC GGAGGAAACUCC CU UCAAGGACAUCGUCCGGG UUUCCAGA | 3485 |
| 1869 | CUGGAAAG G GGGGAGCU | 2919 | AGCUCCCC GGAGGAAACUCC CU UCAAGGACAUCGUCCGGG CUUUCCAG | 3486 |
| 1870 | UGGAAAGG G GGGAGCUG | 2920 | CAGCUCCC GGAGGAAACUCC CU UCAAGGACAUCGUCCGGG CCUUUCCA | 3487 |
| 1871 | GGAAAGGG G GGAGCUGG | 2921 | CCAGCUCC GGAGGAAACUCC CU UCAAGGACAUCGUCCGGG CCCUUUCC | 3488 |
| 1872 | GAAAGGGG G GAGCUGGG | 2922 | CCCAGCUC GGAGGAAACUCC CU UCAAGGACAUCGUCCGGG CCCCUUUC | 3489 |
| 1873 | AAAGGGGG G AGCUGGGG | 2923 | CCCCAGCU GGAGGAAACUCC CU UCAAGGACAUCGUCCGGG CCCCCUUU | 3490 |
| 1875 | AGGGGGGA G CUGGGGAA | 1651 | UUCCCCAG GGAGGAAACUCC CU UCAAGGACAUCGUCCGGG UCCCCCCU | 3491 |
| 1878 | GGGGAGCU G GGGAAACU | 2924 | AGUUUCCC GGAGGAAACUCC CU UCAAGGACAUCGUCCGGG AGCUCCCC | 3492 |
| 1879 | GGGAGCUG G GGAAACUC | 2925 | GAGUUUCC GGAGGAAACUCC CU UCAAGGACAUCGUCCGGG CAGCUCCC | 3493 |
| 1880 | GGAGCUGG G GAAACUCA | 2926 | UGAGUUUC GGAGGAAACUCC CU UCAAGGACAUCGUCCGGG CCAGCUCC | 3494 |
| 1881 | GAGCUGGG G AAACUCAA | 2927 | UUGAGUUU GGAGGAAACUCC CU UCAAGGACAUCGUCCGGG CCCAGCUC | 3495 |
| 1901 | UUUCCCCU G UCCUGAUG | 1652 | CAUCAGGA GGAGGAAACUCC CU UCAAGGACAUCGUCCGGG AGGGGAAA | 3496 |
| 1906 | CCUGUCCU G AUGGUCAG | 2928 | CUGACCAU GGAGGAAACUCC CU UCAAGGACAUCGUCCGGG AGGACAGG | 3497 |
| 1909 | GUCCUGAU G GUCAGCUC | 2929 | GAGCUGAC GGAGGAAACUCC CU UCAAGGACAUCGUCCGGG AUCAGGAC | 3498 |
| 1910 | UCCUGAUG G UCAGCUCC | 1653 | GGAGCUGA GGAGGAAACUCC CU UCAAGGACAUCGUCCGGG CAUCAGGA | 3499 |
| 1914 | GAUGGUCA G CUCCCUUC | 1654 | GAAGGGAG GGAGGAAACUCC CU UCAAGGACAUCGUCCGGG UGACCAUC | 3500 |
| 1926 | CCUUCUCU G UAGGGAAC | 1655 | GUUCCCUA GGAGGAAACUCC CU UCAAGGACAUCGUCCGGG AGAGAAGG | 3501 |
| 1929 | UCUCUGUA G GGAACUGU | 2930 | ACAGUUCC GGAGGAAACUCC CU UCAAGGACAUCGUCCGGG UACAGAGA | 3502 |
| 1930 | CUCUGUAG G GAACUGUG | 2931 | CACAGUUC GGAGGAAACUCC CU UCAAGGACAUCGUCCGGG CUACAGAG | 3503 |
| 1931 | UCUGUAGG G AACUGUGG | 2932 | CCACAGUU GGAGGAAACUCC CU UCAAGGACAUCGUCCGGG CCUACAGA | 3504 |
| 1936 | AGGGAACU G UGGGGUCC | 1656 | GGACCCCA GGAGGAAACUCC CU UCAAGGACAUCGUCCGGG AGUUCCCU | 3505 |
| 1938 | GGAACUGU G GGGUCCCC | 2933 | GGGGACCC GGAGGAAACUCC CU UCAAGGACAUCGUCCGGG ACAGUUCC | 3506 |
| 1939 | GAACUGUG G GGUCCCCC | 2934 | GGGGGACC GGAGGAAACUCC CU UCAAGGACAUCGUCCGGG CACAGUUC | 3507 |
| 1940 | AACUGUGG G GUCCCCCA | 2935 | UGGGGGAC GGAGGAAACUCC CU UCAAGGACAUCGUCCGGG CCACAGUU | 3508 |
| 1941 | ACUGUGGG G UCCCCCAU | 1657 | AUGGGGGA GGAGGAAACUCC CU UCAAGGACAUCGUCCGGG CCCACAGU | 3509 |
| 1962 | AUCCUCCA G CUUCUGGU | 1658 | ACCAGAAG GGAGGAAACUCC CU UCAAGGACAUCGUCCGGG UGGAGGAU | 3510 |
| 1968 | CAGCUUCU G GUACUCUC | 2936 | GAGAGUAC GGAGGAAACUCC CU UCAAGGACAUCGUCCGGG AGAAGCUG | 3511 |
| 1969 | AGCUUCUG G UACUCUCC | 1659 | GGAGAGUA GGAGGAAACUCC CU UCAAGGACAUCGUCCGGG CAGAAGCU | 3512 |
| 1980 | CUCUCCUA G AGACAGAA | 2937 | UUCUGUCU GGAGGAAACUCC CU UCAAGGACAUCGUCCGGG UAGGAGAG | 3513 |
| 1982 | CUCCUAGA G ACAGAAGC | 2938 | GCUUCUGU GGAGGAAACUCC CU UCAAGGACAUCGUCCGGG UCUAGGAG | 3514 |
| 1986 | UAGAGACA G AAGCAGGC | 2939 | GCCUGCUU GGAGGAAACUCC CU UCAAGGACAUCGUCCGGG UGUCUCUA | 3515 |
| 1989 | AGACAGAA G CAGGCUGG | 1660 | CCAGCCUG GGAGGAAACUCC CU UCAAGGACAUCGUCCGGG UUCUGUCU | 3516 |
| 1992 | CAGAAGCA G GCUGGAGG | 2940 | CCUCCAGC GGAGGAAACUCC CU UCAAGGACAUCGUCCGGG UGCUUCUG | 3517 |
| 1993 | AGAAGCAG G CUGGAGGU | 1661 | ACCUCCAG GGAGGAAACUCC CU UCAAGGACAUCGUCCGGG CUGCUUCU | 3518 |
| 1996 | AGCAGGCU G GAGGUAAG | 2941 | CUUACCUC GGAGGAAACUCC CU UCAAGGACAUCGUCCGGG AGCCUGCU | 3519 |
| 1997 | GCAGGCUG G AGGUAAGG | 2942 | CCUUACCU GGAGGAAACUCC CU UCAAGGACAUCGUCCGGG CAGCCUGC | 3520 |
| 1999 | AGGCUGGA G GUAAGGCC | 2943 | GGCCUUAC GGAGGAAACUCC CU UCAAGGACAUCGUCCGGG UCCAGCCU | 3521 |
| 2000 | GGCUGGAG G UAAGGCCU | 1662 | AGGCCUUA GGAGGAAACUCC CU UCAAGGACAUCGUCCGGG CUCCAGCC | 3522 |
| 2004 | GGAGGUAA G GCCUUUGA | 2944 | UCAAAGGC GGAGGAAACUCC CU UCAAGGACAUCGUCCGGG UUACCUCC | 3523 |
| 2005 | GAGGUAAG G CCUUUGAG | 1663 | CUCAAAGG GGAGGAAACUCC CU UCAAGGACAUCGUCCGGG CUUACCUC | 3524 |
| 2011 | AGGCCUUU G AGCCCACA | 2945 | UGUGGGCU GGAGGAAACUCC CU UCAAGGACAUCGUCCGGG AAAGGCCU | 3525 |
| 2013 | GCCUUUGA G CCCACAAA | 1664 | UUUGUGGG GGAGGAAACUCC CU UCAAGGACAUCGUCCGGG UCAAAGGC | 3526 |
| 2022 | CCCACAAA G CCUUAUCA | 1665 | UGAUAAGG GGAGGAAACUCC CU UCAAGGACAUCGUCCGGG UUUGUGGG | 3527 |
| 2032 | CUUAUCAA G UGUCUUCC | 1666 | GGAAGACA GGAGGAAACUCC CU UCAAGGACAUCGUCCGGG UUGAUAAG | 3528 |
| 2034 | UAUCAAGU G UCUUCCAU | 1667 | AUGGAAGA GGAGGAAACUCC CU UCAAGGACAUCGUCCGGG ACUUGAUA | 3529 |
| 2046 | UCCAUCAU G GAUUCAUU | 2946 | AAUGAAUC GGAGGAAACUCC CU UCAAGGACAUCGUCCGGG AUGAUGGA | 3530 |
| 2047 | CCAUCAUG G AUUCAUUA | 2947 | UAAUGAAU GGAGGAAACUCC CU UCAAGGACAUCGUCCGGG CAUGAUGG | 3531 |
| 2058 | UCAUUACA G CUUAAUCA | 1668 | UGAUUAAG GGAGGAAACUCC CU UCAAGGACAUCGUCCGGG UGUAAUGA | 3532 |
| 2074 | AAAAUAAC G CCCCAGAU | 1669 | AUCUGGGG GGAGGAAACUCC CU UCAAGGACAUCGUCCGGG GUUAUUUU | 3533 |
| 2080 | ACGCCCCA G AUACCAGC | 2948 | GCUGGUAU GGAGGAAACUCC CU UCAAGGACAUCGUCCGGG UGGGGCGU | 3534 |
| 2087 | AGAUACCA G CCCCUGUA | 1670 | UACAGGGG GGAGGAAACUCC CU UCAAGGACAUCGUCCGGG UGGUAUCU | 3535 |
| 2093 | CAGCCCCU G UAUGGCAC | 1671 | GUGCCAUA GGAGGAAACUCC CU UCAAGGACAUCGUCCGGG AGGGGCUG | 3536 |
| 2097 | CCCUGUAU G GCACUGGC | 2949 | GCCAGUGC GGAGGAAACUCC CU UCAAGGACAUCGUCCGGG AUACAGGG | 3537 |
| 2098 | CCUGUAUG G CACUGGCA | 1672 | UGCCAGUG GGAGGAAACUCC CU UCAAGGACAUCGUCCGGG CAUACAGG | 3538 |
| 2103 | AUGGCACU G GCAUUGUC | 2950 | GACAAUGC GGAGGAAACUCC CU UCAAGGACAUCGUCCGGG AGUGCCAU | 3539 |
| 2104 | UGGCACUG G CAUUGUCC | 1673 | GGACAAUG GGAGGAAACUCC CU UCAAGGACAUCGUCCGGG CAGUGCCA | 3540 |
| 2109 | CUGGCAUU G UCCCUGUG | 1674 | CACAGGGA GGAGGAAACUCC CU UCAAGGACAUCGUCCGGG AAUGCCAG | 3541 |
| 2115 | UUGUCCCU G UGCCUAAC | 1675 | GUUAGGCA GGAGGAAACUCC CU UCAAGGACAUCGUCCGGG AGGGACAA | 3542 |
| 2117 | GUCCCUGU G CCUAACAC | 1676 | GUGUUAGG GGAGGAAACUCC CU UCAAGGACAUCGUCCGGG ACAGGGAC | 3543 |
| 2128 | UAACACCA G CGUUUGAG | 1677 | CUCAAACG GGAGGAAACUCC CU UCAAGGACAUCGUCCGGG UGGUGUUA | 3544 |
| 2130 | ACACCAGC G UUUGAGGG | 1678 | CCCUCAAA GGAGGAAACUCC CU UCAAGGACAUCGUCCGGG GCUGGUGU | 3545 |
| 2134 | CAGCGUUU G AGGGGCUG | 2951 | CAGCCCCU GGAGGAAACUCC CU UCAAGGACAUCGUCCGGG AAACGCUG | 3546 |
| 2136 | GCGUUUGA G GGGCUGCC | 2952 | GGCAGCCC GGAGGAAACUCC CU UCAAGGACAUCGUCCGGG UCAAACGC | 3547 |
| 2137 | CGUUUGAG G GGCUGCCU | 2953 | AGGCAGCC GGAGGAAACUCC CU UCAAGGACAUCGUCCGGG CUCAAACG | 3548 |
| 2138 | GUUUGAGG G GCUGCCUU | 2954 | AAGGCAGC GGAGGAAACUCC CU UCAAGGACAUCGUCCGGG CCUCAAAC | 3549 |
| 2139 | UUUGAGGG G CUGCCUUC | 1679 | GAAGGCAG GGAGGAAACUCC CU UCAAGGACAUCGUCCGGG CCCUCAAA | 3550 |
| 2142 | GAGGGGCU G CCUUCCUG | 1680 | CAGGAAGG GGAGGAAACUCC CU UCAAGGACAUCGUCCGGG AGCCCCUC | 3551 |
| 2150 | GCCUUCCU G CCCUACAG | 1681 | CUGUAGGG GGAGGAAACUCC CU UCAAGGACAUCGUCCGGG AGGAAGGC | 3552 |
| 2158 | GCCCUACA G AGGUCUCU | 2955 | AGAGACCU GGAGGAAACUCC CU UCAAGGACAUCGUCCGGG UGUAGGGC | 3553 |
| 2160 | CCUACAGA G GUCUCUGC | 2956 | GCAGAGAC GGAGGAAACUCC CU UCAAGGACAUCGUCCGGG UCUGUAGG | 3554 |
| 2161 | CUACAGAG G UCUCUGCC | 1682 | GGCAGAGA GGAGGAAACUCC CU UCAAGGACAUCGUCCGGG CUCUGUAG | 3555 |
| 2167 | AGGUCUCU G CCGGCUCU | 1683 | AGAGCCGG GGAGGAAACUCC CU UCAAGGACAUCGUCCGGG AGAGACCU | 3556 |
| 2170 | UCUCUGCC G GCUCUUUC | 2957 | GAAAGAGC GGAGGAAACUCC CU UCAAGGACAUCGUCCGGG GGCAGAGA | 3557 |
| 2171 | CUCUGCCG G CUCUUUCC | 1684 | GGAAAGAG GGAGGAAACUCC CU UCAAGGACAUCGUCCGGG CGGCAGAG | 3558 |
| 2182 | CUUUCCUU G CUCAACCA | 1685 | UGGUUGAG GGAGGAAACUCC CU UCAAGGACAUCGUCCGGG AAGGAAAG | 3559 |
| 2192 | UCAACCAU G GCUGAAGG | 2958 | CCUUCAGC GGAGGAAACUCC CU UCAAGGACAUCGUCCGGG AUGGUUGA | 3560 |
| 2193 | CAACCAUG G CUGAAGGA | 1686 | UCCUUCAG GGAGGAAACUCC CU UCAAGGACAUCGUCCGGG CAUGGUUG | 3561 |
| 2196 | CCAUGGCU G AAGGAAAC | 2959 | CUUUCCUU GGAGGAAACUCC CU UCAAGGACAUCGUCCGGG AGCCAUGG | 3562 |
| 2199 | UGGCUGAA G GAAACAGU | 2960 | ACUGUUUC GGAGGAAACUCC CU UCAAGGACAUCGUCCGGG UUCAGCCA | 3563 |
| 2200 | GGCUGAAG G AAACAGUG | 2961 | CACUGUUU GGAGGAAACUCC CU UCAAGGACAUCGUCCGGG CUUCAGCC | 3564 |
| 2206 | AGGAAACA G UGCAACAG | 1687 | CUGUUGCA GGAGGAAACUCC CU UCAAGGACAUCGUCCGGG UGUUUCCU | 3565 |
| 2208 | GAAACAGU G CAACAGCA | 1688 | UGCUGUUG GGAGGAAACUCC CU UCAAGGACAUCGUCCGGG ACUGUUUC | 3566 |
| 2214 | GUGCAACA G CACUGGCU | 1689 | AGCCAGUG GGAGGAAACUCC CU UCAAGGACAUCGUCCGGG UGUUGCAC | 3567 |
| 2219 | ACAGCACU G GCUCUCUC | 2962 | GAGAGAGC GGAGGAAACUCC CU UCAAGGACAUCGUCCGGG AGUGCUGU | 3568 |
| 2220 | CAGCACUG G CUCUCUCC | 1690 | GGAGAGAG GGAGGAAACUCC CU UCAAGGACAUCGUCCGGG CAGUGCUG | 3569 |
| 2230 | UCUCUCCA G GAUCCAGA | 2963 | UCUGGAUC GGAGGAAACUCC CU UCAAGGACAUCGUCCGGG UGGAGAGA | 3570 |
| 2231 | CUCUCCAG G AUCCAGAA | 2964 | UUCUGGAU GGAGGAAACUCC CU UCAAGGACAUCGUCCGGG CUGGAGAG | 3571 |
| 2237 | AGGAUCCA G AAGGGGUU | 2965 | AACCCCUU GGAGGAAACUCC CU UCAAGGACAUCGUCCGGG UGGAUCCU | 3572 |
| 2240 | AUCCAGAA G GGGUUUGG | 2966 | CCAAACCC GGAGGAAACUCC CU UCAAGGACAUCGUCCGGG UUCUGGAU | 3573 |
| 2241 | UCCAGAAG G GGUUUGGU | 2967 | ACCAAACC GGAGGAAACUCC CU UCAAGGACAUCGUCCGGG CUUCUGGA | 3574 |
| 2242 | CCAGAAGG G GUUUGGUC | 2968 | GACCAAAC GGAGGAAACUCC CU UCAAGGACAUCGUCCGGG CCUUCUGG | 3575 |
| 2243 | CAGAAGGG G UUUGGUCU | 1691 | AGACCAAA GGAGGAAACUCC CU UCAAGGACAUCGUCCGGG CCCUUCUG | 3576 |
| 2247 | AGGGGUUU G GUCUGGAC | 2969 | GUCCAGAC GGAGGAAACUCC CU UCAAGGACAUCGUCCGGG AAACCCCU | 3577 |
| 2248 | GGGGUUUG G UCUGGACU | 1692 | AGUCCAGA GGAGGAAACUCC CU UCAAGGACAUCGUCCGGG CAAACCCC | 3578 |
| 2252 | UUUGGUCU G GACUUCCU | 2970 | AGGAAGUC GGAGGAAACUCC CU UCAAGGACAUCGUCCGGG AGACCAAA | 3579 |
| 2253 | UUGGUCUG G ACUUCCUU | 2971 | AAGGAAGU GGAGGAAACUCC CU UCAAGGACAUCGUCCGGG CAGACCAA | 3580 |
| 2262 | ACUUCCUU G CUCUCCCC | 1693 | GGGGAGAG GGAGGAAACUCC CU UCAAGGACAUCGUCCGGG AAGGAAGU | 3581 |
| 2280 | CUUCUCAA G UGCCUUAA | 1694 | UUAAGGCA GGAGGAAACUCC CU UCAAGGACAUCGUCCGGG UUGAGAAG | 3582 |
| 2282 | UCUCAAGU G CCUUAAUA | 1695 | UAUUAAGG GGAGGAAACUCC CU UCAAGGACAUCGUCCGGG ACUUGAGA | 3583 |
| 2291 | CCUUAAUA G UAGGGUAA | 1696 | UUACCCUA GGAGGAAACUCC CU UCAAGGACAUCGUCCGGG UAUUAAGG | 3584 |
| 2294 | UAAUAGUA G GGUAAGUU | 2972 | AACUUACC GGAGGAAACUCC CU UCAAGGACAUCGUCCGGG UACUAUUA | 3585 |
| 2295 | AAUAGUAG G GUAAGUUG | 2973 | CAACUUAC GGAGGAAACUCC CU UCAAGGACAUCGUCCGGG CUACUAUU | 3586 |
| 2296 | AUAGUAGG G UAAGUUGU | 1697 | ACAACUUA GGAGGAAACUCC CU UCAAGGACAUCGUCCGGG CCUACUAU | 3587 |
| 2300 | UAGGGUAA G UUGUUAAG | 1698 | CUUAACAA GGAGGAAACUCC CU UCAAGGACAUCGUCCGGG UUACCCUA | 3588 |
| 2303 | GGUAAGUU G UUAAGAGU | 1699 | ACUCUUAA GGAGGAAACUCC CU UCAAGGACAUCGUCCGGG AACUUACC | 3589 |
| 2308 | GUUGUUAA G AGUGGGGG | 2974 | CCCCCACU GGAGGAAACUCC CU UCAAGGACAUCGUCCGGG UUAACAAC | 3590 |
| 2310 | UGUUAAGA G UGGGGGAG | 1700 | CUCCCCCA GGAGGAAACUCC CU UCAAGGACAUCGUCCGGG UCUUAACA | 3591 |
| 2312 | UUAAGAGU G GGGGAGAC | 2975 | CUCUCCCC GGAGGAAACUCC CU UCAAGGACAUCGUCCGGG ACUCUUAA | 3592 |
| 2313 | UAAGAGUG G GGGAGAGC | 2976 | GCUCUCCC GGAGGAAACUCC CU UCAAGGACAUCGUCCGGG CACUCUUA | 3593 |
| 2314 | AAGAGUGG G GGAGAGCA | 2977 | UGCUCUCC GGAGGAAACUCC CU UCAAGGACAUCGUCCGGG CCACUCUU | 3594 |
| 2315 | AGAGUGGG G GAGAGCAG | 2978 | CUGCUCUC GGAGGAAACUCC CU UCAAGGACAUCGUCCGGG CCCACUCU | 3595 |
| 2316 | GAGUGGGG G AGAGCAGG | 2979 | CCUGCUCU GGAGGAAACUCC CU UCAAGGACAUCGUCCGGG CCCCACUC | 3596 |
| 2318 | GUGGGGGA G AGCAGGCU | 2980 | AGCCUGCU GGAGGAAACUCC CU UCAAGGACAUCGUCCGGG UCCCCCAC | 3597 |
| 2320 | GGGGGAGA G CAGGCUGG | 1701 | CCAGCCUG GGAGGAAACUCC CU UCAAGGACAUCGUCCGGG UCUCCCCC | 3598 |
| 2323 | GGAGAGCA G GCUGGCAG | 2981 | CUGCCAGC GGAGGAAACUCC CU UCAAGGACAUCGUCCGGG UGCUCUCC | 3599 |
| 2324 | GAGAGCAG G CUGGCAGC | 1702 | GCUGCCAG GGAGGAAACUCC CU UCAAGGACAUCGUCCGGG CUGCUCUC | 3600 |
| 2327 | AGCAGGCU G GCAGCUCU | 2982 | AGAGCUGC GGAGGAAACUCC CU UCAAGGACAUCGUCCGGG AGCCUGCU | 3601 |
| 2328 | GCAGGCUG G CAGCUCUC | 1703 | GAGAGCUG GGAGGAAACUCC CU UCAAGGACAUCGUCCGGG CAGCCUGC | 3602 |
| 2331 | GGCUGGCA G CUCUCCAG | 1704 | CUGGAGAG GGAGGAAACUCC CU UCAAGGACAUCGUCCGGG UGCCAGCC | 3603 |
| 2339 | GCUCUCCA G UCAGGAGG | 1705 | CCUCCUGA GGAGGAAACUCC CU UCAAGGACAUCGUCCGGG UGGAGAGC | 3604 |
| 2343 | UCCAGUCA G GAGGCAUA | 2983 | UAUGCCUC GGAGGAAACUCC CU UCAAGGACAUCGUCCGGG UGACUGGA | 3605 |
| 2344 | CCAGUCAG G AGGCAUAG | 2984 | CUAUGCCU GGAGGAAACUCC CU UCAAGGACAUCGUCCGGG CUGACUGG | 3606 |
| 2346 | AGUCAGGA G GCAUAGUU | 2985 | AACUAUGC GGAGGAAACUCC CU UCAAGGACAUCGUCCGGG UCCUGACU | 3607 |
| 2347 | GUCAGGAG G CAUAGUUU | 1706 | AAACUAUG GGAGGAAACUCC CU UCAAGGACAUCGUCCGGG CUCCUGAC | 3608 |
| 2352 | GAGGCAUA G UUUUUAGU | 1707 | ACUAAAAA GGAGGAAACUCC CU UCAAGGACAUCGUCCGGG UAUGCCUC | 3609 |
| 2359 | AGUUUUUA G UGAACAAU | 1708 | AUUGUUCA GGAGGAAACUCC CU UCAAGGACAUCGUCCGGG UAAAAACU | 3610 |
| 2361 | UUUUUAGU G AACAAUCA | 2986 | UGAUUGUU GGAGGAAACUCC CU UCAAGGACAUCGUCCGGG ACUAAAAA | 3611 |
| 2372 | CAAUCAAA G CACUUGGA | 1709 | UCCAAGUG GGAGGAAACUCC CU UCAAGGACAUCGUCCGGG UUUGAUUG | 3612 |
| 2378 | AAGCACUU G GACUCUUG | 2987 | CAAGAGUC GGAGGAAACUCC CU UCAAGGACAUCGUCCGGG AAGUGCUU | 3613 |
| 2379 | AGCACUUG G ACUCUUGC | 2988 | GCAAGAGU GGAGGAAACUCC CU UCAAGGACAUCGUCCGGG CAAGUGCU | 3614 |
| 2386 | GGACUCUU G CUCUUUCU | 1710 | AGAAAGAG GGAGGAAACUCC CU UCAAGGACAUCGUCCGGG AAGAGUCC | 3615 |
| 2400 | UCUACUCU G AACUAAUA | 2989 | UAUUAGUU GGAGGAAACUCC CU UCAAGGACAUCGUCCGGG AGAGUAGA | 3616 |
| 2411 | CUAAUAAA G CUGUUGCC | 1711 | GGCAACAG GGAGGAAACUCC CU UCAAGGACAUCGUCCGGG UUUAUUAG | 3617 |
| 2414 | AUAAAGCU G UUGCCAAG | 1712 | CUUGGCAA GGAGGAAACUCC CU UCAAGGACAUCGUCCGGG AGCUUUAU | 3618 |
| 2417 | AAGCUGUU G CCAAGCUG | 1713 | CAGCUUGG GGAGGAAACUCC CU UCAAGGACAUCGUCCGGG AACAGCUU | 3619 |
| 2422 | GUUGCCAA G CUGGACGG | 1714 | CCGUCCAG GGAGGAAACUCC CU UCAAGGACAUCGUCCGGG UUGGCAAC | 3620 |
| 2425 | GCCAAGCU G GACGGCAC | 2990 | GUGCCGUC GGAGGAAACUCC CU UCAAGGACAUCGUCCGGG AGCUUGGC | 3621 |
| 2426 | CCAAGCUG G ACGGCACG | 2991 | CGUGCCGU GGAGGAAACUCC CU UCAAGGACAUCGUCCGGG CAGCUUGG | 3622 |
| 2429 | AGCUGGAC G GCACGAGC | 2992 | GCUCGUGC GGAGGAAACUCC CU UCAAGGACAUCGUCCGGG GUCCAGCU | 3623 |
| 2430 | GCUGGACG G CACGAGCU | 1715 | AGCUCGUG GGAGGAAACUCC CU UCAAGGACAUCGUCCGGG CGUCCAGC | 3624 |
| 2434 | GACGGCAC G AGCUCGUG | 2993 | CACGAGCU GGAGGAAACUCC CU UCAAGGACAUCGUCCGGG GUGCCGUC | 3625 |
| 2436 | CGGCACGA G CUCGUGCC | 1716 | GGCACGAG GGAGGAAACUCC CU UCAAGGACAUCGUCCGGG UCGUGCCG | 3626 |

Input Sequence = NM_021975. Cut Site = G/. Arm Length = 8. Core Sequence = GGAGGAAACUCC CU UCAAGGACAUCGUCCGGG NM_021975 (Homo sapiens p65 RelA (NFKB), mRNA; 2444 bp)

TABLE VII

Human REL-A Nucleic Acid and Target molecules

| | | | | | | |
|---|---|---|---|---|---|---|
| Pos | Target | Seq ID | RPI# | Enzymatic Nucleic Acid | Seq ID | Alias |

| | | | | | | |
|---|---|---|---|---|---|---|
| 262 | CCACCAUCAAGAUCA | 3627 | 6167 | UGAUCUUCUGAUGAGGCCG | 3770 | NFKB-262 Rz-7 |
| | | | | AAAGGCCGAAAUGGUGG | | |
| 303 | GCAUCUCCCUGGU | 3628 | 6168 | ACCAGGCUGAUGAGGCCGA | 3772 | NFKB-303 Rz-6 |
| | | | | AAGGCCGAAAGAUGC | | |
| 508 | CCAAGUUCCUAUA | 3629 | 6169 | UAUAGGCUGAUGAGGCCGA | 3772 | NFKB-508 Rz-6 |
| | | | | AAGGCCGAAACUUGG | | |
| 661 | CGAGCUCAAGAUC | 3630 | 6170 | GAUCUUCUGAUGAGGCCGA | 3773 | NFKB-661 Rz-6 |
| | | | | AAGGCCGAAAGCUCG | | |
| 759 | AGGUGUAUUUCAC | 3631 | 6171 | GUGAAACUGAUGAGGCCGA | 3774 | NFKB-759 Rz-6 |
| | | | | AAGGCCGAAACACCU | | |
| 883 | CGUGUCUCCAU | 3632 | 6172 | AUGGACUGAUGAGGCCGAA | 3775 | NFKB-883 Rz-5 |
| | | | | AGGCCGAAACACG | | |
| 1028 | AAGAGUCCUUUCA | 3633 | 6173 | UGAAAGCUGAUGAGGCCGA | 3776 | NFKB-1028 Rz-6 |
| | | | | AAGGCCGAAACUCUU | | |
| 1579 | GCUAUAACUCG | 3634 | 6174 | CGAGUCUGAUGAGGCCGAA | 3777 | NFKB-1579 Rz-5 |
| | | | | AGGCCGAAAUAGC | | |
| 1683 | ACUUCUCOUCCAU | 3635 | 6175 | AUGGAGCUGAUGAGGCCGA | 3778 | NFKB-1683 Rz-6 |
| | | | | AAGGCCGAAAGAAGU | | |
| 1726 | GUCAGAUCAGCUCCU | 3636 | 6176 | AGGAGCUCUGAUGAGGCCG | 3779 | NFKB-1726 Rz-7 |
| | | | | AAAGGCCGAAAUCUGAC | | |
| 413 | CCACAGUUUCCAGA | 3637 | 6178 | UCUGGAAGAAGUGGACCAGAGAAAC | 3780 | NFKB-413 HP-4/6 |
| | | | | ACACGUUGUGGUACAUUACCUGGUA | | |
| 159 | ACAGAUACCACCA | 3638 | 24047 | uₛgₛgₛuₛggcUGAuGagg | 3781 | NFKB-159 Rz-6 allyl stab1 |
| | | | | ccguuaggccGaaAucuguB | | |
| 159 | CACAGAUACCACCAA | 3639 | 24048 | uₛuₛgₛgₛuggcUGAuGag | 3782 | NFKB-159 Rz-7 allyl stab1 |
| | | | | gccguuaggccGaaAucugugB | | |
| 196 | AUGGCUACACAGG | 3640 | 24049 | cₛcₛuₛgₛugcUGAuGagg | 3783 | NFKB-196 Rz-6 allyl stab1 |
| | | | | ccguuaggccGaaAgccauB | | |
| 581 | CGAGCUCAAGAUC | 3630 | 24050 | gₛaₛuₛcₛuucUGAuGagg | 3784 | NFK8-581 Rz-6 allyl stab1 |
| | | | | ccguuaggccGaaAgcucgB | | |
| 581 | CCGAGCUCAAGAUCU | 3641 | 24051 | aₛgₛaₛuₛcuucUGAuGag | 3785 | NFKB-581 Rz-7 allyl stab1 |
| | | | | gccguuaggccGaaAgcucggB | | |
| 679 | AGGUGUAUUUCAC | 3631 | 24052 | gₛuₛgₛaₛaacUGAuGaggccg | 3786 | NFKB-679 Rz-6 allyl stab1 |
| | | | | uuaggccGaaAcaccuB | | |
| 682 | UGUAUUUCACGGG | 3642 | 24053 | cₛcₛcₛgₛugcUGAuGaggccg | 3787 | NFKB-682 Rz-6 allyl stab1 |
| | | | | uuaggccGaaAauacaB | | |
| 682 | GUGUAUUUCACGGGA | 3643 | 24054 | uₛcₛcₛcₛgugcUGAuGaggcc | 3788 | NFKB-682 Rz-7 allyl stab1 |
| | | | | guuaggccGaaAauacacB | | |
| 683 | UGUAUUUCACGGGAC | 3644 | 24055 | gₛuₛcₛcₛcgucUGAuGaggcc | 3789 | NFKB-683 Rz-7 allyl stab1 |
| | | | | guuaggccGaaAaauacaB | | |
| 712 | GAGGCUCCUUUUC | 3645 | 24056 | gₛaₛaₛaₛagcUGAuGaggccg | 3790 | NFKB-712 Rz-6 allyl stab1 |
| | | | | uuaggccGaaAgccucB | | |
| 754 | UUGUGUUCCGGAC | 3646 | 24057 | gₛuₛcₛcₛggcUGAuGaggccg | 3791 | NFKB-754 Rz-6 allyl stab1 |
| | | | | uuaggccGaaAcacaaB | | |
| 925 | AGACCUUCAAGAG | 3647 | 24058 | cₛuₛcₛuₛugcUGAuGaggcog | 3792 | NFKB-925 Rz-6 allyl stab1 |
| | | | | uuaggccGaaAggucuB | | |
| 1022 | UUCUGUCCCCAAG | 3648 | 24059 | cₛuₛuₛgₛggcUGAuGaggccg | 3793 | NFKB-1022 Rz-6 allyl stab1 |
| | | | | uuaggccGaaAcagaaB | | |
| 1022 | CUUCUGUCCCCAAGC | 3649 | 24060 | gₛcₛuₛuₛgggcUGAuGaggcc | 3794 | NFKB-1022 Rz-7 allyl stab1 |
| | | | | guuaggccGaaAcagaagB | | |
| 1486 | UGGAGUACOCUGA | 3650 | 24061 | uₛcₛaₛgₛggcUGAuGaggccg | 3795 | NFKB-1486 Rz-6 allyl stab1 |
| | | | | uuaggccGaaAcuccaB | | |
| 1600 | GACUUCUCCUCCAUU | 3651 | 24062 | aₛaₛuₛgₛgagcUGAUGaggcc | 3796 | NFkB-1600 Rz-7 allyl stab1 |
| | | | | guuaggccGaaAgaagucB | | |
| 1603 | UUCUCCUCCAUUGCG | 3652 | 24063 | cₛgₛcₛaₛaugcUGAuGaggccg | 3797 | NFKB-1603 Rz-7 allyl stab1 |
| | | | | uuaggccGaaAggagaaB | | |
| 1643 | GUCAGAUCAGCUCCU | 3636 | 24064 | aₛgₛgₛaₛgcucuGAuGaggccg | 3798 | NFKB-1643 Rz-7 allyl stab1 |
| | | | | uuaggccGaaAucugacB | | |
| 2383 | UGGACUCUUGCUC | 3653 | 24065 | gₛaₛgₛcₛaacuGAuGaggccgu | 3799 | NFKB-2383 Rz-6 allyl stab1 |
| | | | | uaggccGaaAguccaB | | |
| 2383 | UUGGACUCUUGCUCU | 3654 | 24066 | aₛgₛaₛgₛcaacUGAuGaggccg | 3800 | NFKB-2383 Rz-7 allyl stab1 |
| | | | | uuaggccGaaAguccaaB | | |
| 2385 | GACUCUUGCUCUU | 3655 | 24067 | aₛaₛgₛaₛgccUGAuGaggccgu | 3801 | NFKB-2385 Rz-6 allyl stab1 |
| | | | | uaggccGaaAgagucB | | |
| 2385 | GGACUCUUGCUCUUU | 3656 | 24068 | aₛaₛaₛgₛagccUGAuGaggccg | 3802 | NFKB-2385 Rz-7 allyl stab1 |
| | | | | uuaggccGaaAgaguccB | | |
| 2389 | CUUGCUCUUUCUA | 3657 | 24069 | uₛaₛgₛaₛaacUGAuGaggccgu | 3803 | NFKB-2389 Rz-6 allyl stab1 |
| | | | | uaggccGaaAgcaagB | | |
| control | ACGACUCGUUCGA | 3658 | 24070 | uₛcₛgₛaₛaccUAGuGacgcc | 3804 | NFKB-CtrI Rz-6 allyl (SAC) |
| | | | | guuaggcgGaaAgucguB | | |
| control | AGCCUGUAUACCGCG | 3659 | 24071 | cₛgₛoₛgₛguacUAGUGacg | 3805 | NFKB-CtrI Rz-7 allyl (SAC) |
| | | | | ccguuaggcgGaaAcaggcuB | | |
| 162 | GAUACCACCAAGA | 3660 | 24092 | uₛcₛuₛuₛggcUGAuGaggccg | 3806 | NFKB-162 CHz-6 allyl stab1 |
| | | | | uUaggccGaaIguaucB | | |
| 162 | AGAUACCACCAAGAC | 3661 | 24093 | gₛuₛcₛuₛuggoUGAUGaggc | 3807 | NFKB-162 CHz-7 allyl stab1 |
| | | | | cguuaggccGaaIguaucuB | | |
| 180 | CCCACCAUCAAGA | 3662 | 24094 | uₛcₛuₛuₛgacUGAuGaggcc | 3808 | NFKB-180 CHz-6 allyl stab1 |
| | | | | guuaggccGaaIgugggB | | |
| 183 | ACCAUCAAGAUCA | 3663 | 24095 | uₛgₛaₛuₛcucUGAuGaggcc | 3809 | NFKB-183 CHz-6 allyl stab1 |
| | | | | guuaggccGaaIaugguB | | |
| 183 | CACCAUCAAGAUCAA | 3664 | 24096 | uₛuₛgₛaₛuoucUGAuGaggc | 3810 | NFKB-183 CHz-7 allyl stab1 |
| | | | | cguuaggcoGaaIauggugB | | |
| 189 | AAGAUCAAUGGCU | 3665 | 24097 | aₛgₛcₛcₛaucUGAuGaggccg | 3811 | NFKB-189 CHz-6 allyl stab1 |
| | | | | uuagccGaaIaucuuB | | |
| 189 | CAAGAUCAAUGGCUA | 3666 | 24098 | uₛaₛgₛcₛcaucUGAuGaggc | 3812 | NFKB-189 CHz-7 allyl stab1 |
| | | | | cguuaggccGaaIaucuugB | | |
| 195 | AAUGGCUACACAG | 3667 | 24099 | cₛuₛgₛuₛgucUGAuGaggcc | 3813 | NFKB-195 CHz-6 allyl stab1 |
| | | | | guuaggccGaaIccauuB | | |
| 195 | CAAUGGCUACACAGG | 3668 | 24100 | cₛcₛuₛgₛugucUGAuGaggcc | 3814 | NFKB-195 CHz-7 allyl stab1 |
| | | | | guuaggccGaaIccauugB | | |
| 285 | GACUGCCGGGAUG | 3669 | 24101 | cₛaₛuₛcₛcccUGAuGaggcc | 3815 | NFKB-285 CHz-6 allyl stab1 |
| | | | | guuaggccGaaIcagucB | | |
| 480 | CUCUGCUUCCAGG | 3670 | 24102 | cₛcₛuₛgₛgacUGAuGaggcc | 3816 | NFKB-480 CHz-6 allyl stab1 |
| | | | | guuaggocGaalcagagB | | |
| 491 | GGUGACAGUGCGG | 3671 | 24103 | cₛcₛgₛcₛaccUGAuGaggcc | 3817 | NFKB-491 CHz-6 allyl stab1 |
| | | | | guuaggccGaaIucaccB | | |
| 491 | AGGUGACAGUGCGGG | 3672 | 24104 | cₛcₛcₛgₛcaccUGAUGaggcc | 3818 | NFKB-491 CHz-7 allyl stab1 |
| | | | | guuaggccGaaIucaccuB | | |
| 575 | CACUGCCGAGCUC | 3673 | 24105 | gₛaₛgₛcₛuccUGAuGaggcc | 3819 | NFKB-575 CHz-6 allyl stab1 |
| | | | | guuaggccGaaIcagugB | | |
| 575 | ACACUGCCGAGCUCA | 3674 | 24106 | uₛgₛaₛgₛcuccUGAuGaggcc | 3820 | NFKB-575 CHz-7 allyl stab1 |
| | | | | guuaggccGaaIcaguguB | | |
| 580 | CCGAGCUCAAGAU | 3675 | 24107 | aₛuₛcₛuₛugcUGAUGaggcc | 3821 | NFKB-580 CHz-6 allyl stab1 |
| | | | | guuaggccGaaIcucggB | | |
| 582 | GAGCUCAAGAUCU | 3676 | 24108 | aₛgₛaₛuₛcuoUGAuGaggccg | 3822 | NFKB-582 CHz-6 allyl stab1 |
| | | | | uuaggccGaaIagcucB | | |
| 658 | AGGUGCAGAAAGA | 3677 | 24109 | uₛcₛuₛuₛuccUGAuGaggcc | 3823 | NFKB-658 CHz-6 allyl stab1 |
| | | | | guuaggccGaaIcaccuB | | |
| 684 | GUAUUUCACGGGACC | 3678 | 24110 | gₛgₛuₛcₛccgcUGAuGaggcc | 3824 | NFKB-684 CHz-7 allyl stab1 |
| | | | | guuaggcoGaaIaaauaaB | | |
| 692 | GGGACCAGGCUGG | 3679 | 24111 | cₛcₛaₛgₛcccUGAuGaggcc | 3825 | NFKB-692 CHz-6 allyl stab1 |
| | | | | guuaggccGaaIgucccB | | |
| 746 | AGUGGCCAUUGUG | 3680 | 24112 | cₛaₛcₛaₛaucUGAuGaggcc | 3826 | NFkB-746 CHz-6 allyl stab1 |
| | | | | guuaggcoGaaIccacuB | | |
| 746 | AAGUGGCCAUUGUGU | 3681 | 24113 | aₛ0ₛaₛcₛaucUGAuGaggccg | 3827 | NFKB-746 CHz-7 allyl stab1 |
| | | | | uuaggccGaaIccacuuB | | |
| 747 | GUGGCCAUUGUGU | 3682 | 24114 | aₛcₛaₛcₛaacUGAuGaggcc | 3828 | NFKB-747 CHz-6 allyl stab1 |
| | | | | guuaggccGaaIgccacB | | |
| 747 | AGUGGCCAUUGUGUU | 3683 | 24115 | aₛₛₛcₛaₛcaacUGAuGaggc | 3829 | NFkB-747 CHz-7 allyl stab1 |
| | | | | cguuaggccGaaIgccacuB | | |
| 807 | GUCUCCAUGOAGO | 3684 | 24116 | gₛcₛuₛgₛcacUGAuGaggcc | 3830 | NFKB-807 CHz-6 allyl stab1 |
| | | | | guuaggccGaalgagacB | | |
| 847 | GUGAGCCCAUGGA | 3685 | 24117 | uₛcₛcₛaₛugcUGAuGaggcc | 3831 | NFKB-847 CHz-6 allyl stab1 |
| | | | | guuaggccGaaIcucacB | | |
| 864 | CAGUACCUGOCAG | 3686 | 24118 | cₛuₛgₛgₛcacUGAuGaggccg | 3832 | NFKB-864 cHz-6 allyl stab1 |
| | | | | uuaggcCGaaIUaCUgB | | |
| 864 | CCAGUACCUGCCAGA | 3687 | 24119 | uₛcₛuₛgₛgcacUGAuGaggcc | 3833 | NFKB-864 CHz-7 allyl stab1 |
| | | | | guuaggccGaaIuacuggB | | |
| 914 | AAGGACAUAUGAG | 3688 | 24120 | cₛuₛcₛaₛuacUGAuGaggccg | 3834 | NFKB-914 CHz-6 allyl stab1 |
| | | | | uuaggccGaaIuccuuB | | |
| 914 | AAAGGACAUAUGAGA | 3689 | 24121 | uₛcₛuₛcₛauacUGAuGaggcc | 3835 | NFKB-914 CHz-7 allyl stab1 |
| | | | | guuaggccGaaIuccuuuB | | |
| 1023 | UCUGUCCCCAAGC | 3690 | 24122 | gₛcₛuₛuₛggcUGAuGaggccg | 3836 | NFKB-1023 CHz-6 allyl stab1 |
| | | | | uuaggccGaaIacagaB | | |
| 1024 | CUGUCCCCAAGCC | 3691 | 24123 | gₛgₛoₛuₛugcUGAUGaggccg | 3837 | NFKB-1024 CHz-6 allyl stab1 |
| | | | | uuaggccGaaIgacagB | | |
| 1024 | UCUGUCCCCAAGCCA | 3692 | 24124 | uₛgₛgₛcₛuugcUGAuGaggcc | 3838 | NFKB-1024 CHz-7 allyl stab1 |
| | | | | guuaggccGaaIgacagaB | | |
| 1071 | AGCACCAUCAACU | 3693 | 24125 | aₛgₛuₛuₛgacUGAuGaggccg | 3839 | NFKB-1071 CHz-6 allyl stab1 |
| | | | | uuaggccGaaIgugcuB | | |
| 1347 | GAAGACCUGGGGG | 3694 | 24126 | cₛcₛcₛcₛcacUGAuGaggccg | 3840 | NFKB-1347 CHz-6 allyl stab1 |
| | | | | uuaggccGaaIucuucB | | |
| 1347 | UGAAGACCUGGGGGC | 3695 | 24127 | gₛcₛcₛcₛccacUGAuGaggcc | 3841 | NFKB-1347 CHz-7 allyl stab1 |
| | | | | guuaggccGaaIucuucaB | | |
| 1371 | AACAGCACAGACC | 3696 | 24128 | gₛgₛuₛcₛugcUGAuGaggccg | 3842 | NFKB-1371 CHz-6 allyl stab1 |
| | | | | uuaggccGaaIcuguuB | | |
| 1371 | CAACAGCACAGACCC | 3697 | 24129 | gₛgₛgₛuₛCugCUGAUGaggcc | 3843 | NFKB-1371 CHz-7 allyl stab1 |
| | | | | guuaggccGaaIcuguugB | | |
| 1373 | CAGCACAGACCCA | 3698 | 24130 | uₛgₛgₛgₛuccUGAuGaggccg | 3844 | NFKB-1373 CHz-6 allyl stab1 |
| | | | | uuaggccGaaIugcugB | | |
| 1373 | ACAGCACAGACCCAG | 3699 | 24131 | cₛuₛgₛgₛguccUGAuGaggcc | 3845 | NFKB-1373 CHz-7 allyl stab1 |
| | | | | guuaggccGaaIugcuguB | | |
| 1389 | GUGUUCACAGACC | 3700 | 24132 | gₛgₛuₛcₛugcUGAUGaggccg | 3846 | NFKB-1389 CHz-6 allyl stab1 |
| | | | | uuaggccGaaIaacacB | | |
| 1391 | GUUCACAGACCUG | 3701 | 24133 | cₛaₛgₛgₛuccUGALIGaggcc | 3847 | NFKB-1391 CHz-6 allyl stab1 |
| | | | | guuaggccGaaIugaacB | | |
| 1391 | UGUUCACAGACCUGG | 3702 | 24134 | cₛcₛaₛgₛguccUGAuGaggcc | 3848 | NFKB-1391 CHz-7 allyl stab1 |
| | | | | guuaggccGaaIugaacaB | | |
| 1395 | ACAGACCUGGCAU | 3703 | 24135 | aₛuₛgₛcₛcacUGAuGaggccg | 3849 | NFKB-1395 CHz-6 allyl stab1 |
| | | | | uuaggccGaaIucuguB | | |
| 1395 | CACAGACCUGGCAUC | 3704 | 24136 | gₛaₛuₛgₛccacUGAuGaggcc | 3850 | NFKB-1395 CHz-7 allyl stab1 |
| | | | | guuaggccGaaIucugugB | | |
| 1396 | CAGACCUGGCAUC | 3705 | 24137 | gₛaₛuₛgₛcccUGAuGaggccg | 3851 | NFKB-1396 CHz-6 allyl stab1 |
| | | | | uuaggccGaaIgucugB | | |
| 1601 | ACUUCUCCUCCAUUG | 3706 | 24138 | cₛaₛaₛuₛggacUGAuGaggcc | 3852 | NFKB-1 601 CHz-7 allyl stab1 |
| | | | | guuaggccGaaIagaaguB | | |
| 1602 | CUUCUCCUCCAUUGC | 3707 | 24139 | gₛoₛaₛaₛuggcUGAuGaggcc | 3853 | NFKB-1602 CHz-7 allyl stab1 |
| | | | | guuaggccGaalgagaagB | | |
| 1604 | UCUCCUCCAUUGCGG | 3708 | 24140 | cₛcₛgₛcₛaaucUGAuGaggcc | 3854 | NFKB-1604 CHz-7 allyl stab1 |
| | | | | guuaggccGaalaggagaB | | |
| 1605 | UCCUCCAUUGCGG | 3709 | 24141 | cₛcₛgₛcₛaacUGAuGaggccg | 3855 | NFKB-1605 CHz-6 allyl stab1 |
| | | | | uuaggccGaalgaggaB | | |
| 1614 | GCGGACAUGGACU | 3710 | 24142 | aₛgₛuₛcₛcacUGAuGaggccg | 3856 | NFKB-1614 CHz-6 allyl stab1 |
| | | | | uuaggccGaaIuccgcB | | |
| 1614 | UGCGGACAUGGACUU | 3711 | 24143 | aₛaₛgₛuₛccacUGAuGaggcc | 3857 | NFKB-1614 CHz-7 allyl stab1 |
| | | | | guuaggccGaaIuccgcaB | | |
| 1644 | CAGAUCAGCUCCU | 3712 | 24144 | aₛgₛgₛaₛgccUGAUGaggccg | 3858 | NFKB-1644 CHz-6 allyl stab1 |
| | | | | uuaggccGaaIaucugB | | |
| 1644 | UCAGAUCAGCUCCUA | 3713 | 24145 | uₛaₛgₛgₛagCcUGALIGaggc | 3859 | NFKB-1644 CHz-7 allyl stab1 |
| | | | | cguuaggccGaaIaucugaB | | |
| 2382 | UUGGACUCUUGCU | 3714 | 24146 | aₛgₛcₛaₛagcUGAuGaggccg | 3860 | NFKB-2382 CHz-6 allyl stab1 |
| | | | | uuaggccGaaluccaaB | | |
| 2384 | GGACUCUUGCUCU | 3715 | 24147 | aₛgₛaₛgₛcacUGAuGaggccg | 3861 | NFKB-2384 CHz-6 allyl stab1 |
| | | | | uuaggccGaaIaguccB | | |
| 2384 | UGGACUCUUGCUCUU | 3716 | 24148 | aₛaₛgₛaₛgcacUGAuGaggcc | 3862 | NFKB-2384 CHz-7 allyl stab1 |
| | | | | guuaggccGaaIaguccaB | | |
| 2388 | UCUUGOUCUUUCU | 3717 | 24149 | aₛgₛaₛaₛagcUGAuGaggccg | 3863 | NFKB-2388 CHz-6 allyl stab1 |
| | | | | uuaggccGaaIcaagaB | | |
| 2388 | CUCUUGCUCUUUCUA | 3718 | 24150 | uₛaₛgₛaₛaagcUGAuGaggcc | 3864 | NFKB-2388 CHz-7 allyl stab1 |
| | | | | guuaggccGaalcaagagB | | |
| Control | CUAUGCUACGGCA | 3719 | 24151 | uₛgₛcₛcₛgucUAGuGacgcc | 3865 | NFKB-Ctrl CHz-6 allyl (SAC) |
| | | | | guuaggCgGaaIGauagB | | |
| Control | AUGCUCCCGGGUCAA | 3720 | 24152 | uₛuₛgₛaₛccccUAGuGacgc | 3866 | NFKB-Ctrl CHz-7 allyl (SAC) |
| | | | | cguuaggcgGaaIgagcauB | | |
| 193 | AUCAAUGGCUACACA | 3721 | 24184 | uₛgₛuₛgₛuaggccgaaa | 3867 | NFKB-193 Zin.Rz-7 amino |
| | | | | ggCgagugaGguCucauugauB | | |
| 358 | UCOAGUGUGUGAA | 3722 | 24185 | uₛuₛcₛaₛcagccgaaa | 3868 | NFKB-358 Zin.Rz-6 amino |
| | | | | ggCgagUgaGguCuacuggaB | | |
| 358 | AUCCAGUGUGUGAAG | 3723 | 24186 | cₛuₛuₛcₛacagccgaaa | 3869 | NFKB-358 Zin.Rz-7 amino |
| | | | | ggCgagugaGguCuacuggauB | | |
| 360 | CCAGUGUGUGAAGAA | 3724 | 24187 | uₛuₛcₛuₛucagccgaaa | 3870 | NFKB-360 Zin.Rz-7 amino |
| | | | | ggCgagugaGguCuacacuggB | | |
| 486 | UUCCAGGUGACAG | 3725 | 24188 | cₛuₛgₛuₛcagccgaaa | 3871 | NFKB-486 Zin.Rz-6 amino |
| | | | | ggCgagugaGguCucuggaaB | | |
| 486 | cuuCCAGGUGACAGU | 3726 | 24189 | aₛcₛuₛgₛucagccgaaa | 3872 | NFKB-486 Zin.Rz-7 amino |
| | | | | ggCgagugaGguCucuggaagB | | |
| 492 | GUGACAGUGCGGG | 3727 | 24190 | cₛcₛcₛgₛcagccgaaa | 3873 | NFKB-492 Zin.Rz-6 amino |
| | | | | ggCgagugaGguCuugucacB | | |
| 492 | GGUGACAGUGCGGGA | 3728 | 24191 | uₛcₛcₛcₛgcagccgaaa | 3874 | NFKB-492 Zin.Rz-7 amino |
| | | | | ggCgagugaGguCuugucaccB | | |
| 494 | GACAGUGCGGGAC | 3729 | 24192 | gₛuₛcₛcₛcggccgaaa | 3875 | NFKB-494 Zin.Rz-6 amino |
| | | | | ggCgagugaGguCuacugucB | | |
| 494 | UGACAGUGCGGGACC | 3730 | 24193 | gₛgₛuₛcₛccggccgaaa | 3876 | NFKB-494 Zin.Rz-7 amino |
| | | | | ggCgagugaGguCuacugucaB | | |
| 573 | AACACUGCCGAGC | 3731 | 24194 | gₛcₛuₛcₛgggccgaaag | 3877 | NFKB-573 Zin.Rz-6 amino |
| | | | | ggCgagugaGguCuagugggB | | |
| 573 | CAACACUGCCGAGCU | 3732 | 24195 | aₛgₛcₛuₛcgggccgaaa | 3878 | NFKB-573 Zin.Rz-7 amino |
| | | | | ggCgagugaGguguaguguugB | | |
| 578 | UGCCGAGCUCAAG | 3733 | 24196 | cₛuₛuₛgₛaggccgaaa | 3879 | NFKB-578 Zin.Rz-6 amino |
| | | | | ggCgagugaGguCuucggcaB | | |
| 578 | CUGCCGAGCUCAAGA | 3734 | 24197 | uₛcₛuₛuₛgaggccgaaa | 3880 | NFKB-578 Zin.Rz-7 amino |
| | | | | ggCgagugaGguCuucggcagB | | |
| 654 | GACAAGGUGCAGA | 3735 | 24198 | uₛcₛuₛgₛcagccgaaa | 3881 | NFKB-654 Zin.Rz-6 amino |
| | | | | CgggagugaGguCucuugucB | | |
| 656 | ACAAGGUGCAGAAAG | 3736 | 24199 | cₛuₛuₛuₛcuggccgaaa | 3882 | NFKB-656 Zin.Rz-7 amino |
| | | | | ggCgagugaGguCuaccuuguB | | |
| 677 | UGAGGUGUAUUUC | 3737 | 24200 | gₛaₛaₛaₛuagccgaaa | 3883 | NFKB-677 Zin.Rz-6 amino |
| | | | | ggCgagugaGguCuaccucaB | | |
| 750 | GCCAUUGUGUUCC | 3738 | 24201 | gₛgₛaₛaₛcagccgaaa | 3884 | NFKB-750 Zin.Rz-6 amino |
| | | | | ggCgagugaGguCuaauggcB | | |
| 750 | GGCCAUUGUGUUCCG | 3739 | 24202 | cₛgₛgₛaₛacagccgaaa | 3885 | NFKB-750 Zin.Rz-7 amino |
| | | | | ggCgagugaGguCuaauggccB | | |
| 752 | CCAUUGUGUUCCGGA | 3740 | 24203 | uₛcₛcₛgₛgaagccgaaa | 3886 | NFKB-752 Zin.Rz-7 amino |
| | | | | ggCgagugaGguCuaCaauggB | | |
| 1475 | GCCCAUGCUGAUG | 3741 | 24204 | cₛaₛuₛcₛaggccgaaa | 3887 | NFKB-1 475 Zin.Rz-6 amino |
| | | | | ggCgagugaGguCuaugggcB | | |
| 1645 | CAGAUCAGCUCCUAA 3742 | 24205 | uₛuₛaₛgₛgaggccgaaa | 3888 | NFKB-1 645 Zin.Rz-7 amino | |
| | | | | ggCgagugaGguCuugaucugB | | |
| 2386 | ACUCUUGCUCUUU | 3743 | 24206 | aₛaₛaₛgₛaggccgaaa | 3889 | NFKB-2386 Zin.Rz-6 amino |
| | | | | ggCgagugaGguCuaagaguB | | |
| 2386 | GACUCUUGCUCUUUC 3744 | 24207 | gₛaₛaₛaₛgaggccgaaa | 3890 | NFKB-2386 Zin.Rz-7 amino | |
| | | | | ggCgagugaGguCuaagagucB | | |
| Control | UCCGACGCGAAUU | 3745 | 24208 | aₛaₛuₛuₛcggccgaaa | 3891 | NFKB-ctrl Zin. Rz-6 (SAC) |
| | | | | ggCuCugGagugaggucggaB | | |
| Control | ACAUGACGUCCGUGC | 3746 | 24209 | gₛcₛaₛcₛggagccgaaa | 3892 | NFKB-ctrl Zin.Rz-7 (SAC) |
| | | | | ggCuCugGagugaggucauguB | | |
| 269 | ACGAGCUUGUAGGAA | 3747 | 24367 | uuccuaccUGAUGAggc | 3893 | NFKB-269 Rz-7 Ome stab1 |
| | | | | cguuaggccGAAAgcucguB | | |
| 536 | CUGUCCUUUCUCAUC | 3748 | 24368 | gaugagaoUGAUGAggc | 3894 | NFKB-536 Rz-7 Ome stab1 |
| | | | | cguuaggcoGAAAggacagB | | |
| 671 | AGGACAUUGAGGUGU | 3749 | 24369 | acaccuccUGAUGAggc | 3895 | NFKB-671 Rz-7 Ome stab1 |
| | | | | cguuaggccGAAAuguccuB | | |
| 951 | GAGUCCUUUCAGCGG | 3750 | 24370 | ccgcugacUGAUGAggcc | 3896 | NFKB-951 Rz-7 Ome stab1 |
| | | | | guuaggccGAAAggacucB | | |
| 2391 | UUGCUCUUUCUACUC | 3751 | 24371 | gaguagacUGAUGAggccg | 3897 | NFKB-2391 Rz-7 Ome stab1 |
| | | | | uuaggccGAAAgagcaaB | | |
| 327 | CCGCUGCAUCCACAG | 3752 | 24372 | cuguggacUGAUGAggccg | 3898 | NFKB-327 CHz-7 Ome stab1 |
| | | | | uuaggccGAAIcagcggB | | |
| 535 | CCUGUCCUUUCUCAU | 3753 | 24373 | augagaacUGAUGAggccg | 3899 | NFKB-535 CHz-7 Ome stab1 |
| | | | | uuaggccGMIgacaggB | | |
| 1062 | GUCAUCOCUGAGOAC | 3754 | 24374 | gugcucacUGAuGAggccg | 3900 | NFKB-1062 CHz-7 Ome stab1 |
| | | | | uuaggccGpAIgaugacB | | |
| 1114 | UCUGGGCAGAUCAGC | 3755 | 24375 | gcugauccUGAUGAggccg | 3901 | NFKB-1114 CHz-7 Ome stab1 |
| | | | | uuaggccGMIcccagaB | | |
| 1231 | CCUGUCCCAGUCCUA | 3756 | 24376 | uaggacucUGAUGAggccg | 3902 | NFKB-1231 CHz-7 Ome stab1 |
| | | | | uuaggccGMIgacaggB | | |
| 325 | GACCGCUGCAUCCAC | 3757 | 24377 | guggauggccgaaaggC | 3903 | NFKB-325 Zin.Rz-7 amino |
| | | | | gagugaGGuCuagcggucB | | |
| 368 | UGAAGAAGCGGGAcC | 3758 | 24378 | ggucocggccgaaaggC | 3904 | NFKB-368 Zin.Rz-7 amino |
| | | | | gagugaGGuCuuucuucaB | | |
| 799 | CCUGUGCGUGUCUCC | 3759 | 24379 | ggagacagccgaaaggC | 3905 | NFKB-799 Zin.Rz-7 amino |
| | | | | gagugaGGuCugcacaggB | | |
| 801 | UGUGCGUGUCUCCAU | 3760 | 24380 | auggagagccgaaaggC | 3906 | NFKB-801 Zin.Rz-7 amino |
| | | | | gagugaGGuCuacgcacaB | | |
| 1233 | UGUCCCAGUCCUAGC | 3761 | 24381 | gcuaggagccgaaaggC | 3907 | NFKB-1233 Zin.Rz-7 amino |
| | | | | gagugaGGuCuugggacaB | | |
| 157 | AGCACAGAUACCACC | 3762 | 24382 | ggugguaGGcTAGcTAc | 3908 | NFKB-1 57 Dz-7 |
| | | | | AAcGAcugugcuB | | |
| 190 | AAGAUCAAUGGCUAC | 3763 | 24383 | guagccaGGcTAGcTAc | 3909 | NFKB-1 90 Dz-7 |
| | | | | AAcGAugaucuuB | | |
| 399 | UCAGCGCAUCCAGAC | 3764 | 24384 | gucuggaGGcTAGcTAc | 3910 | NFKB-399 Dz-7 |
| | | | | AAcGAgcgcugaB | | |
| 799 | CCUGUGCGUGUCUCC | 3759 | 24385 | ggagacaGGcTAGcTAc | 3911 | NFKB-799 Dz-7 |
| | | | | AAcGAgcacaggB | | |
| 1614 | UGCGGACAUGGACUU | 3711 | 24386 | aaGUccaGGcTAGcTAc | 3912 | NFKB-1614 Dz-7 |
| | | | | AAcGAgUccgcaB | | |
| 156 | GAGCACAGAUACCAC | 3765 | 24387 | gugguauGgaggaaacucCCUUCaa | 3913 | NFKB-156 Amb.Rz-7 |
| | | | | ggacaucgucCGGGugugcucB | | |
| 896 | GGAUUGAGGAGAAAC | 3766 | 24388 | guuucucGgaggaaacucCCUUCaa | 3914 | NFKB-896 Amb.Rz-7 |
| | | | | ggacaucgucCGGGucaauccB | | |
| 1341 | UGAUGAUGAAGACCU | 3767 | 24389 | aggucuuGgaggaaacucCCUUCaa | 3915 | NFKB-1 341 Amb.Rz-7 |
| | | | | ggacaucgucCGGGaucaucaB | | |
| 2314 | AGAGUGGGGGAGAGC | 3768 | 24390 | gcucuccGgaggaaacucCCUUCaa | 3916 | NFKB-2314 Amb.Rz-7 |
| | | | | ggacaucgucCGGGccacucuB | | |
| 2318 | UGGGGGAGAGCAGGC | 3769 | 24391 | gccugcuGgaggaaacucCCUUCaa | 3917 | NFKB-2318 Amb.Rz-7 |
| | | | | ggacaucgucCGGGucccccaB | | |

A, G, C, U = Ribo
A, G, C, T (italic) = deoxy
lower case = 2′-O-methyl
s = phosphorothioate 3′-internucleotide linkage
U = 2′-deoxy-2′-C-allyl uridine
U = 2′-deoxy-2′-Amino uridine
C = 2′-deoxy-2′-Amino cytidine
I = Inosine
B = inverted deoxyabasic derivative

What we claim is:

1. An enzymatic nucleic acid molecule which down regulates expression of a sequence encoding a subunit of NFKB, wherein said enzymatic nucleic acid molecule is in an Inozyme, Zinzyme, G-cleaver, or Amberzyme configuration.

2. An enzymatic nucleic acid molecule comprising a sequence selected from the group consisting of SEQ ID NOs. 711-1420, 1717-2012, 2151-2656, 2994-3626, and 3770-3917.

3. An enzymatic nucleic acid molecule comprising at least one binding arm wherein one or more of said binding arms comprises a sequence complementary to a sequence selected from the group consisting of SEQ ID NOs. 1-30, 32-48, 50-108, 100-136, 138-183, 185-306, 308-450, 452-497, 499-710, 1421-1428, 1430-1454, 1456-1464, 1466-1475, 1477-1482, 1484-1501, 1504-1535, 1537-1543, 1545-1548, 1550-1563, 1565-1575, 1578-1586, 1588-1601, 1603-1607, 1609-1716, 2013-2015, 2017-2056, 2058-2064, 2066-2076, 2078-2082, 2084, 2086-2150, 2657-2993, and 3627-3769.

4. An antisense nucleic acid molecule comprising a sequence complementary to a sequence selected from the group consisting of SEQ ID NOs. 1-710, 1421-1716, 2013-2150, 2657-2993, and 3627-3769.

5. The enzymatic nucleic acid of any of claims 1-3, wherein said enzymatic nucleic acid molecule is adapted to treat cancer.

6. The antisense nucleic acid molecule of claim 4, wherein said antisense nucleic acid molecule is adapted to treat cancer.

7. The enzymatic nucleic acid molecule of any of claims 1-3, wherein said enzymatic nucleic acid molecule has an endonuclease activity to cleave RNA having REL-A sequence.

8. The enzymatic nucleic acid molecule of claim 1, wherein said enzymatic nucleic acid molecule is in an Inozyme configuration.

9. The enzymatic nucleic acid molecule of claim 1, wherein said enzymatic nucleic acid molecule is in a Zinzyme configuration.

10. The enzymatic nucleic acid molecule of claim 1, wherein said enzymatic nucleic acid molecule is in a G-cleaver configuration.

11. The enzymatic nucleic acid molecule of claim 1, wherein said enzymatic nucleic acid molecule is in an Amberzyme configuration.

12. The enzymatic nucleic acid molecule of claim 3, wherein said enzymatic nucleic acid molecule is in a DNAzyme configuration.

13. The enzymatic nucleic acid molecule of claim 8, wherein said Inozyme comprises a sequence complementary to a sequence selected from the group consisting of SEQ ID NOs. 1-710, 3752-3756, and 3660-3720.

14. The enzymatic nucleic acid molecule of claim 8, wherein said Inozyme comprises a sequence selected from the group consisting of SEQ ID NOs. 711-1420, 3898-3902, and 3806-3866.

15. The enzymatic nucleic acid molecule of claim 9, wherein said Zinzyme comprises a sequence complementary to a sequence selected from the group consisting of SEQ ID NOs. 1421-1716, 3721-3746, and 3757-3761.

16. The enzymatic nucleic acid molecule of claim 9, wherein said Zinzyme comprises a sequence selected from the group consisting of SEQ ID NOs 1717-2012, 3867-3892, and 3903-3907.

17. The enzymatic nucleic acid molecule of claim 11, wherein said Amberzyme comprises a sequence complementary to a sequence selected from the group consisting of SEQ ID NOs. 1421-1716, 2657-2993, and 3765-3769.

18. The enzymatic nucleic acid molecule of claim 11, wherein said Amberzyme comprises a sequence selected from the group consisting of SEQ ID NOs 2994-3626, and 3913-3917.

19. The enzymatic nucleic acid molecule of any of claims 1-3, wherein said enzymatic nucleic acid molecule comprises between 12 and 100 bases complementary to RNA sequence encoding a subunit of NFKB.

20. The enzymatic nucleic acid molecule of any of claims 1-3, wherein said enzymatic nucleic acid molecule comprises between 14 and 24 bases complementary to RNA sequence encoding a subunit of NFKB.

21. The enzymatic nucleic acid molecule of any of claims 1-3, wherein said enzymatic nucleic acid molecule is chemically synthesized.

22. The antisense nucleic acid molecule of claim 4, wherein said antisense nucleic acid molecule is chemically synthesized.

23. The enzymatic nucleic acid molecule of any of claims 1-3, wherein said enzymatic nucleic acid molecule comprises at least one 2′-sugar modification.

24. The antisense nucleic acid molecule of claim 4, wherein said antisense nucleic acid molecule comprises at least one 2′-sugar modification.

25. The enzymatic nucleic acid molecule of any of claims 1-3, wherein said enzymatic nucleic acid molecule comprises at least one nucleic acid base modification.

26. The antisense nucleic acid molecule of claim 4, wherein said antisense nucleic acid molecule comprises at least one nucleic acid base modification.

27. The enzymatic nucleic acid molecule of any of claims 1-3, wherein said enzymatic nucleic acid molecule comprises at least one phosphate backbone modification.

28. The antisense nucleic acid molecule of claim 4, wherein said antisense nucleic acid molecule comprises at least one phosphate backbone modification.

29. A mammalian cell including the enzymatic nucleic acid molecule of any of claims 1-3.

30. The mammalian cell of claim 29, wherein said mammalian cell is a human cell.

31. A method of down-regulating REL-A activity in a cell, comprising contacting said cell with the enzymatic nucleic acid molecule of any of claims 1-3, under conditions suitable for down-regulating of REL-A activity.

32. A method of down-regulating REL-A activity in a cell, comprising contacting said cell with the antisense nucleic acid molecule of claim 4 under conditions suitable for said reduction of REL-A activity.

33. A method of treatment of a patient having a condition associated with the level of REL-A, comprising contacting cells of said patient with the enzymatic nucleic acid molecule of any of claims 1-3, under conditions suitable for said treatment.

34. A method of treatment of a patient having a condition associated with the level of REL-A, comprising contacting cells of said patient with the antisense nucleic acid molecule of claim 4, under conditions suitable for said treatment.

35. The method of claim 31 further comprising the use of one or more drug therapies under conditions suitable for said treatment.

36. The method of claim 32 further comprising the use of one or more drug therapies under conditions suitable for said treatment.

37. The method of claim 33 further comprising the use of one or more drug therapies under conditions suitable for said treatment.

38. The method of claim 34 further comprising the use of one or more drug therapies under conditions suitable for said treatment.

39. A method of cleaving RNA comprising a sequence of REL-A gene comprising contacting an enzymatic nucleic acid molecule of any of claims 1-3 with said RNA of REL-A gene under conditions suitable for the cleavage.

40. The method of claim 39, wherein said cleavage is carried out in the presence of a divalent cation.

41. The method of claim 40, wherein said divalent cation is Mg²⁺.

42. The enzymatic nucleic acid molecule of any of claims 1-3, wherein said enzymatic nucleic acid comprises a cap structure, wherein the cap structure is at the 5′-end, or 3′-end, or both the 5′-end and the 3′-end.

43. The antisense nucleic acid molecule of claim 4, wherein said antisense nucleic acid comprises a cap structure, wherein the cap structure is at the 5′-end, or 3′-end, or both the 5′-end and the 3′-end.

44. The enzymatic nucleic acid molecule of claim 42, wherein the cap structure at the 5′-end, 3′-end, or both the 5′-end and the 3′-end comprises a 3′,3′-linked or 5′,5′-linked deoxyabasic ribose derivative.

45. The antisense nucleic acid molecule of claim 43, wherein the cap structure at the 5′-end, 3′-end, or both the 5′-end and the 3′-end comprises a 3′,3′-linked or 5′,5′-linked deoxyabasic ribose derivative.

46. The method of claim 31, wherein said enzymatic nucleic acid molecule is in a Zinzyme configuration.

47. An expression vector comprising a nucleic acid sequence encoding at least one enzymatic nucleic acid molecule of claim 1 or claim 3 in a manner which allows expression of the nucleic acid molecule.

48. A mammalian cell including an expression vector of claim 47.

49. The mammalian cell of claim 48, wherein said mammalian cell is a human cell.

50. The expression vector of claim 47, wherein said enzymatic nucleic acid molecule is in a hammerhead configuration.

51. The expression vector of claim 47, wherein said expression vector further comprises a sequence for an antisense nucleic acid molecule complementary to the RNA of a subunit of NFKB.

52. The expression vector of claim 47, wherein said expression vector comprises a nucleic acid sequence encoding two or more of said enzymatic nucleic acid molecules, which may be the same or different.

53. The expression vector of claim 52, wherein said expression vector further comprises a sequence encoding an antisense nucleic acid molecule complementary to the RNA of REL-A gene.

54. A method for treatment of cancer comprising administering to a patient the enzymatic nucleic acid molecule of any of claims 1-3 under conditions suitable for said treatment.

55. The method of claim 54, wherein said cancer is breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer.

56. A method for treatment of cancer comprising administering to a patient the antisense nucleic acid molecule of claim 4 under conditions suitable for said treatment.

57. The method of claim 56, wherein said cancer is breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancer.

58. The method of claim 54, wherein said enzymatic nucleic acid molecule is in a Zinzyme configuration.

59. The method of claim 54, wherein said method further comprises administering to said patient one or more other therapies.

60. The method of claim 56, wherein said method further comprises administering to said patient one or more other therapies.

61. The nucleic acid molecule of claim 1 or claim 3, wherein said nucleic acid molecule comprises at least five ribose residues, at least ten 2′-O-methyl modifications, and a 3′-end modification.

62. The nucleic acid molecule of claim 61, wherein said nucleic acid molecule further comprises phosphorothioate linkages on at least three of the 5′ terminal nucleotides.

63. The nucleic acid molecule of claim 61, wherein said 3′-end modification is a 3′-3′ inverted abasic moiety.

64. The method of claim 35 wherein said other drug therapies are monoclonal antibodies, REL-A-specific inhibitors, or chemotherapy, or radiation therapy.

65. The method of claim 64, wherein said chemotherapy is paclitaxel, docetaxel, cisplatin, methotrexate, cyclophosphamide, doxorubin, fluorouracil carboplatin, edatrexate, gemcitabine, or vinorelbine.

66. The method of claim 36 wherein said other drug therapies are monoclonal antibodies, REL-A-specific inhibitors, or chemotherapy, or radiation therapy.

67. The method of claim 66, wherein said chemotherapy is paclitaxel, docetaxel, cisplatin, methotrexate, cyclophosphamide, doxorubin, fluorouracil carboplatin, edatrexate, gemcitabine, or vinorelbine.

68. The method of claim 37 wherein said other drug therapies are monoclonal antibodies, REL-A-specific inhibitors, or chemotherapy, or radiation therapy.

69. The method of claim 68, wherein said chemotherapy is paclitaxel, docetaxel, cisplatin, methotrexate, cyclophosphamide, doxorubin, fluorouracil carboplatin, edatrexate, gemcitabine, or vinorelbine.

70. The method of claim 38 wherein said other drug therapies are monoclonal antibodies, REL-A-specific inhibitors, or chemotherapy, or radiation therapy.

71. The method of claim 70, wherein said chemotherapy is paclitaxel, docetaxel, cisplatin, methotrexate, cyclophosphamide, doxorubin, fluorouracil carboplatin, edatrexate, gemcitabine, or vinorelbine.

72. The method of claim 59, wherein said other therapies are monoclonal antibodies, REL-A-specific inhibitors, chemotherapy, or radiation therapy.

73. The method of claim 72, wherein said chemotherapy is paclitaxel, docetaxel, cisplatin, methotrexate, cyclophosphamide, doxorubin, fluorouracil carboplatin, edatrexate, gemcitabine, or vinorelbine.

74. The method of claim 60, wherein said other therapies are monoclonal antibodies, REL-A-specific inhibitors, chemotherapy, or radiation therapy.

75. The method of claim 74, wherein said chemotherapy is paclitaxel, docetaxel, cisplatin, methotrexate, cyclophosphamide, doxorubin, fluorouracil carboplatin, edatrexate, gemcitabine, or vinorelbine.

76. A method for treatment of an inflammatory disease comprising the step of administering to a patient the enzymatic nucleic acid molecule of any of claims 1-3 under conditions suitable for said treatment.

77. The method of claim 76, wherein said inflammatory disease is rheumatoid arthritis, restenosis, asthma, Crohn's disease, diabetes, obesity, autoimmune disease, lupus, multiple sclerosis, transplant/graft rejection, gene therapy applications, ischemia/reperfusion injury, glomerulonephritis, sepsis, allergic airway inflammation, inflammatory bowel disease, or infection.

78. A method for treatment of an inflammatory disease comprising the step of administering to a patient the antisense nucleic acid molecule of claim 4 under conditions suitable for said treatment.

79. The method of claim 78, wherein said inflammatory disease is rheumatoid arthritis, restenosis, asthma, Crohn's disease, diabetes, obesity, autoimmune disease, lupus, multiple sclerosis, transplant/graft rejection, gene therapy applications, ischemia/reperfusion injury (CNS and myocardial), glomerulonephritis, sepsis, allergic airway inflammation, inflammatory bowel disease, or infection.

80. The method of claim 76, wherein said enzymatic nucleic acid molecule is in a Zinzyme configuration.

81. The method of claim 76, wherein said method further comprises administering to said patient one or more other therapies.

82. The method of claim 78, wherein said method further comprises administering to said patient one or more other therapies.

83. A pharmaceutical composition comprising an enzymatic nucleic acid molecule of any of claims 1-3 in a pharmaceutically acceptable carrier.

84. A pharmaceutical composition comprising an antisense nucleic acid molecule of claim 4 in a pharmaceutically acceptable carrier.

85. The enzymatic nucleic acid molecule of claim 1, wherein said subunit of NFKB is REL-A.

86. The enzymatic nucleic acid molecule of claim 1, wherein said subunit of NFKB is REL-B.

87. The enzymatic nucleic acid molecule of claim 1, wherein said subunit of NFKB is REL.

88. The enzymatic nucleic acid molecule of claim 1, wherein said subunit of NFKB is NFKB1.

89. The enzymatic nucleic acid molecule of claim 1, wherein said subunit of NFKB is NFKB2.

90. The enzymatic nucleic acid molecule of claim 20, wherein said subunit of NFKB is REL-A.

91. The enzymatic nucleic acid molecule of claim 20, wherein said subunit of NFKB is REL-B.

92. The enzymatic nucleic acid molecule of claim 20, wherein said subunit of NFKB is REL.

93. The enzymatic nucleic acid molecule of claim 20, wherein said subunit of NFKB is NFKB1.

94. The enzymatic nucleic acid molecule of claim 20, wherein said subunit of NFKB is NFKB2.
